SEQUENCE LISTING (868 sequences)

SEQ ID NO: 1 - protein, 43 aa, Unknown
  betacoronavirus epitope
  PEPTIDE (1)..(43): betacoronavirus epitope
HANEYRLYLDAYNMMISAGFSLWVYKQFDTYNLWNTFTRLQSL

SEQ ID NO: 2 - protein, 61 aa, Unknown
  betacoronavirus epitope
  PEPTIDE (1)..(61): betacoronavirus epitope
YTNDKACPLIAAVITREVGFVVPGLPGTILRTTNGDFLHFLPRVFSAVGNICYTPSKLIEY

SEQ ID NO: 3 - protein, 33 aa, Unknown
  betacoronavirus epitope
  PEPTIDE (1)..(33): betacoronavirus epitope
NVLEGSVAYESLRPDTRYVLMDGSIIQFPNTYL

SEQ ID NO: 4 - protein, 41 aa, Unknown
  betacoronavirus epitope
  PEPTIDE (1)..(41): betacoronavirus epitope
QEYADVFHLYLQYIRKLHDELTGHMLDMYSVMLTNDNTSRY

SEQ ID NO: 5 - protein, 42 aa, Unknown
  betacoronavirus epitope
  PEPTIDE (1)..(42): betacoronavirus epitope
LTDEMIAQYTSALLAGTITSGWTFGAGAALQIPFAMQMAYRF

SEQ ID NO: 6 - protein, 35 aa, Unknown
  betacoronavirus epitope
  PEPTIDE (1)..(35): betacoronavirus epitope
GVVDYGARFYFYTSKTTVASLINTLNDLNETLVTM

SEQ ID NO: 7 - protein, 26 aa, Unknown
  betacoronavirus epitope
  PEPTIDE (1)..(26): betacoronavirus epitope
YSFLPGVYSVIYLYLTFYLTNDVSFL

SEQ ID NO: 8 - protein, 35 aa, Unknown
  betacoronavirus epitope
  PEPTIDE (1)..(35): betacoronavirus epitope
FLLNKEMYLKLRSDVLLPLTQYNRYLALYNKYKYF

SEQ ID NO: 9 - protein, 29 aa, Unknown
  betacoronavirus epitope
  PEPTIDE (1)..(29): betacoronavirus epitope
VQMAPISAMVRMYIFFASFYYVWKSYVHV

SEQ ID NO: 10 - protein, 25 aa, Unknown
  betacoronavirus epitope
  PEPTIDE (1)..(25): betacoronavirus epitope
YYTSNPTTFHLDGEVITFDNLKTLL

SEQ ID NO: 11 - protein, 34 aa, Unknown
  betacoronavirus epitope
  PEPTIDE (1)..(34): betacoronavirus epitope
RTIKVFTTVDNINLHTQVVDMSMTYGQQFGPTYL

SEQ ID NO: 12 - protein, 30 aa, Unknown
  betacoronavirus epitope
  PEPTIDE (1)..(30): betacoronavirus epitope
NLHSSRLSFKELLVYAADPAMHAASGNLLL

SEQ ID NO: 13 - protein, 24 aa, Unknown
  betacoronavirus epitope
  PEPTIDE (1)..(24): betacoronavirus epitope
RAFGEYSHVVAFNTLLFLMSFTVL

SEQ ID NO: 14 - protein, 34 aa, Unknown
  betacoronavirus epitope
  PEPTIDE (1)..(34): betacoronavirus epitope
ILFTRFFYVLGLAAIMQLFFSYFAVHFISNSWLM

SEQ ID NO: 15 - protein, 24 aa, Unknown
  betacoronavirus epitope
  PEPTIDE (1)..(24): betacoronavirus epitope
RTNVYLAVFDKNLYDKLVSSFLEM

SEQ ID NO: 16 - protein, 20 aa, Unknown
  betacoronavirus epitope
  PEPTIDE (1)..(20): betacoronavirus epitope
WTAGAAAYYVGYLQPRTFLL

SEQ ID NO: 17 - protein, 25 aa, Unknown
  betacoronavirus epitope
  PEPTIDE (1)..(25): betacoronavirus epitope
EMLDNRATLQAIASEFSSLPSYAAF

SEQ ID NO: 18 - protein, 25 aa, Unknown
  betacoronavirus epitope
  PEPTIDE (1)..(25): betacoronavirus epitope
NLHPDSATLVSDIDITFLKKDAPYI

SEQ ID NO: 19 - protein, 25 aa, Unknown
  betacoronavirus epitope
  PEPTIDE (1)..(25): betacoronavirus epitope
MTYRRLISMMGFKMNYQVNGYPNMF

SEQ ID NO: 20 - protein, 23 aa, Unknown
  betacoronavirus epitope
  PEPTIDE (1)..(23): betacoronavirus epitope
SVAYSNNSIAIPTNFTISVTTEI

SEQ ID NO: 21 - protein, 26 aa, Unknown
  betacoronavirus epitope
  PEPTIDE (1)..(26): betacoronavirus epitope
TVYTKVDGVDVELFENKTTLPVNVAF

SEQ ID NO: 22 - protein, 25 aa, Unknown
  betacoronavirus epitope
  PEPTIDE (1)..(25): betacoronavirus epitope
HFAIGLALYYPSARIVYTACSHAAV

SEQ ID NO: 23 - protein, 19 aa, Unknown
  betacoronavirus epitope
  PEPTIDE (1)..(19): betacoronavirus epitope
VQQESPFVMMSAPPAQYEL

SEQ ID NO: 24 - protein, 27 aa, Unknown
  betacoronavirus epitope
  PEPTIDE (1)..(27): betacoronavirus epitope
KATEETFKLSYGIATVREVLSDRELHL

SEQ ID NO: 25 - protein, 28 aa, Unknown
  betacoronavirus epitope
  PEPTIDE (1)..(28): betacoronavirus epitope
AMSAFAMMFVKHKHAFLCLFLLPSLATV

SEQ ID NO: 26 - protein, 28 aa, Unknown
  betacoronavirus epitope
  PEPTIDE (1)..(28): betacoronavirus epitope
SSGDATTAYANSVFNICQAVTANVNALL

SEQ ID NO: 27 - protein, 25 aa, Unknown
  betacoronavirus epitope
  PEPTIDE (1)..(25): betacoronavirus epitope
QLIKVTLVFLFVAAIFYLITPVHVM

SEQ ID NO: 28 - protein, 19 aa, Unknown
  betacoronavirus epitope
  PEPTIDE (1)..(19): betacoronavirus epitope
VMYASAVVLLILMTARTVY

SEQ ID NO: 29 - protein, 24 aa, Unknown
  betacoronavirus epitope
  PEPTIDE (1)..(24): betacoronavirus epitope
TTIQTIVEVQPQLEMELTPVVQTI

SEQ ID NO: 30 - protein, 18 aa, Unknown
  betacoronavirus epitope
  PEPTIDE (1)..(18): betacoronavirus epitope
ASIKNFKSVLYYQNNVFM

SEQ ID NO: 31 - protein, 18 aa, Unknown
  betacoronavirus epitope
  PEPTIDE (1)..(18): betacoronavirus epitope
FVSEETGTLIVNSVLLFL

SEQ ID NO: 32 - protein, 24 aa, Unknown
  betacoronavirus epitope
  PEPTIDE (1)..(24): betacoronavirus epitope
MLSDTLKNLSDRVVFVLWAHGFEL

SEQ ID NO: 33 - protein, 33 aa, Unknown
  betacoronavirus epitope
  PEPTIDE (1)..(33): betacoronavirus epitope
TFFKLVNKFLALCADSIIIGGAKLKALNLGETF

SEQ ID NO: 34 - protein, 16 aa, Unknown
  betacoronavirus epitope
  PEPTIDE (1)..(16): betacoronavirus epitope
KMFDAYVNTFSSTFNV

SEQ ID NO: 35 - protein, 15 aa, Unknown
  betacoronavirus epitope
  PEPTIDE (1)..(15): betacoronavirus epitope
FAYANRNRFLYIIKL

SEQ ID NO: 36 - protein, 25 aa, Unknown
  betacoronavirus epitope
  PEPTIDE (1)..(25): betacoronavirus epitope
FGDDTVIEVQGYKSVNITFELDERI

SEQ ID NO: 37 - protein, 15 aa, Unknown
  betacoronavirus epitope
  PEPTIDE (1)..(15): betacoronavirus epitope
FLYENAFLPFAMGII

SEQ ID NO: 38 - protein, 20 aa, Unknown
  betacoronavirus epitope
  PEPTIDE (1)..(20): betacoronavirus epitope
TANPKTPKYKFVRIQPGQTF

SEQ ID NO: 39 - protein, 16 aa, Unknown
  betacoronavirus epitope
  PEPTIDE (1)..(16): betacoronavirus epitope
AQFAPSASAFFGMSRI

SEQ ID NO: 40 - protein, 16 aa, Unknown
  betacoronavirus epitope
  PEPTIDE (1)..(16): betacoronavirus epitope
FVNEFYAYLRKHFSMM

SEQ ID NO: 41 - protein, 15 aa, Unknown
  betacoronavirus epitope
  PEPTIDE (1)..(15): betacoronavirus epitope
NYMPYFFTLLLQLCT

SEQ ID NO: 42 - protein, 15 aa, Unknown
  betacoronavirus epitope
  PEPTIDE (1)..(15): betacoronavirus epitope
RVWTLMNVLTLVYKV

SEQ ID NO: 43 - protein, 21 aa, Unknown
  betacoronavirus epitope
  PEPTIDE (1)..(21): betacoronavirus epitope
SLIYSTAALGVLMSNLGMPSY

SEQ ID NO: 44 - protein, 22 aa, Unknown
  betacoronavirus epitope
  PEPTIDE (1)..(22): betacoronavirus epitope
LVATAEAELAKNVSLDNVLSTF

SEQ ID NO: 45 - protein, 16 aa, Unknown
  betacoronavirus epitope
  PEPTIDE (1)..(16): betacoronavirus epitope
TLKGVEAVMYMGTLSY

SEQ ID NO: 46 - protein, 16 aa, Unknown
  betacoronavirus epitope
  PEPTIDE (1)..(16): betacoronavirus epitope
EAFEKMVSLLSVLLSM

SEQ ID NO: 47 - protein, 20 aa, Unknown
  betacoronavirus epitope
  PEPTIDE (1)..(20): betacoronavirus epitope
RSLKVPATVSVSSPDAVTAY

SEQ ID NO: 48 - protein, 19 aa, Unknown
  betacoronavirus epitope
  PEPTIDE (1)..(19): betacoronavirus epitope
TVYSHLLLVAAGLEAPFLY

SEQ ID NO: 49 - protein, 21 aa, Unknown
  betacoronavirus epitope
  PEPTIDE (1)..(21): betacoronavirus epitope
STQDLFLPFFSNVTWFHAIHV

SEQ ID NO: 50 - protein, 15 aa, Unknown
  betacoronavirus epitope
  PEPTIDE (1)..(15): betacoronavirus epitope
MVYMPASWVMRIMTW

SEQ ID NO: 51 - protein, 16 aa, Unknown
  betacoronavirus epitope
  PEPTIDE (1)..(16): betacoronavirus epitope
RTAPHGHVMVELVAEL

SEQ ID NO: 52 - protein, 17 aa, Unknown
  betacoronavirus epitope
  PEPTIDE (1)..(17): betacoronavirus epitope
GALDISASIVAGGIVAI

SEQ ID NO: 53 - protein, 17 aa, Unknown
  betacoronavirus epitope
  PEPTIDE (1)..(17): betacoronavirus epitope
HVISTSHKLVLSVNPYV

SEQ ID NO: 54 - protein, 15 aa, Unknown
  betacoronavirus epitope
  PEPTIDE (1)..(15): betacoronavirus epitope
AVFQSASKIITLKKR

SEQ ID NO: 55 - protein, 17 aa, Unknown
  betacoronavirus epitope
  PEPTIDE (1)..(17): betacoronavirus epitope
FLNRFTTTLNDFNLVAM

SEQ ID NO: 56 - protein, 15 aa, Unknown
  betacoronavirus epitope
  PEPTIDE (1)..(15): betacoronavirus epitope
HSYFTSDYYQLYSTQ

SEQ ID NO: 57 - protein, 15 aa, Unknown
  betacoronavirus epitope
  PEPTIDE (1)..(15): betacoronavirus epitope
RAMPNMLRIMASLVL

SEQ ID NO: 58 - protein, 15 aa, Unknown
  betacoronavirus epitope
  PEPTIDE (1)..(15): betacoronavirus epitope
VASQSIIAYTMSLGA

SEQ ID NO: 59 - protein, 15 aa, Unknown
  betacoronavirus epitope
  PEPTIDE (1)..(15): betacoronavirus epitope
EIVDTVSALVYDNKL

SEQ ID NO: 60 - protein, 19 aa, Unknown
  betacoronavirus epitope
  PEPTIDE (1)..(19): betacoronavirus epitope
FVFKNIDGYFKIYSKHTPI

SEQ ID NO: 61 - protein, 15 aa, Unknown
  betacoronavirus epitope
  PEPTIDE (1)..(15): betacoronavirus epitope
KVTSAMQTMLFTMLR

SEQ ID NO: 62 - protein, 15 aa, Unknown
  betacoronavirus epitope
  PEPTIDE (1)..(15): betacoronavirus epitope
YLNTLTLAVPYNMRV

SEQ ID NO: 63 - protein, 19 aa, Unknown
  betacoronavirus epitope
  PEPTIDE (1)..(19): betacoronavirus epitope
TLDSKTQSLLIVNNATNVV

SEQ ID NO: 64 - protein, 18 aa, Unknown
  betacoronavirus epitope
  PEPTIDE (1)..(18): betacoronavirus epitope
SSVELKHFFFAQDGNAAI

SEQ ID NO: 65 - protein, 15 aa, Unknown
  betacoronavirus epitope
  PEPTIDE (1)..(15): betacoronavirus epitope
SFSASTSAFVETVKG

SEQ ID NO: 66 - protein, 15 aa, Unknown
  betacoronavirus epitope
  PEPTIDE (1)..(15): betacoronavirus epitope
YLATALLTLQQIELK

SEQ ID NO: 67 - protein, 15 aa, Unknown
  betacoronavirus epitope
  PEPTIDE (1)..(15): betacoronavirus epitope
RSFIEDLLFNKVTLA

SEQ ID NO: 68 - protein, 17 aa, Unknown
  betacoronavirus epitope
  PEPTIDE (1)..(17): betacoronavirus epitope
SILSPLYAFASEAARVV

SEQ ID NO: 69 - protein, 15 aa, Unknown
  betacoronavirus epitope
  PEPTIDE (1)..(15): betacoronavirus epitope
VMFTPLVPFWITIAY

SEQ ID NO: 70 - protein, 15 aa, Unknown
  betacoronavirus epitope
  PEPTIDE (1)..(15): betacoronavirus epitope
LLLDDFVEIIKSQDL

SEQ ID NO: 71 - protein, 15 aa, Unknown
  betacoronavirus epitope
  PEPTIDE (1)..(15): betacoronavirus epitope
SLLMPILTLTRALTA

SEQ ID NO: 72 - protein, 15 aa, Unknown
  betacoronavirus epitope
  PEPTIDE (1)..(15): betacoronavirus epitope
YFIASFRLFARTRSM

SEQ ID NO: 73 - protein, 15 aa, Unknown
  betacoronavirus epitope
  PEPTIDE (1)..(15): betacoronavirus epitope
YSLFDMSKFPLKLRG

SEQ ID NO: 74 - protein, 15 aa, Unknown
  betacoronavirus epitope
  PEPTIDE (1)..(15): betacoronavirus epitope
KAYKIEELFYSYATH

SEQ ID NO: 75 - protein, 15 aa, Unknown
  betacoronavirus epitope
  PEPTIDE (1)..(15): betacoronavirus epitope
LVKPSFYVYSRVKNL

SEQ ID NO: 76 - protein, 15 aa, Unknown
  betacoronavirus epitope
  PEPTIDE (1)..(15): betacoronavirus epitope
TTFTYASALWEIQQV

SEQ ID NO: 77 - protein, 15 aa, Unknown
  betacoronavirus epitope
  PEPTIDE (1)..(15): betacoronavirus epitope
RVYSTGSNVFQTRAG

SEQ ID NO: 78 - protein, 15 aa, Unknown
  betacoronavirus epitope
  PEPTIDE (1)..(15): betacoronavirus epitope
LMIERFVSLAIDAYP

SEQ ID NO: 79 - protein, 15 aa, Unknown
  betacoronavirus epitope
  PEPTIDE (1)..(15): betacoronavirus epitope
HSIGFDYVYNPFMID

SEQ ID NO: 80 - protein, 16 aa, Unknown
  betacoronavirus epitope
  PEPTIDE (1)..(16): betacoronavirus epitope
RTILGSALLEDEFTPF

SEQ ID NO: 81 - protein, 15 aa, Unknown
  betacoronavirus epitope
  PEPTIDE (1)..(15): betacoronavirus epitope
VVYRGTTTYKLNVGD

SEQ ID NO: 82 - protein, 15 aa, Unknown
  betacoronavirus epitope
  PEPTIDE (1)..(15): betacoronavirus epitope
QINDMILSLLSKGRL

SEQ ID NO: 83 - protein, 15 aa, Unknown
  betacoronavirus epitope
  PEPTIDE (1)..(15): betacoronavirus epitope
TGVEHVTFFIYNKIV

SEQ ID NO: 84 - protein, 15 aa, Unknown
  betacoronavirus epitope
  PEPTIDE (1)..(15): betacoronavirus epitope
VTLAILTALRLCAYC

SEQ ID NO: 85 - protein, 15 aa, Unknown
  betacoronavirus epitope
  PEPTIDE (1)..(15): betacoronavirus epitope
YTVELGTEVNEFACV

SEQ ID NO: 86 - protein, 18 aa, Unknown
  betacoronavirus epitope
  PEPTIDE (1)..(18): betacoronavirus epitope
RTIKGTHHWLLLTILTSL

SEQ ID NO: 87 - protein, 15 aa, Unknown
  betacoronavirus epitope
  PEPTIDE (1)..(15): betacoronavirus epitope
YFIKGLNNLNRGMVL

SEQ ID NO: 88 - protein, 25 aa, Unknown
  betacoronavirus epitope
  PEPTIDE (1)..(25): betacoronavirus epitope
VTDVTQLYLGGMSYYCKSHKPPISF

SEQ ID NO: 89 - protein, 16 aa, Unknown
  betacoronavirus epitope
  PEPTIDE (1)..(16): betacoronavirus epitope
SQAWQPGVAMPNLYKM

SEQ ID NO: 90 - protein, 15 aa, Unknown
  betacoronavirus epitope
  PEPTIDE (1)..(15): betacoronavirus epitope
QSAPHGVVFLHVTYV

SEQ ID NO: 91 - protein, 17 aa, Unknown
  betacoronavirus epitope
  PEPTIDE (1)..(17): betacoronavirus epitope
YVYIGDPAQLPAPRTLL

SEQ ID NO: 92 - protein, 15 aa, Unknown
  betacoronavirus epitope
  PEPTIDE (1)..(15): betacoronavirus epitope
YTMADLVYALRHFDE

SEQ ID NO: 93 - protein, 19 aa, Unknown
  betacoronavirus epitope
  PEPTIDE (1)..(19): betacoronavirus epitope
YGFQPTNGVGYQPYRVVVL

SEQ ID NO: 94 - protein, 15 aa, Unknown
  betacoronavirus epitope
  PEPTIDE (1)..(15): betacoronavirus epitope
FVDGVPFVVSTGYHF

SEQ ID NO: 95 - protein, 19 aa, Unknown
  betacoronavirus epitope
  PEPTIDE (1)..(19): betacoronavirus epitope
KGVAPGTAVLRQWLPTGTL

SEQ ID NO: 96 - protein, 15 aa, Unknown
  betacoronavirus epitope
  PEPTIDE (1)..(15): betacoronavirus epitope
YYLFDESGEFKLASH

SEQ ID NO: 97 - protein, 17 aa, Unknown
  betacoronavirus epitope
  PEPTIDE (1)..(15): betacoronavirus epitope
ISMDNSPNLAWPLIVTA

SEQ ID NO: 98 - protein, 17 aa, Unknown
  betacoronavirus epitope
  PEPTIDE (1)..(17): betacoronavirus epitope
YYHTTDPSFLGRYMSAL

SEQ ID NO: 99 - protein, 16 aa, Unknown
  betacoronavirus epitope
  PEPTIDE (1)..(16): betacoronavirus epitope
VVADAVIKTLQPVSEL

SEQ ID NO: 100 - protein, 15 aa, Unknown
  betacoronavirus epitope
  PEPTIDE (1)..(15): betacoronavirus epitope
RFDNPVLPFNDGVYF

SEQ ID NO: 101 - protein, 16 aa, Unknown
  betacoronavirus epitope
  PEPTIDE (1)..(16): betacoronavirus epitope
TTAAKLMVVIPDYNTY

SEQ ID NO: 102 - protein, 15 aa, Unknown
  betacoronavirus epitope
  PEPTIDE (1)..(15): betacoronavirus epitope
LQKAAITILDGISQY

SEQ ID NO: 103 - protein, 15 aa, Unknown
  betacoronavirus epitope
  PEPTIDE (1)..(15): betacoronavirus epitope
ATIPIQASLPFGWLI

SEQ ID NO: 104 - protein, 17 aa, Unknown
  betacoronavirus epitope
  PEPTIDE (1)..(17): betacoronavirus epitope
KQGDDYVYLPYPDPSRI

SEQ ID NO: 105 - protein, 15 aa, Unknown
  betacoronavirus epitope
  PEPTIDE (1)..(15): betacoronavirus epitope
STVFPPTSFGPLVRK

SEQ ID NO: 106 - protein, 19 aa, Unknown
  betacoronavirus epitope
  PEPTIDE (1)..(19): betacoronavirus epitope
IVNGVRRSFYVYANGGKGF

SEQ ID NO: 107 - protein, 16 aa, Unknown
  betacoronavirus epitope
  PEPTIDE (1)..(16): betacoronavirus epitope
DTYPSLETIQITISSF

SEQ ID NO: 108 - protein, 15 aa, Unknown
  betacoronavirus epitope
  PEPTIDE (1)..(15): betacoronavirus epitope
VLVPHVGEIPVAYRK

SEQ ID NO: 109 - protein, 16 aa, Unknown
  betacoronavirus epitope
  PEPTIDE (1)..(16): betacoronavirus epitope
LQTPFEIKLAKKFDTF

SEQ ID NO: 110 - protein, 15 aa, Unknown
  betacoronavirus epitope
  PEPTIDE (1)..(15): betacoronavirus epitope
NVIPTITQMNLKYAI

SEQ ID NO: 111 - protein, 15 aa, Unknown
  betacoronavirus epitope
  PEPTIDE (1)..(15): betacoronavirus epitope
SVLYNSASFSTFKCY

SEQ ID NO: 112 - protein, 15 aa, Unknown
  betacoronavirus epitope
  PEPTIDE (1)..(15): betacoronavirus epitope
ATLPKGIMMNVAKYT

SEQ ID NO: 113 - protein, 15 aa, Unknown
  betacoronavirus epitope
  PEPTIDE (1)..(15): betacoronavirus epitope
KMADQAMTQMYKQAR

SEQ ID NO: 114 - protein, 15 aa, Unknown
  betacoronavirus epitope
  PEPTIDE (1)..(15): betacoronavirus epitope
SVGPKQASLNGVTLI

SEQ ID NO: 115 - protein, 20 aa, Unknown
  betacoronavirus epitope
  PEPTIDE (1)..(20): betacoronavirus epitope
VIAWNSNNLDSKVGGNYNYL

SEQ ID NO: 116 - protein, 15 aa, Unknown
  betacoronavirus epitope
  PEPTIDE (1)..(15): betacoronavirus epitope
FIAGLIAIVMVTIML

SEQ ID NO: 117 - protein, 16 aa, Unknown
  betacoronavirus epitope
  PEPTIDE (1)..(16): betacoronavirus epitope
LTYTGAIKLDDKDPNF

SEQ ID NO: 118 - protein, 15 aa, Unknown
  betacoronavirus epitope
  PEPTIDE (1)..(15): betacoronavirus epitope
YVLPNDDTLRVEAFE

SEQ ID NO: 119 - protein, 15 aa, Unknown
  betacoronavirus epitope
  PEPTIDE (1)..(15): betacoronavirus epitope
AMDEFIERYKLEGYA

SEQ ID NO: 120 - protein, 15 aa, Unknown
  betacoronavirus epitope
  PEPTIDE (1)..(15): betacoronavirus epitope
NMMVTNNTFTLKGGA

SEQ ID NO: 121 - protein, 15 aa, Unknown
  betacoronavirus epitope
  PEPTIDE (1)..(15): betacoronavirus epitope
VMPLSAPTLVPQEHY

SEQ ID NO: 122 - protein, 15 aa, Unknown
  betacoronavirus epitope
  PEPTIDE (1)..(15): betacoronavirus epitope
KLFDRYFKYWDQTYH

SEQ ID NO: 123 - protein, 15 aa, Unknown
  betacoronavirus epitope
  PEPTIDE (1)..(15): betacoronavirus epitope
YLNSTNVTIATYCTG

SEQ ID NO: 124 - protein, 15 aa, Unknown
  betacoronavirus epitope
  PEPTIDE (1)..(15): betacoronavirus epitope
YSDVENPHLMGWDYP

SEQ ID NO: 125 - protein, 15 aa, Unknown
  betacoronavirus epitope
  PEPTIDE (1)..(15): betacoronavirus epitope
RLYYDSMSYEDQDAL

SEQ ID NO: 126 - protein, 15 aa, Unknown
  betacoronavirus epitope
  PEPTIDE (1)..(15): betacoronavirus epitope
SALEPLVDLPIGINI

SEQ ID NO: 127 - protein, 15 aa, Unknown
  betacoronavirus epitope
  PEPTIDE (1)..(15): betacoronavirus epitope
YVDNSSLTIKKPNEL

SEQ ID NO: 128 - protein, 15 aa, Unknown
  betacoronavirus epitope
  PEPTIDE (1)..(15): betacoronavirus epitope
IVRFPNITNLCPFGE

SEQ ID NO: 129 - protein, 15 aa, Unknown
  betacoronavirus epitope
  PEPTIDE (1)..(15): betacoronavirus epitope
MFLARGIVFMCVEYC

SEQ ID NO: 130 - protein, 15 aa, Unknown
  betacoronavirus epitope
  PEPTIDE (1)..(15): betacoronavirus epitope
TVYDPLQPELDSFKE

SEQ ID NO: 131 - protein, 15 aa, Unknown
  betacoronavirus epitope
  PEPTIDE (1)..(15): betacoronavirus epitope
YTNSFTRGVYYPDKV

SEQ ID NO: 132 - protein, 15 aa, Unknown
  betacoronavirus epitope
  PEPTIDE (1)..(15): betacoronavirus epitope
YTVEEAKTVLKKCKS

SEQ ID NO: 133 - protein, 15 aa, Unknown
  betacoronavirus epitope
  PEPTIDE (1)..(15): betacoronavirus epitope
AMYTPHTVLQAVGAC

SEQ ID NO: 134 - protein, 15 aa, Unknown
  betacoronavirus epitope
  PEPTIDE (1)..(15): betacoronavirus epitope
KVDGVVQQLPETYFT

SEQ ID NO: 135 - protein, 16 aa, Unknown
  betacoronavirus epitope
  PEPTIDE (1)..(16): betacoronavirus epitope
TVVIGTSKFYGGWHNM

SEQ ID NO: 136 - protein, 16 aa, Unknown
  betacoronavirus epitope
  PEPTIDE (1)..(16): betacoronavirus epitope
YAKPFLNKVVSTTTNI

SEQ ID NO: 137 - protein, 15 aa, Unknown
  betacoronavirus epitope
  PEPTIDE (1)..(15): betacoronavirus epitope
RIFTIGTVTLKQGEI

SEQ ID NO: 138 - protein, 17 aa, Unknown
  betacoronavirus epitope
  PEPTIDE (1)..(17): betacoronavirus epitope
HTIDGSSGVVNPVMEPI

SEQ ID NO: 139 - protein, 15 aa, Unknown
  betacoronavirus epitope
  PEPTIDE (1)..(15): betacoronavirus epitope
TVYEKLKPVLDWLEE

SEQ ID NO: 140 - protein, 15 aa, Unknown
  betacoronavirus epitope
  PEPTIDE (1)..(15): betacoronavirus epitope
FLTENLLLYIDINGN

SEQ ID NO: 141 - protein, 15 aa, Unknown
  betacoronavirus epitope
  PEPTIDE (1)..(15): betacoronavirus epitope
SLYVNKHAFHTPAFD

SEQ ID NO: 142 - protein, 15 aa, Unknown
  betacoronavirus epitope
  PEPTIDE (1)..(15): betacoronavirus epitope
KAPKEIIFLEGETLP

SEQ ID NO: 143 - protein, 16 aa, Unknown
  betacoronavirus epitope
  PEPTIDE (1)..(16): betacoronavirus epitope
VLSEARQHLKDGTCGL

SEQ ID NO: 144 - protein, 15 aa, Unknown
  betacoronavirus epitope
  PEPTIDE (1)..(15): betacoronavirus epitope
RFKESPFELEDFIPM

SEQ ID NO: 145 - protein, 15 aa, Unknown
  betacoronavirus epitope
  PEPTIDE (1)..(15): betacoronavirus epitope
SLSSTASALGKLQDV

SEQ ID NO: 146 - protein, 15 aa, Unknown
  betacoronavirus epitope
  PEPTIDE (1)..(15): betacoronavirus epitope
SQLGGLHLLIGLAKR

SEQ ID NO: 147 - protein, 15 aa, Unknown
  betacoronavirus epitope
  PEPTIDE (1)..(15): betacoronavirus epitope
AVASKILGLPTQTVD

SEQ ID NO: 148 - protein, 15 aa, Unknown
  betacoronavirus epitope
  PEPTIDE (1)..(15): betacoronavirus epitope
FGADPIHSLRVCVDT

SEQ ID NO: 149 - protein, 15 aa, Unknown
  betacoronavirus epitope
  PEPTIDE (1)..(15): betacoronavirus epitope
FVFPLNSIIKTIQPR

SEQ ID NO: 150 - protein, 15 aa, Unknown
  betacoronavirus epitope
  PEPTIDE (1)..(15): betacoronavirus epitope
FVSNGTHWFVTQRNF

SEQ ID NO: 151 - protein, 15 aa, Unknown
  betacoronavirus epitope
  PEPTIDE (1)..(15): betacoronavirus epitope
SGFAAYSRYRIGNYK

SEQ ID NO: 152 - protein, 15 aa, Unknown
  betacoronavirus epitope
  PEPTIDE (1)..(15): betacoronavirus epitope
FVSDADSTLIGDCAT

SEQ ID NO: 153 - protein, 15 aa, Unknown
  betacoronavirus epitope
  PEPTIDE (1)..(15): betacoronavirus epitope
LQAENVTGLFKDCSK

SEQ ID NO: 154 - protein, 15 aa, Unknown
  betacoronavirus epitope
  PEPTIDE (1)..(15): betacoronavirus epitope
VATSRTLSYYKLGAS

SEQ ID NO: 155 - protein, 17 aa, Unknown
  betacoronavirus epitope
  PEPTIDE (1)..(17): betacoronavirus epitope
YLKSPNFSKLINIIIWF

SEQ ID NO: 156 - protein, 15 aa, Unknown
  betacoronavirus epitope
  PEPTIDE (1)..(15): betacoronavirus epitope
FVVEVVDKYFDCYDG

SEQ ID NO: 157 - protein, 15 aa, Unknown
  betacoronavirus epitope
  PEPTIDE (1)..(15): betacoronavirus epitope
ILKPANNSLKITEEV

SEQ ID NO: 158 - protein, 15 aa, Unknown
  betacoronavirus epitope
  PEPTIDE (1)..(15): betacoronavirus epitope
LQLPQGTTLPKGFYA

SEQ ID NO: 159 - protein, 15 aa, Unknown
  betacoronavirus epitope
  PEPTIDE (1)..(15): betacoronavirus epitope
LLYDANYFLCWHTNC

SEQ ID NO: 160 - protein, 15 aa, Unknown
  betacoronavirus epitope
  PEPTIDE (1)..(15): betacoronavirus epitope
SVVSKVVKVTIDYTE

SEQ ID NO: 161 - protein, 15 aa, Unknown
  betacoronavirus epitope
  PEPTIDE (1)..(15): betacoronavirus epitope
VLYENQKLIANQFNS

SEQ ID NO: 162 - protein, 16 aa, Unknown
  betacoronavirus epitope
  PEPTIDE (1)..(16): betacoronavirus epitope
VVNQNAQALNTLVKQL

SEQ ID NO: 163 - protein, 15 aa, Unknown
  betacoronavirus epitope
  PEPTIDE (1)..(15): betacoronavirus epitope
LLADKFPVLHDIGNP

SEQ ID NO: 164 - protein, 15 aa, Unknown
  betacoronavirus epitope
  PEPTIDE (1)..(15): betacoronavirus epitope
KVQIGEYTFEKGDYG

SEQ ID NO: 165 - protein, 15 aa, Unknown
  betacoronavirus epitope
  PEPTIDE (1)..(15): betacoronavirus epitope
LALSKGVHFVCNLLL

SEQ ID NO: 166 - protein, 15 aa, Unknown
  betacoronavirus epitope
  PEPTIDE (1)..(15): betacoronavirus epitope
MLNPNYEDLLIRKSN

SEQ ID NO: 167 - protein, 15 aa, Unknown
  betacoronavirus epitope
  PEPTIDE (1)..(15): betacoronavirus epitope
AVANGDSEVVLKKLK

SEQ ID NO: 168 - protein, 15 aa, Unknown
  betacoronavirus epitope
  PEPTIDE (1)..(15): betacoronavirus epitope
RLNEVAKNLNESLID

SEQ ID NO: 169 - protein, 15 aa, Unknown
  betacoronavirus epitope
  PEPTIDE (1)..(15): betacoronavirus epitope
SAKSASVYYSQLMCQ

SEQ ID NO: 170 - protein, 15 aa, Unknown
  betacoronavirus epitope
  PEPTIDE (1)..(15): betacoronavirus epitope
STDVVYRAFDIYNDK

SEQ ID NO: 171 - protein, 15 aa, Unknown
  betacoronavirus epitope
  PEPTIDE (1)..(15): betacoronavirus epitope
VFAQVKQIYKTPPIK

SEQ ID NO: 172 - protein, 15 aa, Unknown
  betacoronavirus epitope
  PEPTIDE (1)..(15): betacoronavirus epitope
VLNDILSRLDKVEAE

SEQ ID NO: 173 - protein, 15 aa, Unknown
  betacoronavirus epitope
  PEPTIDE (1)..(15): betacoronavirus epitope
FLRDGWEIVKFISTC

SEQ ID NO: 174 - protein, 15 aa, Unknown
  betacoronavirus epitope
  PEPTIDE (1)..(15): betacoronavirus epitope
GLNDNLLEILQKEKV

SEQ ID NO: 175 - protein, 15 aa, Unknown
  betacoronavirus epitope
  PEPTIDE (1)..(15): betacoronavirus epitope
KIADYNYKLPDDFTG

SEQ ID NO: 176 - protein, 15 aa, Unknown
  betacoronavirus epitope
  PEPTIDE (1)..(15): betacoronavirus epitope
LLLDRLNQLESKMSG

SEQ ID NO: 177 - protein, 15 aa, Unknown
  betacoronavirus epitope
  PEPTIDE (1)..(15): betacoronavirus epitope
VVNVVTTKIALKGGK

SEQ ID NO: 178 - protein, 15 aa, Unknown
  betacoronavirus epitope
  PEPTIDE (1)..(15): betacoronavirus epitope
YVRNLQHRLYECLYR

SEQ ID NO: 179 - protein, 15 aa, Unknown
  betacoronavirus epitope
  PEPTIDE (1)..(15): betacoronavirus epitope
CTDDNALAYYNTTKG

SEQ ID NO: 180 - protein, 15 aa, Unknown
  betacoronavirus epitope
  PEPTIDE (1)..(15): betacoronavirus epitope
RVVFNGVSFSTFEEA

SEQ ID NO: 181 - protein, 15 aa, Unknown
  betacoronavirus epitope
  PEPTIDE (1)..(15): betacoronavirus epitope
TQAPTHLSVDTKFKT

SEQ ID NO: 182 - protein, 15 aa, Unknown
  betacoronavirus epitope
  PEPTIDE (1)..(15): betacoronavirus epitope
VVNAANVYLKHGGGV

SEQ ID NO: 183 - protein, 15 aa, Unknown
  betacoronavirus epitope
  PEPTIDE (1)..(15): betacoronavirus epitope
YAFEHIVYGDFSHSQ

SEQ ID NO: 184 - protein, 15 aa, Unknown
  betacoronavirus epitope
  PEPTIDE (1)..(15): betacoronavirus epitope
YYRYNLPTMCDIRQL

SEQ ID NO: 185 - protein, 15 aa, Unknown
  betacoronavirus epitope
  PEPTIDE (1)..(15): betacoronavirus epitope
YIVDSVTVKNGSIHL

SEQ ID NO: 186 - protein, 15 aa, Unknown
  betacoronavirus epitope
  PEPTIDE (1)..(15): betacoronavirus epitope
HVQLSLPVLQVRDVL

SEQ ID NO: 187 - protein, 15 aa, Unknown
  betacoronavirus epitope
  PEPTIDE (1)..(15): betacoronavirus epitope
ILDITPCSFGGVSVI

SEQ ID NO: 188 - protein, 15 aa, Unknown
  betacoronavirus epitope
  PEPTIDE (1)..(15): betacoronavirus epitope
MVMCGGSLYVKPGGT

SEQ ID NO: 189 - protein, 15 aa, Unknown
  betacoronavirus epitope
  PEPTIDE (1)..(15): betacoronavirus epitope
RITGLYPTLNISDEF

SEQ ID NO: 190 - protein, 15 aa, Unknown
  betacoronavirus epitope
  PEPTIDE (1)..(15): betacoronavirus epitope
YLPQNAVVKIYCPAC

SEQ ID NO: 191 - protein, 15 aa, Unknown
  betacoronavirus epitope
  PEPTIDE (1)..(15): betacoronavirus epitope
RIIPARARVECFDKF

SEQ ID NO: 192 - protein, 15 aa, Unknown
  betacoronavirus epitope
  PEPTIDE (1)..(15): betacoronavirus epitope
ASFDNFKFVCDNIKF

SEQ ID NO: 193 - protein, 15 aa, Unknown
  betacoronavirus epitope
  PEPTIDE (1)..(15): betacoronavirus epitope
TIKPVTYKLDGVVCT

SEQ ID NO: 194 - protein, 15 aa, Unknown
  betacoronavirus epitope
  PEPTIDE (1)..(15): betacoronavirus epitope
VARDLSLQFKRPINP

SEQ ID NO: 195 - protein, 15 aa, Unknown
  betacoronavirus epitope
  PEPTIDE (1)..(15): betacoronavirus epitope
YLASGGQPITNCVKM

SEQ ID NO: 196 - protein, 15 aa, Unknown
  betacoronavirus epitope
  PEPTIDE (1)..(15): betacoronavirus epitope
FSNSGSDVLYQPPQT

SEQ ID NO: 197 - protein, 15 aa, Unknown
  betacoronavirus epitope
  PEPTIDE (1)..(15): betacoronavirus epitope
KAYNVTQAFGRRGPE

SEQ ID NO: 198 - protein, 15 aa, Unknown
  betacoronavirus epitope
  PEPTIDE (1)..(15): betacoronavirus epitope
NTASWFTALTQHGKE

SEQ ID NO: 199 - protein, 15 aa, Unknown
  betacoronavirus epitope
  PEPTIDE (1)..(15): betacoronavirus epitope
RVDGQVDLFRNARNG

SEQ ID NO: 200 - protein, 15 aa, Unknown
  betacoronavirus epitope
  PEPTIDE (1)..(15): betacoronavirus epitope
SAFYILPSIISNEKQ

SEQ ID NO: 201 - protein, 15 aa, Unknown
  betacoronavirus epitope
  PEPTIDE (1)..(15): betacoronavirus epitope
SLPINVIVFDGKSKC

SEQ ID NO: 202 - protein, 15 aa, Unknown
  betacoronavirus epitope
  PEPTIDE (1)..(15): betacoronavirus epitope
YITGGVVQLTSQWLT

SEQ ID NO: 203 - protein, 15 aa, Unknown
  betacoronavirus epitope
  PEPTIDE (1)..(15): betacoronavirus epitope
YVMHANYIFWRNTNP

SEQ ID NO: 204 - protein, 15 aa, Unknown
  betacoronavirus epitope
  PEPTIDE (1)..(15): betacoronavirus epitope
ANDPVGFTLKNTVCT

SEQ ID NO: 205 - protein, 15 aa, Unknown
  betacoronavirus epitope
  PEPTIDE (1)..(15): betacoronavirus epitope
LQAGNATEVPANSTV

SEQ ID NO: 206 - protein, 15 aa, Unknown
  betacoronavirus epitope
  PEPTIDE (1)..(15): betacoronavirus epitope
HLRIAGHHLGRCDIK

SEQ ID NO: 207 - protein, 15 aa, Unknown
  betacoronavirus epitope
  PEPTIDE (1)..(15): betacoronavirus epitope
HSLSHFVNLDNLRAN

SEQ ID NO: 208 - protein, 15 aa, Unknown
  betacoronavirus epitope
  PEPTIDE (1)..(15): betacoronavirus epitope
ITHDVSSAINRPQIG

SEQ ID NO: 209 - protein, 15 aa, Unknown
  betacoronavirus epitope
  PEPTIDE (1)..(15): betacoronavirus epitope
KSFDLGDELGTDPYE

SEQ ID NO: 210 - protein, 15 aa, Unknown
  betacoronavirus epitope
  PEPTIDE (1)..(15): betacoronavirus epitope
LATHGLAAVNSVPWD

SEQ ID NO: 211 - protein, 15 aa, Unknown
  betacoronavirus epitope
  PEPTIDE (1)..(15): betacoronavirus epitope
KTIGPDMFLGTCRRC

SEQ ID NO: 212 - protein, 15 aa, Unknown
  betacoronavirus epitope
  PEPTIDE (1)..(15): betacoronavirus epitope
LALGGSVAIKITEHS

SEQ ID NO: 213 - protein, 15 aa, Unknown
  betacoronavirus epitope
  PEPTIDE (1)..(15): betacoronavirus epitope
KRVDWTIEYPIIGDE

SEQ ID NO: 214 - protein, 15 aa, Unknown
  betacoronavirus epitope
  PEPTIDE (1)..(15): betacoronavirus epitope
KSDGTGTIYTELEPP

SEQ ID NO: 215 - protein, 15 aa, Unknown
  betacoronavirus epitope
  PEPTIDE (1)..(15): betacoronavirus epitope
YVFCTVNALPETTAD

SEQ ID NO: 216 - protein, 15 aa, Unknown
  betacoronavirus epitope
  PEPTIDE (1)..(15): betacoronavirus epitope
LTYNKVENMTPRDLG

SEQ ID NO: 217 - protein, 15 aa, Unknown
  betacoronavirus epitope
  PEPTIDE (1)..(15): betacoronavirus epitope
LVQAGNVQLRVIGHS

SEQ ID NO: 218 - protein, 15 aa, Unknown
  betacoronavirus epitope
  PEPTIDE (1)..(15): betacoronavirus epitope
NVYADSFVIRGDEVR

SEQ ID NO: 219 - protein, 15 aa, Unknown
  betacoronavirus epitope
  PEPTIDE (1)..(15): betacoronavirus epitope
YFVVKRHTFSNYQHE

SEQ ID NO: 220 - protein, 15 aa, Unknown
  betacoronavirus epitope
  PEPTIDE (1)..(15): betacoronavirus epitope
FVLAAVYRINWITGG

SEQ ID NO: 221 - protein, 15 aa, Unknown
  betacoronavirus epitope
  PEPTIDE (1)..(15): betacoronavirus epitope
YVDTPNNTDFSRVSA

SEQ ID NO: 222 - protein, 15 aa, Unknown
  betacoronavirus epitope
  PEPTIDE (1)..(15): betacoronavirus epitope
IFFITGNTLQCIMLV

SEQ ID NO: 223 - protein, 15 aa, Unknown
  betacoronavirus epitope
  PEPTIDE (1)..(15): betacoronavirus epitope
NVNRFNVAITRAKVG

SEQ ID NO: 224 - protein, 15 aa, Unknown
  betacoronavirus epitope
  PEPTIDE (1)..(15): betacoronavirus epitope
TLKEILVTYNCCDDD

SEQ ID NO: 225 - protein, 15 aa, Unknown
  betacoronavirus epitope
  PEPTIDE (1)..(15): betacoronavirus epitope
FITESKPSVEQRKQD

SEQ ID NO: 226 - protein, 15 aa, Unknown
  betacoronavirus epitope
  PEPTIDE (1)..(15): betacoronavirus epitope
TSNQVAVLYQDVNCT

SEQ ID NO: 227 - protein, 15 aa, Unknown
  betacoronavirus epitope
  PEPTIDE (1)..(15): betacoronavirus epitope
YLFQHANLDSCKRVL

SEQ ID NO: 228 - protein, 15 aa, Unknown
  betacoronavirus epitope
  PEPTIDE (1)..(15): betacoronavirus epitope
WTNAGDYILANTCTE

SEQ ID NO: 229 - protein, 15 aa, Unknown
  betacoronavirus epitope
  PEPTIDE (1)..(15): betacoronavirus epitope
SLSHRFYRLANECAQ

SEQ ID NO: 230 - protein, 1273 aa, Unknown
  spike protein of Severe acute respiratory syndrome coronavirus 2
  SITE (1)..(1273): spike protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 231 - protein, 253 aa, Unknown
  RBD of spike protein
  SITE (1)..(253): RBD
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLE

SEQ ID NO: 232 - protein, 38 aa, Unknown
  HR2 domain of spike protein
  SITE (1)..(38): HR2
DVDLGDISGINASVVNIQKEIDRLNEVAKNLNESLIDL

SEQ ID NO: 233 - protein, 243 aa, Unknown
  AA sequence of B4001
  SITE: B4001
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTIEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGL

SEQ ID NO: 234 - protein, 93 aa, homo sapiens
MQVSTAALAVLLCTMALCNQVLSAPLAADTPTACCFSYTSRQIPQNFIADYFETSSQCSKPSVIFLTKRGRQVCADPSEEWVQKYVSDLELSA

SEQ ID NO: 235 - protein, 19 aa, Unknown
  Signal peptide
  SIGNAL (1)..(19): Signal peptide
MNFGLRLIFLVLTLKGVQC

SEQ ID NO: 236 - protein, 22 aa, Unknown
  Signal peptide
  SIGNAL (1)..(22): signal peptide
MDAMKRGLCCVLLLCGAVFVSP

SEQ ID NO: 237 - protein, 428 aa, Unknown
  antigenic unit
  SITE (1)..(428): VB2040
MTYRRLISMMGFKMNYQVNGYPNMFGGGGSGGGGSFLNRFTTTLNDFNLVAMSSVELKHFFFAQDGNAAIGGGGSGGGGSRSFIEDLLFNKVTLALMIERFVSLAIDAYPGGGGSGGGGSRAMPNMLRIMASLVLMVYMPASWVMRIMTWGGGGSGGGGSHFAIGLALYYPSARIVYTACSHAAVGGGGSGGGGSYFIKGLNNLNRGMVLYLNTLTLAVPYNMRVGGGGSGGGGSNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPAT

SEQ ID NO: 238 - protein, 534 aa, Unknown
  antigenic unit
  SITE (1)..(534): VB2041
MTYRRLISMMGFKMNYQVNGYPNMFGGGGSGGGGSHVISTSHKLVLSVNPYVGGGGSGGGGSFLNRFTTTLNDFNLVAMSSVELKHFFFAQDGNAAIGGGGSGGGGSHFAIGLALYYPSARIVYTACSHAAVGGGGSGGGGSRAMPNMLRIMASLVLMVYMPASWVMRIMTWGGGGSGGGGSRSFIEDLLFNKVTLALMIERFVSLAIDAYPGGGGSGGGGSAQFAPSASAFFGMSRIEIVDTVSALVYDNKLSGGGGSGGGGYFIKGLNNLNRGMVLYLNTLTLAVPYNMRVSGGGGSGGGGSSGDATTAYANSVFNICQAVTANVNALLGGGGSGGGGSNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPAT

SEQ ID NO: 239 - protein, 672 aa, Unknown
  antigenic unit
  SITE (1)..(672): VB2042
MTYRRLISMMGFKMNYQVNGYPNMFGGGGSGGGGSHVISTSHKLVLSVNPYVASIKNFKSVLYYQNNVFMGGGGSGGGGSYFIKGLNNLNRGMVLYLNTLTLAVPYNMRVGGGGSGGGGSMLSDTLKNLSDRVVFVLWAHGFELSGGGGSGGGGHFAIGLALYYPSARIVYTACSHAAVGGGGSGGGGSRSFIEDLLFNKVTLALMIERFVSLAIDAYPGGGGSGGGGSRAMPNMLRIMASLVLMVYMPASWVMRIMTWGGGGSGGGGSRVWTLMNVLTLVYKVFAYANRNRFLYIIKLSGGGGSGGGGAQFAPSASAFFGMSRIEIVDTVSALVYDNKLSGGGGSGGGGFLNRFTTTLNDFNLVAMSSVELKHFFFAQDGNAAISGGGGSGGGGSSGDATTAYANSVFNICQAVTANVNALLSGGGGSGGGGTANPKTPKYKFVRIQPGQTFFVNEFYAYLRKHFSMMGGGGSGGGGSNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPAT

SEQ ID NO: 240 - protein, 787 aa, Unknown
  antigenic unit
  SITE (1)..(787): VB2043
MTYRRLISMMGFKMNYQVNGYPNMFGGGGSGGGGSHVISTSHKLVLSVNPYVASIKNFKSVLYYQNNVFMSGGGGSGGGGSSGDATTAYANSVFNICQAVTANVNALLSGGGGSGGGGFLNRFTTTLNDFNLVAMSSVELKHFFFAQDGNAAIGGGGSGGGGSMLSDTLKNLSDRVVFVLWAHGFELSGGGGSGGGGHFAIGLALYYPSARIVYTACSHAAVGGGGSGGGGSRSFIEDLLFNKVTLALMIERFVSLAIDAYPGGGGSGGGGSRAMPNMLRIMASLVLMVYMPASWVMRIMTWGGGGSGGGGSVQMAPISAMVRMYIFFASFYYVWKSYVHVGGGGSGGGGSRVWTLMNVLTLVYKVFAYANRNRFLYIIKLSGGGGSGGGGAQFAPSASAFFGMSRIEIVDTVSALVYDNKLSGGGGSGGGGSLLMPILTLTRALTAYTVELGTEVNEFACVGGGGSGGGGSYFIKGLNNLNRGMVLYLNTLTLAVPYNMRVGGGGSGGGGSTVYTKVDGVDVELFENKTTLPVNVAFGGGGSGGGGSTANPKTPKYKFVRIQPGQTFFVNEFYAYLRKHFSMMGGGGSGGGGSNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPAT

SEQ ID NO: 241 - protein, 940 aa, Unknown
  antigenic unit
  SITE (1)..(940): VB2044
KMFDAYVNTFSSTFNVRVYSTGSNVFQTRAGGSSGGGSSGGSVAYSNNSIAIPTNFTISVTTEIGGGGSGGGGSTANPKTPKYKFVRIQPGQTFFVNEFYAYLRKHFSMMGGGGSGGGGSTVYTKVDGVDVELFENKTTLPVNVAFGGGGSGGGGSYFIKGLNNLNRGMVLYLNTLTLAVPYNMRVGGGGSGGGGSLVKPSFYVYSRVKNLVQQESPFVMMSAPPAQYELSGGGGSGGGGMLSDTLKNLSDRVVFVLWAHGFELSGGGGSGGGGHFAIGLALYYPSARIVYTACSHAAVGGGGSGGGGSRSFIEDLLFNKVTLALMIERFVSLAIDAYPGGGGSGGGGSRAMPNMLRIMASLVLMVYMPASWVMRIMTWGGGGSGGGGSVQMAPISAMVRMYIFFASFYYVWKSYVHVGGGGSGGGGSRVWTLMNVLTLVYKVFAYANRNRFLYIIKLSGGGGSGGGGAQFAPSASAFFGMSRIEIVDTVSALVYDNKLSGGGGSGGGGSLLMPILTLTRALTAYTVELGTEVNEFACVGGGGSGGGGSFLNRFTTTLNDFNLVAMSSVELKHFFFAQDGNAAISGGGGSGGGGSSGDATTAYANSVFNICQAVTANVNALLSGGGGSGGGGHVISTSHKLVLSVNPYVASIKNFKSVLYYQNNVFMGGGGSGGGGSMTYRRLISMMGFKMNYQVNGYPNMFGGGGSGGGGSFGDDTVIEVQGYKSVNITFELDERIGGGGSGGGGSNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPAT

SEQ ID NO: 242 - protein, 672 aa, Unknown
  antigenic unit
  SITE (1)..(672): VB2045
MTYRRLISMMGFKMNYQVNGYPNMFGGGGSGGGGSHVISTSHKLVLSVNPYVASIKNFKSVLYYQNNVFMGGGGSGGGGSYFIKGLNNLNRGMVLYLNTLTLAVPYNMRVGGGGSGGGGSMLSDTLKNLSDRVVFVLWAHGFELSGGGGSGGGGHFAIGLALYYPSARIVYTACSHAAVGGGGSGGGGSRSFIEDLLFNKVTLALMIERFVSLAIDAYPGGGGSGGGGSNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATGGGGSGGGGSRAMPNMLRIMASLVLMVYMPASWVMRIMTWGGGGSGGGGSRVWTLMNVLTLVYKVFAYANRNRFLYIIKLSGGGGSGGGGAQFAPSASAFFGMSRIEIVDTVSALVYDNKLSGGGGSGGGGFLNRFTTTLNDFNLVAMSSVELKHFFFAQDGNAAISGGGGSGGGGSSGDATTAYANSVFNICQAVTANVNALLSGGGGSGGGGTANPKTPKYKFVRIQPGQTFFVNEFYAYLRKHFSMM

SEQ ID NO: 243 - protein, 672 aa, Unknown
  antigenic unit
  SITE (1)..(672): VB2046
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATGGGGSGGGGSMTYRRLISMMGFKMNYQVNGYPNMFGGGGSGGGGSHVISTSHKLVLSVNPYVASIKNFKSVLYYQNNVFMGGGGSGGGGSYFIKGLNNLNRGMVLYLNTLTLAVPYNMRVGGGGSGGGGSMLSDTLKNLSDRVVFVLWAHGFELSGGGGSGGGGHFAIGLALYYPSARIVYTACSHAAVGGGGSGGGGSRSFIEDLLFNKVTLALMIERFVSLAIDAYPGGGGSGGGGSRAMPNMLRIMASLVLMVYMPASWVMRIMTWGGGGSGGGGSRVWTLMNVLTLVYKVFAYANRNRFLYIIKLSGGGGSGGGGAQFAPSASAFFGMSRIEIVDTVSALVYDNKLSGGGGSGGGGFLNRFTTTLNDFNLVAMSSVELKHFFFAQDGNAAISGGGGSGGGGSSGDATTAYANSVFNICQAVTANVNALLSGGGGSGGGGTANPKTPKYKFVRIQPGQTFFVNEFYAYLRKHFSMM

SEQ ID NO: 244 - protein, 552 aa, Unknown
  antigenic unit
  SITE (1)..(552): VB2047
MTYRRLISMMGFKMNYQVNGYPNMFHVISTSHKLVLSVNPYVASIKNFKSVLYYQNNVFMYFIKGLNNLNRGMVLYLNTLTLAVPYNMRVMLSDTLKNLSDRVVFVLWAHGFELHFAIGLALYYPSARIVYTACSHAAVRSFIEDLLFNKVTLALMIERFVSLAIDAYPRAMPNMLRIMASLVLMVYMPASWVMRIMTWRVWTLMNVLTLVYKVFAYANRNRFLYIIKLAQFAPSASAFFGMSRIEIVDTVSALVYDNKLFLNRFTTTLNDFNLVAMSSVELKHFFFAQDGNAAISSGDATTAYANSVFNICQAVTANVNALLTANPKTPKYKFVRIQPGQTFFVNEFYAYLRKHFSMMNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPAT

SEQ ID NO: 245 - protein, 469 aa, Unknown
  antigenic unit
  SITE (1)..(469): VB2048
MTYRRLISMMGFKMNYQVNGYPNMFGGGGSGGGGSHVISTSHKLVLSVNPYVASIKNFKSVLYYQNNVFMGGGGSGGGGSYFIKGLNNLNRGMVLYLNTLTLAVPYNMRVGGGGSGGGGSMLSDTLKNLSDRVVFVLWAHGFELSGGGGSGGGGHFAIGLALYYPSARIVYTACSHAAVGGGGSGGGGSRSFIEDLLFNKVTLALMIERFVSLAIDAYPGGGGSGGGGSRAMPNMLRIMASLVLMVYMPASWVMRIMTWGGGGSGGGGSRVWTLMNVLTLVYKVFAYANRNRFLYIIKLSGGGGSGGGGAQFAPSASAFFGMSRIEIVDTVSALVYDNKLSGGGGSGGGGFLNRFTTTLNDFNLVAMSSVELKHFFFAQDGNAAISGGGGSGGGGSSGDATTAYANSVFNICQAVTANVNALLSGGGGSGGGGTANPKTPKYKFVRIQPGQTFFVNEFYAYLRKHFSMM

SEQ ID NO: 246 - protein, 193 aa, Unknown
  antigenic unit
  SITE (1)..(193): VB2049
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPAT

SEQ ID NO: 247 - protein, 737 aa, Unknown
  antigenic unit
  SITE (1)..(737): VB2050
KMFDAYVNTFSSTFNVRVYSTGSNVFQTRAGGSSGGGSSGGSVAYSNNSIAIPTNFTISVTTEIGGGGSGGGGSTANPKTPKYKFVRIQPGQTFFVNEFYAYLRKHFSMMGGGGSGGGGSTVYTKVDGVDVELFENKTTLPVNVAFGGGGSGGGGSYFIKGLNNLNRGMVLYLNTLTLAVPYNMRVGGGGSGGGGSLVKPSFYVYSRVKNLVQQESPFVMMSAPPAQYELSGGGGSGGGGMLSDTLKNLSDRVVFVLWAHGFELSGGGGSGGGGHFAIGLALYYPSARIVYTACSHAAVGGGGSGGGGSRSFIEDLLFNKVTLALMIERFVSLAIDAYPGGGGSGGGGSRAMPNMLRIMASLVLMVYMPASWVMRIMTWGGGGSGGGGSVQMAPISAMVRMYIFFASFYYVWKSYVHVGGGGSGGGGSRVWTLMNVLTLVYKVFAYANRNRFLYIIKLSGGGGSGGGGAQFAPSASAFFGMSRIEIVDTVSALVYDNKLSGGGGSGGGGSLLMPILTLTRALTAYTVELGTEVNEFACVGGGGSGGGGSFLNRFTTTLNDFNLVAMSSVELKHFFFAQDGNAAISGGGGSGGGGSSGDATTAYANSVFNICQAVTANVNALLSGGGGSGGGGHVISTSHKLVLSVNPYVASIKNFKSVLYYQNNVFMGGGGSGGGGSMTYRRLISMMGFKMNYQVNGYPNMFGGGGSGGGGSFGDDTVIEVQGYKSVNITFELDERI

SEQ ID NO: 248 - protein, 1273 aa, Unknown; identical to SEQ ID NO: 230
  antigenic unit
  SITE (1)..(1273): VB2051

SEQ ID NO: 249 - protein, 49 aa, Unknown
  Alternative HR2 domain
  SITE (1)..(49): HR2
DVDLGDISGINASVVNIQKEIDRLNEVAKNLNESLIDLQELGKYEQYIK

SEQ ID NO: 250 - protein, 796 aa, Unknown
  antigenic unit
  SITE (1)..(796): VB2053
KMFDAYVNTFSSTFNVRVYSTGSNVFQTRAGGSSGGGSSGGSVAYSNNSIAIPTNFTISVTTEIGGGGSGGGGSTANPKTPKYKFVRIQPGQTFFVNEFYAYLRKHFSMMGGGGSGGGGSTVYTKVDGVDVELFENKTTLPVNVAFGGGGSGGGGSYFIKGLNNLNRGMVLYLNTLTLAVPYNMRVGGGGSGGGGSLVKPSFYVYSRVKNLVQQESPFVMMSAPPAQYELSGGGGSGGGGMLSDTLKNLSDRVVFVLWAHGFELSGGGGSGGGGHFAIGLALYYPSARIVYTACSHAAVGGGGSGGGGSRSFIEDLLFNKVTLALMIERFVSLAIDAYPGGGGSGGGGSRAMPNMLRIMASLVLMVYMPASWVMRIMTWGGGGSGGGGSVQMAPISAMVRMYIFFASFYYVWKSYVHVGGGGSGGGGSRVWTLMNVLTLVYKVFAYANRNRFLYIIKLSGGGGSGGGGAQFAPSASAFFGMSRIEIVDTVSALVYDNKLSGGGGSGGGGSLLMPILTLTRALTAYTVELGTEVNEFACVGGGGSGGGGSFLNRFTTTLNDFNLVAMSSVELKHFFFAQDGNAAISGGGGSGGGGSSGDATTAYANSVFNICQAVTANVNALLSGGGGSGGGGHVISTSHKLVLSVNPYVASIKNFKSVLYYQNNVFMGGGGSGGGGSMTYRRLISMMGFKMNYQVNGYPNMFGGGGSGGGGSFGDDTVIEVQGYKSVNITFELDERIGGGGSGGGGSDVDLGDISGINASVVNIQKEIDRLNEVAKNLNESLIDLQELGKYEQYIK

SEQ ID NO: 251 - protein, 999 aa, Unknown
  antigenic unit
  SITE (1)..(999): VB2054
DVDLGDISGINASVVNIQKEIDRLNEVAKNLNESLIDLQELGKYEQYIKGGGGSGGGGSKMFDAYVNTFSSTFNVRVYSTGSNVFQTRAGGSSGGGSSGGSVAYSNNSIAIPTNFTISVTTEIGGGGSGGGGSTANPKTPKYKFVRIQPGQTFFVNEFYAYLRKHFSMMGGGGSGGGGSTVYTKVDGVDVELFENKTTLPVNVAFGGGGSGGGGSYFIKGLNNLNRGMVLYLNTLTLAVPYNMRVGGGGSGGGGSLVKPSFYVYSRVKNLVQQESPFVMMSAPPAQYELSGGGGSGGGGMLSDTLKNLSDRVVFVLWAHGFELSGGGGSGGGGHFAIGLALYYPSARIVYTACSHAAVGGGGSGGGGSRSFIEDLLFNKVTLALMIERFVSLAIDAYPGGGGSGGGGSRAMPNMLRIMASLVLMVYMPASWVMRIMTWGGGGSGGGGSVQMAPISAMVRMYIFFASFYYVWKSYVHVGGGGSGGGGSRVWTLMNVLTLVYKVFAYANRNRFLYIIKLSGGGGSGGGGAQFAPSASAFFGMSRIEIVDTVSALVYDNKLSGGGGSGGGGSLLMPILTLTRALTAYTVELGTEVNEFACVGGGGSGGGGSFLNRFTTTLNDFNLVAMSSVELKHFFFAQDGNAAISGGGGSGGGGSSGDATTAYANSVFNICQAVTANVNALLSGGGGSGGGGHVISTSHKLVLSVNPYVASIKNFKSVLYYQNNVFMGGGGSGGGGSMTYRRLISMMGFKMNYQVNGYPNMFGGGGSGGGGSFGDDTVIEVQGYKSVNITFELDERIGGGGSGGGGSNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPAT

SEQ ID NO: 252 - DNA, 1305 bp, Artificial sequence
  VB construct
atgcaggtctccactgctgcccttgccgtcctcctctgcaccatggctctctgcaaccaggtcctctctgcaccacttgctgctgacacgccgaccgcctgctgcttcagctacacctcccgacagattccacagaatttcatagctgactactttgagacgagcagccagtgctccaagcccagtgtcatcttcctaaccaagagaggccggcaggtctgtgctgaccccagtgaggagtgggtccagaaatacgtcagtgacctggagctgagtgccgagctcaaaaccccacttggtgacacaactcacacagagcccaaatcttgtgacacacctcccccgtgcccaaggtgcccaggcggtggaagcagcggaggtggaagtggaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaaggcctcggtggcctgaatattactaacctgtgcccattcggcgaggtgttcaacgcaacccggtttgcaagcgtgtacgcatggaacagaaagcggattagcaactgcgtcgccgactacagcgtgctctacaacagcgcatcattctccacattcaagtgttacggagtgtcacccactaagctgaacgacctctgcttcacaaatgtctacgccgattccttcgtgatccggggagatgaagtcaggcagattgcaccaggacagactggcaaaatcgctgactacaactataagctgccagacgatttcactggctgcgtgattgcatggaacagcaataacctggactcaaaggtcggcggtaattataactacctgtatcggctgttcagaaagagcaacctgaagcccttcgagcgggacatctccaccgaaatctaccaggcaggctcaacaccctgcaacggagtggaaggcttcaactgctatttccccctgcagtcttacggttttcagccaacaaacggtgtgggctatcagccatacagggtggtcgtgctgagcttcgaactgctccatgcccccgctaca

SEQ ID NO: 253 - protein, 435 aa, Artificial sequence
  VB construct
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPAT

SEQ ID NO: 254 - DNA, 1395 bp, Artificial sequence
  VB construct
atgcaggtctccactgctgcccttgccgtcctcctctgcaccatggctctctgcaaccaggtcctctctgcaccacttgctgctgacacgccgaccgcctgctgcttcagctacacctcccgacagattccacagaatttcatagctgactactttgagacgagcagccagtgctccaagcccagtgtcatcttcctaaccaagagaggccggcaggtctgtgctgaccccagtgaggagtgggtccagaaatacgtcagtgacctggagctgagtgccgagctcaaaaccccacttggtgacacaactcacacagagcccaaatcttgtgacacacctcccccgtgcccaaggtgcccaggcggtggaagcagcggaggtggaagtggaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaaggcctcggtggcctgcgagtccagcccacagaatcaatcgtgcggtttccaaacatcaccaatctctgccccttcggagaagtgtttaatgccacacggtttgcctcagtgtatgcctggaaccggaagagaatctccaactgcgtggcagactattctgtgctgtacaacagcgcctccttctctactttcaagtgctacggcgtgtctcctactaagctgaacgatctctgcttcacaaacgtgtatgccgattcctttgtgatcagaggagacgaagtgcgacagattgctcctggacagaccgggaagattgccgactacaactataagctgccagacgatttcactggctgcgttattgcctggaattcaaacaacctcgattccaaagtgggcgggaattataactacctctatagactgttccgtaagtctaacctgaagccattcgagcgtgatattagcaccgaaatctaccaggcaggttcaactccttgtaacggagtggagggattcaactgctacttcccactgcagtcctatgggttccagcctacaaacggagtggggtaccagccttatcgggtcgttgtgctgtcctttgagctgctccacgctcctgcaactgtttgcggacctaagaaatccaccaacctggtgaagaacaagtgcgtgaatttc

SEQ ID NO: 255 - protein, 465 aa, Artificial sequence
  VB construct
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 256 - DNA, 4311 bp, Artificial sequence
  VB construct
atgcaggtctccactgctgcccttgccgtcctcctctgcaccatggctctctgcaaccaggtcctctctgcaccacttgctgctgacacgccgaccgcctgctgcttcagctacacctcccgacagattccacagaatttcatagctgactactttgagacgagcagccagtgctccaagcccagtgtcatcttcctaaccaagagaggccggcaggtctgtgctgaccccagtgaggagtgggtccagaaatacgtcagtgacctggagctgagtgccgagctcaaaaccccacttggtgacacaactcacacagagcccaaatcttgtgacacacctcccccgtgcccaaggtgcccaggcggtggaagcagcggaggtggaagtggaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaaggcctcggtggcctggtgaacctgacaaccagaacccagctgcctcctgcttataccaatagttttactcggggcgtctactaccctgacaaagtgtttcggagcagtgtgctgcatagcacccaggacctgttcctccccttcttcagtaatgtcacttggttccatgcaatccacgtgtcaggtactaatggtacaaaaaggttcgacaacccagttctgcccttcaatgacggcgtttacttcgccagtaccgaaaagtctaatatcattcgtggttggatcttcggaacaaccctggacagtaagacccagtctctgctcattgttaataacgccacaaatgtcgtgatcaaagtttgtgaatttcagttttgtaacgatccattcctgggcgtctactaccataagaataataagagctggatggaaagcgagtttcgagtctacagcagtgccaataattgtacattcgagtacgtttctcagcctttcctgatggacctggagggcaagcagggaaatttcaaaaatctccgtgaatttgtctttaagaatattgacggctacttcaaaatctacagcaagcacaccccgattaatctggttcgagacctgccacagggcttcagtgcactggagcccctggttgatctgccaatcggaattaatatcactaggttccagaccctgctggcactgcatcgtagttacctcacacctggggattctagttccggctggactgctggggcagccgcctactatgtgggatatctccagcctcgcacatttctgctgaagtataatgagaacggtactattaccgacgcagtggactgtgcactcgacccactgagtgagactaaatgcaccctcaaaagtttcaccgtcgagaagggtatctaccagacatccaatttccgcgtgcagcctactgagtccatcgtgcgcttcccaaatatcaccaatctgtgcccattcggtgaagttttcaatgctactcgctttgctagtgtttacgcatggaacagaaagaggatctcaaactgcgtggctgactatagcgttctgtacaattctgcaagtttttctacttttaaatgttacggcgttagtcctaccaaactgaacgacctctgtttcaccaacgtctacgctgactctttcgtcatccggggcgatgaagttcgacagatcgccccaggccagacaggaaagatcgcagactacaattacaagctgccagatgacttcaccggctgcgttattgcctggaactccaataatctggacagcaaagtgggtgggaactacaactacctctaccgcctcttcagaaagagtaatctgaagccgtttgagcgcgatatttccaccgaaatctatcaggccggaagcacaccgtgcaatggagtcgaagggttcaattgttactttccgctccagagctacgggttccagcccacaaatggggttggatatcagccttacagggtggttgttctgtcttttgagctgctgcatgcaccagctaccgtctgcgggcccaagaagagcaccaatctcgtcaagaacaagtgcgtgaatttcaacttcaacgggctcaccgggaccggcgtcctgacagagtctaataagaaatttctgcccttccagcagtttggaagagacatcgctgataccacagacgccgtccgggatccccagacactggaaatcctcgacatcacaccatgtagctttgggggagttagcgtcattacaccaggaactaatactagcaatcaggtggccgtcctgtaccaggacgtgaattgtacagaagttccagtcgctatccacgcagaccagctgaccccgacttggcgcgtgtactccacaggatctaacgtgttccagacacgtgccggctgcctgattggggctgagcacgtcaacaattcttacgagtgtgacattcccatcggagccggaatctgtgcttcttaccagacccagacaaactctccttcaggagccggatccgttgcttctcagtcaatcatcgcctatactatgagcctgggtgccgaaaactctgttgcttattctaacaatagcattgctatcccgaccaatttcaccatctctgtcaccactgagatcctgccagtctccatgaccaagacctccgtcgactgtacaatgtatatttgtggagactcaaccgagtgctctaacctcctgctgcagtatggatccttctgcacccagctcaacagggcactgaccggaattgctgtcgaacaggacaagaacactcaggaggtcttcgcacaggtcaagcagatatacaagaccccccctatcaaagactttgggggattcaacttctcccagatcctccccgatccttctaagccatcaaagcggagtttcattgaggatctgctcttcaacaaggtcaccctcgctgacgctggcttcattaagcagtacggagactgtctgggggacatcgcagcaagagacctgatctgcgcccagaagttcaacggactgacagtcctgccgcccctgctcactgatgaaatgatcgcccagtatacaagtgctctgctcgccggaaccatcacttccgggtggaccttcggagcaggcgcagccctgcagatccctttcgccatgcagatggcctaccggttcaatggcatcggcgtgacccagaatgtcctgtatgagaatcagaagctgatcgcaaatcagttcaacagcgccatcggaaaaattcaggactcactctctagtaccgcctcagccctgggaaaactccaggatgttgtgaaccagaatgctcaggctctcaacactctggttaaacagctcagcagcaatttcggtgccattagctccgtcctgaatgacatcctcagccgtctcgatccaccggaagctgaagtccagatcgatcgtctcatcacaggacgactgcagagcctgcagacctacgtgacccagcagctcatccgcgctgccgaaattcgagccagcgccaatctcgcagctacaaaaatgagcgagtgcgtcctgggacagtccaagagagttgatttctgcgggaagggctatcatctcatgtctttcccacagtctgcaccacacggcgtggttttcctgcacgtcacctacgtcccagcccaggagaagaactttaccaccgctccagccatttgtcatgacggcaaagcccattttccgcgcgaaggagttttcgtcagcaatgggactcactggtttgtcactcagcgcaatttctatgaaccacagattattacaaccgataataccttcgtcagtggtaattgtgacgtagtgatcggcatcgttaacaatacagtctatgatccgctgcagcctgagctcgacagcttcaaggaggagctggacaagtacttcaagaaccacaccagccctgacgtggatctcggcgacatttctggcattaatgcctctgtcgtcaatatccagaaggaaatcgaccgactgaatgaagtcgcaaagaatctgaacgagagtctgattgatctgcaggagctgggaaaatacgaacagtacatc

SEQ ID NO: 257 - protein, 1437 aa, Artificial sequence
  VB construct
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPSGAGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYI

SEQ ID NO: 258 - DNA, 2133 bp, Artificial sequence
  VB construct
atgcaggtctccactgctgcccttgccgtcctcctctgcaccatggctctctgcaaccaggtcctctctgcaccacttgctgctgacacgccgaccgcctgctgcttcagctacacctcccgacagattccacagaatttcatagctgactactttgagacgagcagccagtgctccaagcccagtgtcatcttcctaaccaagagaggccggcaggtctgtgctgaccccagtgaggagtgggtccagaaatacgtcagtgacctggagctgagtgccgagctcaaaaccccacttggtgacacaactcacacagagcccaaatcttgtgacacacctcccccgtgcccaaggtgcccaggcggtggaagcagcggaggtggaagtggaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaaggcctcggtggcctgatgacttatcgcagactgatctccatgatggggttcaagatgaactaccaggtcaatggctatcccaacatgttcggtgggggtggttctggcgggggtggaagccacgtgatctctacctcccacaagctggtcctgtccgtgaacccctacgtggcctccatcaaaaactttaagtccgtgctctattatcagaataacgtgttcatgggtggagggggctctggaggtggcgggtcatactttattaagggcctgaataacctcaacagggggatggtgctgtatctcaacaccctgactctcgccgtgccttacaatatgcgcgtgggtggtggtggctctggcggtggcggttctatgctgtctgatacactgaagaatctgtccgaccgagtggtgttcgtcctgtgggcacacggtttcgagctgtcagggggtggagggtctggcggaggcgggcatttcgccatcggtctcgccctgtattacccttccgcacggatcgtctatactgcctgttcccatgcagcagtcgggggtggagggtccggcggaggtggctccaggtcttttattgaggacctcctgttcaacaaggtgaccctggctctgatgatcgagcggtttgtctccctggctattgatgcatacccaggtggcggagggtcaggtggaggcgggtcccgggctatgcccaacatgctgagaatcatggcatctctggtgctcatggtgtacatgcctgcaagctgggtcatgcgaattatgacatgggggggaggcggtagcggtggcggaggctcaagagtctggacactcatgaacgtgctgactctcgtgtacaaagtgtttgcttacgccaatcgcaacaggtttctgtacattatcaagctctccggtggcggaggttccgggggagggggcgcccagttcgctccatccgcctcagcattcttcggtatgagtcggatcgagattgtcgataccgtgtctgcactggtctatgacaataagctgagcggaggtggtgggtccggcggtggggggttcctgaacaggttcacaaccactctcaacgactttaatctggtcgccatgtcaagcgtcgaactcaagcatttcttctttgcccaggatggcaacgcagctatctccggtggaggaggtagtggcggcggtggatcttccggcgatgctaccaccgcctacgctaattccgtgtttaacatttgccaggctgtgactgctaacgtgaatgcactgctgtctggtggcgggggcagcgggggcgggggtactgccaaccctaagactcccaagtacaaattcgtccgcatccagcctggccagactttcttcgtgaacgagttttacgcttatctgcggaagcactttagcatgatg

SEQ ID NO: 259 - protein, 711 aa, Artificial sequence
  VB construct
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLMTYRRLISMMGFKMNYQVNGYPNMFGGGGSGGGGSHVISTSHKLVLSVNPYVASIKNFKSVLYYQNNVFMGGGGSGGGGSYFIKGLNNLNRGMVLYLNTLTLAVPYNMRVGGGGSGGGGSMLSDTLKNLSDRVVFVLWAHGFELSGGGGSGGGGHFAIGLALYYPSARIVYTACSHAAVGGGGSGGGGSRSFIEDLLFNKVTLALMIERFVSLAIDAYPGGGGSGGGGSRAMPNMLRIMASLVLMVYMPASWVMRIMTWGGGGSGGGGSRVWTLMNVLTLVYKVFAYANRNRFLYIIKLSGGGGSGGGGAQFAPSASAFFGMSRIEIVDTVSALVYDNKLSGGGGSGGGGFLNRFTTTLNDFNLVAMSSVELKHFFFAQDGNAAISGGGGSGGGGSSGDATTAYANSVFNICQAVTANVNALLSGGGGSGGGGTANPKTPKYKFVRIQPGQTFFVNEFYAYLRKHFSMM

SEQ ID NO: 260 - DNA, 1911 bp, Artificial sequence
  VB construct
atgaactttggtctgcggctgattttcctcgtcctgactctcaagggcgtccagtgccaggtccagctcactcagagccccgcctctctggcagtgagcctggggcagagagctactattagctgcagggctagtaaaagcgtgtcaaccagcggttattcatacatgcattggtatcagcagaaacccggtcagccacctaagctcctcatctacctcacttccaacctggaatctggtgtgccagcacgcttttcaggctccggaagtggcacagacttcaccctgaatatccacccagtggaagaagaggacgccgctacatactattgtcagcacagccgggaactgccatggacttttggtgggggcactaagctggagattaagagagggggtggaggttctggcggagggggttcaggaggaggcgggtcccaggtccagctccagcagtctggtccagacctcgtgaaacctggtgcttcagtcaccatcagttgcaaagctagtggctatgctttctcaagtagctggatgagctggctcaaacagcgtcccggcaaaggactggagtggattgggtggatcttcccaagagacggtgacaccaactataacggtaaattcaaggggaaagccactctgaccgccgacaagtcaagcagtacagcttatatgcagctcagtagcctcactagtgaggacagcgccgtgtatttttgcgcaaggaggggcgactatcactatgggatggactattggggacagggcacctctgtcaccgtgagttctgagctgaaaacccccctcggtgacactactcacaccgaacccaaatcctgtgacacccctcccccttgccctcgctgtcctggtggtggcagttcagggggtgggagcgggggccagcctagagagccacaggtctatacactccctccctctagagaagagatgaccaaaaatcaggtgtcactgacatgcctggtcaagggtttctacccaagcgacattgcagtggaatgggagtcaagtggccagccagagaacaattataacactacacctccaatgctcgacagcgatggtagtttctttctctattccaagctgacagtcgacaaatctagatggcagcagggcaatatcttcagctgttcagtgatgcatgaggccctgcacaaccggttcactcagaagagcctgtcactctcccccgggaagggcctcggaggtctgagggtccagcctactgaatccatcgtccggttcccaaatattaccaacctctgccctttcggagaggtcttcaatgccacacgctttgccagtgtctatgcctggaatcgcaagaggattagtaactgcgtggcagactatagtgtgctgtacaactcagcttctttctcaacattcaaatgctatggagtgtcaccaaccaaactcaacgatctgtgcttcactaatgtctatgctgactcctttgtcattaggggggatgaggtccgccagattgcccctggtcagaccgggaagattgccgactataactataagctcccagatgacttcactggctgcgtgatcgcctggaatagcaacaacctcgattccaaggtgggcggcaattacaactacctctatagactgtttcgaaagagtaatctcaaaccatttgagcgggacatcagcacagaaatctatcaggctggatctacaccttgcaacggcgtcgaagggttcaactgttacttcccactccagtcttacggttttcagcccactaacggggtggggtaccagccctatcgggtggtcgtcctgagtttcgaactcctgcacgcccccgcaacagtctgcgggcccaaaaagtccactaacctggtgaagaacaagtgcgtgaacttc

SEQ ID NO: 261 - protein, 637 aa, Artificial sequence
  VB construct
MNFGLRLIFLVLTLKGVQCQVQLTQSPASLAVSLGQRATISCRASKSVSTSGYSYMHWYQQKPGQPPKLLIYLTSNLESGVPARFSGSGSGTDFTLNIHPVEEEDAATYYCQHSRELPWTFGGGTKLEIKRGGGGSGGGGSGGGGSQVQLQQSGPDLVKPGASVTISCKASGYAFSSSWMSWLKQRPGKGLEWIGWIFPRDGDTNYNGKFKGKATLTADKSSSTAYMQLSSLTSEDSAVYFCARRGDYHYGMDYWGQGTSVTVSSELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 262 - DNA, 4827 bp, Artificial sequence
  VB construct
atgaatttcggcctgaggctgatttttctcgtgctgactctgaaaggagtgcagtgtcaggtccagctgactcagtctccagcctcactggccgtgtcactgggacagcgcgccactatctcatgcagggcctctaaaagcgtgtcaactagcggatattcctacatgcattggtaccagcagaaacccggccagccacctaaactgctcatctatctcacatccaacctggaatccggagtgccagccaggttcagtggaagcggatccggtaccgacttcaccctgaatatccacccagtcgaagaggaagatgcagctacatattactgccagcattcacgggaactcccatggacctttggaggcgggacaaaactggaaatcaaacggggcgggggcggatctggaggcgggggaagcgggggcggaggtagtcaggtccagctccagcagtcaggccctgacctcgtcaagcccggcgccagtgtcacaattagttgcaaggcttccgggtacgcttttagctcctcatggatgtcctggctcaagcagcgccctggcaaaggcctcgaatggatcggctggatcttcccaagggacggcgatacaaactataatggcaagtttaaaggcaaggccactctcaccgccgacaaatctagctctaccgcatatatgcagctctcttccctgacctcagaagactccgctgtctatttctgcgcccgaaggggagactaccactacgggatggattactggggacagggcaccagcgtgacagtctctagcgaactcaagactccactgggcgataccacccatacagagcctaagagttgtgataccccaccaccctgcccacggtgccctgggggtggaagctccgggggcggttccggtggacagcctcgtgaaccccaggtgtacaccctgccaccttcacgagaagagatgacaaagaatcaggtctcactgacctgtctcgtcaaggggttctacccttcagacattgcagtggagtgggagtcaagcggacagcctgagaataactacaacaccacccctccaatgctggactctgatggaagcttcttcctgtatagcaagctgactgtcgataaatcacgctggcagcagggcaatatcttcagttgctcagtgatgcacgaagctctgcacaatcgtttcactcagaaatctctctctctgtcccctggcaagggcctcggtggcctggtcaacctcactacaaggacccagctccctccagcctacactaactcattcactcggggcgtctactacccagataaagtcttcagaagcagcgttctccattctacccaggacctcttcctgcctttcttttcaaatgtcacttggtttcacgcaatccacgtctccgggaccaacggcacaaaacggttcgataatccagttctccctttcaatgatggggtctatttcgcttctaccgaaaagtctaacatcattcggggttggatctttgggactacactggattccaaaactcagtctctcctcattgtcaataatgcaactaatgttgttattaaggtctgtgagtttcagttctgcaatgacccattcctgggggtctattaccacaagaataacaagtcctggatggagtccgaatttcgagtctattccagcgccaataactgtacattcgaatacgttagtcagcctttcctgatggatctggagggtaaacagggcaacttcaagaacctgcgcgagtttgtttttaagaatattgatgggtatttcaagatatactccaagcatacacctatcaacctggttagagacctgccacagggtttttcagcactggaacctctggtcgacctccctattggaatcaacatcacacgattccagaccctcctcgctctccaccgttcatatctgacccccggcgacagtagctctggctggaccgcaggagcagctgcatactatgtcgggtacctgcagcctaggacattcctgctgaagtacaacgaaaatggaacaatcacagatgctgtggattgtgcactcgatccactgtcagagaccaagtgtactctcaaaagcttcaccgtcgaaaaggggatctaccagacttcaaattttcgggtgcagccaactgagtctatcgtccgatttcctaacatcactaatctctgtccatttggcgaggtgtttaacgctacccgctttgcaagtgtttacgcctggaacaggaagcggatctccaattgcgtggctgattattcagtcctgtacaacagcgccagtttttctacttttaaatgctatggagtgagtcccaccaagctgaatgacctctgcttcactaatgtttatgcagattcatttgtgatccgaggggatgaagtgagacagatcgcccctggacagaccggaaaaattgccgactataactacaaactcccagacgatttcacaggttgcgttatcgcatggaactcaaacaatctggactctaaagtcggcggaaactataactacctctacaggctcttcagaaagtctaacctgaaaccttttgagagggatatcagcacagagatatatcaggctggttccacaccctgtaacggcgttgagggttttaattgttacttccctctccagtcttacggtttccagcctacaaatggagtgggttatcagccctaccgcgtcgtggtgctcagctttgaactgctccatgctcctgcaactgtttgtggtcccaaaaagtccaccaatctggttaagaacaagtgtgtgaactttaactttaatgggctgaccggaacaggcgtcctcacagaaagcaacaaaaaattcctccctttccagcagttcggtagggatatcgccgatacaaccgacgcagtgagggatcctcagacactggaaatcctggacatcacaccatgctccttcggaggcgtttccgttatcacaccaggaactaatacctccaaccaggtggccgtcctctaccaggacgtgaactgtacagaagtgcccgtcgctatccatgctgaccagctgacccccacatggcgggtctactcaactggttctaacgtgttccagacaagggctggctgtctcattggtgccgagcacgtgaacaactcttatgaatgcgacatccctatcggggccggaatttgcgcttcataccagacccagactaatagtccctccggagccggctctgtcgcatcccagtcaatcattgcctatactatgagtctgggtgccgagaacagcgtggcatactctaacaattcaatcgctatccctacaaatttcactatttctgtgacaacagaaatcctgccagtttccatgacaaaaacctcagttgactgcacaatgtacatttgtggagactccaccgagtgcagtaatctcctgctgcagtacggttccttttgcactcagctcaatcgggccctcaccggtatcgctgtcgaacaggataaaaacactcaggaagtttttgcacaggtgaaacagatatataagacacctcccatcaaggactttggtggcttcaacttttcccagatcctgcctgatccaagtaaaccatctaaacggagctttattgaagacctcctgttcaacaaagtgacactggccgatgccggcttcattaagcagtatggagattgcctcggggacatcgcagcccgggacctgatctgtgctcagaagtttaacggtctgactgttctcccccctctgctgactgatgaaatgattgcacagtatacaagcgccctcctcgcaggcaccatcacctcagggtggacctttggtgctggagcagccctgcagatccctttcgccatgcagatggcctacaggttcaatggaatcggggtgacccagaatgtgctctacgaaaatcagaagctcattgccaatcagtttaattccgctattggaaaaatccaggactccctgtcatccacagctagtgctctcggaaaactgcaggacgtggtgaaccagaacgctcaggcactgaacaccctggtcaagcagctctccagtaactttggagccatctcttcagtcctgaacgacattctgtcccgtctcgatccccccgaggctgaagtgcagattgatcggctgattacagggcggctgcagtccctgcagacatacgtgacccagcagctgatccgcgccgcagagattcgggcaagcgccaatctcgccgctaccaagatgtctgagtgcgttctggggcagagtaaacgggtggacttctgtggaaaaggataccatctcatgtcttttcctcagagtgccccccacggcgtggtcttcctgcatgtcacatacgtccctgctcaggaaaagaactttaccacagcaccagctatctgtcatgatggcaaagcccattttccacgagagggtgttttcgtttctaatgggacccactggtttgtgactcagagaaacttttacgaaccacagattatcaccaccgacaatactttcgtcagcggaaactgtgatgtggttatcggaatcgtcaacaataccgtgtacgatcctctgcagccagagctggacagcttcaaggaagaactggataagtactttaagaaccatacttcacccgatgtggacctcggagatatctcaggaattaatgcatccgtcgtcaacatccagaaggagatcgatcggctgaatgaagttgctaaaaatctgaacgaatcactcattgacctccaggaactcggcaaatacgaacagtacatt

SEQ ID NO: 263 - protein, 1609 aa, Artificial sequence
  VB construct
MNFGLRLIFLVLTLKGVQCQVQLTQSPASLAVSLGQRATISCRASKSVSTSGYSYMHWYQQKPGQPPKLLIYLTSNLESGVPARFSGSGSGTDFTLNIHPVEEEDAATYYCQHSRELPWTFGGGTKLEIKRGGGGSGGGGSGGGGSQVQLQQSGPDLVKPGASVTISCKASGYAFSSSWMSWLKQRPGKGLEWIGWIFPRDGDTNYNGKFKGKATLTADKSSSTAYMQLSSLTSEDSAVYFCARRGDYHYGMDYWGQGTSVTVSSELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPSGAGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYI

SEQ ID NO: 264 - DNA, 1500 bp, Artificial sequence
  VB construct
atgcaggtctccactgctgcccttgccgtcctcctctgcaccatggctctctgcaaccaggtcctctctgcaccacttgctgctgacacgccgaccgcctgctgcttcagctacacctcccgacagattccacagaatttcatagctgactactttgagacgagcagccagtgctccaagcccagtgtcatcttcctaaccaagagaggccggcaggtctgtgctgaccccagtgaggagtgggtccagaaatacgtcagtgacctggagctgagtgccgagctcaaaaccccacttggtgacacaactcacacagagcccaaatcttgtgacacacctcccccgtgcccaaggtgcccaggcggtggaagcagcggaggtggaagtggaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaaggcctcggtggcctgcactttgccatcggactggccctctactatccctctgccagaatcgtgtacactgcctgtagccatgccgctgtgggtggcgggggatcaggtggcggagggtcccgagtccagcccacagaatcaatcgtgcggtttccaaacatcaccaatctctgccccttcggagaagtgtttaatgccacacggtttgcctcagtgtatgcctggaaccggaagagaatctccaactgcgtggcagactattctgtgctgtacaacagcgcctccttctctactttcaagtgctacggcgtgtctcctactaagctgaacgatctctgcttcacaaacgtgtatgccgattcctttgtgatcagaggagacgaagtgcgacagattgctcctggacagaccgggaagattgccgactacaactataagctgccagacgatttcactggctgcgttattgcctggaattcaaacaacctcgattccaaagtgggcgggaattataactacctctatagactgttccgtaagtctaacctgaagccattcgagcgtgatattagcaccgaaatctaccaggcaggttcaactccttgtaacggagtggagggattcaactgctacttcccactgcagtcctatgggttccagcctacaaacggagtggggtaccagccttatcgggtcgttgtgctgtcctttgagctgctccacgctcctgcaactgtttgcggacctaagaaatccaccaacctggtgaagaacaagtgcgtgaatttc

SEQ ID NO: 265 - protein, 500 aa, Artificial sequence
  VB construct
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLHFAIGLALYYPSARIVYTACSHAAVGGGGSGGGGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 266 - DNA, 1473 bp, Artificial sequence
  VB construct
atgcaggtctccactgctgcccttgccgtcctcctctgcaccatggctctctgcaaccaggtcctctctgcaccacttgctgctgacacgccgaccgcctgctgcttcagctacacctcccgacagattccacagaatttcatagctgactactttgagacgagcagccagtgctccaagcccagtgtcatcttcctaaccaagagaggccggcaggtctgtgctgaccccagtgaggagtgggtccagaaatacgtcagtgacctggagctgagtgccgagctcaaaaccccacttggtgacacaactcacacagagcccaaatcttgtgacacacctcccccgtgcccaaggtgcccaggcggtggaagcagcggaggtggaagtggaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaaggcctcggtggcctgttcgtgaacgagttctacgcctatctgagaaagcacttctccatgatggggggcggaggtagtggcggaggtggctcccgagtccagcccacagaatcaatcgtgcggtttccaaacatcaccaatctctgccccttcggagaagtgtttaatgccacacggtttgcctcagtgtatgcctggaaccggaagagaatctccaactgcgtggcagactattctgtgctgtacaacagcgcctccttctctactttcaagtgctacggcgtgtctcctactaagctgaacgatctctgcttcacaaacgtgtatgccgattcctttgtgatcagaggagacgaagtgcgacagattgctcctggacagaccgggaagattgccgactacaactataagctgccagacgatttcactggctgcgttattgcctggaattcaaacaacctcgattccaaagtgggcgggaattataactacctctatagactgttccgtaagtctaacctgaagccattcgagcgtgatattagcaccgaaatctaccaggcaggttcaactccttgtaacggagtggagggattcaactgctacttcccactgcagtcctatgggttccagcctacaaacggagtggggtaccagccttatcgggtcgttgtgctgtcctttgagctgctccacgctcctgcaactgtttgcggacctaagaaatccaccaacctggtgaagaacaagtgcgtgaatttc

SEQ ID NO: 267 - protein, 491 aa, Artificial sequence
  VB construct
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLFVNEFYAYLRKHFSMMGGGGSGGGGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 268 - DNA, 1578 bp, Artificial sequence
  VB construct
atgcaggtctccactgctgcccttgccgtcctcctctgcaccatggctctctgcaaccaggtcctctctgcaccacttgctgctgacacgccgaccgcctgctgcttcagctacacctcccgacagattccacagaatttcatagctgactactttgagacgagcagccagtgctccaagcccagtgtcatcttcctaaccaagagaggccggcaggtctgtgctgaccccagtgaggagtgggtccagaaatacgtcagtgacctggagctgagtgccgagctcaaaaccccacttggtgacacaactcacacagagcccaaatcttgtgacacacctcccccgtgcccaaggtgcccaggcggtggaagcagcggaggtggaagtggaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaaggcctcggtggcctgcacttcgcaatcgggctcgcactgtactatcctagtgccaggatcgtgtataccgcttgtagtcacgcagctgtgggcggcggaggttctggcggtgggggatctttcgtgaacgagttctatgcttacctgcgcaaacacttctccatgatgggcggtggagggtctggcgggggaggctcacgggtccagcctacagagtctattgtgcggtttcccaatatcaccaatctgtgcccatttggcgaggtgtttaacgcaaccaggttcgcctctgtctatgcatggaatcggaagcggatcagtaactgcgtcgctgattactctgtcctgtataacagcgcctctttcagcacctttaagtgctacggcgtctctcccaccaagctgaacgacctgtgcttcactaacgtgtacgccgatagctttgtgatccggggcgacgaggtccgccagattgctcccggccagaccggaaagatcgctgactacaactacaaactgcctgacgatttcaccggttgcgtgatcgcttggaatagtaacaatctcgacagcaaagtcggcgggaactataactacctctaccggctctttagaaagagcaacctgaagccttttgaacgggatattagcaccgaaatctaccaggcaggcagcaccccttgcaatggcgtcgagggcttcaactgctatttcccactccagtcttatggcttccagcctaccaatggcgtgggatatcagccatatcgcgtggtggtcctctcctttgagctcctgcatgctccagccaccgtgtgcggtccaaaaaagagcactaacctcgtcaagaacaagtgcgtcaacttt

SEQ ID NO: 269 - protein, 526 aa, Artificial sequence
  VB construct
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLHFAIGLALYYPSARIVYTACSHAAVGGGGSGGGGSFVNEFYAYLRKHFSMMGGGGSGGGGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 270 - DNA, 1653 bp, Artificial sequence
  VB construct
atgcaggtctccactgctgcccttgccgtcctcctctgcaccatggctctctgcaaccaggtcctctctgcaccacttgctgctgacacgccgaccgcctgctgcttcagctacacctcccgacagattccacagaatttcatagctgactactttgagacgagcagccagtgctccaagcccagtgtcatcttcctaaccaagagaggccggcaggtctgtgctgaccccagtgaggagtgggtccagaaatacgtcagtgacctggagctgagtgccgagctcaaaaccccacttggtgacacaactcacacagagcccaaatcttgtgacacacctcccccgtgcccaaggtgcccaggcggtggaagcagcggaggtggaagtggaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaaggcctcggtggcctgcacttcgccattggcctcgccctgtattacccttctgcccggatcgtgtatacagcatgctctcacgctgcagtggggggcggtggctccggcggtggcggaagctttgtgaacgagttctatgcctacctgagaaagcacttctctatgatgggtggcggaggctccggagggggtggctctctggtgaagccttctttctacgtctactccagagtgaagaatctcggaggtggcggttctggtggcggaggttctagggtccagccaaccgaatccatcgtgcgattccccaatatcacaaacctgtgcccttttggggaggtgttcaatgctaccagatttgcctcagtgtacgcatggaacagaaagaggattagtaactgcgtggccgactatagcgtgctgtataacagcgccagcttctctaccttcaagtgctacggcgtgtctcctaccaagctcaacgatctctgcttcaccaacgtgtatgccgattccttcgtcattaggggcgatgaggtgcgacagatcgcccctggacagaccggaaagatcgccgattataactacaaactgcctgatgacttcaccggttgcgtgatcgcctggaactccaacaacctggactccaaggtcggtggcaactataattacctgtacaggctgttcaggaagtccaatctgaagccttttgaacgagatatcagcaccgaaatctaccaggccggatctaccccatgcaacggtgtcgaaggctttaactgttactttcctctccagagttatggcttccagccaaccaatggggtgggctatcagccttatcgagtggtcgtgctgtcctttgaactcctgcatgccccagctactgtctgtggccccaagaaatccaccaatctcgtgaaaaacaagtgcgtcaatttc

SEQ ID NO: 271 - protein, 551 aa, Artificial sequence
  VB construct
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLHFAIGLALYYPSARIVYTACSHAAVGGGGSGGGGSFVNEFYAYLRKHFSMMGGGGSGGGGSLVKPSFYVYSRVKNLGGGGSGGGGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 272 - DNA, 1485 bp, Artificial sequence
  VB construct
atgcaggtctccactgctgcccttgccgtcctcctctgcaccatggctctctgcaaccaggtcctctctgcaccacttgctgctgacacgccgaccgcctgctgcttcagctacacctcccgacagattccacagaatttcatagctgactactttgagacgagcagccagtgctccaagcccagtgtcatcttcctaaccaagagaggccggcaggtctgtgctgaccccagtgaggagtgggtccagaaatacgtcagtgacctggagctgagtgccgagctcaaaaccccacttggtgacacaactcacacagagcccaaatcttgtgacacacctcccccgtgcccaaggtgcccaggcggtggaagcagcggaggtggaagtggaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaaggcctcggtggcctgcactttgccatcggactggccctgtactatccttctgccagaatcgtgtacaccgcctgttctcatgccgctgtgggtctgggcggactgcgagtccagcccacagaatcaatcgtgcggtttccaaacatcaccaatctctgccccttcggagaagtgtttaatgccacacggtttgcctcagtgtatgcctggaaccggaagagaatctccaactgcgtggcagactattctgtgctgtacaacagcgcctccttctctactttcaagtgctacggcgtgtctcctactaagctgaacgatctctgcttcacaaacgtgtatgccgattcctttgtgatcagaggagacgaagtgcgacagattgctcctggacagaccgggaagattgccgactacaactataagctgccagacgatttcactggctgcgttattgcctggaattcaaacaacctcgattccaaagtgggcgggaattataactacctctatagactgttccgtaagtctaacctgaagccattcgagcgtgatattagcaccgaaatctaccaggcaggttcaactccttgtaacggagtggagggattcaactgctacttcccactgcagtcctatgggttccagcctacaaacggagtggggtaccagccttatcgggtcgttgtgctgtcctttgagctgctccacgctcctgcaactgtttgcggacctaagaaatccaccaacctggtgaagaacaagtgcgtgaatttc

SEQ ID NO: 273 - protein, 495 aa, Artificial sequence
  VB construct
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLHFAIGLALYYPSARIVYTACSHAAVGLGGLRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 274 - DNA, 1500 bp, Artificial sequence
  VB construct
atgcaggtctccactgctgcccttgccgtcctcctctgcaccatggctctctgcaaccaggtcctctctgcaccacttgctgctgacacgccgaccgcctgctgcttcagctacacctcccgacagattccacagaatttcatagctgactactttgagacgagcagccagtgctccaagcccagtgtcatcttcctaaccaagagaggccggcaggtctgtgctgaccccagtgaggagtgggtccagaaatacgtcagtgacctggagctgagtgccgagctcaaaaccccacttggtgacacaactcacacagagcccaaatcttgtgacacacctcccccgtgcccaaggtgcccaggcggtggaagcagcggaggtggaagtggaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaaggcctcggtggcctgcacttcgccattggcctggccctgtactatccctccgccagaatcgtgtacaccgcttgtagtcacgccgcagtggggctcggaggcctgggtctcggcggactgcgagtccagcccacagaatcaatcgtgcggtttccaaacatcaccaatctctgccccttcggagaagtgtttaatgccacacggtttgcctcagtgtatgcctggaaccggaagagaatctccaactgcgtggcagactattctgtgctgtacaacagcgcctccttctctactttcaagtgctacggcgtgtctcctactaagctgaacgatctctgcttcacaaacgtgtatgccgattcctttgtgatcagaggagacgaagtgcgacagattgctcctggacagaccgggaagattgccgactacaactataagctgccagacgatttcactggctgcgttattgcctggaattcaaacaacctcgattccaaagtgggcgggaattataactacctctatagactgttccgtaagtctaacctgaagccattcgagcgtgatattagcaccgaaatctaccaggcaggttcaactccttgtaacggagtggagggattcaactgctacttcccactgcagtcctatgggttccagcctacaaacggagtggggtaccagccttatcgggtcgttgtgctgtcctttgagctgctccacgctcctgcaactgtttgcggacctaagaaatccaccaacctggtgaagaacaagtgcgtgaatttc

SEQ ID NO: 275 - protein, 500 aa, Artificial sequence
  VB construct
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLHFAIGLALYYPSARIVYTACSHAAVGLGGLGLGGLRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 276 - DNA, 1458 bp, Artificial sequence
  VB construct
atgcaggtctccactgctgcccttgccgtcctcctctgcaccatggctctctgcaaccaggtcctctctgcaccacttgctgctgacacgccgaccgcctgctgcttcagctacacctcccgacagattccacagaatttcatagctgactactttgagacgagcagccagtgctccaagcccagtgtcatcttcctaaccaagagaggccggcaggtctgtgctgaccccagtgaggagtgggtccagaaatacgtcagtgacctggagctgagtgccgagctcaaaaccccacttggtgacacaactcacacagagcccaaatcttgtgacacacctcccccgtgcccaaggtgcccaggcggtggaagcagcggaggtggaagtggaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaaggcctcggtggcctgttcgtgaacgagttctacgcctatctgagaaagcacttcagcatgatgggactgggcggtctgcgagtccagcccacagaatcaatcgtgcggtttccaaacatcaccaatctctgccccttcggagaagtgtttaatgccacacggtttgcctcagtgtatgcctggaaccggaagagaatctccaactgcgtggcagactattctgtgctgtacaacagcgcctccttctctactttcaagtgctacggcgtgtctcctactaagctgaacgatctctgcttcacaaacgtgtatgccgattcctttgtgatcagaggagacgaagtgcgacagattgctcctggacagaccgggaagattgccgactacaactataagctgccagacgatttcactggctgcgttattgcctggaattcaaacaacctcgattccaaagtgggcgggaattataactacctctatagactgttccgtaagtctaacctgaagccattcgagcgtgatattagcaccgaaatctaccaggcaggttcaactccttgtaacggagtggagggattcaactgctacttcccactgcagtcctatgggttccagcctacaaacggagtggggtaccagccttatcgggtcgttgtgctgtcctttgagctgctccacgctcctgcaactgtttgcggacctaagaaatccaccaacctggtgaagaacaagtgcgtgaatttc

SEQ ID NO: 277 - protein, 486 aa, Artificial sequence
  VB construct
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLFVNEFYAYLRKHFSMMGLGGLRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 278 - DNA, 1563 bp, Artificial sequence
  VB construct
atgcaggtctccactgctgcccttgccgtcctcctctgcaccatggctctctgcaaccaggtcctctctgcaccacttgctgctgacacgccgaccgcctgctgcttcagctacacctcccgacagattccacagaatttcatagctgactactttgagacgagcagccagtgctccaagcccagtgtcatcttcctaaccaagagaggccggcaggtctgtgctgaccccagtgaggagtgggtccagaaatacgtcagtgacctggagctgagtgccgagctcaaaaccccacttggtgacacaactcacacagagcccaaatcttgtgacacacctcccccgtgcccaaggtgcccaggcggtggaagcagcggaggtggaagtggaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaaggcctcggtggcctgcattttgccattggactggcactctattacccaagtgccaggatcgtctacactgcctgcagtcacgcagccgtcgggggaggcggttctggagggggtggctctttcgtgaacgagttttacgcctacctgaggaagcactttagcatgatgggcctcgggggtctccgcgtgcagccaacagaatccattgtccgcttccccaatattacaaacctgtgcccattcggagaagtgtttaatgccaccagattcgcctccgtgtatgcttggaacagaaagcgcatctctaactgcgtcgccgactacagtgtgctgtacaacagcgcaagtttttccacattcaagtgttatggggtgtcaccaaccaaactgaacgatctctgcttcacaaacgtgtacgccgactccttcgtcatccgcggcgacgaagtgagacagatcgcacccgggcagactggaaagatcgcagactataactacaagctgccagacgatttcaccgggtgcgtcattgcctggaacagcaataacctcgacagcaaagtggggggcaattacaactatctgtatagactgttccgcaagtccaacctgaaaccctttgagagagacatcagtacagaaatctatcaggccggttctacaccatgtaatggtgtcgaagggttcaactgctacttccctctgcagtcatatggcttccagccaactaatggggtgggataccagccttatagggtggtcgtgctgagctttgagctgctgcacgcacccgccaccgtgtgcgggccaaaaaagtccactaacctggtgaagaacaagtgcgtcaatttc

SEQ ID NO: 279 - protein, 521 aa, Artificial sequence
  VB construct
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLHFAIGLALYYPSARIVYTACSHAAVGGGGSGGGGSFVNEFYAYLRKHFSMMGLGGLRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 280 - DNA, 1638 bp, Artificial sequence
  VB construct
atgcaggtctccactgctgcccttgccgtcctcctctgcaccatggctctctgcaaccaggtcctctctgcaccacttgctgctgacacgccgaccgcctgctgcttcagctacacctcccgacagattccacagaatttcatagctgactactttgagacgagcagccagtgctccaagcccagtgtcatcttcctaaccaagagaggccggcaggtctgtgctgaccccagtgaggagtgggtccagaaatacgtcagtgacctggagctgagtgccgagctcaaaaccccacttggtgacacaactcacacagagcccaaatcttgtgacacacctcccccgtgcccaaggtgcccaggcggtggaagcagcggaggtggaagtggaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaaggcctcggtggcctgcacttcgcaatcggactcgcactgtattacccctcagccaggatcgtgtatactgcatgcagtcacgctgccgtcggcggcgggggtagtgggggaggcggatcctttgtgaatgagttctatgcctacctgagaaagcacttcagcatgatgggcggtgggggaagcggcggtggaggatccctcgtgaaaccctccttctatgtctactcacgggtgaagaacctgggcctcggaggtctcagagtccagcctaccgagtctatcgtgaggtttccaaacatcactaatctgtgccctttcggagaagtgtttaacgccacccggtttgcctcagtgtacgcttggaataggaagcggatctcaaattgcgtggcagattacagcgtgctgtacaactccgcctcattcagtacattcaagtgctatggcgtgagcccaaccaagctgaacgatctgtgcttcacaaacgtgtacgcagattccttcgtcatcagaggggatgaagtcagacagatcgctccaggccagacaggaaagatcgcagattataattataaactccctgacgatttcaccggatgcgtgatcgcctggaacagtaataacctggattcaaaagtgggcggtaactataattacctgtacagactgttcaggaagagcaacctgaagccatttgaacgcgacatttccaccgaaatctaccaggcaggaagcacaccctgtaatggcgtggagggctttaactgctatttccccctgcagtcctatggttttcagcccacaaatggggtggggtaccagccctaccgggtcgtggtgctgagcttcgaactcctgcacgcaccagccactgtctgtggacccaagaaatccactaacctcgtgaagaacaagtgcgtgaacttc

SEQ ID NO: 281 - protein, 546 aa, Artificial sequence
  VB construct
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLHFAIGLALYYPSARIVYTACSHAAVGGGGSGGGGSFVNEFYAYLRKHFSMMGGGGSGGGGSLVKPSFYVYSRVKNLGLGGLRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 282 - DNA, 1518 bp, Artificial sequence
  VB construct
atgcaggtctccactgctgcccttgccgtcctcctctgcaccatggctctctgcaaccaggtcctctctgcaccacttgctgctgacacgccgaccgcctgctgcttcagctacacctcccgacagattccacagaatttcatagctgactactttgagacgagcagccagtgctccaagcccagtgtcatcttcctaaccaagagaggccggcaggtctgtgctgaccccagtgaggagtgggtccagaaatacgtcagtgacctggagctgagtgccgagctcaaaaccccacttggtgacacaactcacacagagcccaaatcttgtgacacacctcccccgtgcccaaggtgcccaggcggtggaagcagcggaggtggaagtggaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaaggcctcggtggcctgcacttcgctatcggactggccctctactatccttccgctcggattgtctacactgcctgcagccatgctgcagtgacccagaaatcactgagcctcagccctggcaaaggtctgggaggcctcagagtccagcctactgagagcatcgtgcgcttcccaaacatcacaaacctgtgcccttttggagaagtcttcaatgccactcggtttgcttcagtctatgcttggaataggaagcggattagcaattgcgtggccgactactcagtgctgtacaatagcgcaagtttcagcaccttcaagtgctatggagtctccccaaccaaactcaatgacctgtgcttcaccaacgtgtacgctgatagctttgtgatcagaggtgacgaagtgcgccagattgcccctggtcagaccggaaagattgccgactacaattacaaactgccagacgatttcacaggatgcgtgatcgcttggaatagcaacaacctggactctaaagtggggggcaactacaattacctgtaccggctctttcgcaagtctaacctgaagccttttgagagagacattagcaccgaaatctaccaggctggaagcacaccatgcaacggagtggagggcttcaattgctacttccctctccagagctatggttttcagcccactaacggtgtcggttaccagccatacagagtggtcgtgctgtcatttgagctgctccatgcccccgctacagtctgtggacctaaaaagagcaccaatctggtgaagaacaaatgcgtcaacttc

SEQ ID NO: 283 - protein, 506 aa, Artificial sequence
  VB construct
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLHFAIGLALYYPSARIVYTACSHAAVTQKSLSLSPGKGLGGLRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 284 - DNA, 1671 bp, Artificial sequence
  VB construct
atgcaggtctccactgctgcccttgccgtcctcctctgcaccatggctctctgcaaccaggtcctctctgcaccacttgctgctgacacgccgaccgcctgctgcttcagctacacctcccgacagattccacagaatttcatagctgactactttgagacgagcagccagtgctccaagcccagtgtcatcttcctaaccaagagaggccggcaggtctgtgctgaccccagtgaggagtgggtccagaaatacgtcagtgacctggagctgagtgccgagctcaaaaccccacttggtgacacaactcacacagagcccaaatcttgtgacacacctcccccgtgcccaaggtgcccaggcggtggaagcagcggaggtggaagtggaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaaggcctcggtggcctgcatttcgccattggtctggcactctactacccatcagccagaatcgtgtacaccgcctgctcccatgctgcagtgggtggagggggaagcggaggcgggggatctttcgtcaacgaattctacgcctacctgcggaagcacttcagcatgatgggcggcgggggcagcggagggggtgggtcactcgtgaagccaagcttctacgtctactctagagtgaagaacctgacccagaagtcactgtccctgtcacctggcaagggtctcggaggcctcagggtgcagccaaccgaaagcattgtgcgatttcccaacatcactaatctctgtccctttggcgaagtgttcaacgcaacccggtttgcatcagtgtatgcctggaatcggaagaggatctccaactgcgtggccgattacagcgtgctgtataattcagccagcttctccactttcaagtgctacggggtctctcctaccaaactcaacgacctctgctttaccaatgtctacgctgacagctttgtgatccggggcgatgaagtccggcagattgcaccaggacagaccgggaaaatcgccgattacaactataagctgcccgacgatttcacaggctgcgtgatcgcctggaattctaacaatctggatagcaaggtgggcggaaattacaactatctgtaccgactgttccggaagtcaaatctgaagccctttgaacgggatatctccaccgaaatctatcaggccggaagtactccctgcaacggggtggaagggttcaactgttactttcccctccagtcttacggattccagcctaccaacggagtgggctaccagccctatagagtggtcgtgctgtcctttgagctgctgcacgccccagctacagtgtgcggccctaagaagtcaaccaacctcgtgaaaaataagtgcgtcaatttt

SEQ ID NO: 285 - protein, 557 aa, Artificial sequence
  VB construct
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLHFAIGLALYYPSARIVYTACSHAAVGGGGSGGGGSFVNEFYAYLRKHFSMMGGGGSGGGGSLVKPSFYVYSRVKNLTQKSLSLSPGKGLGGLRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 286 - DNA, 1509 bp, Artificial sequence
  VB construct
atgcaggtctccactgctgcccttgccgtcctcctctgcaccatggctctctgcaaccaggtcctctctgcaccacttgctgctgacacgccgaccgcctgctgcttcagctacacctcccgacagattccacagaatttcatagctgactactttgagacgagcagccagtgctccaagcccagtgtcatcttcctaaccaagagaggccggcaggtctgtgctgaccccagtgaggagtgggtccagaaatacgtcagtgacctggagctgagtgccgagctcaaaaccccacttggtgacacaactcacacagagcccaaatcttgtgacacacctcccccgtgcccaaggtgcccaggcggtggaagcagcggaggtggaagtggaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaaggcctcggtggcctgcactttgccatcggcctggccctgtactatccctctgccagaatcgtgtacactgcctgctctcatgctgccgtgagcctgtctctgagccctggcaaaggactgggtggcctccgagtccagcccacagaatcaatcgtgcggtttccaaacatcaccaatctctgccccttcggagaagtgtttaatgccacacggtttgcctcagtgtatgcctggaaccggaagagaatctccaactgcgtggcagactattctgtgctgtacaacagcgcctccttctctactttcaagtgctacggcgtgtctcctactaagctgaacgatctctgcttcacaaacgtgtatgccgattcctttgtgatcagaggagacgaagtgcgacagattgctcctggacagaccgggaagattgccgactacaactataagctgccagacgatttcactggctgcgttattgcctggaattcaaacaacctcgattccaaagtgggcgggaattataactacctctatagactgttccgtaagtctaacctgaagccattcgagcgtgatattagcaccgaaatctaccaggcaggttcaactccttgtaacggagtggagggattcaactgctacttcccactgcagtcctatgggttccagcctacaaacggagtggggtaccagccttatcgggtcgttgtgctgtcctttgagctgctccacgctcctgcaactgtttgcggacctaagaaatccaccaacctggtgaagaacaagtgcgtgaatttc

SEQ ID NO: 287 - protein, 503 aa, Artificial sequence
  VB construct
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLHFAIGLALYYPSARIVYTACSHAAVSLSLSPGKGLGGLRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 288 - DNA, 1662 bp, Artificial sequence
  VB construct
atgcaggtctccactgctgcccttgccgtcctcctctgcaccatggctctctgcaaccaggtcctctctgcaccacttgctgctgacacgccgaccgcctgctgcttcagctacacctcccgacagattccacagaatttcatagctgactactttgagacgagcagccagtgctccaagcccagtgtcatcttcctaaccaagagaggccggcaggtctgtgctgaccccagtgaggagtgggtccagaaatacgtcagtgacctggagctgagtgccgagctcaaaaccccacttggtgacacaactcacacagagcccaaatcttgtgacacacctcccccgtgcccaaggtgcccaggcggtggaagcagcggaggtggaagtggaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaaggcctcggtggcctgcacttcgccattgggctggctctgtattacccctcagccagaatcgtctataccgcatgctcccatgcagctgtcgggggtggcggtagtggggggggcggtagcttcgtcaatgagttctacgcctacctgaggaaacatttctccatgatgggtggcgggggaagtggcgggggtggcagtctggtgaagccctccttctacgtgtattctcgagtgaagaatctctccctcagtctgtcccctggcaaaggtctggggggcctcagggtgcagcccaccgagtccatcgtcaggttccccaacatcacaaacctgtgcccctttggggaggtcttcaacgccactcgctttgcttctgtctacgcttggaacaggaagcgaatcagtaactgcgtcgccgattactccgtgctgtataattccgcctcattcagcaccttcaagtgctacggcgtgtctcctaccaagctcaacgatctctgcttcaccaatgtctacgcagacagtttcgtgatcagaggcgatgaggtccgccagattgccccagggcagaccggaaaaatcgccgactataattacaagctgcctgatgacttcaccgggtgcgtgatcgcatggaattccaacaacctcgactccaaagtgggagggaactacaactatctgtaccgcctgttcagaaagagcaacctgaaaccttttgagagggatatctcaactgaaatctatcaggccgggtccaccccatgtaatggggtggaagggttcaactgctatttccctctgcagtcctacgggttccagcctaccaatggagtcggctaccagccctacagagtcgtggtgctgtctttcgaactcctgcacgcccctgccaccgtctgcggcccaaagaaatccaccaatctggtgaaaaataagtgcgtcaacttt

SEQ ID NO: 289 - protein, 554 aa, Artificial sequence
  VB construct
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLHFAIGLALYYPSARIVYTACSHAAVGGGGSGGGGSFVNEFYAYLRKHFSMMGGGGSGGGGSLVKPSFYVYSRVKNLSLSLSPGKGLGGLRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 290 - DNA, 1731 bp, Artificial sequence
  VB construct
atgcaggtctccactgctgcccttgccgtcctcctctgcaccatggctctctgcaaccaggtcctctctgcaccacttgctgctgacacgccgaccgcctgctgcttcagctacacctcccgacagattccacagaatttcatagctgactactttgagacgagcagccagtgctccaagcccagtgtcatcttcctaaccaagagaggccggcaggtctgtgctgaccccagtgaggagtgggtccagaaatacgtcagtgacctggagctgagtgccgagctcaaaaccccacttggtgacacaactcacacagagcccaaatcttgtgacacacctcccccgtgcccaaggtgcccaggcggtggaagcagcggaggtggaagtggaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaaggcctcggtggcctgcactttgcaatcggactcgccctgtactaccctagcgccaggattgtctataccgcatgcagccacgccgctgtggggggaggcggtagcgggggaggcggtagttttgtcaacgagttctacgcatatctgcggaagcacttcagcatgatgggagggggtggcagcggagggggaggttctctggtcaagcctagcttttacgtgtacagtcgagtcaagaacctcggtggatcagccggtgggtcaggatctggctctagcggtggaagcagtggtgcatctggaaccggtacagctggcggtactggatctggaagcgggaccggaagcgggagagtgcagcccacagagtcaatcgtgcggtttcctaacatcacaaacctctgcccatttggggaggtgttcaacgccaccaggtttgcttccgtctatgcctggaataggaagcgcattagcaattgcgtggccgactattcagtgctgtacaactctgcttccttcagcacattcaagtgttacggcgtgagccccaccaagctcaatgacctgtgctttaccaacgtgtatgccgactctttcgtgatcaggggggacgaggtccgacagattgcccctggccagacagggaagattgctgactataactacaaactgcccgacgatttcactggctgcgtcatcgcatggaacagcaacaatctggacagcaaagtgggcgggaattacaattacctgtataggctcttccgcaaaagtaatctgaagcccttcgagagggatatcagcactgaaatctatcaggccggctcaactccttgcaatggagtcgaagggttcaactgctatttccctctgcagagctatggttttcagcccactaacggggtgggatatcagccatatcgggtcgtggtgctgtcattcgagctgctgcatgcacctgccacagtgtgcggacctaagaagagcactaacctggtcaagaacaagtgcgtgaatttt

SEQ ID NO: 291 - protein, 577 aa, Artificial sequence
  VB construct
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLHFAIGLALYYPSARIVYTACSHAAVGGGGSGGGGSFVNEFYAYLRKHFSMMGGGGSGGGGSLVKPSFYVYSRVKNLGGSAGGSGSGSSGGSSGASGTGTAGGTGSGSGTGSGRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 292 - DNA, 1731 bp, Artificial sequence
  VB construct
atgcaggtctccactgctgcccttgccgtcctcctctgcaccatggctctctgcaaccaggtcctctctgcaccacttgctgctgacacgccgaccgcctgctgcttcagctacacctcccgacagattccacagaatttcatagctgactactttgagacgagcagccagtgctccaagcccagtgtcatcttcctaaccaagagaggccggcaggtctgtgctgaccccagtgaggagtgggtccagaaatacgtcagtgacctggagctgagtgccgagctcaaaaccccacttggtgacacaactcacacagagcccaaatcttgtgacacacctcccccgtgcccaaggtgcccaggcggtggaagcagcggaggtggaagtggaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaaggcctcggtggcctgcatttcgctattggcctggctctctactacccaagcgcaaggattgtctacaccgcctgtagccatgcagccgtcggtgggggcgggagtgggggcggcgggagctttgtgaacgaattctacgcctatctgaggaagcactttagcatgatgggcggcgggggaagcggcggggggggatccctggtcaaacctagcttttacgtgtactcacgggtcaagaatctgggcgggtctggagggggctccgaaggtggagggagcgagggaggcgggagcgaagggggcgggtccgagggtgggggcagcgagggagggggcagcggcggcggaagtagggtccagcccacagagtccattgtgaggttcccaaacatcaccaatctctgccctttcggcgaagtcttcaacgccactaggtttgccagtgtctacgcttggaatagaaagcgcattagcaactgcgtggccgattactccgtcctgtacaatagcgccagcttttccaccttcaagtgctacggagtgagccctaccaagctgaacgacctctgtttcacaaacgtgtacgccgatagcttcgtgattagaggagacgaggtgcggcagattgcccctgggcagaccggcaagatcgccgactacaattacaagctgcccgacgactttaccggctgcgtcatcgcctggaatagcaacaacctcgacagtaaagtggggggcaattataactacctctatagactgttccgaaagagcaatctgaagccatttgagcgggatatcagcaccgaaatctaccaggctggaagcaccccatgcaacggggtggaagggttcaactgctactttccactgcagagctacggcttccagcccactaatggagtgggctatcagccctatagagtggtggtcctcagcttcgaactgctgcatgctcccgccaccgtgtgcgggcctaagaaatccaccaatctggtgaagaacaagtgcgtgaatttc

SEQ ID NO: 293 - protein, 577 aa, Artificial sequence
  VB construct
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLHFAIGLALYYPSARIVYTACSHAAVGGGGSGGGGSFVNEFYAYLRKHFSMMGGGGSGGGGSLVKPSFYVYSRVKNLGGSGGGSEGGGSEGGGSEGGGSEGGGSEGGGSGGGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 294 - DNA, 1395 bp, Artificial sequence
  VB construct
atgcaggtctccactgctgcccttgccgtcctcctctgcaccatggctctctgcaaccaggtcctctctgcaccacttgctgctgacacgccgaccgcctgctgcttcagctacacctcccgacagattccacagaatttcatagctgactactttgagacgagcagccagtgctccaagcccagtgtcatcttcctaaccaagagaggccggcaggtctgtgctgaccccagtgaggagtgggtccagaaatacgtcagtgacctggagctgagtgccgagctcaaaaccccacttggtgacacaactcacacagagcccaaatcttgtgacacacctcccccgtgcccaaggtgcccaggcggtggaagcagcggaggtggaagtggaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaaggcctcggtggcctgcgagtccagcccacagaatcaatcgtgcggtttccaaacatcaccaatctctgccccttcggagaagtgtttaatgccacacggtttgcctcagtgtatgcctggaaccggaagagaatctccaactgcgtggcagactattctgtgctgtacaacagcgcctccttctctactttcaagtgctacggcgtgtctcctactaagctgaacgatctctgcttcacaaacgtgtatgccgattcctttgtgatcagaggagacgaagtgcgacagattgctcctggacagaccgggaacattgccgactacaactataagctgccagacgatttcactggctgcgttattgcctggaattcaaacaacctcgattccaaagtgggcgggaattataactacctctatagactgttccgtaagtctaacctgaagccattcgagcgtgatattagcaccgaaatctaccaggcaggttcaactccttgtaacggagtgaagggattcaactgctacttcccactgcagtcctatgggttccagcctacatatggagtggggtaccagccttatcgggtcgttgtgctgtcctttgagctgctccacgctcctgcaactgtttgcggacctaagaaatccaccaacctggtgaagaacaagtgcgtgaatttc

SEQ ID NO: 295 - protein, 465 aa, Artificial sequence
  VB construct
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNF

SEQ ID NO: 296 - protein, 724 aa, Artificial sequence
  VB construct
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGGSGGGSEGGGSEGGGSEGGGSEGGGSEGGGSGGGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNF

SEQ ID NO: 297 - protein, 724 aa, Artificial sequence
  VB construct
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGGSAGGSGSGSSGGSSGASGTGTAGGTGSGSGTGSGRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNF

SEQ ID NO: 298 - protein, 704 aa, Artificial sequence
  VB construct
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFTQKSLSLSPGKGLGGLRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNF

SEQ ID NO: 299 - protein, 701 aa, Artificial sequence
  VB construct
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFSLSLSPGKGLGGLRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNF

SEQ ID NO: 300 - protein, 724 aa, Artificial sequence
  VB construct
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFGGSGGGSEGGGSEGGGSEGGGSEGGGSEGGGSGGGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNF

SEQ ID NO: 301 - protein, 724 aa, Artificial sequence
  VB construct
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFGGSAGGSGSGSSGGSSGASGTGTAGGTGSGSGTGSGRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNF

SEQ ID NO: 302 - protein, 724 aa, Artificial sequence
  VB construct
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFGGSGGGSEGGGSEGGGSEGGGSEGGGSEGGGSGGGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 303 - protein, 724 aa, Artificial sequence
  VB construct
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFGGSAGGSGSGSSGGSSGASGTGTAGGTGSGSGTGSGRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 304 - protein, 15 aa, Artificial sequence
  betacoronavirus epitope
AWNRKRISNCVADYS

SEQ ID NO: 305 - protein, 15 aa, Artificial sequence
  betacoronavirus epitope
RKRISNCVADYSVLY

SEQ ID NO: 306 - protein, 15 aa, Artificial sequence
  betacoronavirus epitope
SFSTFKCYGVSPTKL

SEQ ID NO: 307 - protein, 15 aa, Artificial sequence
  betacoronavirus epitope
TFKCYGVSPTKLNDL

SEQ ID NO: 308 - protein, 15 aa, Artificial sequence
  betacoronavirus epitope
CYGVSPTKLNDLCFT

SEQ ID NO: 309 - protein, 15 aa, Artificial sequence
  betacoronavirus epitope
VSPTKLNDLCFTNVY

SEQ ID NO: 310 - protein, 15 aa, Artificial sequence
  betacoronavirus epitope
TKLNDLCFTNVYADS

SEQ ID NO: 311 - protein, 15 aa, Artificial sequence
  betacoronavirus epitope
VGGNYNYLYRLFRKS

SEQ ID NO: 312 - protein, 15 aa, Artificial sequence
  betacoronavirus epitope
NYNYLYRLFRKSNLK

SEQ ID NO: 313 - protein, 15 aa, Artificial sequence
  betacoronavirus epitope
RDISTEIYQAGSTPC

SEQ ID NO: 314 - protein, 15 aa, Artificial sequence
  betacoronavirus epitope
STEIYQAGSTPCNGV

SEQ ID NO: 315 - protein, 15 aa, Artificial sequence
  betacoronavirus epitope
IYQAGSTPCNGVEGF

SEQ ID NO: 316 - protein, 15 aa, Artificial sequence
  betacoronavirus epitope
EGFNCYFPLQSYGFQ

SEQ ID NO: 317 - protein, 15 aa, Artificial sequence
  betacoronavirus epitope
NCYFPLQSYGFQPTN

SEQ ID NO: 318 - protein, 15 aa, Artificial sequence
  betacoronavirus epitope
PTNGVGYQPYRVVVL

SEQ ID NO: 319 - protein, 15 aa, Artificial sequence
  betacoronavirus epitope
GVGYQPYRVVVLSFE

SEQ ID NO: 320 - protein, 15 aa, Artificial sequence
  betacoronavirus epitope
YQPYRVVVLSFELLH

SEQ ID NO: 321 - protein, 260 aa, Artificial sequence
  Pan Hla targeting sequence
MNFGLRLIFLVLTLKGVQCDIQMTQTTSSLSASLGDRVTISCSASQDINNYLNWYQQKPDGTVKLLIYYTSSLHSGVPSRFSGSGSGTDYSLTISNLEPEDIATYYCQQYSKFPRTFGGGTKLEIKRGGGGSGGGGSGGGGSQIQLVQSGPELKKPGETVKISCKASGYTFINYGMNWVKQTPGKGLKWMGWINTYSGEPTYPDDFKGRFAFSLETSASTAYLQLNNLKNEDMATYFCARGDYYGPFDNWGQGTTLTVSS

SEQ ID NO: 322 - protein, 20 aa, Artificial sequence
  betacoronavirus epitope
DLFMRIFTIGTVTLKQGEIK

SEQ ID NO: 323 - protein, 40 aa, Artificial sequence
  betacoronavirus epitope
TIPIQASLPFGWLIVGVALLAVFQSASKIITLKKRWQLAL

SEQ ID NO: 324 - protein, 24 aa, Artificial sequence
  betacoronavirus epitope
VAAGLEAPFLYLYALVYFLQSINF

SEQ ID NO: 325 - protein, 35 aa, Artificial sequence
  betacoronavirus epitope
IPYNSVTSSIVITSGDGTTSPISEHDYQIGGYTEK

SEQ ID NO: 326 - protein, 18 aa, Artificial sequence
  betacoronavirus epitope
VLHSYFTSDYYQLYSTQL

SEQ ID NO: 327 - protein, 16 aa, Artificial sequence
  betacoronavirus epitope
STDTGVEHVTFFIYNK

SEQ ID NO: 328 - protein, 30 aa, Artificial sequence
  betacoronavirus epitope
LPNNTASWFTALTQHGKEDLKFPRGQGVPI

SEQ ID NO: 329 - protein, 14 aa, Artificial sequence
  betacoronavirus epitope
KMKDLSPRWYFYYL

SEQ ID NO: 330 - protein, 19 aa, Artificial sequence
  betacoronavirus epitope
NAAIVLQLPQGTTLPKGFY

SEQ ID NO: 331 - protein, 15 aa, Artificial sequence
  betacoronavirus epitope
DAALALLLLDRLNQL

SEQ ID NO: 332 - protein, 26 aa, Artificial sequence
  betacoronavirus epitope
KSAAEASKKPRQKRTATKAYNVTQAF

SEQ ID NO: 333 - protein, 37 aa, Artificial sequence
  betacoronavirus epitope
DYKHWPQIAQFAPSASAFFGMSRIGMEVTPSGTWLTY

SEQ ID NO: 334 - protein, 16 aa, Artificial sequence
  betacoronavirus epitope
KLDDKDPNFKDQVILL

SEQ ID NO: 335 - protein, 26 aa, Artificial sequence
  betacoronavirus epitope
NFKDQVILLNKHIDAYKTFPPTEPKK

SEQ ID NO: 336 - protein, 33 aa, Artificial sequence
  betacoronavirus epitope
TQALPQRQKKQQTVTLLPAADLDDFSKQLQQSM

SEQ ID NO: 337 - protein, 14 aa, Artificial sequence
  betacoronavirus epitope
LLMPLKAPKEIIFL

SEQ ID NO: 338 - protein, 19 aa, Artificial sequence
  betacoronavirus epitope
FGDDTVIEVQGYKSVNITF

SEQ ID NO: 339 - protein, 25 aa, Artificial sequence
  betacoronavirus epitope
VYLAVFDKNLYDKLVSSFLEMKSEK

SEQ ID NO: 340 - protein, 29 aa, Artificial sequence
  betacoronavirus epitope
KIKPHNSHEGKTFYVLPNDDTLRVEAFEY

SEQ ID NO: 341 - protein, 19 aa, Artificial sequence
  betacoronavirus epitope
TTEVVGDIILKPANNSLKI

SEQ ID NO: 342 - protein, 12 aa, Artificial sequence
  betacoronavirus epitope
TNYMPYFFTLLL

SEQ ID NO: 343 - protein, 24 aa, Artificial sequence
  betacoronavirus epitope
LVQMAPISAMVRMYIFFASFYYVW

SEQ ID NO: 344 - protein, 33 aa, Artificial sequence
  betacoronavirus epitope
RPINPTDQSSYIVDSVTVKNGSIHLYFDKAGQK

SEQ ID NO: 345 - protein, 32 aa, Artificial sequence
  betacoronavirus epitope
DSAEVAVKMFDAYVNTFSSTFNVPMEKLKTLV

SEQ ID NO: 346 - protein, 29 aa, Artificial sequence
  betacoronavirus epitope
FLHFLPRVFSAVGNICYTPSKLIEYTDFA

SEQ ID NO: 347 - protein, 31 aa, Artificial sequence
  betacoronavirus epitope
YESLRPDTRYVLMDGSIIQFPNTYLEGSVRV

SEQ ID NO: 348 - protein, 17 aa, Artificial sequence
  betacoronavirus epitope
VYQCAMRPNFTIKGSFL

SEQ ID NO: 349 - protein, 20 aa, Artificial sequence
  betacoronavirus epitope
FPKSDGTGTIYTELEPPCRF

SEQ ID NO: 350 - protein, 18 aa, Artificial sequence
  betacoronavirus epitope
DGDMVPHISRQRLTKYTM

SEQ ID NO: 351 - protein, 20 aa, Artificial sequence
  betacoronavirus epitope
HVDTDLTKPYIKWDLLKYDF

SEQ ID NO: 352 - protein, 23 aa, Artificial sequence
  betacoronavirus epitope
DRAMPNMLRIMASLVLARKHTTC

SEQ ID NO: 353 - protein, 48 aa, Artificial sequence
  betacoronavirus epitope
TLMIERFVSLAIDAYPLTKHPNQEYADVFHLYLQYIRKLHDELTGHML

SEQ ID NO: 354 - protein, 29 aa, Artificial sequence
  betacoronavirus epitope
RLKLFAAETLKATEETFKLSYGIATVREV

SEQ ID NO: 355 - protein, 21 aa, Artificial sequence
  betacoronavirus epitope
MPLSAPTLVPQEHYVRITGLY

SEQ ID NO: 356 - protein, 15 aa, Artificial sequence
  betacoronavirus epitope
AQLPAPRTLLTKGTL

SEQ ID NO: 357 - protein, 17 aa, Artificial sequence
  betacoronavirus epitope
RPQIGVVREFLTRNPAW

SEQ ID NO: 358 - protein, 11 aa, Artificial sequence
  betacoronavirus epitope
TTLPVNVAFEL

SEQ ID NO: 359 - protein, 23 aa, Artificial sequence
  betacoronavirus epitope
KVDGVVQQLPETYFTQSRNLQEF

SEQ ID NO: 360 - protein, 36 aa, Artificial sequence
  betacoronavirus epitope
FYPKLQSSQAWQPGVAMPNLYKMQRMLLEKCDLQNY

SEQ ID NO: 361 - protein, 18 aa, Artificial sequence
  betacoronavirus epitope
LAVPYNMRVIHFGAGSDK

SEQ ID NO: 362 - protein, 11 aa, Artificial sequence
  betacoronavirus epitope
TLDSKTQSLLI

SEQ ID NO: 363 - protein, 21 aa, Artificial sequence
  betacoronavirus epitope
FVFKNIDGYFKIYSKHTPINL

SEQ ID NO: 364 - protein, 15 aa, Artificial sequence
  betacoronavirus epitope
YYVGYLQPRTFLLKY

SEQ ID NO: 365 - protein, 29 aa, Artificial sequence
  betacoronavirus epitope
ALDPLSETKCTLKSFTVEKGIYQTSNFRV

SEQ ID NO: 366 - protein, 19 aa, Artificial sequence
  betacoronavirus epitope
VQPTESIVRFPNITNLCPF

SEQ ID NO: 367 - protein, 16 aa, Artificial sequence
  betacoronavirus epitope
FNATRFASVYAWNRKR

SEQ ID NO: 368 - protein, 22 aa, Artificial sequence
  betacoronavirus epitope
SNCVADYSVLYNSASFSTFKCY

SEQ ID NO: 369 - protein, 18 aa, Artificial sequence
  betacoronavirus epitope
RQIAPGQTGKIADYNYKL

SEQ ID NO: 370 - protein, 40 aa, Artificial sequence
  betacoronavirus epitope
GPKKSTNLVKNKCVNFNFNGLTGTGVLTESNKKFLPFQQF

SEQ ID NO: 371 - protein, 12 aa, Artificial sequence
  betacoronavirus epitope
HADQLTPTWRVY

SEQ ID NO: 372 - protein, 30 aa, Artificial sequence
  betacoronavirus epitope
IAIPTNFTISVTTEILPVSMTKTSVDCTMY

SEQ ID NO: 373 - protein, 19 aa, Artificial sequence
  betacoronavirus epitope
QEVFAQVKQIYKTPPIKDF

SEQ ID NO: 374 - protein, 27 aa, Artificial sequence
  betacoronavirus epitope
RSFIEDLLFNKVTLADAGFIKQYGDCL

SEQ ID NO: 375 - protein, 42 aa, Artificial sequence
  betacoronavirus epitope
AALQIPFAMQMAYRFNGIGVTQNVLYENQKLIANQFNSAIGK

SEQ ID NO: 376 - protein, 15 aa, Artificial sequence
  betacoronavirus epitope
AQALNTLVKQLSSNF

SEQ ID NO: 377 - protein, 17 aa, Artificial sequence
  betacoronavirus epitope
SVLNDILSRLDKVEAEV

SEQ ID NO: 378 - protein, 13 aa, Artificial sequence
  betacoronavirus epitope
LITGRLQSLQTYV

SEQ ID NO: 379 - protein, 29 aa, Artificial sequence
  betacoronavirus epitope
YHLMSFPQSAPHGVVFLHVTYVPAQEKNF

SEQ ID NO: 380 - protein, 12 aa, Artificial sequence
  betacoronavirus epitope
VYDPLQPELDSF

SEQ ID NO: 381 - protein, 21 aa, Artificial sequence
  betacoronavirus epitope
VSLVKPSFYVYSRVKNLNSSR

SEQ ID NO: 382 - protein, 21 aa, Artificial sequence
  betacoronavirus epitope
DSNGTITVEELKKLLEQWNLV

SEQ ID NO: 383 - protein, 18 aa, Artificial sequence
  betacoronavirus epitope
FAYANRNRFLYIIKLIFL

SEQ ID NO: 384 - protein, 28 aa, Artificial sequence
  betacoronavirus epitope
GLMWLSYFIASFRLFARTRSMWSFNPET

SEQ ID NO: 385 - protein, 33 aa, Artificial sequence
  betacoronavirus epitope
HLRIAGHHLGRCDIKDLPKEITVATSRTLSYYK

SEQ ID NO: 386 - protein, 102 aa, Artificial sequence
  betacoronavirus epitope
PLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTL

SEQ ID NO: 387 - protein, 109 aa, Artificial sequence
  betacoronavirus epitope
DAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNV

SEQ ID NO: 388 - protein, 69 aa, Artificial sequence
  betacoronavirus epitope
EGFNCYFPLQSYGFQPTNGVGYQPYRVVVLSFELLHAPATVCGPKKSTNLVKNKCVNFNFNGLTGTGVL

SEQ ID NO: 389 - protein, 99 aa, Artificial sequence
  betacoronavirus epitope
QELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQK

SEQ ID NO: 390 - protein, 79 aa, Artificial sequence
  betacoronavirus epitope
ASYQTQTNSPRRARSVASQSIIAYTMSLGAENSVAYSNNSIAIPTNFTISVTTEILPVSMTKTSVDCTMYICGDSTECS

SEQ ID NO: 391 - protein, 54 aa, Artificial sequence
  betacoronavirus epitope
SKRVDFCGKGYHLMSFPQSAPHGVVFLHVTYVPAQEKNFTTAPAICHDGKAHFP

SEQ ID NO: 392 - protein, 43 aa, Artificial sequence
  betacoronavirus epitope
DSSSGWTAGAAAYYVGYLQPRTFLLKYNENGTITDAVDCALDP

SEQ ID NO: 393 - protein, 48 aa, Artificial sequence
  betacoronavirus epitope
LPDPSKPSKRSFIEDLLFNKVTLADAGFIKQYGDCLGDIAARDLICAQ

SEQ ID NO: 394 - protein, 52 aa, Artificial sequence
  betacoronavirus epitope
KEIDRLNEVAKNLNESLIDLQELGKYEQYIKWPWYIWLGFIAGLIAIVMVTI

SEQ ID NO: 395 - protein, 80 aa, Artificial sequence
  betacoronavirus epitope
ASWFTALTQHGKEDLKFPRGQGVPINTNSSPDDQIGYYRRATRRIRGGDGKMKDLSPRWYFYYLGTGPEAGLPYGANKDG

SEQ ID NO: 396 - protein, 41 aa, Artificial sequence
  betacoronavirus epitope
NNLDSKVGGNYNYLYRLFRKSNLKPFERDISTEIYQAGSTP

SEQ ID NO: 397 - protein, 59 aa, Artificial sequence
  betacoronavirus epitope
HHLGRCDIKDLPKEITVATSRTLSYYKLGASQRVAGDSGFAAYSRYRIGNYKLNTDHSS

SEQ ID NO: 398 - protein, 69 aa, Artificial sequence
  betacoronavirus epitope
SSIVITSGDGTTSPISEHDYQIGGYTEKWESGVKDCVVLHSYFTSDYYQLYSTQLSTDTGVEHVTFFIY

SEQ ID NO: 399 - protein, 48 aa, Artificial sequence
  betacoronavirus epitope
LLFVTVYSHLLLVAAGLEAPFLYLYALVYFLQSINFVRIIMRLWLCWK

SEQ ID NO: 400 - protein, 59 aa, Artificial sequence
  betacoronavirus epitope
VIKVCEFQFCNDPFLGVYYHKNNKSWMESEFRVYSSANNCTFEYVSQPFLMDLEGKQGN

SEQ ID NO: 401 - protein, 61 aa, Artificial sequence
  betacoronavirus epitope
TITSGWTFGAGAALQIPFAMQMAYRFNGIGVTQNVLYENQKLIANQFNSAIGKIQDSLSST

SEQ ID NO: 402 - protein, 49 aa, Artificial sequence
  betacoronavirus epitope
LFLAFVVFLLVTLAILTALRLCAYCCNIVNVSLVKPSFYVYSRVKNLNS

SEQ ID NO: 403 - protein, 33 aa, Artificial sequence
  betacoronavirus epitope
MDLFMRIFTIGTVTLKQGEIKDATPSDFVRATA

SEQ ID NO: 404 - protein, 31 aa, Artificial sequence
  betacoronavirus epitope
NLREFVFKNIDGYFKIYSKHTPINLVRDLPQ

SEQ ID NO: 405 - protein, 30 aa, Artificial sequence
  betacoronavirus epitope
KAHFPREGVFVSNGTHWFVTQRNFYEPQII

SEQ ID NO: 406 - protein, 42 aa, Artificial sequence
  betacoronavirus epitope
QTVTKKSAAEASKKPRQKRTATKAYNVTQAFGRRGPEQTQGN

SEQ ID NO: 407 - protein, 32 aa, Artificial sequence
  betacoronavirus epitope
VPVAIHADQLTPTWRVYSTGSNVFQTRAGCLI

SEQ ID NO: 408 - protein, 41 aa, Artificial sequence
  betacoronavirus epitope
ICAQKFNGLTVLPPLLTDEMIAQYTSALLAGTITSGWTFGA

SEQ ID NO: 409 - protein, 37 aa, Artificial sequence
  betacoronavirus epitope
SFVIRGDEVRQIAPGQTGKIADYNYKLPDDFTGCVIA

SEQ ID NO: 410 - protein, 44 aa, Artificial sequence
  betacoronavirus epitope
PFGWLIVGVALLAVFQSASKIITLKKRWQLALSKGVHFVCNLLL

SEQ ID NO: 411 - protein, 34 aa, Artificial sequence
  betacoronavirus epitope
VEQDKNTQEVFAQVKQIYKTPPIKDFGGFNFSQI

SEQ ID NO: 412 - protein, 35 aa, Artificial sequence
  betacoronavirus epitope
CLVGLMWLSYFIASFRLFARTRSMWSFNPETNILL

SEQ ID NO: 413 - protein, 36 aa, Artificial sequence
  betacoronavirus epitope
PARMAGNGGDAALALLLLDRLNQLESKMSGKGQQQQ

SEQ ID NO: 414 - protein, 32 aa, Artificial sequence
  betacoronavirus epitope
MADSNGTITVEELKKLLEQWNLVIGFLFLTWI

SEQ ID NO: 415 - protein, 33 aa, Artificial sequence
  betacoronavirus epitope
WICLLQFAYANRNRFLYIIKLIFLWLLWPVTLA

SEQ ID NO: 416 - protein, 16 aa, Artificial sequence
  betacoronavirus epitope
AQFAPSASAFFGMSRI

SEQ ID NO: 417 - protein, 25 aa, Artificial sequence
  betacoronavirus epitope
FGDDTVIEVQGYKSVNITFELDERI

SEQ ID NO: 418 - protein, 15 aa, Artificial sequence
  betacoronavirus epitope
YTVELGTEVNEFACV

SEQ ID NO: 419 - protein, 19 aa, Artificial sequence
  betacoronavirus epitope
VQQESPFVMMSAPPAQYEL

SEQ ID NO: 420 - protein, 29 aa, Artificial sequence
  betacoronavirus epitope
VQMAPISAMVRMYIFFASFYYVWKSYVHV

SEQ ID NO: 421 - protein, 16 aa, Artificial sequence
  betacoronavirus epitope
KMFDAYVNTFSSTFNV

SEQ ID NO: 422 - protein, 20 aa, Artificial sequence
  betacoronavirus epitope
TANPKTPKYKFVRIQPGQTF

SEQ ID NO: 423 - protein, 17 aa, Artificial sequence
  betacoronavirus epitope
FLNRFTTTLNDFNLVAM

SEQ ID NO: 424 - protein, 15 aa, Artificial sequence
  betacoronavirus epitope
MVYMPASWVMRIMTW

SEQ ID NO: 425 - protein, 15 aa, Artificial sequence
  betacoronavirus epitope
RVWTLMNVLTLVYKV

SEQ ID NO: 426 - protein, 15 aa, Artificial sequence
  betacoronavirus epitope
YFIKGLNNLNRGMVL

SEQ ID NO: 427 - protein, 15 aa, Artificial sequence
  betacoronavirus epitope
SLLMPILTLTRALTA

SEQ ID NO: 428 - protein, 18 aa, Artificial sequence
  betacoronavirus epitope
SSVELKHFFFAQDGNAAI

SEQ ID NO: 429 - protein, 15 aa, Artificial sequence
  betacoronavirus epitope
RAMPNMLRIMASLVL

SEQ ID NO: 430 - protein, 28 aa, Artificial sequence
  betacoronavirus epitope
SSGDATTAYANSVFNICQAVTANVNALL

SEQ ID NO: 431 - protein, 16 aa, Artificial sequence
  betacoronavirus epitope
FVNEFYAYLRKHFSMM

SEQ ID NO: 432 - protein, 18 aa, Artificial sequence
  betacoronavirus epitope
ASIKNFKSVLYYQNNVFM

SEQ ID NO: 433 - protein, 15 aa, Artificial sequence
  betacoronavirus epitope
LMIERFVSLAIDAYP

SEQ ID NO: 434 - protein, 17 aa, Artificial sequence
  betacoronavirus epitope
HVISTSHKLVLSVNPYV

SEQ ID NO: 435 - protein, 25 aa, Artificial sequence
  betacoronavirus epitope
HFAIGLALYYPSARIVYTACSHAAV

SEQ ID NO: 436 - protein, 15 aa, Artificial sequence
  betacoronavirus epitope
EIVDTVSALVYDNKL

SEQ ID NO: 437 - protein, 25 aa, Artificial sequence
  betacoronavirus epitope
MTYRRLISMMGFKMNYQVNGYPNMF

SEQ ID NO: 438 - protein, 24 aa, Artificial sequence
  betacoronavirus epitope
MLSDTLKNLSDRVVFVLWAHGFEL

SEQ ID NO: 439 - protein, 26 aa, Artificial sequence
  betacoronavirus epitope
TVYTKVDGVDVELFENKTTLPVNVAF

SEQ ID NO: 440 - protein, 15 aa, Artificial sequence
  betacoronavirus epitope
YLNTLTLAVPYNMRV

SEQ ID NO: 441 - protein, 15 aa, Artificial sequence
  betacoronavirus epitope
RVYSTGSNVFQTRAG

SEQ ID NO: 442 - protein, 23 aa, Artificial sequence
  betacoronavirus epitope
SVAYSNNSIAIPTNFTISVTTEI

SEQ ID NO: 443 - protein, 15 aa, Artificial sequence
  betacoronavirus epitope
RSFIEDLLFNKVTLA

SEQ ID NO: 444 - protein, 15 aa, Artificial sequence
  betacoronavirus epitope
LVKPSFYVYSRVKNL

SEQ ID NO: 445 - protein, 15 aa, Artificial sequence
  betacoronavirus epitope
NITNLCPFGEVFNAT

SEQ ID NO: 446 - protein, 15 aa, Artificial sequence
  betacoronavirus epitope
NLCPFGEVFNATRFA

SEQ ID NO: 447 - protein, 15 aa, Artificial sequence
  betacoronavirus epitope
PFGEVFNATRFASVY

SEQ ID NO: 448 - protein, 15 aa, Artificial sequence
  betacoronavirus epitope
EVFNATRFASVYAWN

SEQ ID NO: 449 - protein, 15 aa, Artificial sequence
  betacoronavirus epitope
NATRFASVYAWNRKR

SEQ ID NO: 450 - protein, 15 aa, Artificial sequence
  betacoronavirus epitope
RFASVYAWNRKRISN

SEQ ID NO: 451 - protein, 15 aa, Artificial sequence
  betacoronavirus epitope
SVYAWNRKRISNCVA

SEQ ID NO: 452 - protein, 15 aa, Artificial sequence
  betacoronavirus epitope
AWNRKRISNCVADYS

SEQ ID NO: 453 - protein, 15 aa, Artificial sequence
  betacoronavirus epitope
RKRISNCVADYSVLY

SEQ ID NO: 454 - protein, 15 aa, Artificial sequence
  betacoronavirus epitope
ISNCVADYSVLYNSA

SEQ ID NO: 455 - protein, 15 aa, Artificial sequence
  betacoronavirus epitope
CVADYSVLYNSASFS

SEQ ID NO: 456 - protein, 15 aa, Artificial sequence
  betacoronavirus epitope
DYSVLYNSASFSTFK

SEQ ID NO: 457 - protein, 15 aa, Artificial sequence
  betacoronavirus epitope
VLYNSASFSTFKCYG

SEQ ID NO: 458 - protein, 15 aa, Artificial sequence
  betacoronavirus epitope
NSASFSTFKCYGVSP

SEQ ID NO: 459 - protein, 15 aa, Artificial sequence
  betacoronavirus epitope
SFSTFKCYGVSPTKL

SEQ ID NO: 460 - protein, 15 aa, Artificial sequence
  betacoronavirus epitope
TFKCYGVSPTKLNDL

SEQ ID NO: 461 - protein, 15 aa, Artificial sequence
  betacoronavirus epitope
CYGVSPTKLNDLCFT

SEQ ID NO: 462 - protein, 15 aa, Artificial sequence
  betacoronavirus epitope
VSPTKLNDLCFTNVY

SEQ ID NO: 463 - protein, 15 aa, Artificial sequence
  betacoronavirus epitope
TKLNDLCFTNVYADS

SEQ ID NO: 464 - protein, 15 aa, Artificial sequence
  betacoronavirus epitope
NDLCFTNVYADSFVI

SEQ ID NO: 465 - protein, 15 aa, Artificial sequence
  betacoronavirus epitope
CFTNVYADSFVIRGD

SEQ ID NO: 466 - protein, 15 aa, Artificial sequence
  betacoronavirus epitope
NVYADSFVIRGDEVR

SEQ ID NO: 467 - protein, 15 aa, Artificial sequence
  betacoronavirus epitope
ADSFVIRGDEVRQIA

SEQ ID NO: 468 - protein, 15 aa, Artificial sequence
  betacoronavirus epitope
FVIRGDEVRQIAPGQ

SEQ ID NO: 469 - protein, 15 aa, Artificial sequence
  betacoronavirus epitope
RGDEVRQIAPGQTGK

SEQ ID NO: 470 - protein, 15 aa, Artificial sequence
  betacoronavirus epitope
EVRQIAPGQTGKIAD

SEQ ID NO: 471 - protein, 15 aa, Artificial sequence
  betacoronavirus epitope
QIAPGQTGKIADYNY

SEQ ID NO: 472 - protein, 15 aa, Artificial sequence
  betacoronavirus epitope
PGQTGKIADYNYKLP

SEQ ID NO: 473 - protein, 15 aa, Artificial sequence
  betacoronavirus epitope
TGKIADYNYKLPDDF

SEQ ID NO: 474 - protein, 15 aa, Artificial sequence
  betacoronavirus epitope
IADYNYKLPDDFTGC

SEQ ID NO: 475 - protein, 15 aa, Artificial sequence
  betacoronavirus epitope
YNYKLPDDFTGCVIA

SEQ ID NO: 476 - protein, 15 aa, Artificial sequence
  betacoronavirus epitope
KLPDDFTGCVIAWNS

SEQ ID NO: 477 - protein, 15 aa, Artificial sequence
  betacoronavirus epitope
DDFTGCVIAWNSNNL

SEQ ID NO: 478 - protein, 15 aa, Artificial sequence
  betacoronavirus epitope
TGCVIAWNSNNLDSK

SEQ ID NO: 479 - protein, 15 aa, Artificial sequence
  betacoronavirus epitope
VIAWNSNNLDSKVGG

SEQ ID NO: 480 - protein, 15 aa, Artificial sequence
  betacoronavirus epitope
WNSNNLDSKVGGNYN

SEQ ID NO: 481 - protein, 15 aa, Artificial sequence
  betacoronavirus epitope
NNLDSKVGGNYNYLY

SEQ ID NO: 482 - protein, 15 aa, Artificial sequence
  betacoronavirus epitope
DSKVGGNYNYLYRLF

SEQ ID NO: 483 - protein, 15 aa, Artificial sequence
  betacoronavirus epitope
VGGNYNYLYRLFRKS

SEQ ID NO: 484 - protein, 15 aa, Artificial sequence
  betacoronavirus epitope
NYNYLYRLFRKSNLK

SEQ ID NO: 485 - protein, 15 aa, Artificial sequence
  betacoronavirus epitope
YLYRLFRKSNLKPFE

SEQ ID NO: 486 - protein, 15 aa, Artificial sequence
  betacoronavirus epitope
RLFRKSNLKPFERDI

SEQ ID NO: 487 - protein, 15 aa, Artificial sequence
  betacoronavirus epitope
RKSNLKPFERDISTE

SEQ ID NO: 488 - protein, 15 aa, Artificial sequence
  betacoronavirus epitope
NLKPFERDISTEIYQ

SEQ ID NO: 489 - protein, 15 aa, Artificial sequence
  betacoronavirus epitope
PFERDISTEIYQAGS

SEQ ID NO: 490 - protein, 15 aa, Artificial sequence
  betacoronavirus epitope
RDISTEIYQAGSTPC

SEQ ID NO: 491 - protein, 15 aa, Artificial sequence
  betacoronavirus epitope
STEIYQAGSTPCNGV

SEQ ID NO: 492 - protein, 15 aa, Artificial sequence
  betacoronavirus epitope
IYQAGSTPCNGVEGF

SEQ ID NO: 493 - protein, 15 aa, Artificial sequence
  betacoronavirus epitope
AGSTPCNGVEGFNCY

SEQ ID NO: 494 - protein, 15 aa, Artificial sequence
  betacoronavirus epitope
TPCNGVEGFNCYFPL

SEQ ID NO: 495 - protein, 15 aa, Artificial sequence
  betacoronavirus epitope
NGVEGFNCYFPLQSY

SEQ ID NO: 496 - protein, 15 aa, Artificial sequence
  betacoronavirus epitope
EGFNCYFPLQSYGFQ

SEQ ID NO: 497 - protein, 15 aa, Artificial sequence
  betacoronavirus epitope
NCYFPLQSYGFQPTN

SEQ ID NO: 498 - protein, 15 aa, Artificial sequence
  betacoronavirus epitope
FPLQSYGFQPTNGVG

SEQ ID NO: 499 - protein, 15 aa, Artificial sequence
  betacoronavirus epitope
QSYGFQPTNGVGYQP

SEQ ID NO: 500 - protein, 15 aa, Artificial sequence
  betacoronavirus epitope
GFQPTNGVGYQPYRV

SEQ ID NO: 501 - protein, 15 aa, Artificial sequence
  betacoronavirus epitope
PTNGVGYQPYRVVVL

SEQ ID NO: 502 - protein, 15 aa, Artificial sequence
  betacoronavirus epitope
GVGYQPYRVVVLSFE

SEQ ID NO: 503 - protein, 15 aa, Artificial sequence
  betacoronavirus epitope
YQPYRVVVLSFELLH

SEQ ID NO: 504 - protein, 15 aa, Artificial sequence
  betacoronavirus epitope
YRVVVLSFELLHAPA

SEQ ID NO: 505 - protein, 13 aa, Artificial sequence
  betacoronavirus epitope
VVLSFELLHAPAT

SEQ ID NO: 506 - protein, 15 aa, Artificial sequence
  betacoronavirus epitope
VNLTTRTQLPPAYTN

SEQ ID NO: 507 - protein, 15 aa, Artificial sequence
  betacoronavirus epitope
TRTQLPPAYTNSFTR

SEQ ID NO: 508 - protein, 15 aa, Artificial sequence
  betacoronavirus epitope
LPPAYTNSFTRGVYY

SEQ ID NO: 509 - protein, 15 aa, Artificial sequence
  betacoronavirus epitope
YTNSFTRGVYYPDKV

SEQ ID NO: 510 - protein, 15 aa, Artificial sequence
  betacoronavirus epitope
FTRGVYYPDKVFRSS

SEQ ID NO: 511 - protein, 15 aa, Artificial sequence
  betacoronavirus epitope
VYYPDKVFRSSVLHS

SEQ ID NO: 512 - protein, 15 aa, Artificial sequence
  betacoronavirus epitope
DKVFRSSVLHSTQDL

SEQ ID NO: 513 - protein, 15 aa, Artificial sequence
  betacoronavirus epitope
RSSVLHSTQDLFLPF

SEQ ID NO: 514 - protein, 15 aa, Artificial sequence
  betacoronavirus epitope
LHSTQDLFLPFFSNV

SEQ ID NO: 515 - protein, 15 aa, Artificial sequence
  betacoronavirus epitope
QDLFLPFFSNVTWFH

SEQ ID NO: 516 - protein, 15 aa, Artificial sequence
  betacoronavirus epitope
LPFFSNVTWFHAIHV

SEQ ID NO: 517 - protein, 15 aa, Artificial sequence
  betacoronavirus epitope
SNVTWFHAIHVSGTN

SEQ ID NO: 518 - protein, 15 aa, Artificial sequence
  betacoronavirus epitope
WFHAIHVSGTNGTKR

SEQ ID NO: 519 - protein, 15 aa, Artificial sequence
  betacoronavirus epitope
IHVSGTNGTKRFDNP

SEQ ID NO: 520 - protein, 15 aa, Artificial sequence
  betacoronavirus epitope
GTNGTKRFDNPVLPF

SEQ ID NO: 521 - protein, 15 aa, Artificial sequence
  betacoronavirus epitope
TKRFDNPVLPFNDGV

SEQ ID NO: 522 - protein, 15 aa, Artificial sequence
  betacoronavirus epitope
DNPVLPFNDGVYFAS

SEQ ID NO: 523 - protein, 15 aa, Artificial sequence
  betacoronavirus epitope
LPFNDGVYFASTEKS

SEQ ID NO: 524 - protein, 15 aa, Artificial sequence
  betacoronavirus epitope
DGVYFASTEKSNIIR

SEQ ID NO: 525 - protein, 15 aa, Artificial sequence
  betacoronavirus epitope
FASTEKSNIIRGWIF

SEQ ID NO: 526 - protein, 15 aa, Artificial sequence
  betacoronavirus epitope
EKSNIIRGWIFGTTL

SEQ ID NO: 527 - protein, 15 aa, Artificial sequence
  betacoronavirus epitope
IIRGWIFGTTLDSKT

SEQ ID NO: 528 - protein, 15 aa, Artificial sequence
  betacoronavirus epitope
WIFGTTLDSKTQSLL

SEQ ID NO: 529 - protein, 15 aa, Artificial sequence
  betacoronavirus epitope
TTLDSKTQSLLIVNN

SEQ ID NO: 530 - protein, 15 aa, Artificial sequence
  betacoronavirus epitope
SKTQSLLIVNNATNV

SEQ ID NO: 531 - protein, 15 aa, Artificial sequence
  betacoronavirus epitope
SLLIVNNATNVVIKV

SEQ ID NO: 532 - protein, 15 aa, Artificial sequence
  betacoronavirus epitope
VNNATNVVIKVCEFQ

SEQ ID NO: 533 - protein, 15 aa, Artificial sequence
  betacoronavirus epitope
TNVVIKVCEFQFCND

SEQ ID NO: 534 - protein, 15 aa, Artificial sequence
  betacoronavirus epitope
IKVCEFQFCNDPFLG

SEQ ID NO: 535 - protein, 15 aa, Artificial sequence
  betacoronavirus epitope
EFQFCNDPFLGVYYH

SEQ ID NO: 536 - protein, 15 aa, Artificial sequence
  betacoronavirus epitope
CNDPFLGVYYHKNNK

SEQ ID NO: 537 - protein, 15 aa, Artificial sequence
  betacoronavirus epitope
FLGVYYHKNNKSWME

SEQ ID NO: 538 - protein, 15 aa, Artificial sequence
  betacoronavirus epitope
YYHKNNKSWMESEFR

SEQ ID NO: 539 - protein, 15 aa, Artificial sequence
  betacoronavirus epitope
NNKSWMESEFRVYSS

SEQ ID NO: 540 - protein, 15 aa, Artificial sequence
  betacoronavirus epitope
WMESEFRVYSSANNC

SEQ ID NO: 541 - protein, 15 aa, Artificial sequence
  betacoronavirus epitope
EFRVYSSANNCTFEY

SEQ ID NO: 542 - protein, 15 aa, Artificial sequence
  betacoronavirus epitope
YSSANNCTFEYVSQP

SEQ ID NO: 543 - protein, 15 aa, Artificial sequence
  betacoronavirus epitope
NNCTFEYVSQPFLMD

SEQ ID NO: 544 - protein, 15 aa, Artificial sequence
  betacoronavirus epitope
FEYVSQPFLMDLEGK

SEQ ID NO: 545 - protein, 15 aa, Artificial sequence
  betacoronavirus epitope
SQPFLMDLEGKQGNF

SEQ ID NO: 546 - protein, 15 aa, Artificial sequence
  betacoronavirus epitope
LMDLEGKQGNFKNLR

SEQ ID NO: 547 - protein, 15 aa, Artificial sequence
  betacoronavirus epitope
EGKQGNFKNLREFVF

SEQ ID NO: 548 - protein, 15 aa, Artificial sequence
  betacoronavirus epitope
GNFKNLREFVFKNID

SEQ ID NO: 549 - protein, 15 aa, Artificial sequence
  betacoronavirus epitope
NLREFVFKNIDGYFK

SEQ ID NO: 550 - protein, 15 aa, Artificial sequence
  betacoronavirus epitope
FVFKNIDGYFKIYSK

SEQ ID NO: 551 - protein, 15 aa, Artificial sequence
  betacoronavirus epitope
NIDGYFKIYSKHTPI

SEQ ID NO: 552 - protein, 15 aa, Artificial sequence
  betacoronavirus epitope
YFKIYSKHTPINLVR

SEQ ID NO: 553 - protein, 15 aa, Artificial sequence
  betacoronavirus epitope
YSKHTPINLVRDLPQ

SEQ ID NO: 554 - protein, 15 aa, Artificial sequence
  betacoronavirus epitope
TPINLVRDLPQGFSA

SEQ ID NO: 555 - protein, 15 aa, Artificial sequence
  betacoronavirus epitope
LVRDLPQGFSALEPL

SEQ ID NO: 556 - protein, 15 aa, Artificial sequence
  betacoronavirus epitope
LPQGFSALEPLVDLP

SEQ ID NO: 557 - protein, 15 aa, Artificial sequence
  betacoronavirus epitope
FSALEPLVDLPIGIN

SEQ ID NO: 558 - protein, 15 aa, Artificial sequence
  betacoronavirus epitope
EPLVDLPIGINITRF

SEQ ID NO: 559 - protein, 15 aa, Artificial sequence
  betacoronavirus epitope
DLPIGINITRFQTLL

SEQ ID NO: 560 - protein, 15 aa, Artificial sequence
  betacoronavirus epitope
GINITRFQTLLALHR

SEQ ID NO: 561 - protein, 15 aa, Artificial sequence
  betacoronavirus epitope
TRFQTLLALHRSYLT

SEQ ID NO: 562 - protein, 15 aa, Artificial sequence
  betacoronavirus epitope
TLLALHRSYLTPGDS

SEQ ID NO: 563 - protein, 15 aa, Artificial sequence
  betacoronavirus epitope
LHRSYLTPGDSSSGW

SEQ ID NO: 564 - protein, 15 aa, Artificial sequence
  betacoronavirus epitope
YLTPGDSSSGWTAGA

SEQ ID NO: 565 - protein, 15 aa, Artificial sequence
  betacoronavirus epitope
GDSSSGWTAGAAAYY

SEQ ID NO: 566 - protein, 15 aa, Artificial sequence
  betacoronavirus epitope
SGWTAGAAAYYVGYL

SEQ ID NO: 567 - protein, 15 aa, Artificial sequence
  betacoronavirus epitope
AGAAAYYVGYLQPRT

SEQ ID NO: 568 - protein, 15 aa, Artificial sequence
  betacoronavirus epitope
AYYVGYLQPRTFLLK

SEQ ID NO: 569 - protein, 15 aa, Artificial sequence
  betacoronavirus epitope
GYLQPRTFLLKYNEN

SEQ ID NO: 570 - protein, 15 aa, Artificial sequence
  betacoronavirus epitope
PRTFLLKYNENGTIT

SEQ ID NO: 571 - protein, 15 aa, Artificial sequence
  betacoronavirus epitope
LLKYNENGTITDAVD

SEQ ID NO: 572 - protein, 15 aa, Artificial sequence
  betacoronavirus epitope
NENGTITDAVDCALD

SEQ ID NO: 573 - protein, 15 aa, Artificial sequence
  betacoronavirus epitope
TITDAVDCALDPLSE

SEQ ID NO: 574 - protein, 15 aa, Artificial sequence
  betacoronavirus epitope
AVDCALDPLSETKCT

SEQ ID NO: 575 - protein, 15 aa, Artificial sequence
  betacoronavirus epitope
ALDPLSETKCTLKSF

SEQ ID NO: 576 - protein, 15 aa, Artificial sequence
  betacoronavirus epitope
LSETKCTLKSFTVEK

SEQ ID NO: 577 - protein, 15 aa, Artificial sequence
  betacoronavirus epitope
KCTLKSFTVEKGIYQ

SEQ ID NO: 578 - protein, 15 aa, Artificial sequence
  betacoronavirus epitope
KSFTVEKGIYQTSNF

SEQ ID NO: 579 - protein, 15 aa, Artificial sequence
  betacoronavirus epitope
VEKGIYQTSNFRVQP

SEQ ID NO: 580 - protein, 15 aa, Artificial sequence
  betacoronavirus epitope
IYQTSNFRVQPTESI

SEQ ID NO: 581 - protein, 15 aa, Artificial sequence
  betacoronavirus epitope
SNFRVQPTESIVRFP

SEQ ID NO: 582 - protein, 15 aa, Artificial sequence
  betacoronavirus epitope
VQPTESIVRFPNITN

SEQ ID NO: 583 - protein, 15 aa, Artificial sequence
  betacoronavirus epitope
ESIVRFPNITNLCPF

SEQ ID NO: 584 - protein, 15 aa, Artificial sequence
  betacoronavirus epitope
RFPNITNLCPFGEVF

SEQ ID NO: 585 - protein, 15 aa, Artificial sequence
  betacoronavirus epitope
ITNLCPFGEVFNATR

SEQ ID NO: 586 - protein, 15 aa, Artificial sequence
  betacoronavirus epitope
CPFGEVFNATRFASV

SEQ ID NO: 587 - protein, 15 aa, Artificial sequence
  betacoronavirus epitope
EVFNATRFASVYAWN

SEQ ID NO: 588 - protein, 15 aa, Artificial sequence
  betacoronavirus epitope
ATRFASVYAWNRKRI

SEQ ID NO: 589 - protein, 15 aa, Artificial sequence
  betacoronavirus epitope
ASVYAWNRKRISNCV

SEQ ID NO: 590 - protein, 15 aa, Artificial sequence
  betacoronavirus epitope
AWNRKRISNCVADYS

SEQ ID NO: 591 - protein, 15 aa, Artificial sequence
  betacoronavirus epitope
KRISNCVADYSVLYN

SEQ ID NO: 592 - protein, 15 aa, Artificial sequence
  betacoronavirus epitope
NCVADYSVLYNSASF

SEQ ID NO: 593 - protein, 15 aa, Artificial sequence
  betacoronavirus epitope
DYSVLYNSASFSTFK

SEQ ID NO: 594 - protein, 15 aa, Artificial sequence
  betacoronavirus epitope
LYNSASFSTFKCYGV

SEQ ID NO: 595 - protein, 15 aa, Artificial sequence
  betacoronavirus epitope
ASFSTFKCYGVSPTK

SEQ ID NO: 596 - protein, 15 aa, Artificial sequence
  betacoronavirus epitope
TFKCYGVSPTKLNDL

SEQ ID NO: 597 - protein, 15 aa, Artificial sequence
  betacoronavirus epitope
YGVSPTKLNDLCFTN

SEQ ID NO: 598 - protein, 15 aa, Artificial sequence
  betacoronavirus epitope
PTKLNDLCFTNVYAD

SEQ ID NO: 599 - protein, 15 aa, Artificial sequence
  betacoronavirus epitope
NDLCFTNVYADSFVI

SEQ ID NO: 600 - protein, 15 aa, Artificial sequence
  betacoronavirus epitope
FTNVYADSFVIRGDE

SEQ ID NO: 601 - protein, 15 aa, Artificial sequence
  betacoronavirus epitope
YADSFVIRGDEVRQI

SEQ ID NO: 602 - protein, 15 aa, Artificial sequence
  betacoronavirus epitope
FVIRGDEVRQIAPGQ

SEQ ID NO: 603 - protein, 15 aa, Artificial sequence
  betacoronavirus epitope
GDEVRQIAPGQTGKI

SEQ ID NO: 604 - protein, 15 aa, Artificial sequence
  betacoronavirus epitope
RQIAPGQTGKIADYN

SEQ ID NO: 605 - protein, 15 aa, Artificial sequence
  betacoronavirus epitope
PGQTGKIADYNYKLP

SEQ ID NO: 606 - protein, 15 aa, Artificial sequence
  betacoronavirus epitope
GKIADYNYKLPDDFT

SEQ ID NO: 607 - protein, 15 aa, Artificial sequence
  betacoronavirus epitope
DYNYKLPDDFTGCVI

SEQ ID NO: 608 - protein, 15 aa, Artificial sequence
  betacoronavirus epitope
KLPDDFTGCVIAWNS

SEQ ID NO: 609 - protein, 15 aa, Artificial sequence
  betacoronavirus epitope
DFTGCVIAWNSNNLD

SEQ ID NO: 610 - protein, 15 aa, Artificial sequence
  betacoronavirus epitope
CVIAWNSNNLDSKVG

SEQ ID NO: 611 - protein, 15 aa, Artificial sequence
  betacoronavirus epitope
WNSNNLDSKVGGNYN

SEQ ID NO: 612 - protein, 15 aa, Artificial sequence
  betacoronavirus epitope
NLDSKVGGNYNYLYR

SEQ ID NO: 613 - protein, 15 aa, Artificial sequence
  betacoronavirus epitope
KVGGNYNYLYRLFRK

SEQ ID NO: 614 - protein, 15 aa, Artificial sequence
  betacoronavirus epitope
NYNYLYRLFRKSNLK

SEQ ID NO: 615 - protein, 15 aa, Artificial sequence
  betacoronavirus epitope
LYRLFRKSNLKPFER

SEQ ID NO: 616 - protein, 15 aa, Artificial sequence
  betacoronavirus epitope
FRKSNLKPFERDIST

SEQ ID NO: 617 - protein, 15 aa, Artificial sequence
  betacoronavirus epitope
NLKPFERDISTEIYQ

SEQ ID NO: 618 - protein, 15 aa, Artificial sequence
  betacoronavirus epitope
FERDISTEIYQAGST

SEQ ID NO: 619 - protein, 15 aa, Artificial sequence
  betacoronavirus epitope
ISTEIYQAGSTPCNG

SEQ ID NO: 620 - protein, 15 aa, Artificial sequence
  betacoronavirus epitope
IYQAGSTPCNGVEGF

SEQ ID NO: 621 - protein, 15 aa, Artificial sequence
  betacoronavirus epitope
GSTPCNGVEGFNCYF

SEQ ID NO: 622 - protein, 15 aa, Artificial sequence
  betacoronavirus epitope
CNGVEGFNCYFPLQS

SEQ ID NO: 623 - protein, 15 aa, Artificial sequence
  betacoronavirus epitope
EGFNCYFPLQSYGFQ

SEQ ID NO: 624 - protein, 15 aa, Artificial sequence
  betacoronavirus epitope
CYFPLQSYGFQPTNG

SEQ ID NO: 625 - protein, 15 aa, Artificial sequence
  betacoronavirus epitope
LQSYGFQPTNGVGYQ

SEQ ID NO: 626 - protein, 15 aa, Artificial sequence
  betacoronavirus epitope
GFQPTNGVGYQPYRV

SEQ ID NO: 627 - protein, 15 aa, Artificial sequence
  betacoronavirus epitope
TNGVGYQPYRVVVLS

SEQ ID NO: 628 - protein, 15 aa, Artificial sequence
  betacoronavirus epitope
GYQPYRVVVLSFELL

SEQ ID NO: 629 - protein, 15 aa, Artificial sequence
  betacoronavirus epitope
YRVVVLSFELLHAPA

SEQ ID NO: 630 - protein, 15 aa, Artificial sequence
  betacoronavirus epitope
VLSFELLHAPATVCG

SEQ ID NO: 631 - protein, 15 aa, Artificial sequence
  betacoronavirus epitope
ELLHAPATVCGPKKS

SEQ ID NO: 632 - protein, 15 aa, Artificial sequence
  betacoronavirus epitope
APATVCGPKKSTNLV

SEQ ID NO: 633 - protein, 15 aa, Artificial sequence
  betacoronavirus epitope
VCGPKKSTNLVKNKC

SEQ ID NO: 634 - protein, 15 aa, Artificial sequence
  betacoronavirus epitope
KKSTNLVKNKCVNFN

SEQ ID NO: 635 - protein, 15 aa, Artificial sequence
  betacoronavirus epitope
NLVKNKCVNFNFNGL

SEQ ID NO: 636 - protein, 15 aa, Artificial sequence
  betacoronavirus epitope
NKCVNFNFNGLTGTG

SEQ ID NO: 637 - protein, 15 aa, Artificial sequence
  betacoronavirus epitope
NFNFNGLTGTGVLTE

SEQ ID NO: 638 - protein, 15 aa, Artificial sequence
  betacoronavirus epitope
NGLTGTGVLTESNKK

SEQ ID NO: 639 - protein, 15 aa, Artificial sequence
  betacoronavirus epitope
GTGVLTESNKKFLPF

SEQ ID NO: 640 - protein, 15 aa, Artificial sequence
  betacoronavirus epitope
LTESNKKFLPFQQFG

SEQ ID NO: 641 - protein, 15 aa, Artificial sequence
  betacoronavirus epitope
NKKFLPFQQFGRDIA

SEQ ID NO: 642 - protein, 15 aa, Artificial sequence
  betacoronavirus epitope
LPFQQFGRDIADTTD

SEQ ID NO: 643 - protein, 15 aa, Artificial sequence
  betacoronavirus epitope
QFGRDIADTTDAVRD

SEQ ID NO: 644 - protein, 15 aa, Artificial sequence
  betacoronavirus epitope
DIADTTDAVRDPQTL

SEQ ID NO: 645 - protein, 15 aa, Artificial sequence
  betacoronavirus epitope
TTDAVRDPQTLEILD

SEQ ID NO: 646 - protein, 15 aa, Artificial sequence
  betacoronavirus epitope
VRDPQTLEILDITPC

SEQ ID NO: 647 - protein, 15 aa, Artificial sequence
  betacoronavirus epitope
QTLEILDITPCSFGG

SEQ ID NO: 648 - protein, 15 aa, Artificial sequence
  betacoronavirus epitope
ILDITPCSFGGVSVI

SEQ ID NO: 649 - protein, 15 aa, Artificial sequence
  betacoronavirus epitope
TPCSFGGVSVITPGT

SEQ ID NO: 650 - protein, 15 aa, Artificial sequence
  betacoronavirus epitope
FGGVSVITPGTNTSN

SEQ ID NO: 651 - protein, 15 aa, Artificial sequence
  betacoronavirus epitope
SVITPGTNTSNQVAV

SEQ ID NO: 652 - protein, 15 aa, Artificial sequence
  betacoronavirus epitope
PGTNTSNQVAVLYQD

SEQ ID NO: 653 - protein, 15 aa, Artificial sequence
  betacoronavirus epitope
TSNQVAVLYQDVNCT

SEQ ID NO: 654 - protein, 15 aa, Artificial sequence
  betacoronavirus epitope
VAVLYQDVNCTEVPV

SEQ ID NO: 655 - protein, 15 aa, Artificial sequence
  betacoronavirus epitope
YQDVNCTEVPVAIHA

SEQ ID NO: 656 - protein, 15 aa, Artificial sequence
  betacoronavirus epitope
NCTEVPVAIHADQLT

SEQ ID NO: 657 - protein, 15 aa, Artificial sequence
  betacoronavirus epitope
VPVAIHADQLTPTWR

SEQ ID NO: 658 - protein, 15 aa, Artificial sequence
  betacoronavirus epitope
IHADQLTPTWRVYST

SEQ ID NO: 659 - protein, 15 aa, Artificial sequence
  betacoronavirus epitope
QLTPTWRVYSTGSNV

SEQ ID NO: 660 - protein, 15 aa, Artificial sequence
  betacoronavirus epitope
TWRVYSTGSNVFQTR

SEQ ID NO: 661 - protein, 15 aa, Artificial sequence
  betacoronavirus epitope
YSTGSNVFQTRAGCL

SEQ ID NO: 662 - protein, 15 aa, Artificial sequence
  betacoronavirus epitope
SNVFQTRAGCLIGAE

SEQ ID NO: 663 - protein, 15 aa, Artificial sequence
  betacoronavirus epitope
QTRAGCLIGAEHVNN

SEQ ID NO: 664 - protein, 15 aa, Artificial sequence
  betacoronavirus epitope
GCLIGAEHVNNSYEC

SEQ ID NO: 665 - protein, 15 aa, Artificial sequence
  betacoronavirus epitope
GAEHVNNSYECDIPI

SEQ ID NO: 666 - protein, 15 aa, Artificial sequence
  betacoronavirus epitope
VNNSYECDIPIGAGI

SEQ ID NO: 667 - protein, 15 aa, Artificial sequence
  betacoronavirus epitope
YECDIPIGAGICASY

SEQ ID NO: 668 - protein, 15 aa, Artificial sequence
  betacoronavirus epitope
IPIGAGICASYQTQT

SEQ ID NO: 669 - protein, 15 aa, Artificial sequence
  betacoronavirus epitope
AGICASYQTQTNSPR

SEQ ID NO: 670 - protein, 15 aa, Artificial sequence
  betacoronavirus epitope
ASYQTQTNSPRRARS

SEQ ID NO: 671 - protein, 15 aa, Artificial sequence
  betacoronavirus epitope
TQTNSPRRARSVASQ

SEQ ID NO: 672 - protein, 15 aa, Artificial sequence
  betacoronavirus epitope
SPRRARSVASQSIIA

SEQ ID NO: 673 - protein, 15 aa, Artificial sequence
  betacoronavirus epitope
ARSVASQSIIAYTMS

SEQ ID NO: 674 - protein, 15 aa, Artificial sequence
  betacoronavirus epitope
ASQSIIAYTMSLGAE

SEQ ID NO: 675 - protein, 15 aa, Artificial sequence
  betacoronavirus epitope
IIAYTMSLGAENSVA

SEQ ID NO: 676 - protein, 15 aa, Artificial sequence
  betacoronavirus epitope
TMSLGAENSVAYSNN

SEQ ID NO: 677 - protein, 15 aa, Artificial sequence
  betacoronavirus epitope
GAENSVAYSNNSIAI

SEQ ID NO: 678 - protein, 15 aa, Artificial sequence
  betacoronavirus epitope
SVAYSNNSIAIPTNF

SEQ ID NO: 679 - protein, 15 aa, Artificial sequence
  betacoronavirus epitope
SNNSIAIPTNFTISV

SEQ ID NO: 680 - protein, 15 aa, Artificial sequence
  betacoronavirus epitope
IAIPTNFTISVTTEI

SEQ ID NO: 681 - protein, 15 aa, Artificial sequence
  betacoronavirus epitope
TNFTISVTTEILPVS

SEQ ID NO: 682 - protein, 15 aa, Artificial sequence
  betacoronavirus epitope
ISVTTEILPVSMTKT

SEQ ID NO: 683 - protein, 15 aa, Artificial sequence
  betacoronavirus epitope
TEILPVSMTKTSVDC

SEQ ID NO: 684 - protein, 15 aa, Artificial sequence
  betacoronavirus epitope
PVSMTKTSVDCTMYI

SEQ ID NO: 685 - protein, 15 aa, Artificial sequence
  betacoronavirus epitope
TKTSVDCTMYICGDS

SEQ ID NO: 686 - protein, 15 aa, Artificial sequence
  betacoronavirus epitope
VDCTMYICGDSTECS

SEQ ID NO: 687 - protein, 15 aa, Artificial sequence
  betacoronavirus epitope
MYICGDSTECSNLLL

SEQ ID NO: 688 - protein, 15 aa, Artificial sequence
  betacoronavirus epitope
GDSTECSNLLLQYGS

SEQ ID NO: 689 - protein, 15 aa, Artificial sequence
  betacoronavirus epitope
ECSNLLLQYGSFCTQ

SEQ ID NO: 690 - protein, 15 aa, Artificial sequence
  betacoronavirus epitope
LLLQYGSFCTQLNRA

SEQ ID NO: 691 - protein, 15 aa, Artificial sequence
  betacoronavirus epitope
YGSFCTQLNRALTGI

SEQ ID NO: 692 - protein, 15 aa, Artificial sequence
  betacoronavirus epitope
CTQLNRALTGIAVEQ

SEQ ID NO: 693 - protein, 15 aa, Artificial sequence
  betacoronavirus epitope
NRALTGIAVEQDKNT

SEQ ID NO: 694 - protein, 15 aa, Artificial sequence
  betacoronavirus epitope
TGIAVEQDKNTQEVF

SEQ ID NO: 695 - protein, 15 aa, Artificial sequence
  betacoronavirus epitope
VEQDKNTQEVFAQVK

SEQ ID NO: 696 - protein, 15 aa, Artificial sequence
  betacoronavirus epitope
KNTQEVFAQVKQIYK

SEQ ID NO: 697 - protein, 15 aa, Artificial sequence
  betacoronavirus epitope
EVFAQVKQIYKTPPI

SEQ ID NO: 698 - protein, 15 aa, Artificial sequence
  betacoronavirus epitope
QVKQIYKTPPIKDFG

SEQ ID NO: 699 - protein, 15 aa, Artificial sequence
  betacoronavirus epitope
IYKTPPIKDFGGFNF

SEQ ID NO: 700 - protein, 15 aa, Artificial sequence
  betacoronavirus epitope
PPIKDFGGFNFSQIL

SEQ ID NO: 701 - protein, 15 aa, Artificial sequence
  betacoronavirus epitope
DFGGFNFSQILPDPS

SEQ ID NO: 702 - protein, 15 aa, Artificial sequence
  betacoronavirus epitope
FNFSQILPDPSKPSK

SEQ ID NO: 703 - protein, 15 aa, Artificial sequence
  betacoronavirus epitope
QILPDPSKPSKRSFI

SEQ ID NO: 704 - protein, 15 aa, Artificial sequence
  betacoronavirus epitope
DPSKPSKRSFIEDLL

SEQ ID NO: 705 - protein, 15 aa, Artificial sequence
  betacoronavirus epitope
PSKRSFIEDLLFNKV

SEQ ID NO: 706 - protein, 15 aa, Artificial sequence
  betacoronavirus epitope
SFIEDLLFNKVTLAD

SEQ ID NO: 707 - protein, 15 aa, Artificial sequence
  betacoronavirus epitope
DLLFNKVTLADAGFI

SEQ ID NO: 708 - protein, 15 aa, Artificial sequence
  betacoronavirus epitope
NKVTLADAGFIKQYG

SEQ ID NO: 709 - protein, 15 aa, Artificial sequence
  betacoronavirus epitope
LADAGFIKQYGDCLG

SEQ ID NO: 710 - protein, 15 aa, Artificial sequence
  betacoronavirus epitope
GFIKQYGDCLGDIAA

SEQ ID NO: 711 - protein, 15 aa, Artificial sequence
  betacoronavirus epitope
QYGDCLGDIAARDLI

SEQ ID NO: 712 - protein, 15 aa, Artificial sequence
  betacoronavirus epitope
CLGDIAARDLICAQK

SEQ ID NO: 713 - protein, 15 aa, Artificial sequence
  betacoronavirus epitope
IAARDLICAQKFNGL

SEQ ID NO: 714 - protein, 15 aa, Artificial sequence
  betacoronavirus epitope
DLICAQKFNGLTVLP

SEQ ID NO: 715 - protein, 15 aa, Artificial sequence
  betacoronavirus epitope
AQKFNGLTVLPPLLT

SEQ ID NO: 716 - protein, 15 aa, Artificial sequence
  betacoronavirus epitope
NGLTVLPPLLTDEMI

SEQ ID NO: 717 - protein, 15 aa, Artificial sequence
  betacoronavirus epitope
VLPPLLTDEMIAQYT

SEQ ID NO: 718 - protein, 15 aa, Artificial sequence
  betacoronavirus epitope
LLTDEMIAQYTSALL

SEQ ID NO: 719 - protein, 15 aa, Artificial sequence
  betacoronavirus epitope
EMIAQYTSALLAGTI

SEQ ID NO: 720 - protein, 15 aa, Artificial sequence
  betacoronavirus epitope
QYTSALLAGTITSGW

SEQ ID NO: 721 - protein, 15 aa, Artificial sequence
  betacoronavirus epitope
ALLAGTITSGWTFGA

SEQ ID NO: 722 - protein, 15 aa, Artificial sequence
  betacoronavirus epitope
GTITSGWTFGAGAAL

SEQ ID NO: 723 - protein, 15 aa, Artificial sequence
  betacoronavirus epitope
SGWTFGAGAALQIPF

SEQ ID NO: 724 - protein, 15 aa, Artificial sequence
  betacoronavirus epitope
FGAGAALQIPFAMQM

SEQ ID NO: 725 - protein, 15 aa, Artificial sequence
  betacoronavirus epitope
AALQIPFAMQMAYRF

SEQ ID NO: 726 - protein, 15 aa, Artificial sequence
  betacoronavirus epitope
IPFAMQMAYRFNGIG

SEQ ID NO: 727 - protein, 15 aa, Artificial sequence
  betacoronavirus epitope
MQMAYRFNGIGVTQN

SEQ ID NO: 728 - protein, 15 aa, Artificial sequence
  betacoronavirus epitope
YRFNGIGVTQNVLYE

SEQ ID NO: 729 - protein, 15 aa, Artificial sequence
  betacoronavirus epitope
GIGVTQNVLYENQKL

SEQ ID NO: 730 - protein, 15 aa, Artificial sequence
  betacoronavirus epitope
TQNVLYENQKLIANQ

SEQ ID NO: 731 - protein, 15 aa, Artificial sequence
  betacoronavirus epitope
LYENQKLIANQFNSA

SEQ ID NO: 732 - protein, 15 aa, Artificial sequence
  betacoronavirus epitope
QKLIANQFNSAIGKI

SEQ ID NO: 733 - protein, 15 aa, Artificial sequence
  betacoronavirus epitope
ANQFNSAIGKIQDSL

SEQ ID NO: 734 - protein, 15 aa, Artificial sequence
  betacoronavirus epitope
NSAIGKIQDSLSSTA

SEQ ID NO: 735 - protein, 15 aa, Artificial sequence
  betacoronavirus epitope
GKIQDSLSSTASALG

SEQ ID NO: 736 - protein, 15 aa, Artificial sequence
  betacoronavirus epitope
DSLSSTASALGKLQD

SEQ ID NO: 737 - protein, 15 aa, Artificial sequence
  betacoronavirus epitope
STASALGKLQDVVNQ

SEQ ID NO: 738 - protein, 15 aa, Artificial sequence
  betacoronavirus epitope
ALGKLQDVVNQNAQA

SEQ ID NO: 739 - protein, 15 aa, Artificial sequence
  betacoronavirus epitope
LQDVVNQNAQALNTL

SEQ ID NO: 740 - protein, 15 aa, Artificial sequence
  betacoronavirus epitope
VNQNAQALNTLVKQL

SEQ ID NO: 741 - protein, 15 aa, Artificial sequence
  betacoronavirus epitope
AQALNTLVKQLSSNF

SEQ ID NO: 742 - protein, 15 aa, Artificial sequence
  betacoronavirus epitope
NTLVKQLSSNFGAIS

SEQ ID NO: 743 - protein, 15 aa, Artificial sequence
  betacoronavirus epitope
KQLSSNFGAISSVLN

SEQ ID NO: 744 - protein, 15 aa, Artificial sequence
  betacoronavirus epitope
SNFGAISSVLNDILS

SEQ ID NO: 745 - protein, 15 aa, Artificial sequence
  betacoronavirus epitope
AISSVLNDILSRLDK

SEQ ID NO: 746 - protein, 15 aa, Artificial sequence
  betacoronavirus epitope
VLNDILSRLDKVEAE

SEQ ID NO: 747 - protein, 15 aa, Artificial sequence
  betacoronavirus epitope
ILSRLDKVEAEVQID

SEQ ID NO: 748 - protein, 15 aa, Artificial sequence
  betacoronavirus epitope
LDKVEAEVQIDRLIT

SEQ ID NO: 749 - protein, 15 aa, Artificial sequence
  betacoronavirus epitope
EAEVQIDRLITGRLQ

SEQ ID NO: 750 - protein, 15 aa, Artificial sequence
  betacoronavirus epitope
QIDRLITGRLQSLQT

SEQ ID NO: 751 - protein, 15 aa, Artificial sequence
  betacoronavirus epitope
LITGRLQSLQTYVTQ

SEQ ID NO: 752 - protein, 15 aa, Artificial sequence
  betacoronavirus epitope
RLQSLQTYVTQQLIR

SEQ ID NO: 753 - protein, 15 aa, Artificial sequence
  betacoronavirus epitope
LQTYVTQQLIRAAEI

SEQ ID NO: 754 - protein, 15 aa, Artificial sequence
  betacoronavirus epitope
VTQQLIRAAEIRASA

SEQ ID NO: 755 - protein, 15 aa, Artificial sequence
  betacoronavirus epitope
LIRAAEIRASANLAA

SEQ ID NO: 756 - protein, 15 aa, Artificial sequence
  betacoronavirus epitope
AEIRASANLAATKMS

SEQ ID NO: 757 - protein, 15 aa, Artificial sequence
  betacoronavirus epitope
ASANLAATKMSECVL

SEQ ID NO: 758 - protein, 15 aa, Artificial sequence
  betacoronavirus epitope
LAATKMSECVLGQSK

SEQ ID NO: 759 - protein, 15 aa, Artificial sequence
  betacoronavirus epitope
KMSECVLGQSKRVDF

SEQ ID NO: 760 - protein, 15 aa, Artificial sequence
  betacoronavirus epitope
CVLGQSKRVDFCGKG

SEQ ID NO: 761 - protein, 15 aa, Artificial sequence
  betacoronavirus epitope
QSKRVDFCGKGYHLM

SEQ ID NO: 762 - protein, 15 aa, Artificial sequence
  betacoronavirus epitope
VDFCGKGYHLMSFPQ

SEQ ID NO: 763 - protein, 15 aa, Artificial sequence
  betacoronavirus epitope
GKGYHLMSFPQSAPH

SEQ ID NO: 764 - protein, 15 aa, Artificial sequence
  betacoronavirus epitope
HLMSFPQSAPHGVVF

SEQ ID NO: 765 - protein, 15 aa, Artificial sequence
  betacoronavirus epitope
FPQSAPHGVVFLHVT

SEQ ID NO: 766 - protein, 15 aa, Artificial sequence
  betacoronavirus epitope
APHGVVFLHVTYVPA

SEQ ID NO: 767 - protein, 15 aa, Artificial sequence
  betacoronavirus epitope
VVFLHVTYVPAQEKN

SEQ ID NO: 768 - protein, 15 aa, Artificial sequence
  betacoronavirus epitope
HVTYVPAQEKNFTTA

SEQ ID NO: 769 - protein, 15 aa, Artificial sequence
  betacoronavirus epitope
VPAQEKNFTTAPAIC

SEQ ID NO: 770 - protein, 15 aa, Artificial sequence
  betacoronavirus epitope
EKNFTTAPAICHDGK

SEQ ID NO: 771 - protein, 15 aa, Artificial sequence
  betacoronavirus epitope
TTAPAICHDGKAHFP

SEQ ID NO: 772 - protein, 15 aa, Artificial sequence
  betacoronavirus epitope
AICHDGKAHFPREGV

SEQ ID NO: 773 - protein, 15 aa, Artificial sequence
  betacoronavirus epitope
DGKAHFPREGVFVSN

SEQ ID NO: 774 - protein, 15 aa, Artificial sequence
  betacoronavirus epitope
HFPREGVFVSNGTHW

SEQ ID NO: 775 - protein, 15 aa, Artificial sequence
  betacoronavirus epitope
EGVFVSNGTHWFVTQ

SEQ ID NO: 776 - protein, 15 aa, Artificial sequence
  betacoronavirus epitope
VSNGTHWFVTQRNFY

SEQ ID NO: 777 - protein, 15 aa, Artificial sequence
  betacoronavirus epitope
THWFVTQRNFYEPQI

SEQ ID NO: 778 - protein, 15 aa, Artificial sequence
  betacoronavirus epitope
VTQRNFYEPQIITTD

SEQ ID NO: 779 - protein, 15 aa, Artificial sequence
  betacoronavirus epitope
NFYEPQIITTDNTFV

SEQ ID NO: 780 - protein, 15 aa, Artificial sequence
  betacoronavirus epitope
PQIITTDNTFVSGNC

SEQ ID NO: 781 - protein, 15 aa, Artificial sequence
  betacoronavirus epitope
TTDNTFVSGNCDVVI

SEQ ID NO: 782 - protein, 15 aa, Artificial sequence
  betacoronavirus epitope
TFVSGNCDVVIGIVN

SEQ ID NO: 783 - protein, 15 aa, Artificial sequence
  betacoronavirus epitope
GNCDVVIGIVNNTVY

SEQ ID NO: 784 - protein, 15 aa, Artificial sequence
  betacoronavirus epitope
VVIGIVNNTVYDPLQ

SEQ ID NO: 785 - protein, 15 aa, Artificial sequence
  betacoronavirus epitope
IVNNTVYDPLQPELD

SEQ ID NO: 786 - protein, 15 aa, Artificial sequence
  betacoronavirus epitope
TVYDPLQPELDSFKE

SEQ ID NO: 787 - protein, 15 aa, Artificial sequence
  betacoronavirus epitope
PLQPELDSFKEELDK

SEQ ID NO: 788 - protein, 15 aa, Artificial sequence
  betacoronavirus epitope
ELDSFKEELDKYFKN

SEQ ID NO: 789 - protein, 15 aa, Artificial sequence
  betacoronavirus epitope
FKEELDKYFKNHTSP

SEQ ID NO: 790 - protein, 15 aa, Artificial sequence
  betacoronavirus epitope
LDKYFKNHTSPDVDL

SEQ ID NO: 791 - protein, 15 aa, Artificial sequence
  betacoronavirus epitope
FKNHTSPDVDLGDIS

SEQ ID NO: 792 - protein, 15 aa, Artificial sequence
  betacoronavirus epitope
TSPDVDLGDISGINA

SEQ ID NO: 793 - protein, 15 aa, Artificial sequence
  betacoronavirus epitope
VDLGDISGINASVVN

SEQ ID NO: 794 - protein, 15 aa, Artificial sequence
  betacoronavirus epitope
DISGINASVVNIQKE

SEQ ID NO: 795 - protein, 15 aa, Artificial sequence
  betacoronavirus epitope
INASVVNIQKEIDRL

SEQ ID NO: 796 - protein, 15 aa, Artificial sequence
  betacoronavirus epitope
VVNIQKEIDRLNEVA

SEQ ID NO: 797 - protein, 15 aa, Artificial sequence
  betacoronavirus epitope
QKEIDRLNEVAKNLN

SEQ ID NO: 798 - protein, 15 aa, Artificial sequence
  betacoronavirus epitope
DRLNEVAKNLNESLI

SEQ ID NO: 799 - protein, 15 aa, Artificial sequence
  betacoronavirus epitope
EVAKNLNESLIDLQE

SEQ ID NO: 800 - protein, 15 aa, Artificial sequence
  betacoronavirus epitope
NLNESLIDLQELGKY

SEQ ID NO: 801 - protein, 15 aa, Artificial sequence
  betacoronavirus epitope
SLIDLQELGKYEQYI

SEQ ID NO: 802 - protein, 223 aa, Artificial sequence
  RBD sequence
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 803 - protein, 223 aa, Artificial sequence
  RBD mutated sequence
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNF

SEQ ID NO: 804 - protein, 223 aa, Artificial sequence
  RBD mutated sequence
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNF

SEQ ID NO: 805 - protein, 223 aa, Artificial sequence
  RBD mutated sequence
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 806 - protein, 5 aa, Artificial sequence
  Linker
GGGGS

SEQ ID NO: 807 - protein, 5 aa, Artificial sequence
  Linker
GGGSS

SEQ ID NO: 808 - protein, 5 aa, Artificial sequence
  Linker
GGGSG

SEQ ID NO: 809 - protein, 10 aa, Artificial sequence
  Linker
GGGGSGGGGS

SEQ ID NO: 810 - protein, 5 aa, Artificial sequence
  Linker
LGGGS

SEQ ID NO: 811 - protein, 5 aa, Artificial sequence
  Linker
GLGGS

SEQ ID NO: 812 - protein, 5 aa, Artificial sequence
  Linker
GGLGS

SEQ ID NO: 813 - protein, 5 aa, Artificial sequence
  Linker
GGGLS

SEQ ID NO: 814 - protein, 5 aa, Artificial sequence
  Linker
GGGGL

SEQ ID NO: 815 - protein, 5 aa, Artificial sequence
  Linker
LGGSG

SEQ ID NO: 816 - protein, 5 aa, Artificial sequence
  Linker
GLGSG

SEQ ID NO: 817 - protein, 5 aa, Artificial sequence
  Linker
GGLSG

SEQ ID NO: 818 - protein, 5 aa, Artificial sequence
  Linker
GGGLG

SEQ ID NO: 819 - protein, 5 aa, Artificial sequence
  Linker
LGGSS

SEQ ID NO: 820 - protein, 5 aa, Artificial sequence
  Linker
GLGSS

SEQ ID NO: 821 - protein, 5 aa, Artificial sequence
  Linker
GGLSS

SEQ ID NO: 822 - protein, 5 aa, Artificial sequence
  Linker
GGGSL

SEQ ID NO: 823 - protein, 5 aa, Artificial sequence
  Linker
LGLGS

SEQ ID NO: 824 - protein, 5 aa, Artificial sequence
  Linker
GLGLS

SEQ ID NO: 825 - protein, 5 aa, Artificial sequence
  Linker
GLLGS

SEQ ID NO: 826 - protein, 5 aa, Artificial sequence
  Linker
LGGLS

SEQ ID NO: 827 - protein, 5 aa, Artificial sequence
  Linker
GLGGL

SEQ ID NO: 828 - protein, 5 aa, Artificial sequence
  Linker
LGLSG

SEQ ID NO: 829 - protein, 5 aa, Artificial sequence
  Linker
GLLSG

SEQ ID NO: 830 - protein, 5 aa, Artificial sequence
  Linker
GGLSL

SEQ ID NO: 831 - protein, 5 aa, Artificial sequence
  Linker
GGLLG

SEQ ID NO: 832 - protein, 5 aa, Artificial sequence
  Linker
GLGSL

SEQ ID NO: 833 - protein, 5 aa, Artificial sequence
  Linker
LGLSS

SEQ ID NO: 834 - protein, 5 aa, Artificial sequence
  Linker
GGLLS

SEQ ID NO: 835 - protein, 10 aa, Artificial sequence
  Linker
LGGGSGGGGS

SEQ ID NO: 836 - protein, 10 aa, Artificial sequence
  Linker
GLGGSGGGGS

SEQ ID NO: 837 - protein, 10 aa, Artificial sequence
  Linker
GGLGSGGGGS

SEQ ID NO: 838 - protein, 10 aa, Artificial sequence
  Linker
GGGLSGGGGS

SEQ ID NO: 839 - protein, 10 aa, Artificial sequence
  Linker
GGGGLGGGGS

SEQ ID NO: 840 - protein, 10 aa, Artificial sequence
  Linker
LGGSGGGGSG

SEQ ID NO: 841 - protein, 10 aa, Artificial sequence
  Linker
GLGSGGGGSG

SEQ ID NO: 842 - protein, 10 aa, Artificial sequence
  Linker
GGLSGGGGSG

SEQ ID NO: 843 - protein, 10 aa, Artificial sequence
  Linker
GGGLGGGGSG

SEQ ID NO: 844 - protein, 10 aa, Artificial sequence
  Linker
GGGSLGGGSG

SEQ ID NO: 845 - protein, 10 aa, Artificial sequence
  Linker
LGGSSGGGSS

SEQ ID NO: 846 - protein, 10 aa, Artificial sequence
  Linker
GLGSSGGGSS

SEQ ID NO: 847 - protein, 10 aa, Artificial sequence
  Linker
GGLSSGGGSS

SEQ ID NO: 848 - protein, 10 aa, Artificial sequence
  Linker
GGGLSGGGSS

SEQ ID NO: 849 - protein, 10 aa, Artificial sequence
  Linker
GGGSLGGGSS

SEQ ID NO: 850 - protein, 10 aa, Artificial sequence
  Linker
LGGGSLGGGS

SEQ ID NO: 851 - protein, 10 aa, Artificial sequence
  Linker
GLGGSGLGGS

SEQ ID NO: 852 - protein, 10 aa, Artificial sequence
  Linker
GGLGSGGLGS

SEQ ID NO: 853 - protein, 10 aa, Artificial sequence
  Linker
GGGLSGGGLS

SEQ ID NO: 854 - protein, 10 aa, Artificial sequence
  Linker
GGGGLGGGGL

SEQ ID NO: 855 - protein, 10 aa, Artificial sequence
  Linker
LGGSGLGGSG

SEQ ID NO: 856 - protein, 10 aa, Artificial sequence
  Linker
GLGSGGLGSG

SEQ ID NO: 857 - protein, 10 aa, Artificial sequence
  Linker
GGLSGGGLSG

SEQ ID NO: 858 - protein, 10 aa, Artificial sequence
  Linker
GGGLGGGGLG

SEQ ID NO: 859 - protein, 10 aa, Artificial sequence
  Linker
GGGSLGGGSL

SEQ ID NO: 860 - protein, 10 aa, Artificial sequence
  Linker
LGGSSLGGSS

SEQ ID NO: 861 - protein, 10 aa, Artificial sequence
  Linker
GLGSSGLGSS

SEQ ID NO: 862 - protein, 10 aa, Artificial sequence
  Linker
GGLSSGGLSS

SEQ ID NO: 863 - protein, 16 aa, Artificial sequence
  Linker
TQKSLSLSPGKGLGGL

SEQ ID NO: 864 - protein, 13 aa, Artificial sequence
  Linker
SLSLSPGKGLGGL

SEQ ID NO: 865 - protein, 4 aa, Artificial sequence
  Linker
GSAT

SEQ ID NO: 866 - protein, 36 aa, Artificial sequence
  Linker
GGSAGGSGSGSSGGSSGASGTGTAGGTGSGSGTGSG

SEQ ID NO: 867 - protein, 36 aa, Artificial sequence
  Linker
GGSGGGSEGGGSEGGGSEGGGSEGGGSEGGGSGGGS

SEQ ID NO: 868 - protein, 5 aa, Artificial sequence
  Linker
GLSGL

(19) United States
(12) Patent Application Publication
(10) Pub. No.: US 2023/0165952 A1
(43) Pub. Date: Jun. 1, 2023

(54) BETACORONAVIRUS PROPHYLAXIS AND THERAPY

(71) Applicant: Nykode Therapeutics ASA, Oslo (NO)

(72) Inventors: Agnete Brunsvik Fredriksen, Rælingen (NO); Monika Sekelja, Oslo (NO); Karoline Schjetne, Lommedalen (NO); Gunnstein Norheim, Oslo (NO); Elisabeth Stubsrud, Oslo (NO)

(73) Assignee: Nykode Therapeutics ASA, Oslo (NO)

(21) Appl. No.: 17/997,407

(22) PCT Filed: May 3, 2021
(86) PCT No.: PCT/EP2021/061602

§ 371 Date: Oct. 28, 2022

(30) Foreign Application Priority Data
May 1, 2020 (DK) PA 2020 70282
May 6, 2020 (DK) PA 2020 70293
Nov. 5, 2020 (DK) PA 2020 70735
Dec. 8, 2020 (DK) PA 2020 70820
Feb. 15, 2021 (DK) PA 2021 70069

(51) Int. Cl. A61K 39/215; A61P 31/14

(52) U.S. Cl. CPC A61K 39/215; A61P 31/14; A61K 2039/6075

(57) ABSTRACT

Disclosed is a vaccine comprising an immunologically effective amount of a polynucleotide comprising a nucleotide sequence encoding a targeting unit, a dimerization unit and an antigenic unit, wherein the antigenic unit comprises at least one betacoronavirus epitope. The vaccine is ideal for pandemic and epidemics as it can induce rapid, strong immune response with lower/fewer doses because the antigen is targeted to antigen presenting cells and the antigen is produced in the body.

### TECHNICAL FIELD

The present invention relates to therapeutic and prophylactic vaccines against betacoronavirus, such as vaccines against corona virus disease 2019 (COVID-19).

### SUMMARY

The present invention relates to a vaccine comprising a vaccibody construct ideal for fighting pandemics and epidemics as it can induce rapid, strong immune response with lower/fewer doses compared to typical vaccines because the antigen is targeted to antigen presenting cells and the antigen preferably is produced in the body. This vaccibody construct is designed to induce an antigenic effect through the full length or a part of the spike protein; or selected T cell epitopes, e.g. those which are conserved between different betacoronaviruses (such as SARS-CoV and SARS-CoV2); or through combinations thereof.

By targeting these antigenic epitopes in the body through e.g. anti-pan HLA class II or MIP-1α, an immune response will be raised through B cells and/or T cells, such that the vaccine can be used in a prophylactic setting and a therapeutic setting.

### DESCRIPTION OF DRAWINGS

FIG. 1:

Full-length spike protein of SARS-CoV-2 (SEQ ID NO: 230)

FIG. 2:

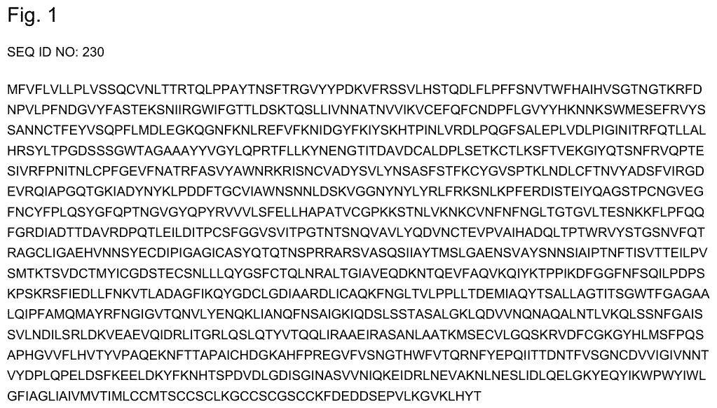

A: Exemplary sequence of RBD of spike protein of SARS-CoV-2 (SEQ ID NO: 231)

B: The RBD sequence of spike protein of SARS-CoV-2 of the Wuhan strain used in the VB10.COV2 constructs of the Examples (SEQ ID NO: 802)

C: The RBD sequence of spike protein of SARS-CoV-2 South African variant B.1.351 used in the VB10.COV2 constructs of the Examples ((SEQ ID NO: 803)

D: The RBD sequence of spike protein of SARS-CoV-2 UK variant B.1.1.7 used in the VB10.COV2 constructs of the Examples (SEQ ID NO: 804)

E: The RBD sequence of spike protein of SARS-CoV-2 Californian variant B.1.427 used in the VB10.COV2 constructs of the Examples ((SEQ ID NO: 805)

FIG. 3:

HR2 domain of spike protein of SARS-CoV-2 and SARS-CoV (SEQ ID NO: 232)

FIG. 4:

Amino acid sequence of signal peptide and mature peptide of hMIP1α (LD78b), human hinge region 1 from IgG3, human hinge region 4 from IgG3, glycine-serine linker, human CH3 domain of IgG3 and glycine-leucine linker (SEQ ID NO: 233).

The sequence is split up by “|” to help distinguish the various parts of the sequence

FIG. 5:

C—C motif chemokine 3-like 1 precursor including signal peptide (amino acids 1-23) and mature peptide (hMIP1α/LD78-beta, amino acids 24-93) (SEQ ID NO: 234)

FIG. 6:

Signal peptide (SEQ ID NO: 235)

FIG. 7:

Signal peptide (SEQ ID NO: 236)

FIG. 8:

Amino acid sequence of the antigenic unit of VB2040 (SEQ ID NO: 237)

FIG. 9:

Amino acid sequence of the antigenic unit of VB2041 (SEQ ID NO: 238)

FIG. 10:

Amino acid sequence of the antigenic unit of VB2042 (SEQ ID NO: 239)

FIG. 11:

Amino acid sequence of the antigenic unit of VB2043 (SEQ ID NO: 240)

FIG. 12:

Amino acid sequence of the antigenic unit of VB2044 (SEQ ID NO: 241)

FIG. 13:

Amino acid sequence of the antigenic unit of VB2045 (SEQ ID NO: 242)

FIG. 14:

Amino acid sequence of the antigenic unit of VB2046 (SEQ ID NO: 243)

FIG. 15:

Amino acid sequence of the antigenic unit of VB2047 (SEQ ID NO: 244)

FIG. 16:

Amino acid sequence of the antigenic unit of VB2048 (SEQ ID NO: 245).

FIG. 17:

Amino acid sequence of the antigenic unit of VB2049 (SEQ ID NO: 246)

FIG. 18:

Amino acid sequence of the antigenic unit of VB2050 (SEQ ID NO: 247)

FIG. 19:

Amino acid sequence of the antigenic unit of VB2051 (SEQ ID NO: 248)

FIG. 20:

Alternative HR2 domain of spike protein of SARS-CoV-2 and SARS-CoV (SEQ ID NO: 249)

FIG. 21:

Amino acid sequence of the antigenic unit of VB2053 (SEQ ID NO: 250)

FIG. 22:

Amino acid sequence of the antigenic unit of VB2054 (SEQ ID NO: 251)

FIG. 23:

A. Nucleotide sequence of VB2049 (SEQ ID NO: 252)

B. Amino acid sequence of VB2049 (SEQ ID NO: 253)

The nucleotide sequence encodes the VB2049 protein with its targeting unit hMIP-1α, the dimerisation unit comprising h1 and h4 and the CH3 domain of hIgG3, and the short RBD domain.

FIG. 24:

A. Nucleotide sequence of VB2060 (SEQ ID NO: 254)

B. Amino acid sequence of VB2060 (SEQ ID NO: 255)

The nucleotide sequence encodes the VB2060 protein with its targeting unit hMIP-1α, the dimerisation unit comprising h1 and h4 and the CH3 domain of hIgG3, and the long RBD domain.

FIG. 25:

A. Nucleotide sequence of VB2065 (SEQ ID NO: 256)

B. Amino acid sequence of VB2065 (SEQ ID NO: 257)

The capitalised nucleotide sequence encodes the VB2065 protein with its targeting unit hMIP-1α, the dimerisation unit comprising h1 and h4 and the CH3 domain of hIgG3, and the spike domain.

FIG. 26:

A. Nucleotide sequence of VB2048 (SEQ ID NO: 258)

B. Amino acid sequence of VB2048 (SEQ ID NO: 259)

The nucleotide sequence encodes the VB2048 protein with its targeting unit hMIP-1α, the dimerisation unit comprising h1 and h4 and the CH3 domain of hIgG3, and 20 predicted T cell epitopes.

FIG. 27:

A. Nucleotide sequence of VB2059 (SEQ ID NO: 260)

B. Amino acid sequence of VB2059 (SEQ ID NO: 261)

The nucleotide sequence encodes the VB2059 protein with its targeting unit anti-mouse MHCII scFv, the dimerisation unit comprising h1 and h4 and the CH3 domain of hIgG3, and the long RBD domain.

FIG. 28:

A. Nucleotide sequence of VB2071 (SEQ ID NO: 262)

B. Amino acid sequence of VB2071 (SEQ ID NO: 263)

The nucleotide sequence encodes the VB2071 protein with its targeting unit anti-mouse MHCII scFv, the dimerisation unit comprising h1 and h4 and the CH3 domain of hIgG3, and the spike protein.

FIG. 29:

A. Nucleotide sequence of VB2081 (SEQ ID NO: 264)

B. Amino acid sequence of VB2081 (SEQ ID NO: 265)

The nucleotide sequence encodes the VB2081 protein with its targeting unit hMIP-1α, the dimerisation unit comprising h1 and h4 and the CH3 domain of hIgG3, and an antigenic unit comprising 1 predicted T cell epitope (pep08) and the long RBD domain linked with a (GGGGS)₂ linker.

FIG. 30:

A. Nucleotide sequence of VB2082 (SEQ ID NO: 266)

B. Amino acid sequence of VB2082 (SEQ ID NO: 267)

The nucleotide sequence encodes the VB2082 protein with its targeting unit hMIP-1α, the dimerisation unit comprising h1 and h4 and the CH3 domain of hIgG3, and an antigenic unit comprising 1 predicted T cell epitope (pep18) and the long RBD domain linked with a (GGGGS)₂ linker.

FIG. 31:

A. Nucleotide sequence of VB2083 (SEQ ID NO: 268)

B. Amino acid sequence of VB2083 (SEQ ID NO: 269)

The nucleotide sequence encodes the VB2083 protein with its targeting unit hMIP-1α, the dimerisation unit comprising h1 and h4 and the CH3 domain of hIgG3, and an antigenic unit comprising 2 predicted T cell epitopes (pep08+pep18 with a (GGGGS)₂ linker in between epitopes) and the long RBD domain, linked with a (GGGGS)₂ linker.

FIG. 32:

A. Nucleotide sequence of VB2084 (SEQ ID NO: 270)

B. Amino acid sequence of VB2084 (SEQ ID NO: 271)

The nucleotide sequence encodes the VB2084 protein with its targeting unit hMIP-1α, the dimerisation unit comprising h1 and h4 and the CH3 domain of hIgG3, and an antigenic unit comprising 3 predicted T cell epitopes (pep08, pep18+pep25 with a (GGGGS)₂ linker in between epitopes) and the long RBD domain, linked with a (GGGGS)₂ linker.

FIG. 33:

A. Nucleotide sequence of VB2085 (SEQ ID NO: 272)

B. Amino acid sequence of VB2085 (SEQ ID NO: 273)

The nucleotide sequence encodes the VB2085 protein with its targeting unit hMIP-1α, the dimerisation unit comprising h1 and h4 and the CH3 domain of hIgG3, and an antigenic unit comprising 1 predicted T cell epitope (pep08) and the long RBD domain, linked with a GLGGL linker.

FIG. 34:

A. Nucleotide sequence of VB2086 (SEQ ID NO: 274)

B. Amino acid sequence of VB2086 (SEQ ID NO: 275)

The nucleotide sequence encodes the VB2086 protein with its targeting unit hMIP-1α, the dimerisation unit comprising h1 and h4 and the CH3 domain of hIgG3, and an antigenic unit comprising 1 predicted T cell epitope (pep08) and the long RBD domain, linked with a (GLGGL)₂ linker.

FIG. 35:

A. Nucleotide sequence of VB2087 (SEQ ID NO: 276)

B. Amino acid sequence of VB2087 (SEQ ID NO: 277)

The nucleotide sequence encodes the VB2087 protein with its targeting unit hMIP-1α, the dimerisation unit comprising h1 and h4 and the CH3 domain of hIgG3, and an antigenic unit comprising 1 predicted T cell epitope (pep18) and the long RBD domain, linked with a GLGGL linker.

FIG. 36:

A. Nucleotide sequence of VB2088 (SEQ ID NO: 278)

B. Amino acid sequence of VB2088 (SEQ ID NO: 279)

The nucleotide sequence encodes the VB2088 protein with its targeting unit hMIP-1α, the dimerisation unit comprising h1 and h4 and the CH3 domain of hIgG3, and an antigenic unit comprising 2 predicted T cell epitopes (pep08+pep18 with a (GGGGS)₂ linker in between epitopes) and the long RBD domain, linked with a GLGGL linker.

FIG. 37:

A. Nucleotide sequence of VB2089 (SEQ ID NO: 280)

B. Amino acid sequence of VB2089 (SEQ ID NO: 281)

The nucleotide sequence encodes the VB2089 protein with its targeting unit hMIP-1α, the dimerisation unit comprising h1 and h4 and the CH3 domain of hIgG3, and an antigenic unit comprising 3 predicted T cell epitopes (pep08, pep18 and pep25 with a (GGGGS)₂ linker in between epitopes) and the long RBD domain, linked with a GLGGL linker.

FIG. 38:

A. Nucleotide sequence of VB2091 (SEQ ID NO: 282)

B. Amino acid sequence of VB2091 (SEQ ID NO: 283)

The nucleotide sequence encodes the VB2091 protein with its targeting unit hMIP-1α, the dimerisation unit comprising h1 and h4 and the CH3 domain of hIgG3, and an antigenic unit comprising 1 predicted T cell epitope (pep08) and the long RBD domain, linked with a TQKSLSLSPGKGLGGL linker.

FIG. 39:

A. Nucleotide sequence of VB2092 (SEQ ID NO: 284)

B. Amino acid sequence of VB2092 (SEQ ID NO: 285)

The nucleotide sequence encodes the VB2092 protein with its targeting unit hMIP-1α, the dimerisation unit comprising h1 and h4 and the CH3 domain of hIgG3, and an antigenic unit comprising 3 predicted T cell epitopes (pep08, pep18 and pep25 with a (GGGGS)₂ linker in between epitopes) and the long RBD domain, linked with a TQKSLSLSPGKGLGGL linker.

FIG. 40:

A. Nucleotide sequence of VB2094 (SEQ ID NO: 286)

B. Amino acid sequence of VB2094 (SEQ ID NO: 287)

The nucleotide sequence encodes the VB2094 protein with its targeting unit hMIP-1α, the dimerisation unit comprising h1 and h4 and the CH3 domain of hIgG3, and an antigenic unit comprising 1 predicted T cell epitope (pep08) and the long RBD domain, linked with a SLSLSPGKGLGGL linker.

FIG. 41:

A. Nucleotide sequence of VB2095 (SEQ ID NO: 288)

B. Amino acid sequence of VB2095 (SEQ ID NO: 289)

The nucleotide sequence encodes the VB2095 protein with its targeting unit hMIP-1α, the dimerisation unit comprising h1 and h4 and the CH3 domain of hIgG3, and an antigenic unit comprising 3 predicted T cell epitopes (pep08, pep18 and pep25 with a (GGGGS)₂ linker in between epitopes) and the long RBD domain, linked with a SLSLSPGKGLGGL linker.

FIG. 42:

A. Nucleotide sequence of VB2097 (SEQ ID NO: 290)

B. Amino acid sequence of VB2097 (SEQ ID NO: 291)

The nucleotide sequence encodes the VB2097 protein with its targeting unit hMIP-1α, the dimerisation unit comprising h1 and h4 and the CH3 domain of hIgG3, and an antigenic unit comprising 3 predicted T cell epitopes (pep08, pep18 and pep25 with a (GGGGS)₂ linker in between epitopes) and the long RBD domain, linked with a GSAT linker.

FIG. 43:

A. Nucleotide sequence of VB2099 (SEQ ID NO: 292)

B. Amino acid sequence of VB2099 (SEQ ID NO: 293)

The nucleotide sequence encodes the VB2099 protein with its targeting unit hMIP-1α, the dimerisation unit comprising h1 and h4 and the CH3 domain of hIgG3, and an antigenic unit comprising 3 predicted T cell epitopes (pep08, pep18 and pep25 with a (GGGGS)₂ linker in between epitopes) and the long RBD domain, linked with a SEG linker.

FIG. 44:

A. Nucleotide sequence of VB2129 (SEQ ID NO: 294)

B. Amino acid sequence of VB2129 (SEQ ID NO: 295)

The nucleotide sequence encodes the VB2129 protein with its targeting unit hMIP-1α, the dimerisation unit comprising h1 and h4 and the CH3 domain of hIgG3, and an antigenic unit comprising the long RBD domain with 3 mutations characterised in the South African variant B.1.351.

FIG. 45:

Amino acid sequence of VB2131 (SEQ ID NO: 296)

The VB2131 protein with its targeting unit hMIP-1α, the dimerisation unit comprising h1 and h4 and the CH3 domain of hIgG3, and an antigenic unit comprising two long RBD domains (RBD from the Wuhan strain and the South African variant B.1.351) linked with a SEG linker.

FIG. 46:

Amino acid sequence of VB2132 (SEQ ID NO: 297)

The VB2132 protein with its targeting unit hMIP-1α, the dimerisation unit comprising h1 and h4 and the CH3 domain of hIgG3, and an antigenic unit comprising two long RBD domains (RBD from the Wuhan strain and the South African variant B.1.351) linked with a GSAT linker.

FIG. 47:

Amino acid sequence of VB2133 (SEQ ID NO: 298)

The VB2133 protein with its targeting unit hMIP-1α, the dimerisation unit comprising h1 and h4 and the CH3 domain of hIgG3, and an antigenic unit comprising two long RBD domains (RBD from the Wuhan strain and the South African variant B.1.351) linked with a TQKSLSLSPGKGLGGL linker.

FIG. 48:

Amino acid sequence of VB2134 (SEQ ID NO: 299)

The VB2134 protein with its targeting unit hMIP-1α, the dimerisation unit comprising h1 and h4 and the CH3 domain of hIgG3, and an antigenic unit comprising two long RBD domains (RBD from the Wuhan strain and the South African variant B.1.351) linked with a SLSLSPGKGLGGL linker.

FIG. 49:

Amino acid sequence of VB2135 (SEQ ID NO: 300)

The VB2135 protein with its targeting unit hMIP-1α, the dimerisation unit comprising h1 and h4 and the CH3 domain of hIgG3, and an antigenic unit comprising two long RBD domains (RBD from the South African variant B.1.351 and the UK variant B.1.1.7) linked with a SEG linker.

FIG. 50:

Amino acid sequence of VB2136 (SEQ ID NO: 301)

The VB2136 protein with its targeting unit hMIP-1α, the dimerisation unit comprising h1 and h4 and the CH3 domain of hIgG3, and an antigenic unit comprising two long RBD domains (RBD from the South African variant B.1.351 and the UK variant B.1.1.7) linked with a GSAT linker.

FIG. 51:

Amino acid sequence of VB2137 (SEQ ID NO: 302)

The VB2137 protein with its targeting unit hMIP-1α, the dimerisation unit comprising h1 and h4 and the CH3 domain of hIgG3, and an antigenic unit comprising two long RBD domains (RBD from the South African variant B.1.351 and the Californian variant B.1.427) linked with a SEG linker.

FIG. 52:

Amino acid sequence of VB2138 (SEQ ID NO: 303)

The VB2138 protein with its targeting unit hMIP-1α, the dimerisation unit comprising h1 and h4 and the CH3 domain of hIgG3, and an antigenic unit comprising two long RBD domains (RBD from the South African variant B.1.351 and the Californian variant B.1.427) linked with a GSAT linker.

FIG. 53:

Overview of protein formats of the VB10.COV2 constructs used in the non-clinical development:

A: VB2049, VB2060, VB2065 and VB2048.

B: VB2059 and VB2071.

C: VB2081-VB2099.

D: VB2129.

E: VB2131-VB2138.

FIG. 54:

VB10.COV2 vaccibody proteins VB2049, VB2060 and VB2065 were produced and secreted as functional homodimers 3 days after transfection of HEK293 cells. Conformational integrity of the proteins was confirmed by binding to antibodies detecting human MIP-1α (targeting unit), human IgG CH3 domain (dimerisation unit) (as capture antibody), the RBD domain or the spike protein (antigenic unit) in ELISA.

FIG. 55:

A: VB2048 vaccibody protein were produced and secreted as a functional homodimer 3 days after transfection of HEK293 cells. Conformational integrity of the proteins was confirmed by binding to antibodies detecting human MIP-1α (targeting unit) and antibodies capturing human IgG CH3 domain (dimerisation unit).

B: VB10.COV2 vaccibody proteins VB2059 and VB2071 were produced and secreted as functional homodimers 3 days after transfection of HEK293 cells. Conformational integrity of the proteins was confirmed by binding to antibodies detecting human IgG CH3 domain (dimerisation unit), the RBD domain or the spike protein (antigenic unit) in ELISA.

C: VB10.COV2 vaccibody proteins VB2081-VB2099 were produced and secreted as functional homodimers 6 days after transfection of HEK293 cells. Conformational integrity of the proteins was confirmed by binding to antibodies capturing human IgG CH3 domain (dimerisation unit) and detecting the RBD domain (antigenic unit) protein in ELISA.

D: VB10.COV2 vaccibody proteins VB2129 and VB2060 were produced and secreted as functional homodimers 3 days after transfection of HEK293 cells. Conformational integrity of the proteins was confirmed by binding to antibodies detecting human MIP-1α (targeting unit), human IgG CH3 domain (dimerisation unit, as capture antibody) and the RBD domain protein (antigenic unit) in ELISA.

E: ELISA carried out on supernatants harvested on day 3 post transient transfection from HEK293 cells which had been co-transfected with VB2048 and VB2049.

Conformational integrity of the proteins was confirmed by binding to antibodies detecting human IgG CH3 domain (dimerisation unit) (as capture antibody) and human MIP-1α (targeting unit), or the RBD domain protein (antigenic unit) in ELISA. Expression of both plasmids are confirmed by these results in combination with data showing an immune response in vivo in mice against VB2048 (response against T cell epitopes) and VB2049 (response against RBD domain) (for example FIG. 76).

FIG. 56:

SDS-PAGE and western blot analysis of VB10.COV2 vaccibody protein VB2060. (A) Supernatant harvested from VB2060-transfected HEK293 cells under reducing (SDS+reducing agent) or non-reducing (SDS) conditions. Supernatant was harvested 6 days after transient transfection and up-concentrated about 4-times before loaded onto gels. Arrows indicate possible bands.

FIG. 57:

A: Anti-RBD IgG immune response in mice vaccinated with VB2049 or VB2060 DNA vaccine according to the invention (bar chart and line chart). Mice were vaccinated by intramuscular administration of DNA immediately followed by electroporation of the injection site. Vaccine, administration days, dose number and dose levels are indicated. Mean of 2 independent experiments are shown.

B: Anti-RBD IgG immune response in mice vaccinated with 2 doses of 50 μg of one of three VB10.COV2 DNA vaccines (VB2049, VB2060, VB2065 and VB2071). Mice were vaccinated by intramuscular administration of DNA on days 0 and 21, immediately followed by electroporation of the injection site. Type of vaccine and controls (PBS) are indicated. Sera obtained at days 7, 14 and 28 post first vaccination were tested for anti-RBD IgG antibodies binding the RBD protein. Mean of up to 5 mice per group is shown.

C: Anti-RBD IgG immune response in mice vaccinated with 1 or 2 doses of either 3, 6, 12.5 or 25 μg of VB2060 DNA vaccine. Mice were vaccinated by intramuscular administration of DNA on day(s) 0 (and 21), immediately followed by electroporation of the injection site. Sera obtained at days 7, 14 and 21 and 28 post first vaccination and at day 7 post boost vaccination at day 21 were tested for anti-RBD IgG antibodies binding the RBD protein. Mean of 4-5 mice per group is shown.

D: Anti-RBD IgG measured in bronchoalveolar lavage (BAL) of mice vaccinated with either 1 dose or 2 doses of 3, 6.25, 12.5 or 25 μg of VB2060 DNA vaccine. Mice were vaccinated by intramuscular administration of DNA on either on day 0, or day 0 and 21, immediately followed by electroporation of the injection site. BAL fluid obtained at days 14, 21 and 28 post first vaccination and day 7 post boost vaccination at day 21 was tested for anti-RBD.

E: Anti-RBD IgG immune response in mice vaccinated with VB2059 DNA vaccine according to the invention. Mice were vaccinated by intramuscular administration of DNA immediately followed by electroporation of the injection site. Vaccine, administration days, dose number and dose levels are indicated. Mean of 2 independent experiments are shown.

F: Anti-RBD IgG immune response in mice vaccinated with 1 dose of 25 μg of the indicated vaccine candidates. Mice were vaccinated by intramuscular administration of DNA on day 0, immediately followed by electroporation of the injection site. Sera obtained at day 14 post vaccination were tested for anti-RBD IgG antibodies binding the RBD protein. Mean of 2-5 mice per group is shown.

G: Anti-RBD IgG immune response in mice vaccinated with 1 or 2 doses of either 1, 6.25, 12.5 or 25 μg of VB2129 and VB2060 DNA vaccine. Mice were vaccinated by intramuscular administration of DNA on day(s) 0 (and 21), immediately followed by electroporation of the injection site. Sera obtained at days 7, 14 and 21 and 28 post first vaccination and at day 7 post boost vaccination at day 21 were tested for anti-RBD IgG antibodies binding the RBD protein. Mean of 4-5 mice per group is shown.

H: Anti-RBD IgG immune response in mice vaccinated with a vaccine comprising DNA plasmids VB2048 and VB2049. 1 dose of 12.5 μg of each plasmid combined in one pharmaceutically acceptable carrier was administered intramuscularly to mice on day 0, immediately followed by electroporation of the injection site. Sera obtained at days 7 and 14 post vaccination were tested for anti-RBD IgG antibodies binding the RBD protein. Mean of 3-5 mice per group is shown.

FIG. 58:

A: VB10.COV2 DNA vaccines VB2049, VB2060 and VB2065 elicit robust neutralizing antibody responses. Mice were vaccinated intramuscularly at day 0, 21 and 89 with 2.5 μg, 25 μg or 50 μg of VB2049, VB2060 or VB2065 (groups tested are indicated). Sera were collected and assessed for neutralizing antibodies against homotypic SARS-CoV-2 live virus strain Australia/VIC01/2020 isolate 44. Sera from PBS vaccinated mice served as a negative control and NIBSC 20/130 served as a positive control. Dotted line indicates the assay limit of detection.

B: VB10.COV2 DNA vaccine VB2060 elicits robust neutralizing antibody responses. Mice were vaccinated intramuscularly at day (s) 0 (and 21) with either 3, 6, 12.5 or 25 μg of VB2060 (groups tested are indicated). Sera were collected and assessed for neutralizing antibodies against homotypic SARS-CoV-2 live virus strain Australia/VIC01/2020 isolate 44. Sera from PBS vaccinated mice served as a negative control and NIBSC 20/130 served as a positive control. Dotted line indicates the assay limit of detection.

FIG. 59:

T cell response induced with different doses and number of doses of VB10.COV2 DNA vaccine VB2049. Total number of IFN-γ positive spots/1×10⁶ splenocytes from mice (5 mice/group) vaccinated intramuscularly with 2.5 μg or 25 μg VB2049 DNA plasmid after re-stimulation with overlapping RBD peptide pools. Splenocytes were harvested at day 14 post first vaccination and at day 7 post boost vaccination at day 21.

FIG. 60:

Induction of CD4+ and CD8+ RBD specific immune responses and T cell epitope mapping after intramuscular vaccination of mice. CD4 and CD8 cell populations were stimulated for 24 hours with 61 individual RBD peptides (15-mer peptides overlapping by 12 amino acid from SARS-COV2 RBD domain) and number of IFN-γ positive spots/1×10⁶ splenocytes were detected in an ELISpot assay

A. Vaccination of mice (5 animals/group) with 2×25 μg of VB2049 on day 0 and 21 (boost vaccination) and ELISpot assay performed on day 28 (7 days post boost vaccination).

B. Vaccination of mice (2-3 animals/group) with 3×50 μg of VB2060 on day 0, 21 and 89 and ELISpot assay performed on day 99 (10 days post last boost vaccination).

C. Map of the SARS-COV2 RBD domain and identification of immunodominant peptides in BALB/c mice.

FIG. 61:

A. Kinetics of T cells responses in mice vaccinated with 25 μg of VB2060. Mice were vaccinated with 1 or 2 doses (days 0 and 7) and the splenocytes were harvested at day 4, day 7, day 11, day 14, day 18 and day 21 5-6 animals/group, except day 21 (2 animals/group) for single immunization group.

B. T cell response induced comparing three VB10.COV2 DNA vaccines, VB2049, VB2059 and VB2060. Total number of IFN-γ positive spots/1×10⁶ splenocytes from mice (4-5 mice/group) vaccinated intramuscularly with 2×2.5 μg of three VB10.COV2 DNA plasmids after re-stimulation with overlapping RBD peptide pools. Splenocytes were harvested at day 28 (7 days post boost vaccination at day 21).

FIG. 62:

T cell response induced with different doses and number of doses of VB2060. Total number of IFN-γ positive spots/1×10⁶ splenocytes from mice (2-3 animals/group) vaccinated intramuscularly with 25 μg or 50 μg VB2060 DNA plasmid after re-stimulation with overlapping RBD peptide pools. Splenocytes were harvested either at day 90 post first vaccination or 10 days after the boost vaccination at day 89.

FIG. 63:

A: Induction of CD4+ and CD8+ spike specific immune responses elicited by vaccination with VB2065 or VB2071 DNA vaccine. Total number of IFN-γ positive spots/1×10⁶ splenocytes from mice (5-6 animals/group) vaccinated intramuscularly with two doses of 50 μg VB2065 DNA plasmid (day 0 and 21) after re-stimulation with spike peptide pools. Splenocytes were harvested at day 28 (7 days post boost vaccination at day 21).

B T cell response induced by the DNA vaccine VB2129. Total number of IFN-γ positive spots/1×10⁶ splenocytes from mice (5 mice/group) vaccinated intramuscularly with 1×1.0, 6.25, 12.5 or 25 μg after re-stimulation with overlapping RBD peptide pools. Splenocytes were harvested at day 7 and 14 days post vaccination.

FIG. 64:

A and B:

Th1/Th2 cytokine profile indicative of RBD specific Th1 responses. Cytokine concentration in supernatant of splenocytes cell culture from mice vaccinated intramuscularly with VB10.COV2 DNA vaccines (A) VB2060, VB2049 or VB2059 and (B) VB2065 or VB2071 and control group (PBS).

FIG. 65:

Gating strategy for identification of T cells: A. All cells were examined using side scatter (SSC) and forward scatter (FSC) parameters. Lymphocyte gate was set based on the relative size (FSC) of the cells. B. Lymphocytes were analyzed for presence of doublets, and a gate was set to include only single cells in further analysis. C. Dead cells were identified using viability dye and a gate was set to include live cells in further analysis. D. In the population of live cells all CD3+ cells were gated for future analysis. E. T cells were defined as CD3+ and γδ TCR T cells were excluded from the analysis. F. All T cells were analyzed for expression of CD4 and CD8 markers.

FIG. 66:

Detection of RBD specific multifunctional T cell responses in VB2060 (A and B) or VB2049 (C and D) vaccinated mice 28 days post first vaccination. A/C. Percent of CD4+ and CD8+ T cells responding to RBD stimulation. Percent of cells expressing each marker (or combinations of markers) is shown as total of each respective population. B/D. Graphical presentation of response type based on the expression of cytokines. CD4 and CD8 graphs were made using SPICE software.

FIG. 67:

Detection of RBD specific multifunctional CD4+ T cell responses in VB2060 vaccinated mice 90 days post first vaccination. A. Percent of CD4⁺ T cells responding to RBD stimulation. Graph shows production of cytokines in three groups—twice vaccinated with medium dose (VB2060 2×25 μg), once vaccinated with high dose (VB2060 1×50 μg) and twice vaccinated with high dose (VB2060 2×50 μg). B. Graphical presentation of response type based on the expression of cytokines. CD4 and CD8 graphs were made using SPICE software.

FIG. 68:

Detection of RBD specific multifunctional CD8+ T cell responses in VB2060 vaccinated mice 90 days post initial vaccination. A. Percent of CD8+ T cells responding to RBD stimulation. Graph shows production of cytokines in three groups—twice vaccinated with medium dose (VB2060 2×25 μg), once vaccinated with high dose (VB2060 1×50 μg) and twice vaccinated with high dose (VB2060 2×50 μg). B. Graphical presentation of response type based on the expression of cytokines. Pie charts were made using SPICE software.

FIG. 69:

Detection of RBD specific multifunctional CD4+ T cell responses in VB2060 vaccinated mice 100 days post initial vaccination. A. Percent of CD4+ T cells responding to RBD stimulation. Graph shows production of cytokines in three groups—thrice vaccinated with medium dose (VB2060 3×25 μg), twice vaccinated with high dose (VB2060 2×50 μg) and thrice vaccinated with high dose (VB2060 3×50 μg). B. Graphical presentation of response type based on the expression of cytokines. Pie charts were made using SPICE software.

FIG. 70:

Detection of RBD specific multifunctional CD8+ T cell responses in VB2060 vaccinated mice 100 days post initial vaccination. A. Percent of CD8+ T cells responding to RBD stimulation. Graph shows production of cytokines in three groups—3× vaccinated with medium dose (VB2060 3×25 μg), 2× vaccinated with high dose (VB2060 2×50 μg) and 3× vaccinated with high dose (VB2060 3×50 μg) B. Graphical presentation of response type based on the expression of cytokines. CD4 and CD8 Graphs were made using SPICE software.

FIG. 71:

T cell responses in lymph node 7 days post vaccination and 7 days post boost. Mice were vaccinated on day 0 and on day 21 with VB2060 DNA vaccine, and T cell responses were analyzed in draining lymph nodes on day 28. The cells were stimulated with RBD peptides for 16 hours, and analyzed using multiparameter flow cytometry. T cells were gated as described in FIG. 65. CD4⁺ and CD8⁺ T cells were examined for expression of TNF-α, IFN-γ, IL-2 and granzyme B. Percentages of positive cells are displayed in bar graphs in panels A, B, D and E.

The Trm cells as shown in FIG. 72 were also examined for expression of TNF-α, IFN-γ, IL-2 and granzyme B, and percentages of positive cells are displayed in panel C and F.

FIG. 72:

CD8⁺ positive T cells in FIG. 71 were analyzed for expression of CD103 and CD69 to define tissue resident memory T cells (Trm).

FIG. 73:

T cell response induced with different doses and number of doses of VB10.COV2 DNA vaccine VB2048. Total number of IFN-γ positive spots/1×10⁶ splenocytes from mice (5 animals/group) vaccinated intramuscularly on day(s) 0 (and day 21) with 2.5 μg or 25 μg VB2048 DNA plasmid after re-stimulation with 20 predicted T cell epitopes. Splenocytes were harvested at day 14 post first vaccination and at day 28 (7 days post boost vaccination at day 21).

FIG. 74:

Induction of CD4+ and CD8+ peptide specific immune responses in mice (5 animals/group) after intramuscular vaccination with 2×25 μg of VB2048 DNA plasmid at days 0 and 21. CD4 and CD8 cell populations were stimulated for 24 hours with 20 predicted peptides and number of IFN-γ positive spots/1×10⁶ splenocytes were detected in an ELISpot assay 7 days post boost vaccination on day 21.

FIG. 75:

T cell response induced by VB10.COV2 constructs with an antigenic unit comprising both predicted T cell epitopes and the RBD domain. Mice (5 animals/group) were vaccinated intramuscularly on day 0 with 25 μg of the indicated VB10.COV2 DNA plasmids. On day 14 post vaccination, the spleens were harvested and splenocytes re-stimulation with either 1-3 predicted T cell epitopes or RBD pools. The figure represents the total number of IFN-γ positive spots/1×10⁶ splenocytes.

FIG. 76:

T cell response induced by single plasmid vaccine compared to a vaccine comprising 2 plasmids. The VB10.COV2 constructs VB2048 (20 T cell epitopes) and VB2049 (RBD) were used for vaccination either as a stand-alone vaccine or a vaccine comprising both VB2048 and VB2049 in a pharmaceutically acceptable carrier (combination vaccine). Mice (5 animals/group) were vaccinated intramuscularly on day 0 with either 25 μg of VB2048 or VB2049 as stand-alone vaccines or the combination vaccine comprising 12.5 μg of each VB2048 and VB2049. On day 14 post vaccination, the spleens were harvested and splenocytes were re-stimulated with either 20 predicted T cell epitopes and/or RBD pools. The figure represents the total number of IFN-γ positive spots/1×10⁶ splenocytes.

FIG. 77:

Amino acid sequence of the signal peptide and anti-pan HLA class II targeting unit. The sequence is split up by “|” to help distinguish the following various parts of the sequence: Ig VH signal peptide|anti-pan HLA class II VL|linker|anti-pan HLA class II VH.

FIG. 78

VB10.COV2 DNA vaccine VB2060 stability data. VB2060 was stored at 37° C. for up to 4 weeks and % supercoil DNA content was determined by HPLC as a stability indicating parameter after week 1 (T1), week 2 (T2), week 3 (T3) and week 4 (T4).

### DETAILED DESCRIPTION

One aspect of the invention relates to a vaccine comprising an immunologically effective amount of:
(i) a polynucleotide comprising a nucleotide sequence encoding a targeting unit, a dimerization unit and an antigenic unit, wherein the antigenic unit comprises at least one betacoronavirus epitope; or
(ii) a polypeptide encoded by the polynucleotide as defined in (i); or
(iii) a dimeric protein consisting of two polypeptides encoded by the polynucleotide as defined in (i); and
a pharmaceutically acceptable carrier.

In general, a vaccibody construct comprises, in sequence, a targeting unit, a dimerization unit, and an antigenic unit. The vaccine induces a rapid, strong immune response, e.g. with few low doses. This makes it ideal for epidemic and pandemic situations.

Herein, a vaccine is capable of eliciting an immune response in a human individual to which it has been administered. In one embodiment, the immune response is a humoral immune response through generation of antibodies by B cells. In another embodiment, the immune response is a cellular immune response through generation of T cells. In yet another embodiment, the immune response is a humoral and cellular immune response.

The human individual may be a healthy human individual and the vaccine is used to provide a prophylactic treatment to said individual, i.e. rendering the individual a certain protection against an infection with a betacoronavirus. Alternatively, the human individual may be an individual who has been infected with a betacoronavirus and the vaccine is used to provide a therapeutic treatment to said individual, i.e. alleviating the symptoms of or curing the infection.

Betacoronaviruses denotes a genus in the subfamily Orthocoronaviridae. Betacoronaviruses are enveloped, positive-sense single-stranded RNA viruses. Within the genus, four lineages are commonly recognized: lineage A (subgenus Embecovirus), lineage B (subgenus Sarbecovirus), lineage C (Merbecovirus) and lineage D (Nobecovirus). Betacoronaviruses include the following viruses which caused/cause epidemics/pandemics in humans or can infect humans: SARS-CoV, which causes severe acute respiratory syndrome (SARS), MERS-CoV, which causes Middle East respiratory syndrome (MERS), SARS-CoV-2, which causes coronavirus disease 2019 (Covid-19), HCoV-OC43 and HCoV-HKU1. SARS-CoV and SARS-CoV-2 belong to the lineage B (subgenus Sarbecovirus), MERS-CoV belongs to the lineage C (Merbecovirus) and HCoV-OC43 and HCoV-HKU1 belong to the lineage A (subgenus Embecovirus).

Thus, in a further embodiment the human individual may be an individual who is at risk to be infected or has been infected with a betacoronavirus belonging to the lineage B (subgenus Sarbecovirus). Alternatively, the human individual may be an individual who is at risk to be infected or has been infected with SARS-CoV or SARS-CoV-2.

It is a common belief, that viral infections should be avoided by raising neutralizing antibodies against the virus. However, an aspect of the present invention relates to a vaccine that, once administered to a human individual, elicits only a T cell response or both a T cell response and a B cell response. The polynucleotides/polypeptides/dimeric proteins presented herein are able to raise cytotoxic T cells. Raised CD8+ T cells will kill virus-infected cells and eliminate the virus, thus curing the/protecting from disease or at least alleviate the severity of the disease, both in a prophylactic and therapeutic setting. The antigenic unit of the vaccine according to the invention may comprise T cell epitopes only or T cell epitopes which are comprised in a betacoronavirus protein, which also comprises B cell epitopes—for example the spike protein, as shown herein. For an antigenic unit comprising T cell epitopes only, these can be from essential intracellular viral proteins that are more conserved than viral surface proteins. Hereby is obtained a vaccine that can be used both for ongoing and future pandemics/epidemics caused by a similar betacoronavirus. For T cell epitopes comprised in a betacoronavirus surface protein, the surface protein can also comprise B cell epitopes that can induce an antibody response, i.e. antibodies binding to the viral surface protein when the virus is in circulation and neutralizing the virus by inhibiting it from entering the host cell. The aforementioned vaccine can be used as a therapeutic vaccine or as a prophylactic vaccine.

In one aspect a human individual suffers from a betacoronavirus infection and the vaccine is a therapeutic vaccine. Then the vaccine is administered to an individual that has been exposed to and may be affected by a betacoronavirus virus to eliminate infected cells and thus minimize the severity of the disease and to produce neutralizing antibodies against infection of further cells.

In another aspect of the invention, a human individual is a healthy individual and the vaccine is a prophylactic vaccine. Typically, this will be used to induce immunity to people where it is desired to raise neutralizing antibodies against the betacoronavirus in a prophylactic setting, e.g. to prevent an infection.

One aspect of the invention relates to a vaccine, wherein the antigenic unit comprises at least one betacoronavirus epitope which is a full-length viral surface protein of a betacoronavirus or a part of such a protein. As such, in one embodiment, the at least one betacoronavirus epitope is a full-length protein or a part thereof, wherein the protein is selected from the group consisting of envelope protein, spike protein, membrane protein and, if the betacoronavirus is an Embecovirus, spike-like protein hemagglutinin esterase.

In one embodiment, the antigenic unit comprises at least a B cell epitope comprised in a full-length viral surface protein of a betacoronavirus, e.g. comprised in any of the aforementioned proteins and preferably comprises several B cell epitopes comprised in a full-length viral surface protein of a betacoronavirus, e.g. comprised in any of the aforementioned proteins.

The term “several” herein is used interchangeably with the term “multiple” and “more than one”.

The B cell epitope may be a linear or a conformational B cell epitope.

Thus, in one aspect, the invention relates to a vaccine comprising an immunologically effective amount of:

(i) a polynucleotide comprising a nucleotide sequence encoding a targeting unit, a dimerization unit and an antigenic unit, wherein the antigenic unit comprises a full-length viral surface protein of a betacoronavirus or a part thereof, preferably a protein selected from the group consisting of envelope protein, spike protein, membrane protein and hemagglutinin esterase, or

(ii) a polypeptide encoded by the polynucleotide as defined in (i), or

(iii) a dimeric protein consisting of two polypeptides encoded by the polynucleotide as defined in (i); and

a pharmaceutically acceptable carrier.

Once administered, a vaccine as described above, i.e. comprising an antigenic unit, wherein the antigenic unit comprises a full-length viral surface protein of a betacoronavirus or a part thereof, elicits a B cell response and T cell response and can be used as a prophylactic or therapeutic vaccine. In one embodiment, the aforementioned vaccine is used a prophylactic vaccine.

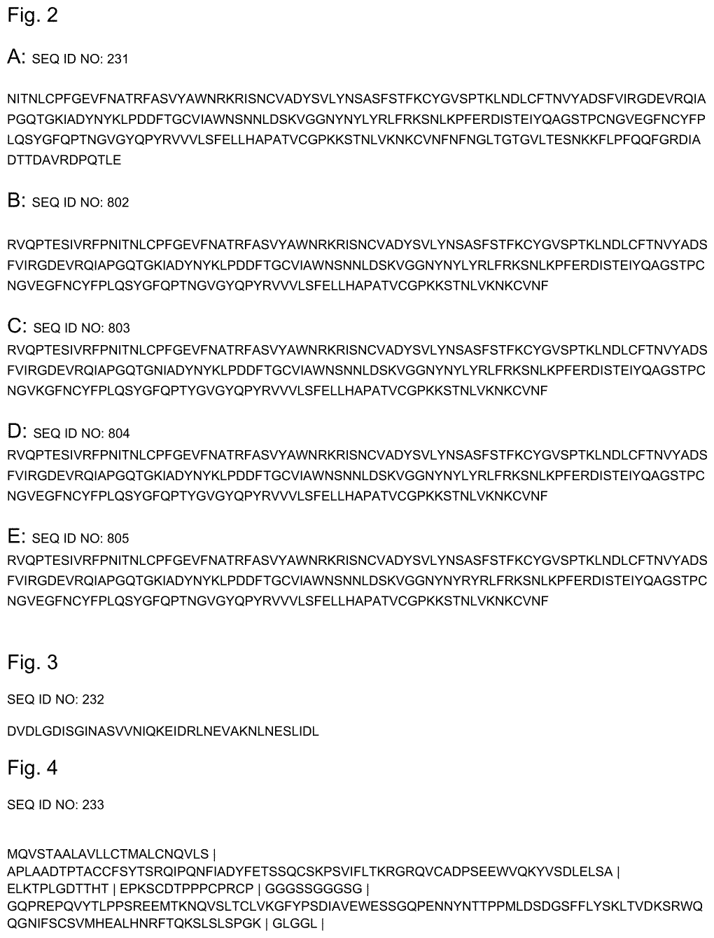

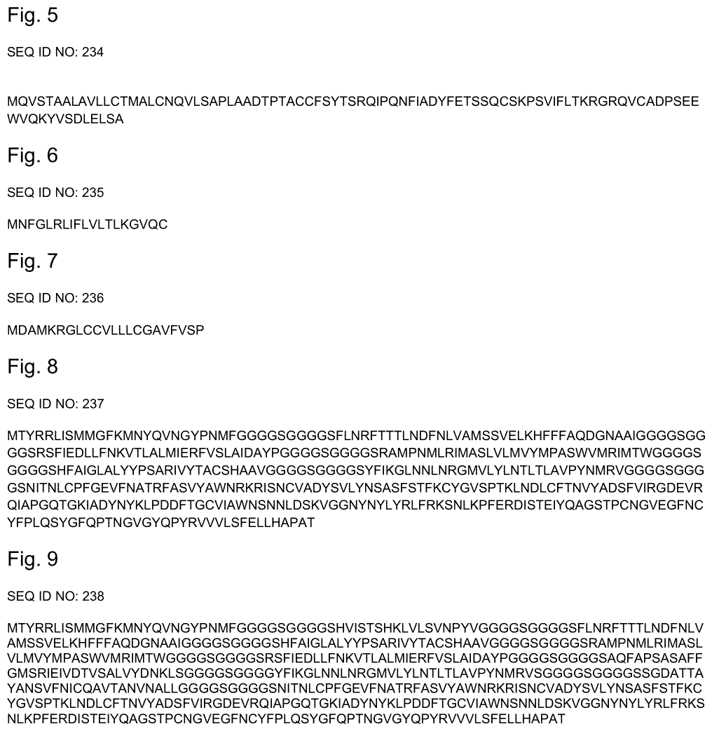

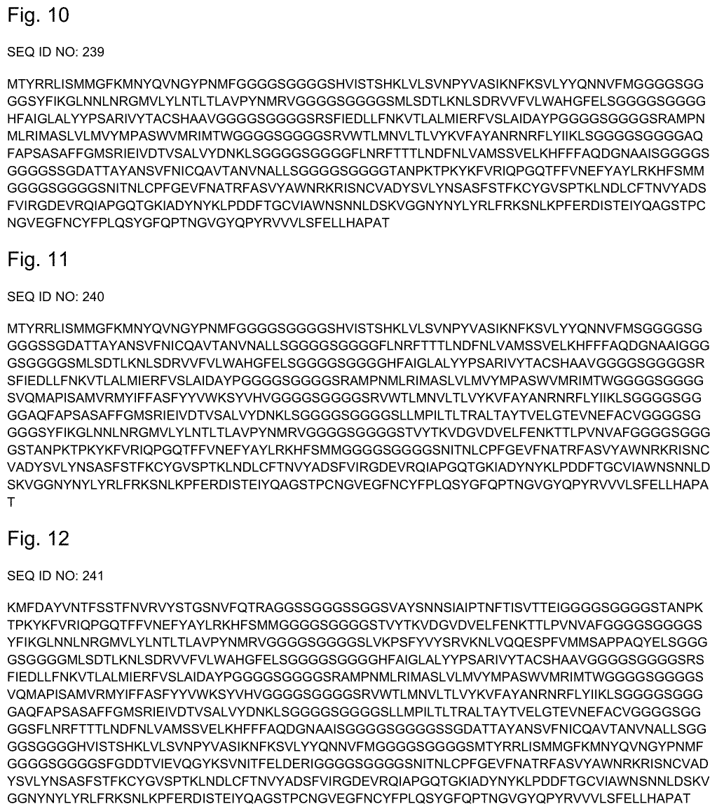

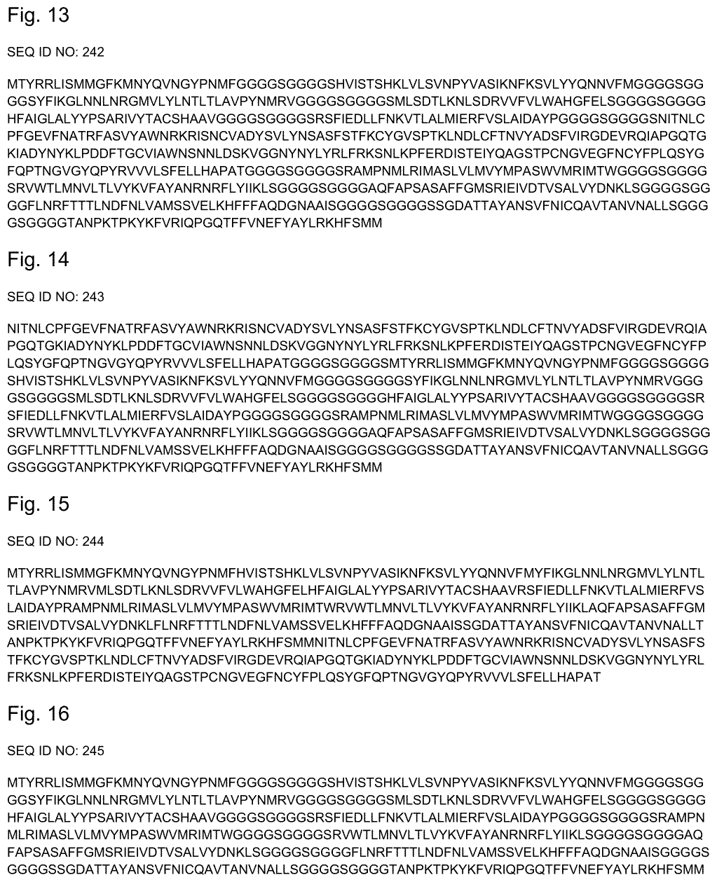

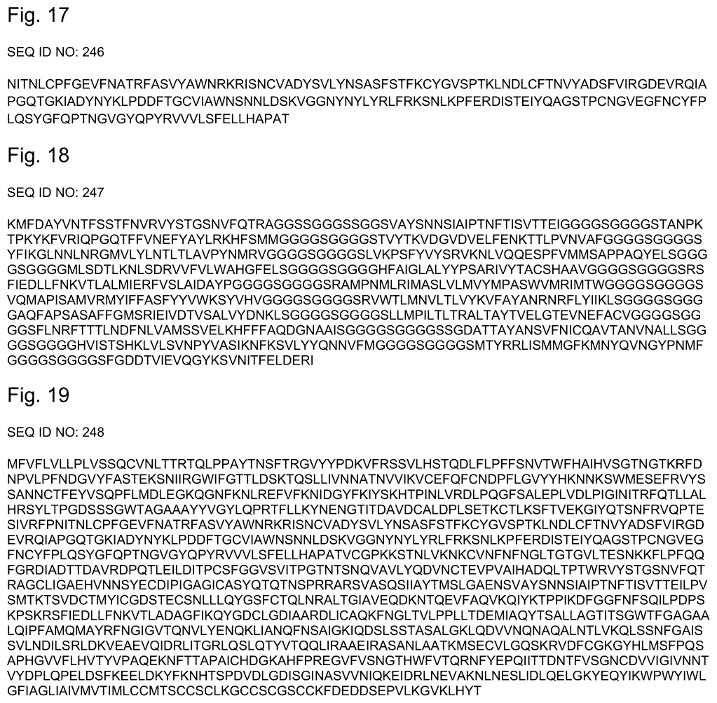

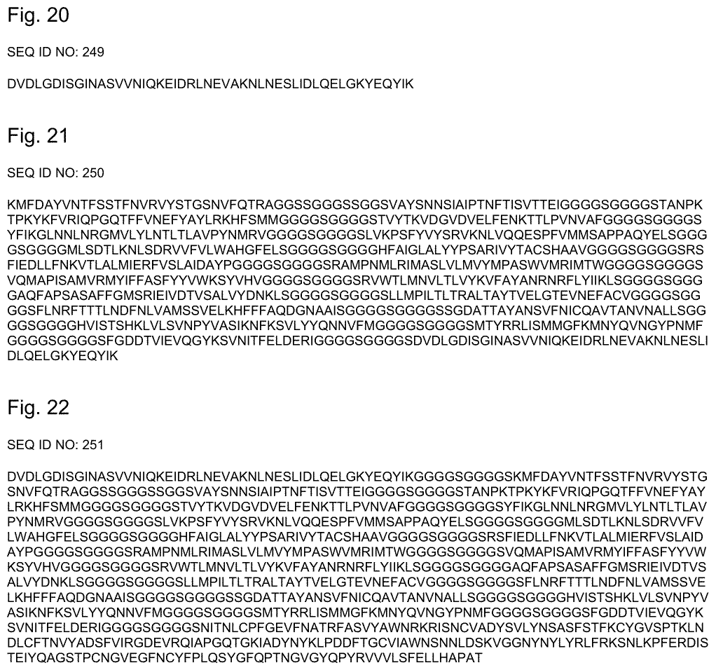

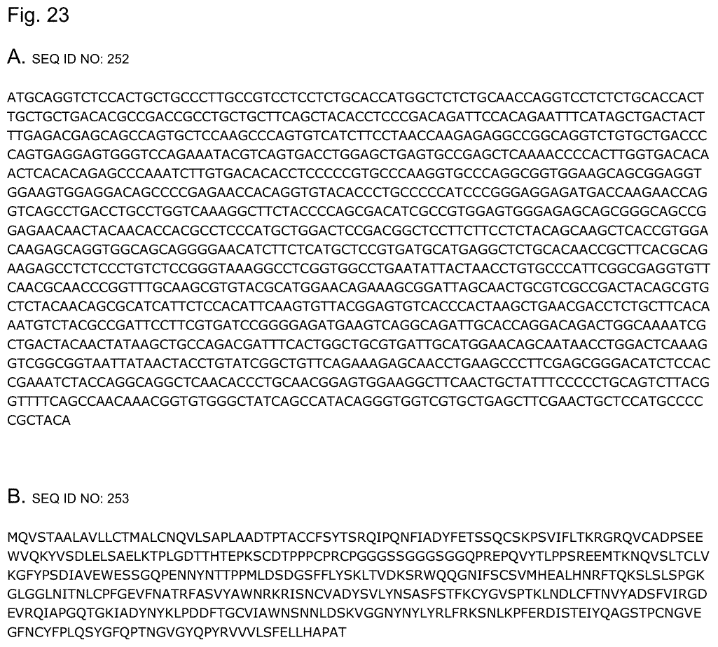

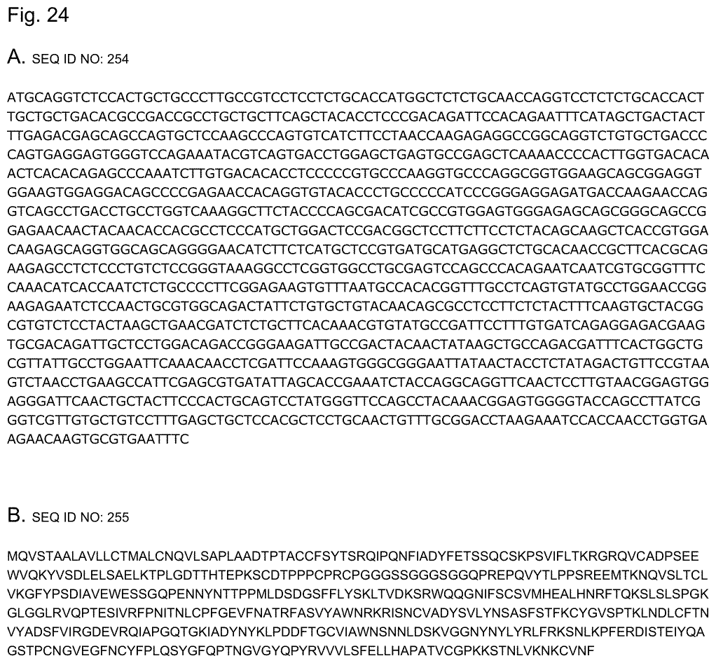

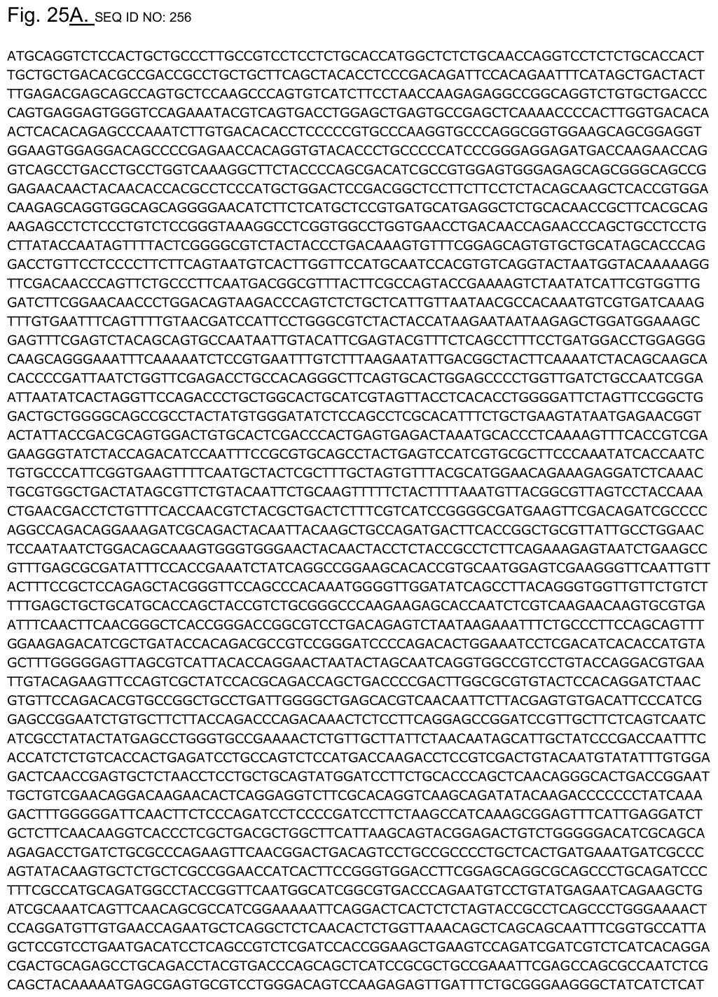

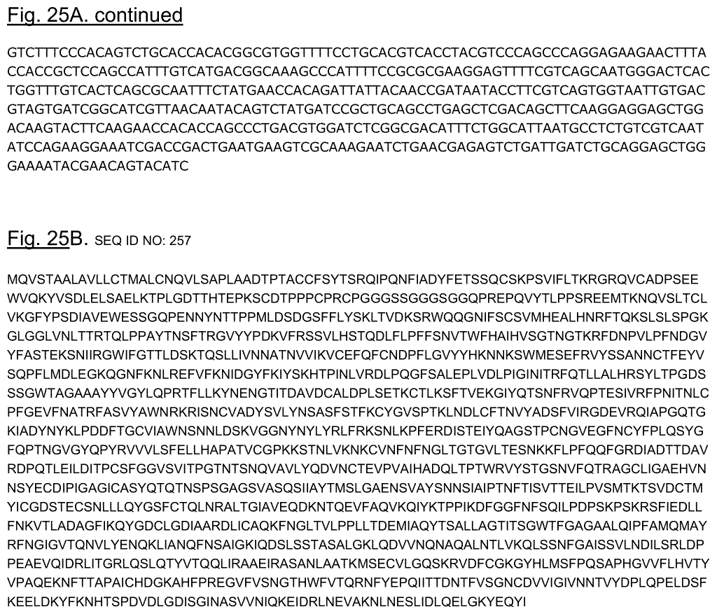

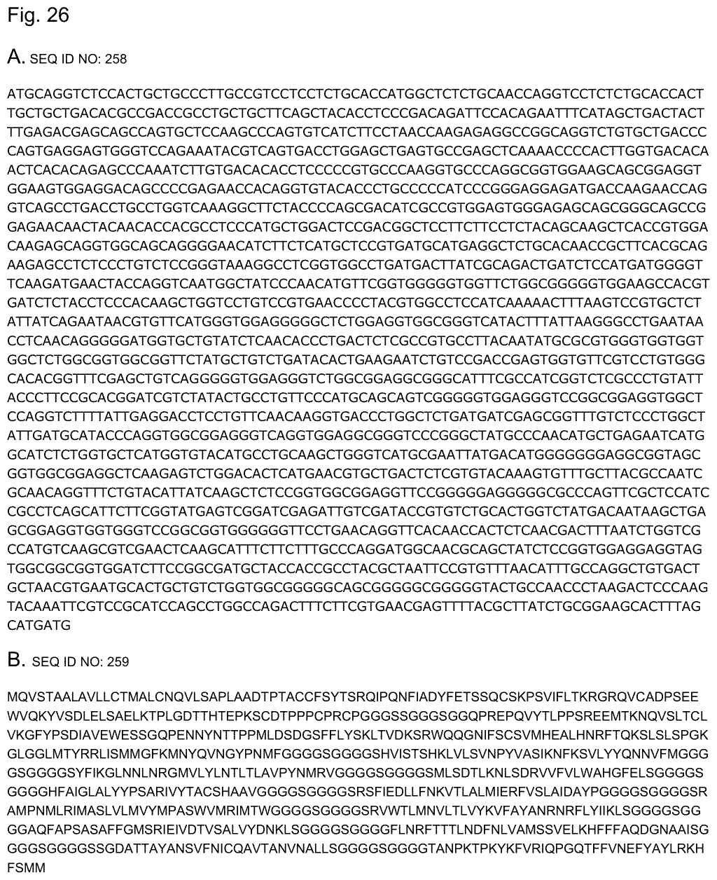

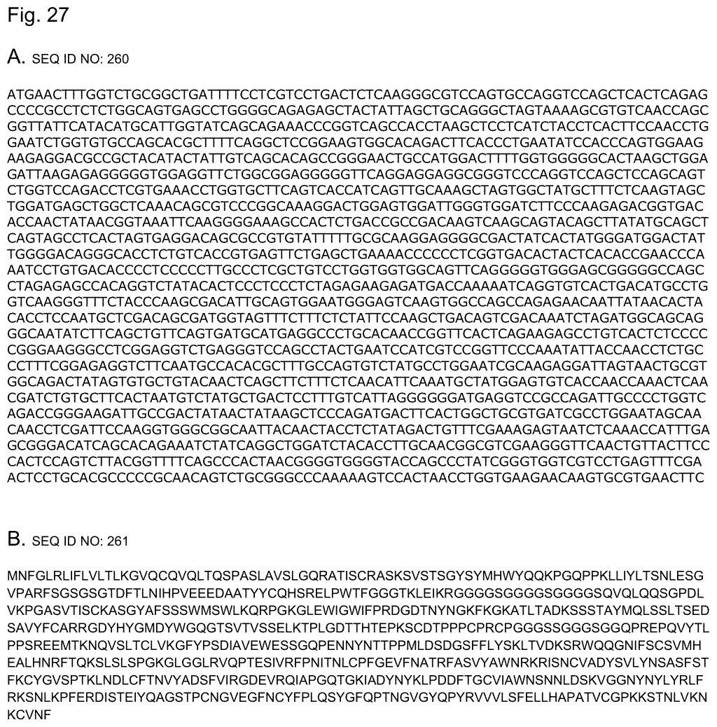

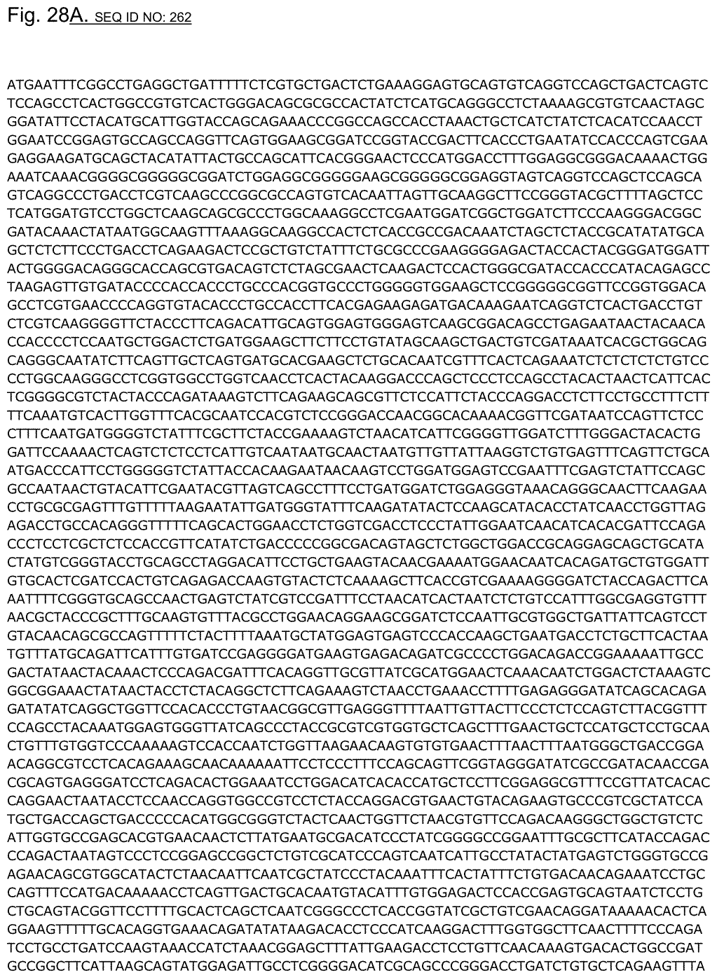

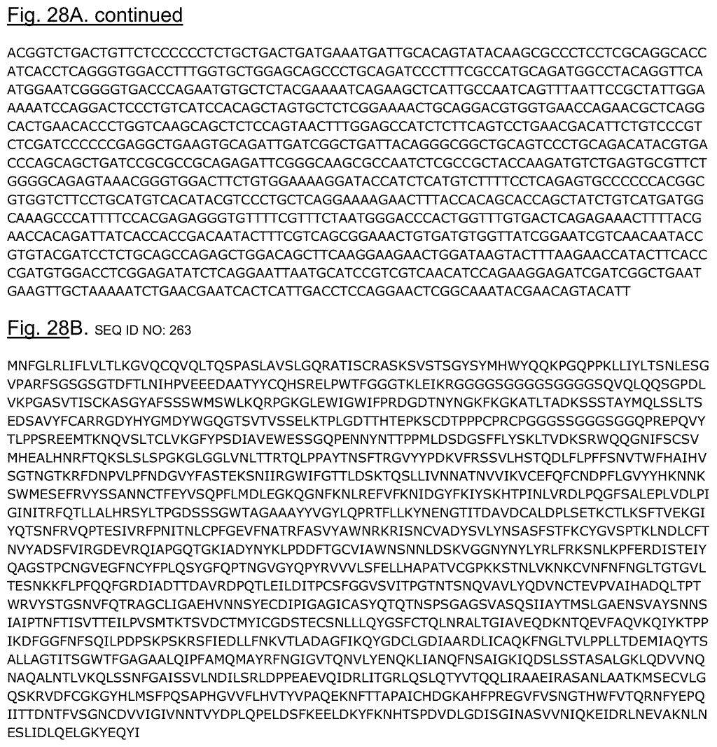

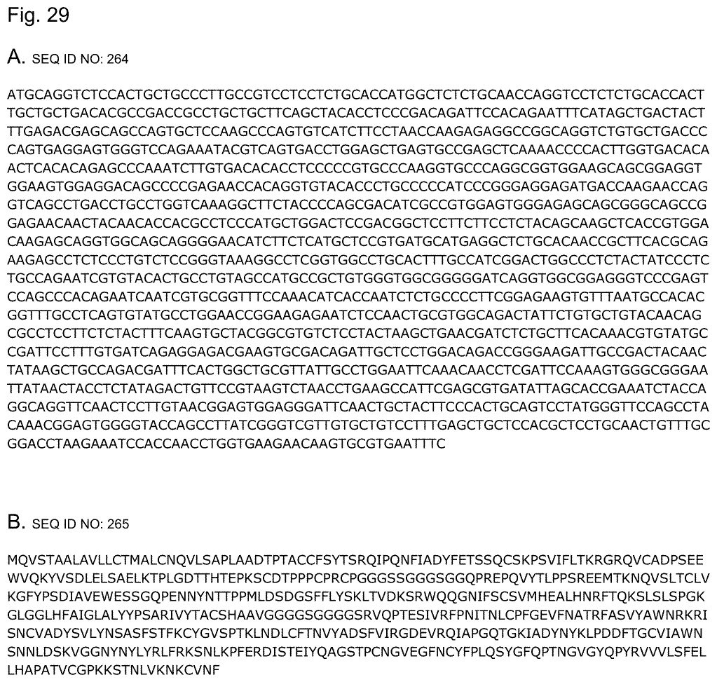

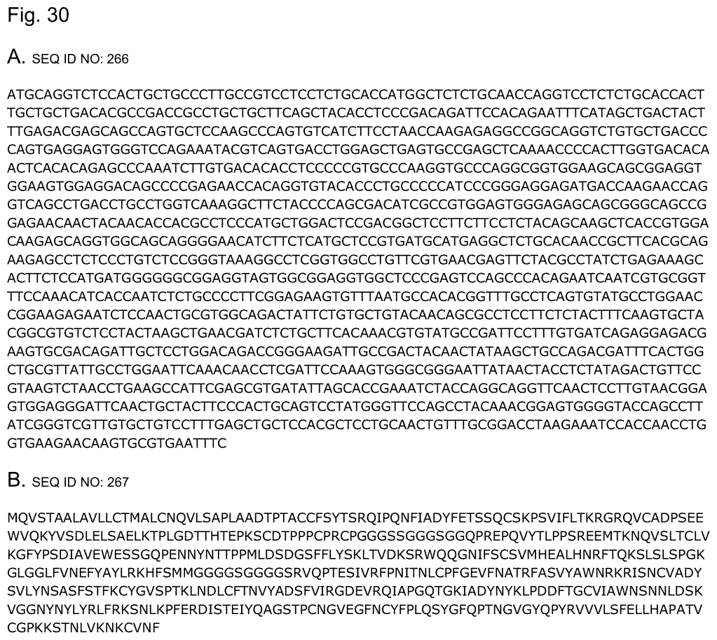

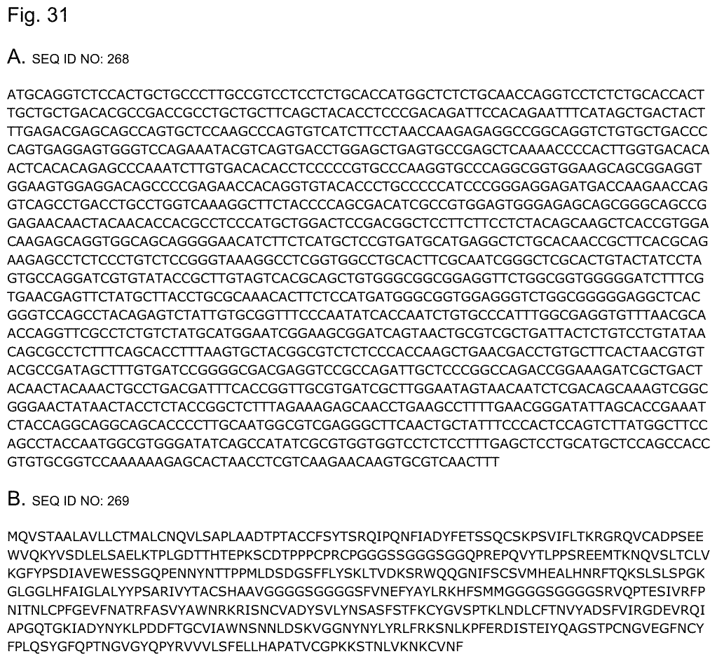

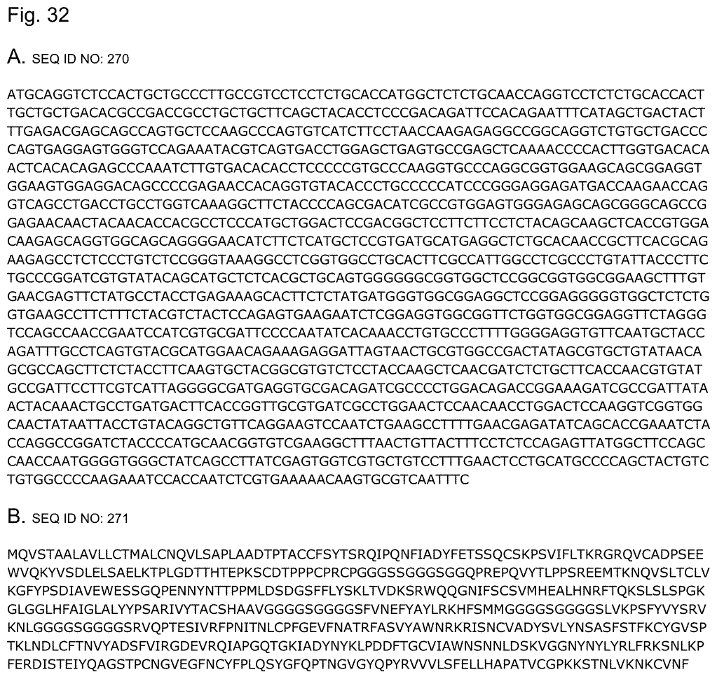

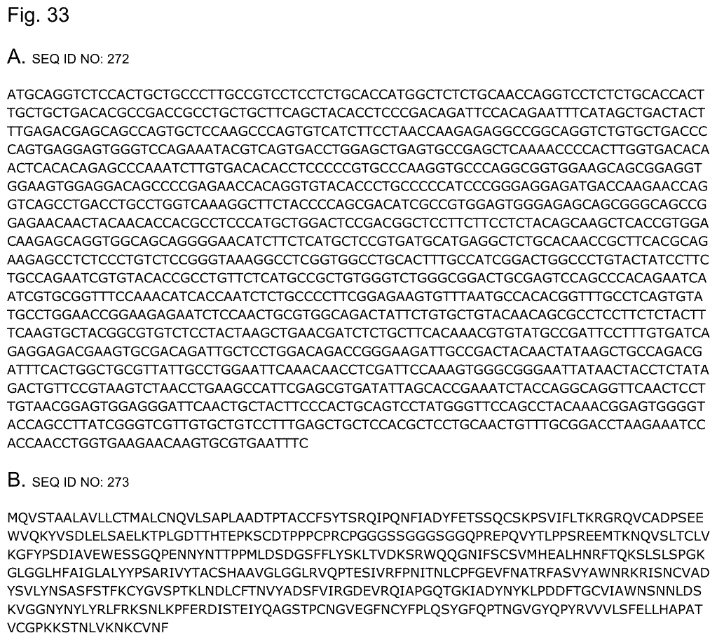

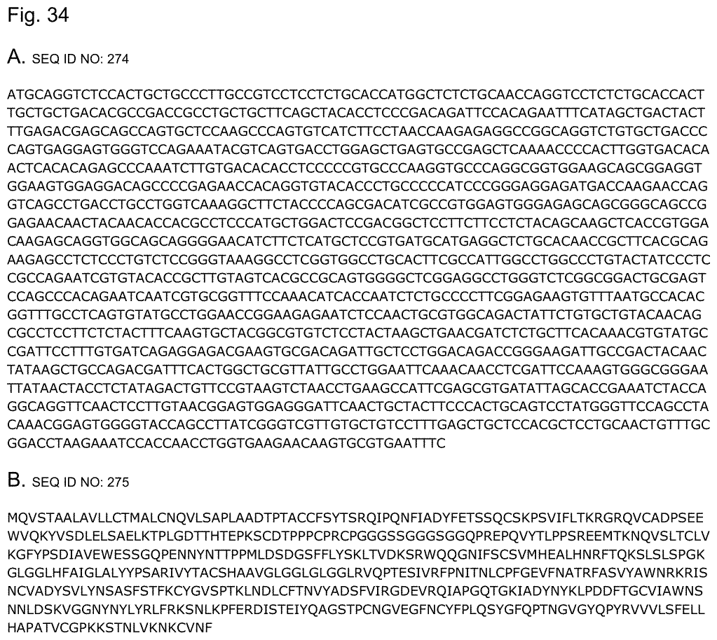

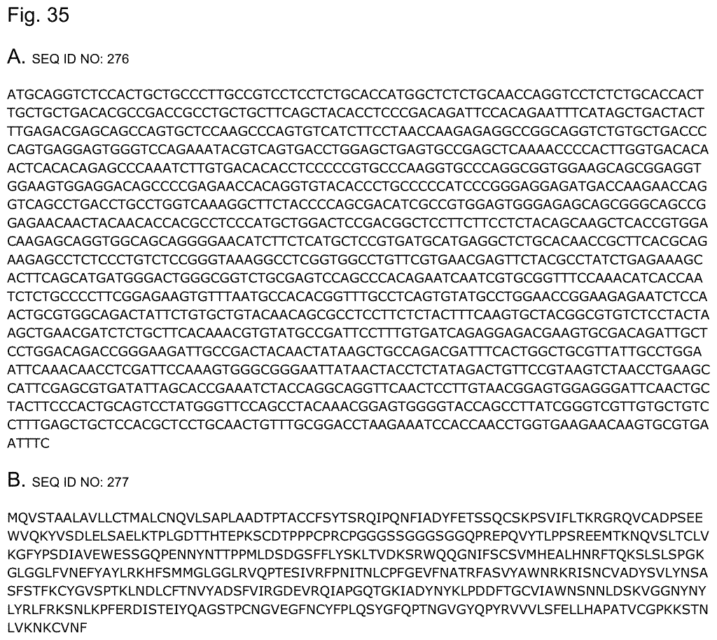

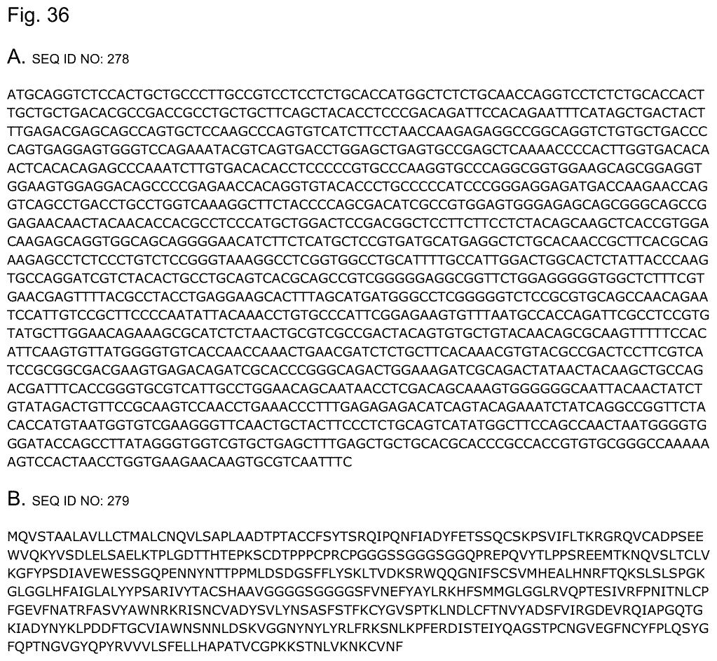

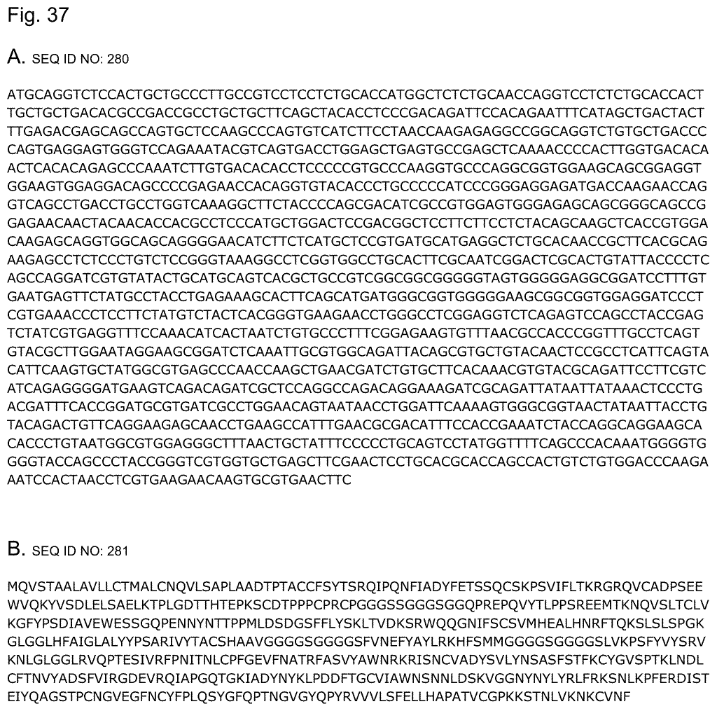

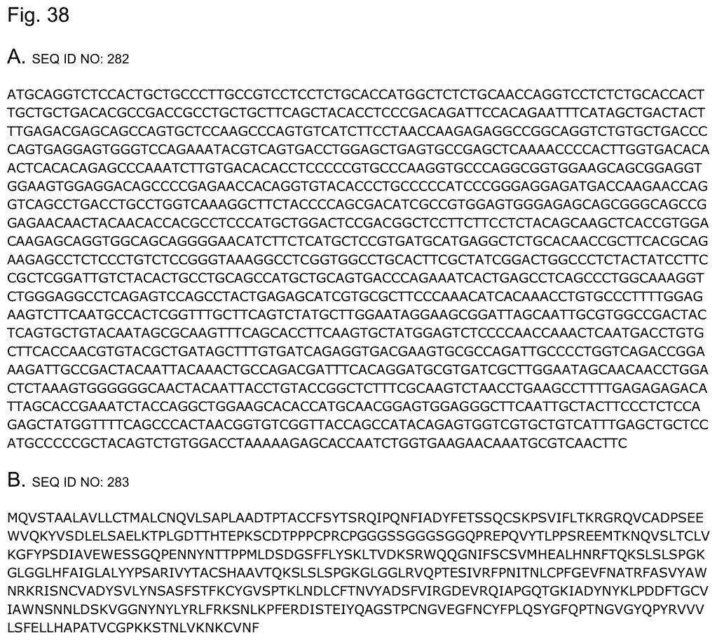

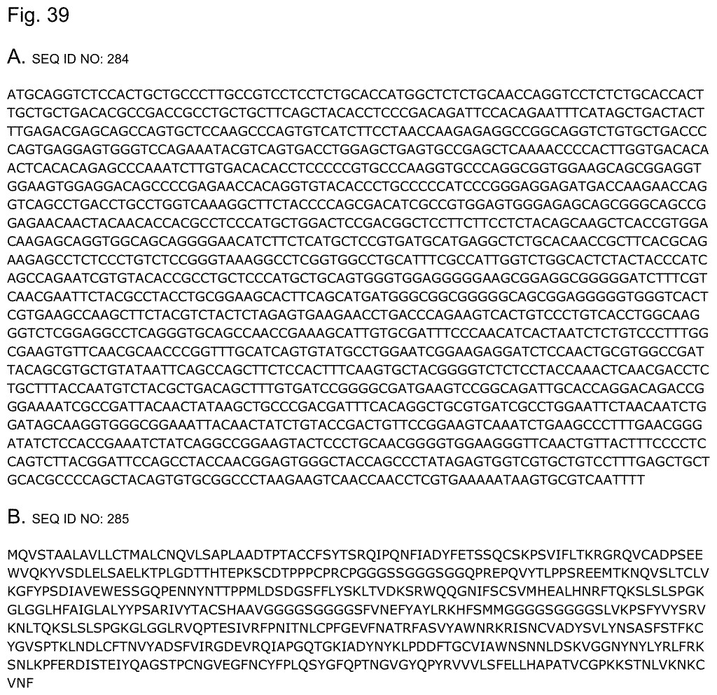

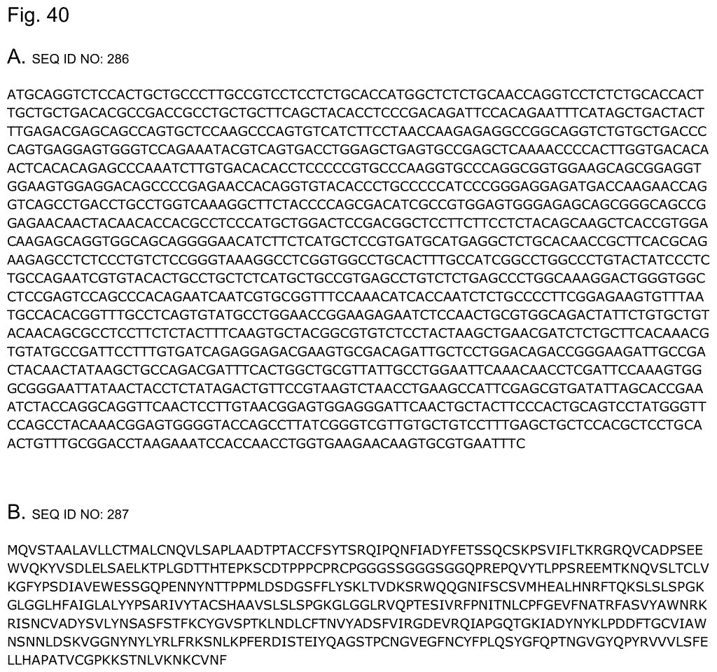

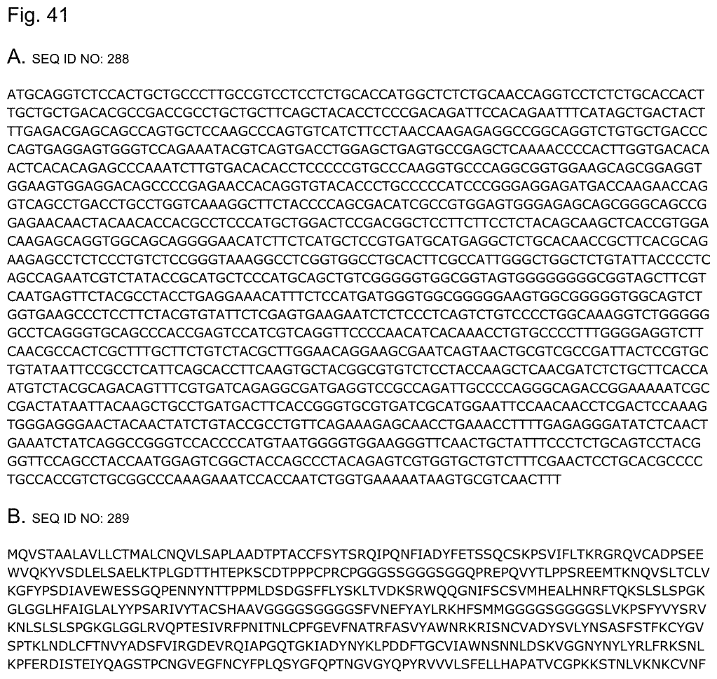

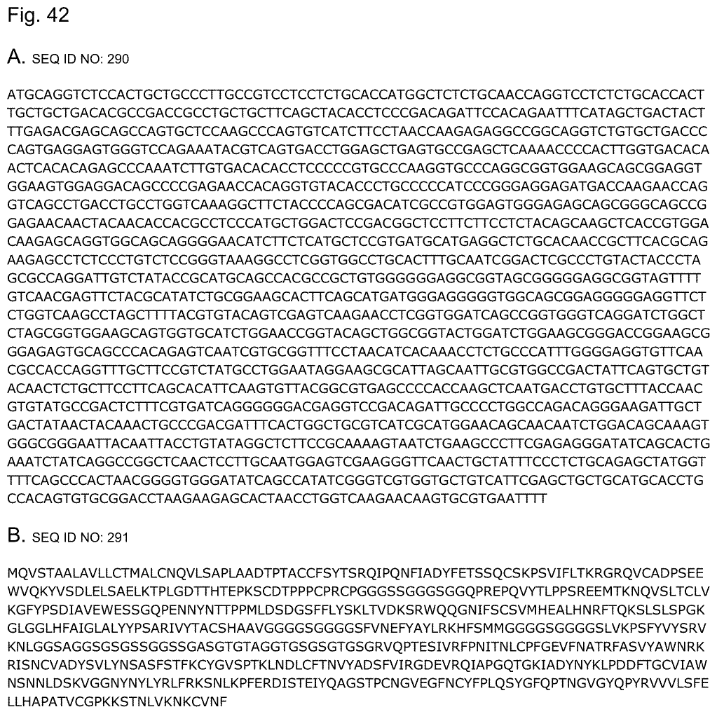

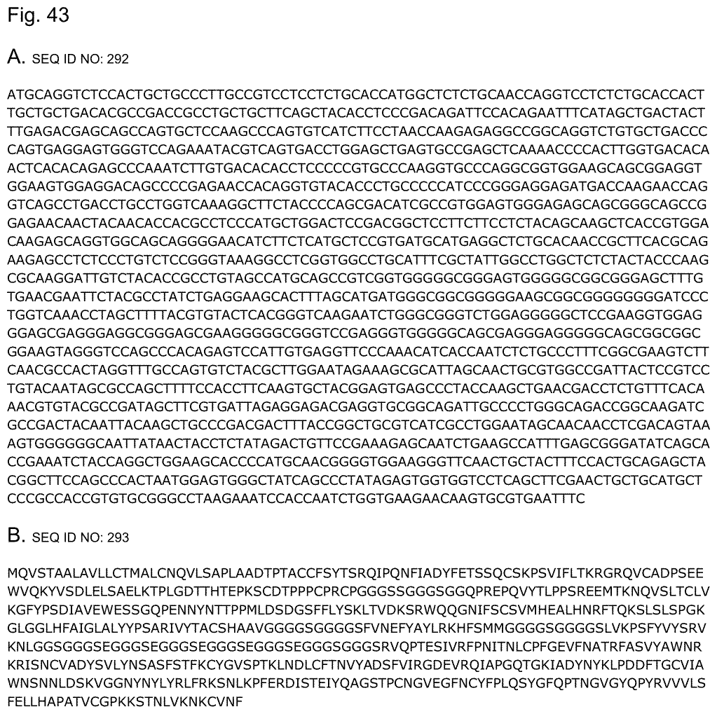

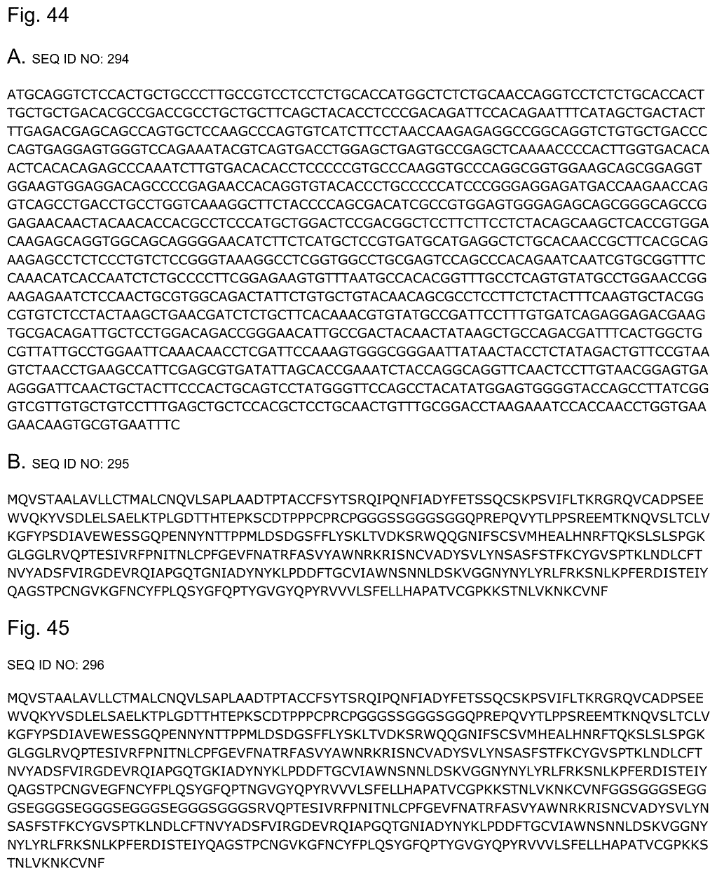

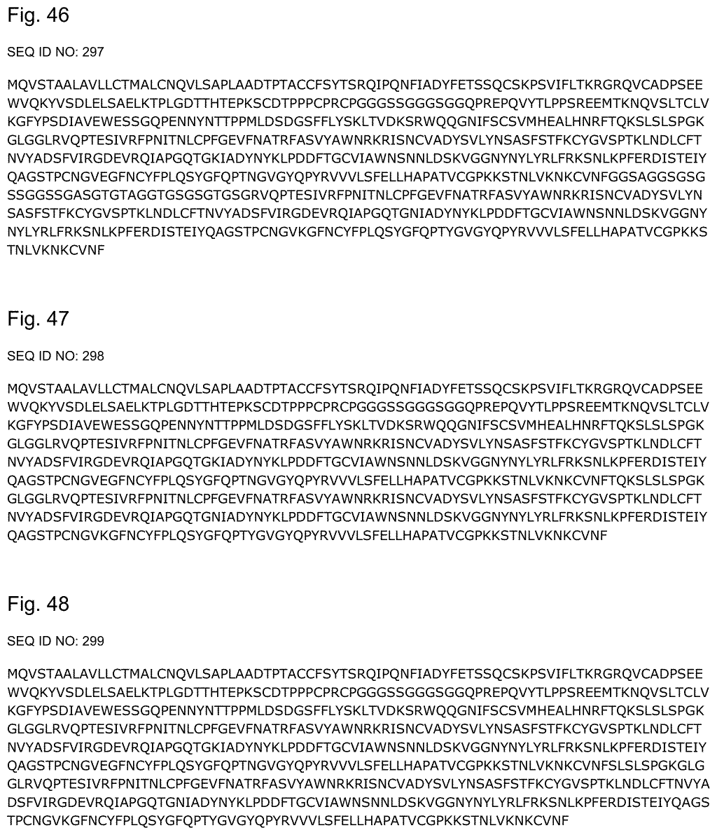

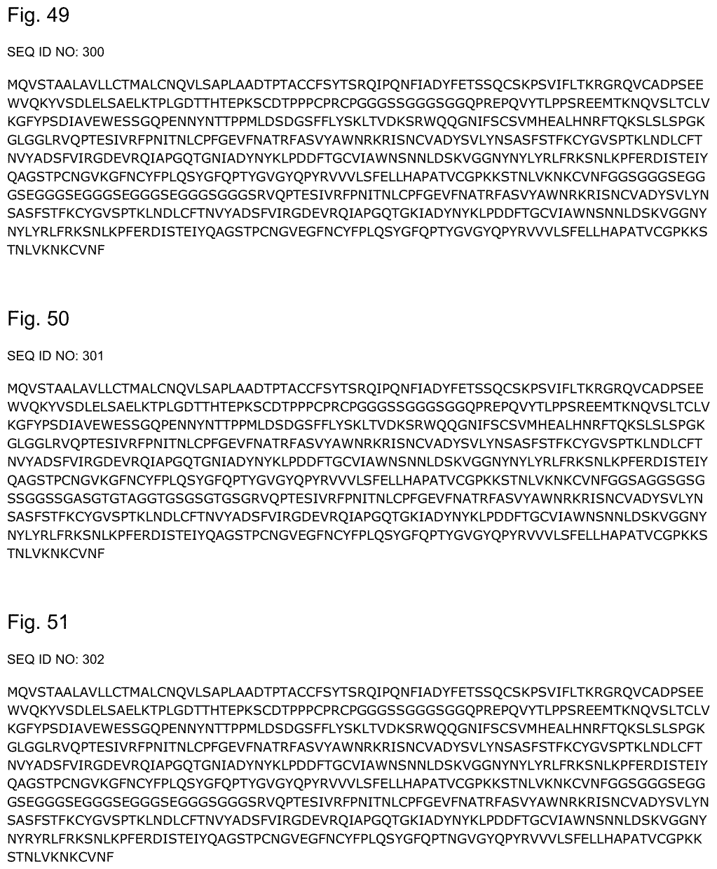

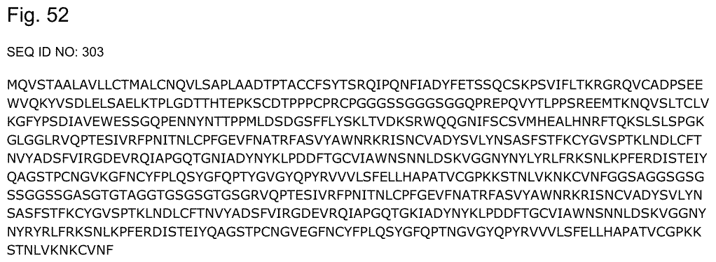

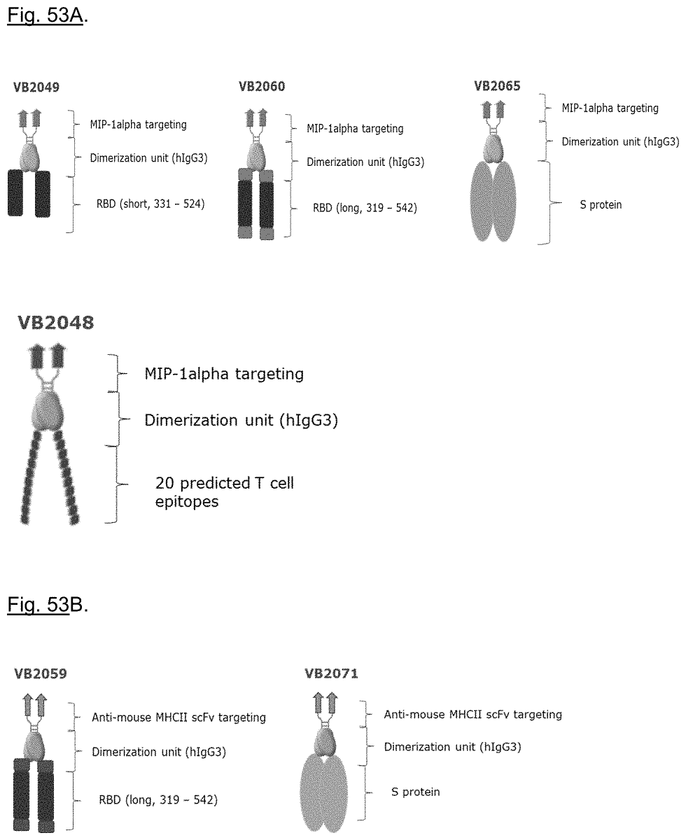

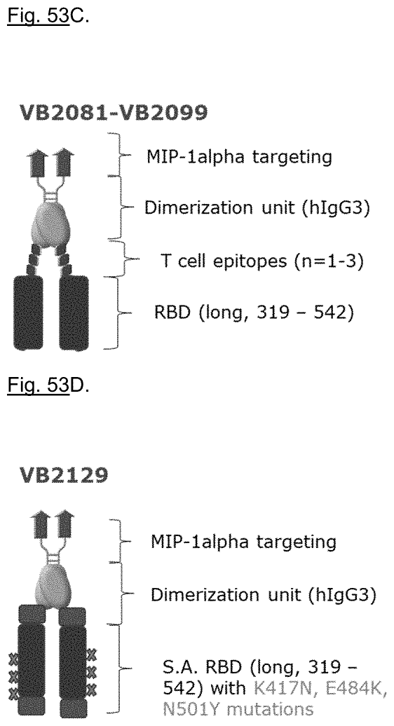

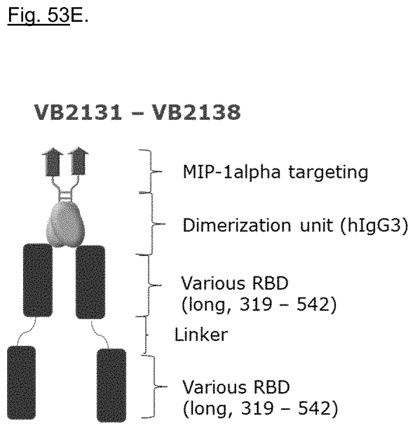

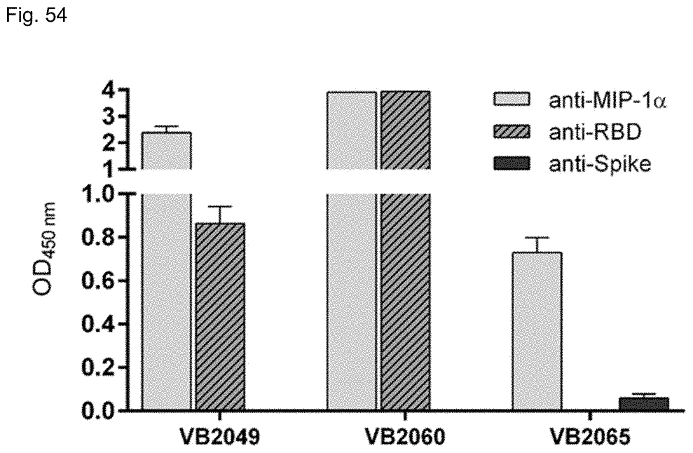

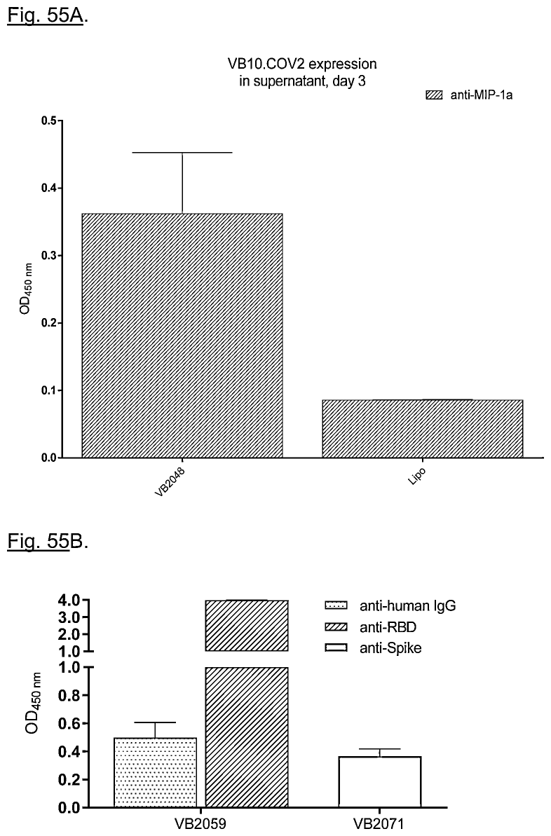

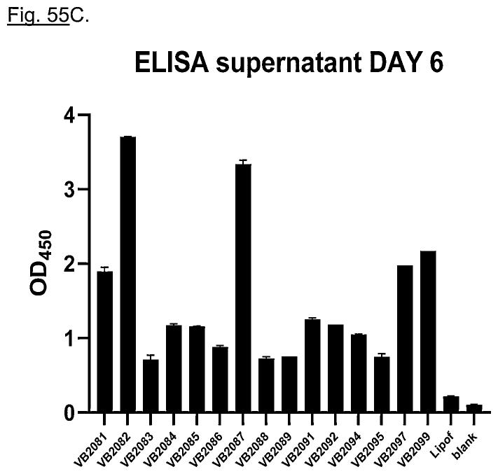

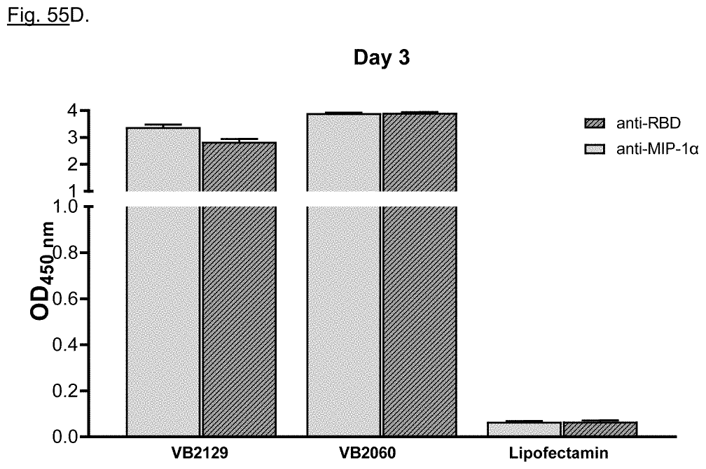

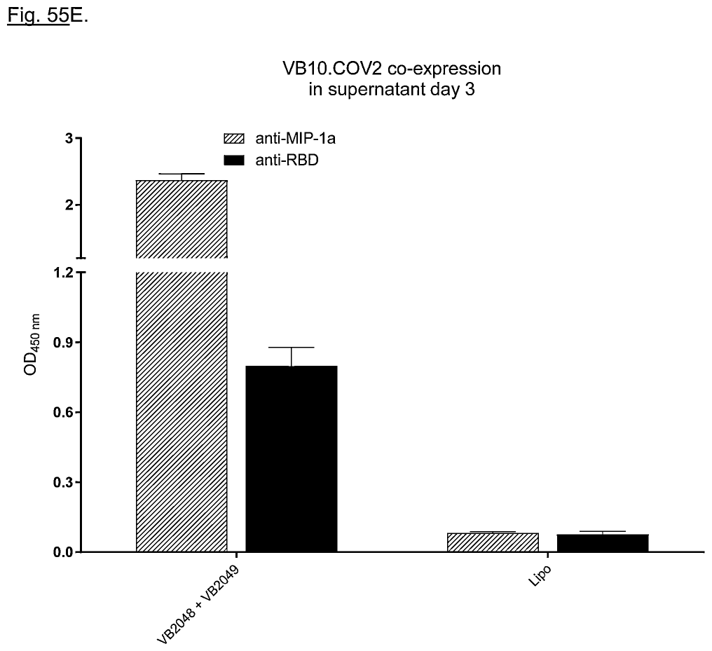

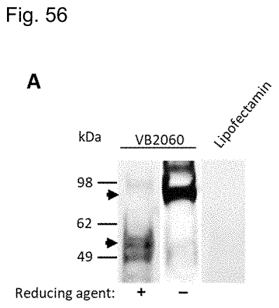

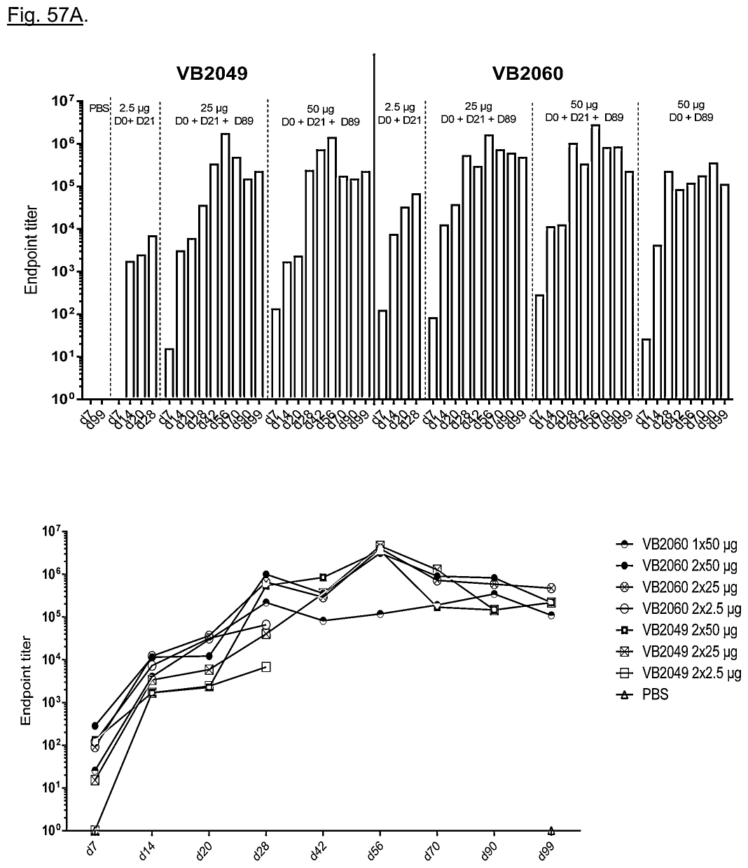

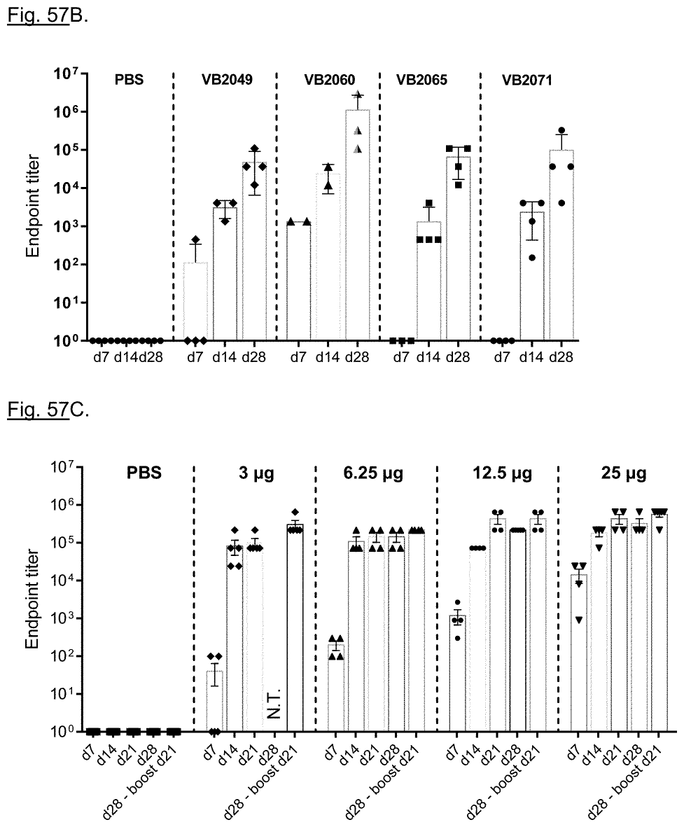

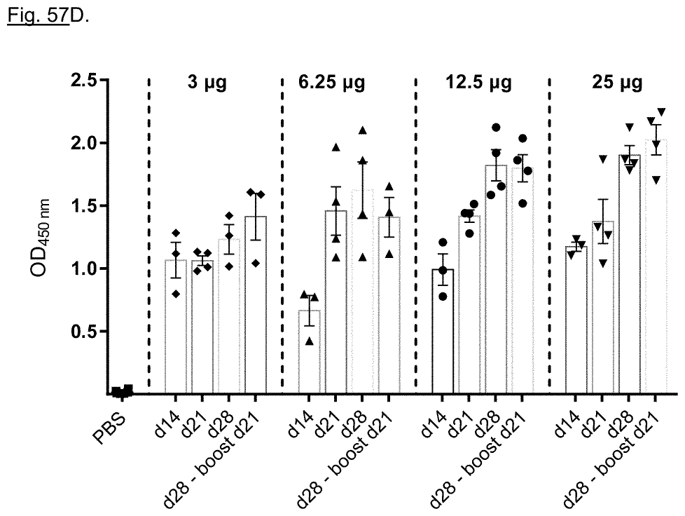

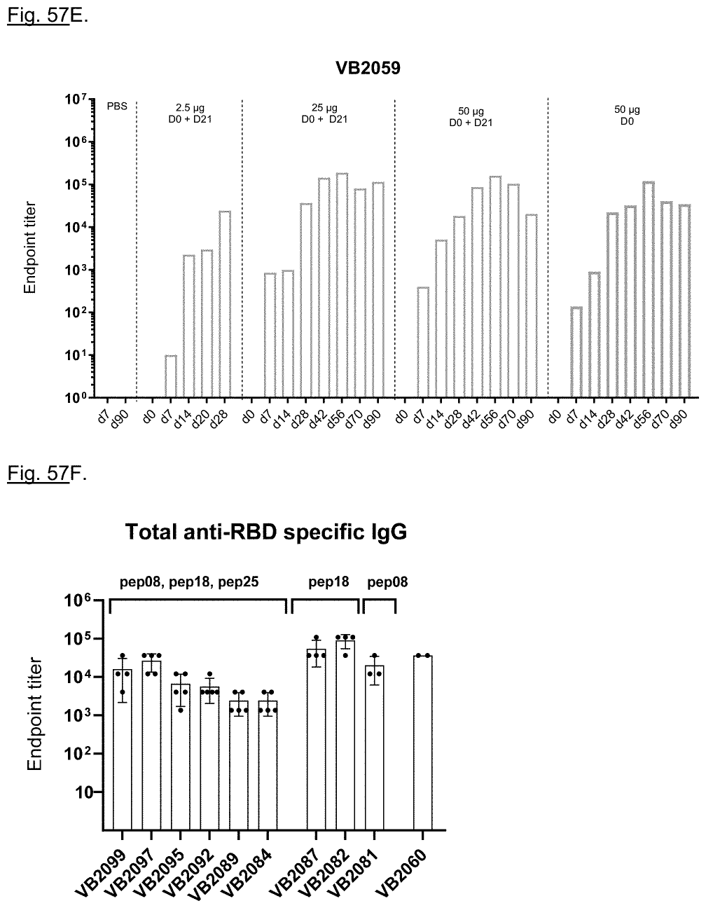

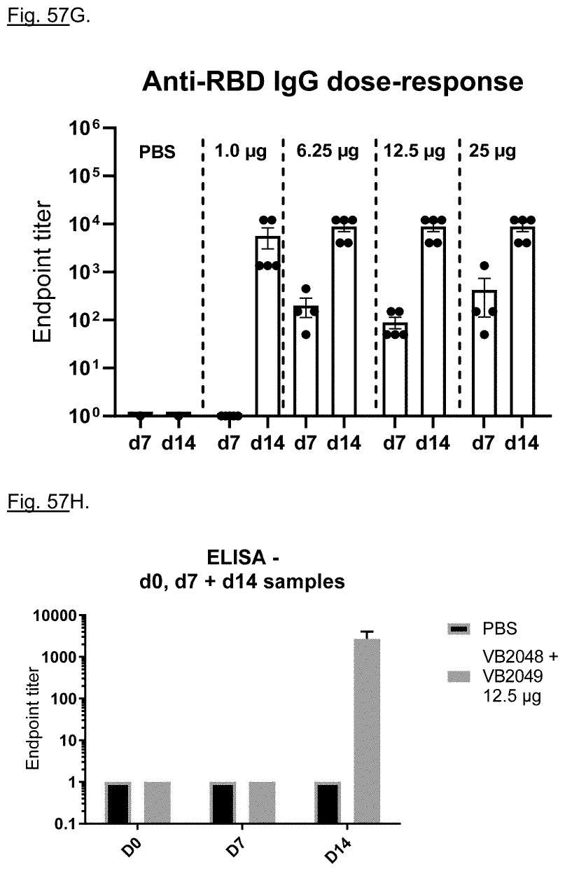

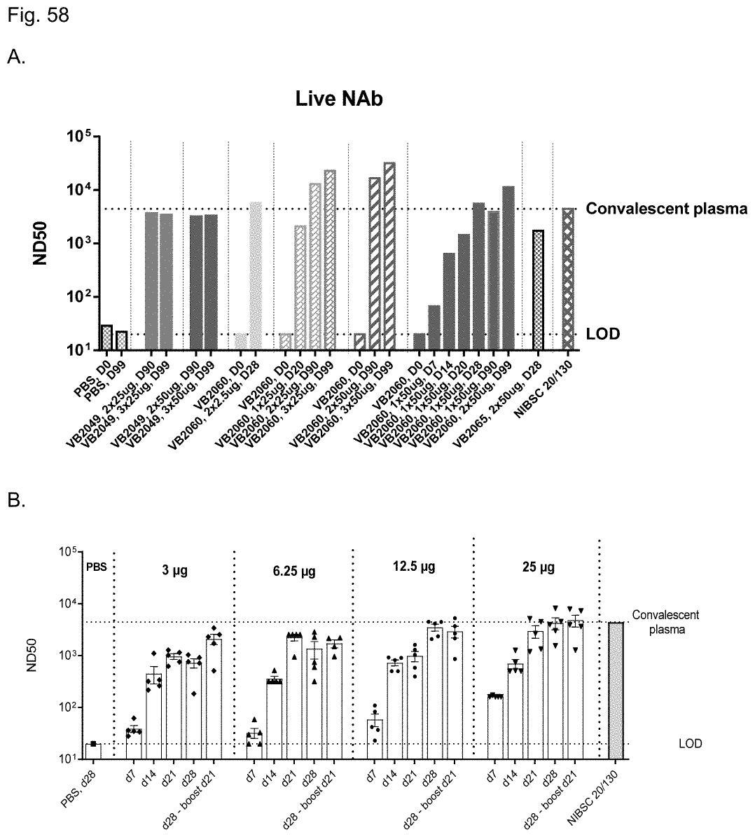

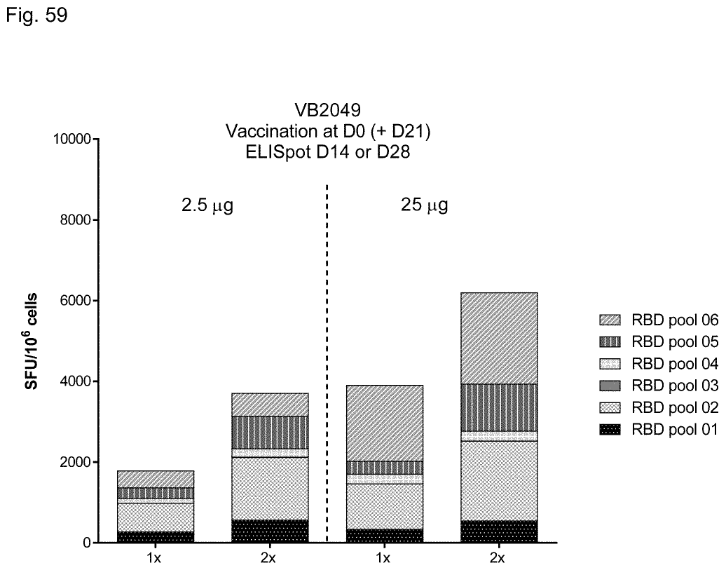

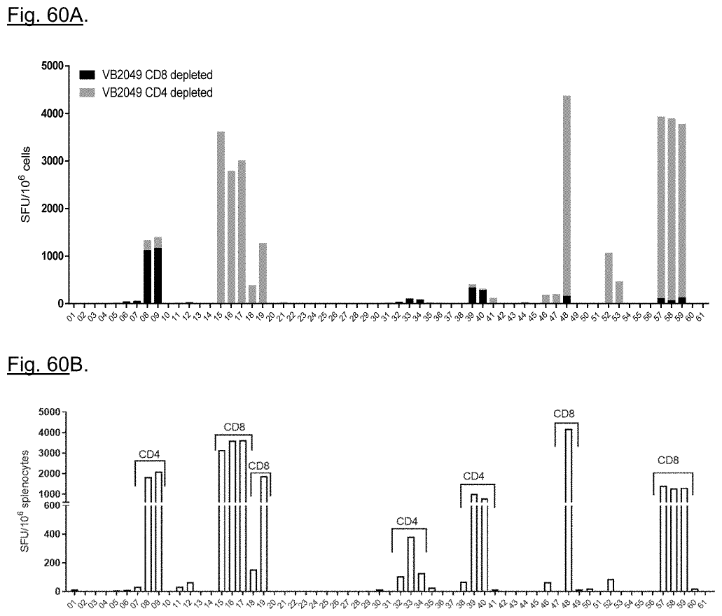

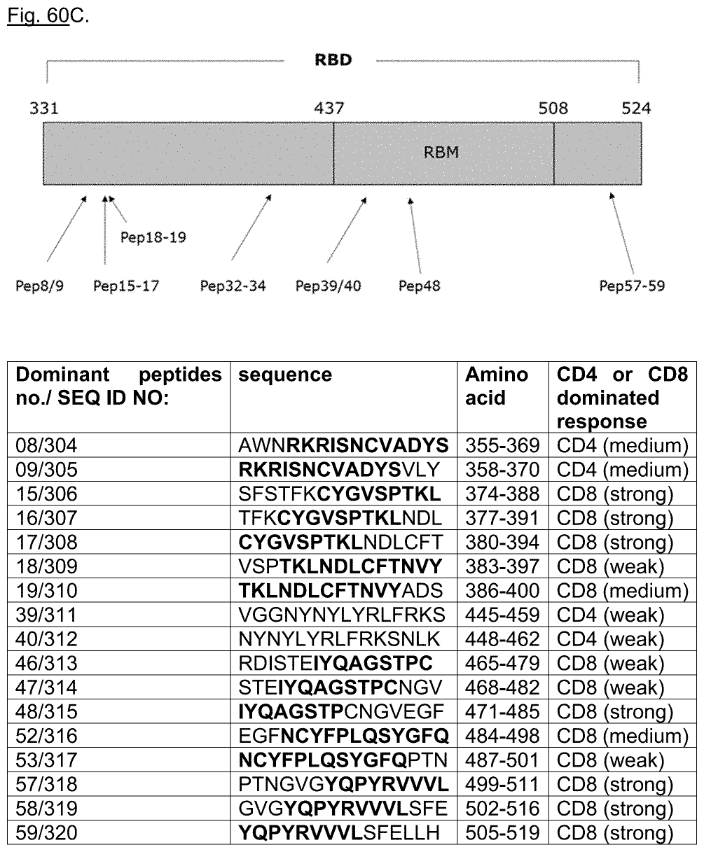

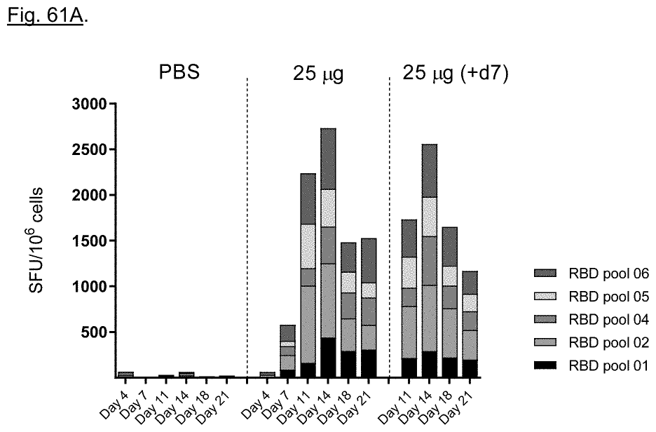

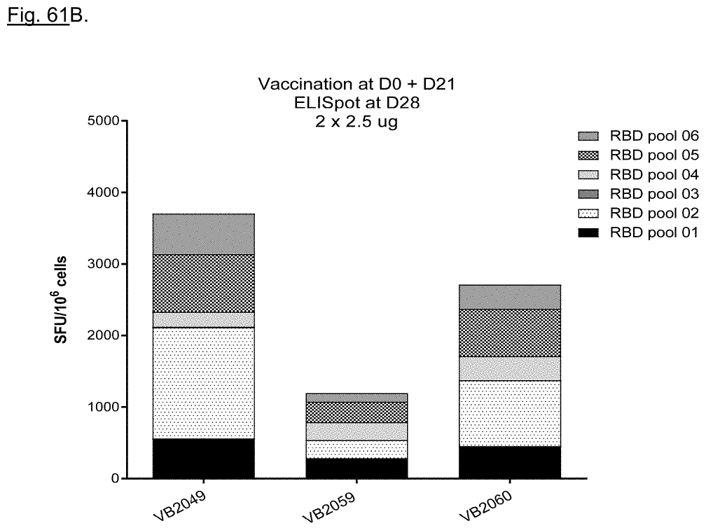

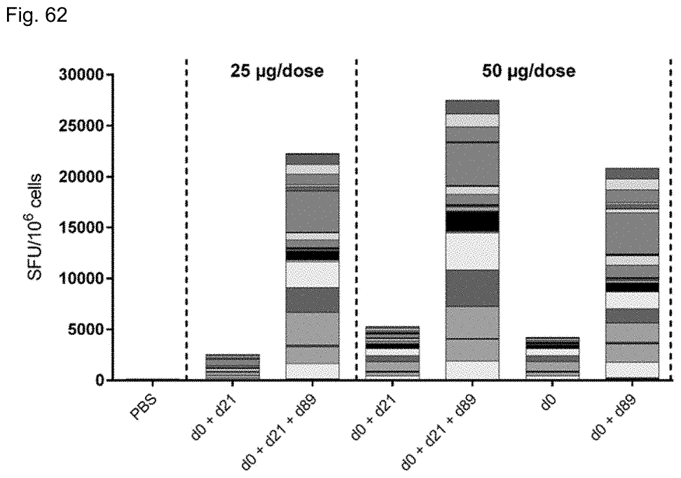

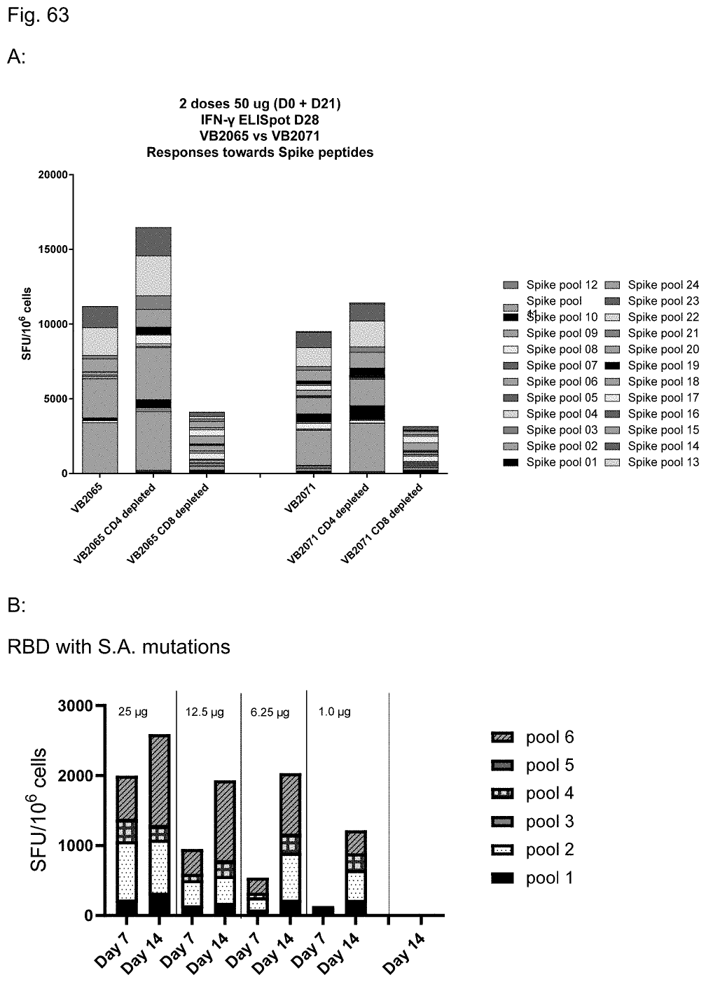

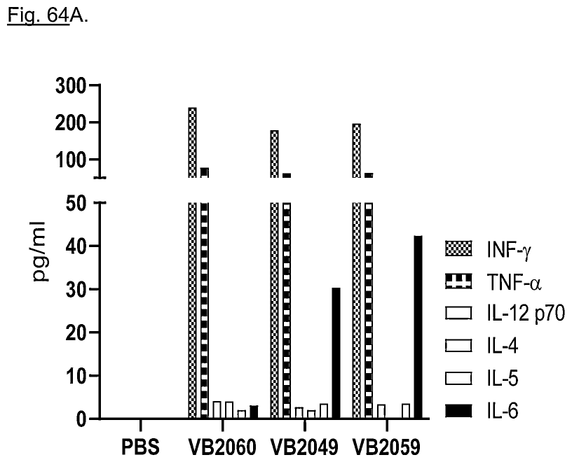

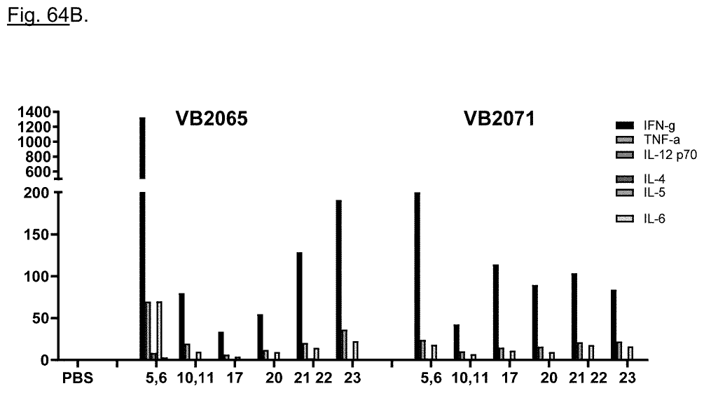

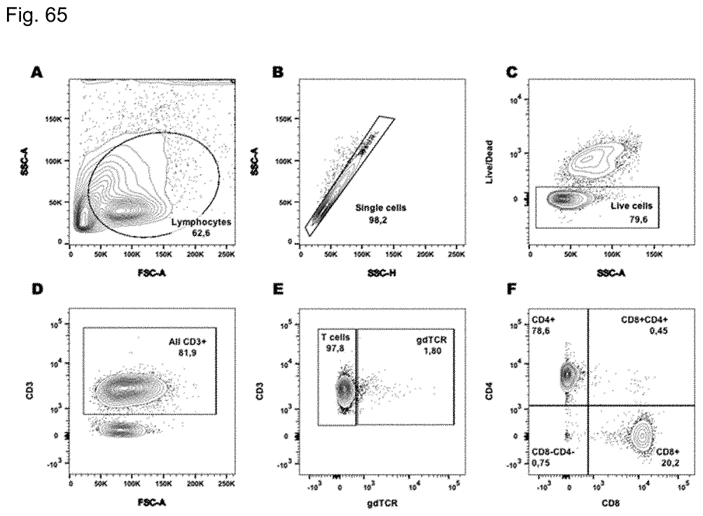

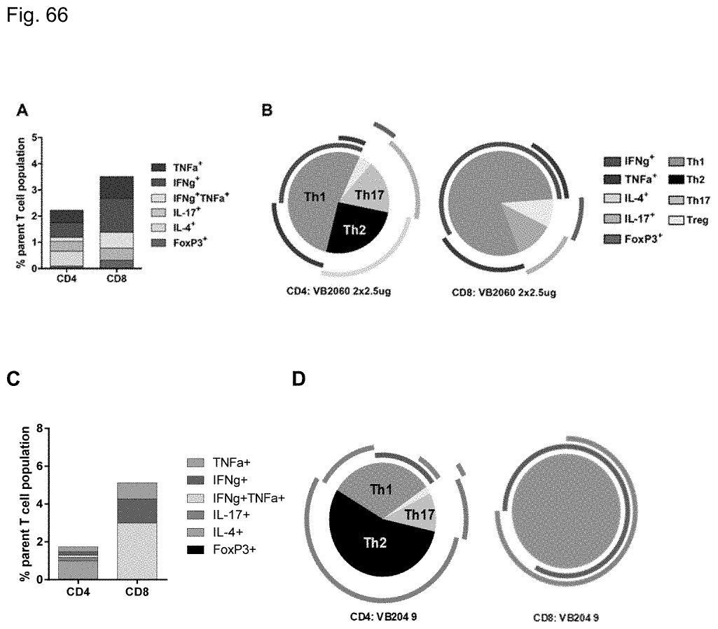

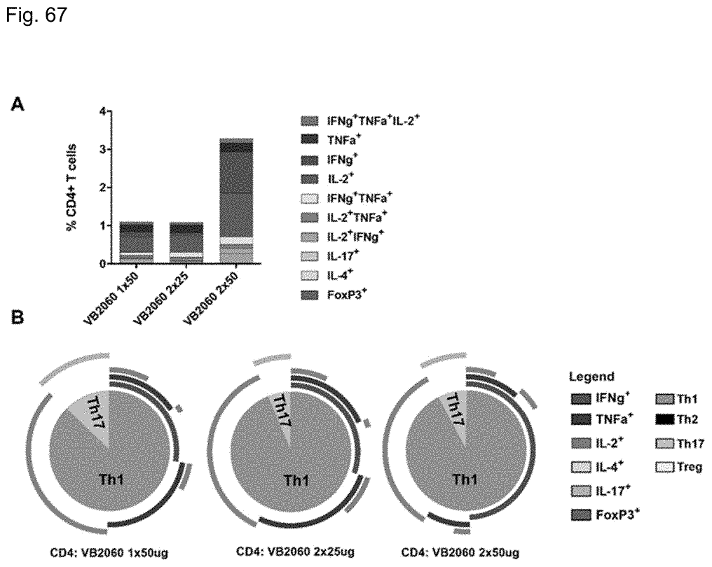

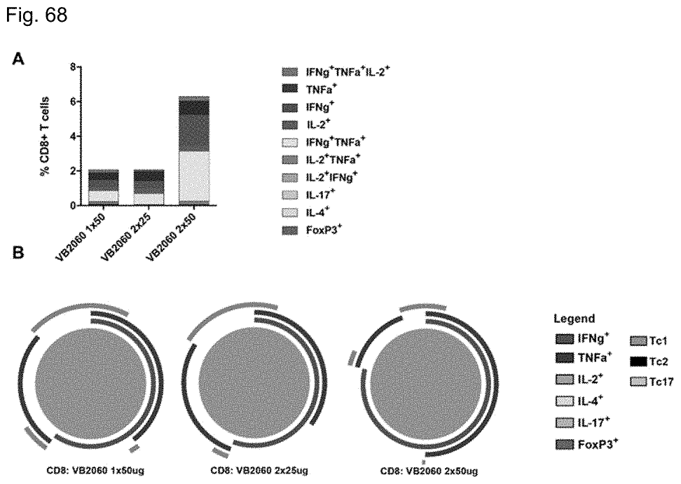

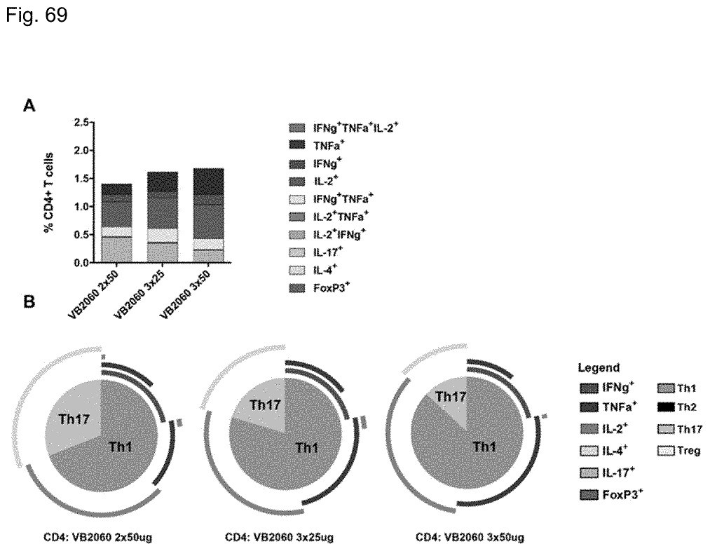

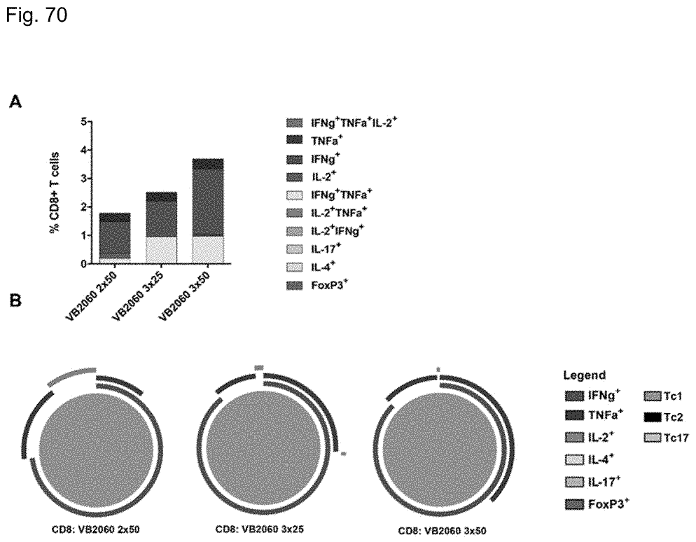

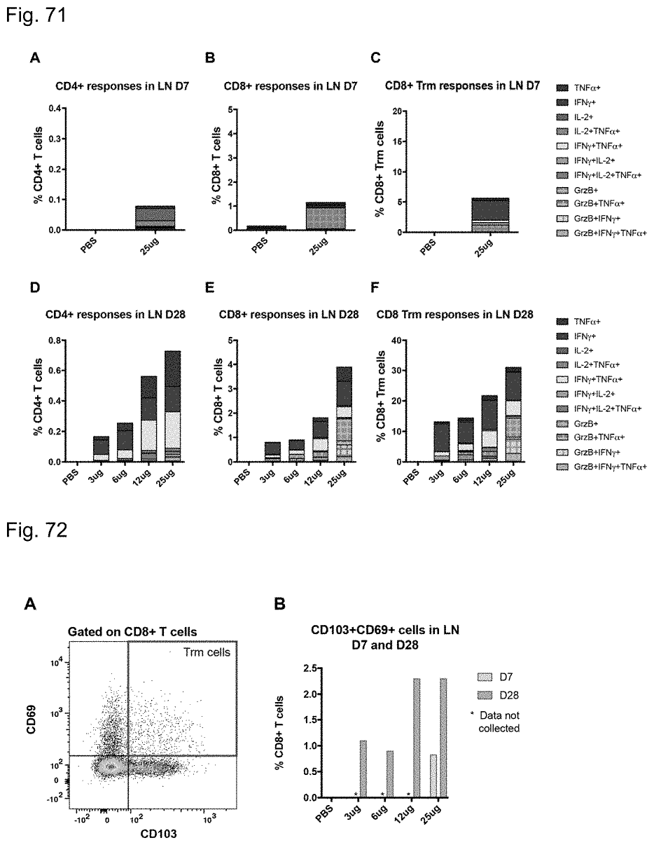

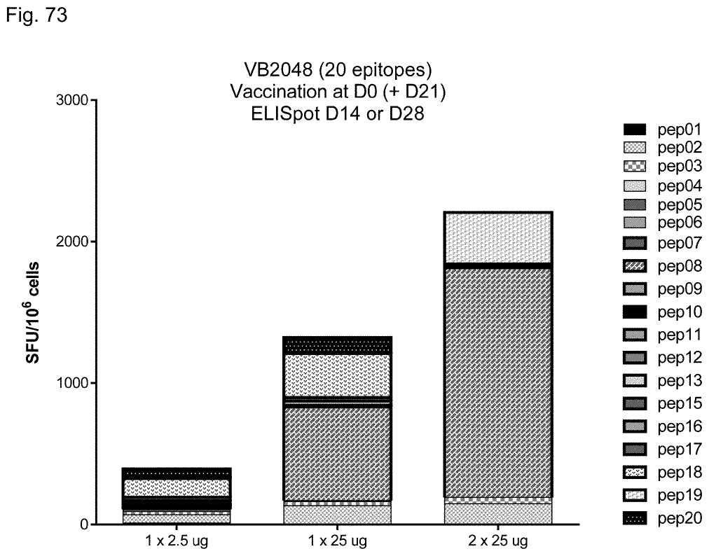

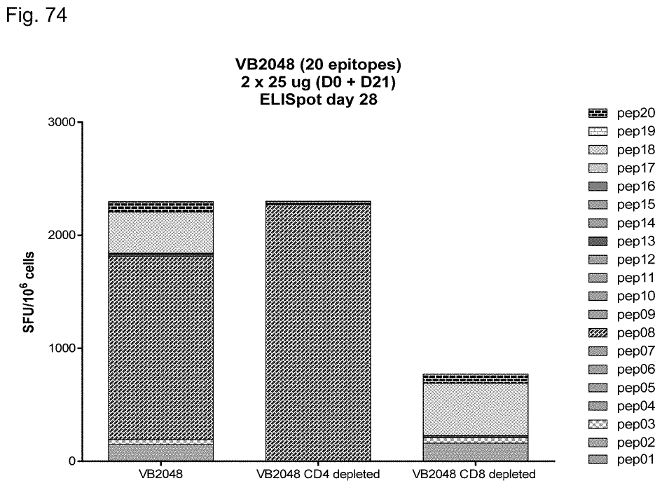

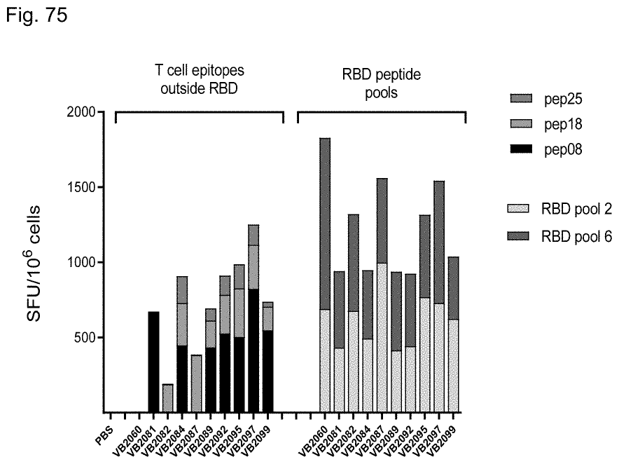

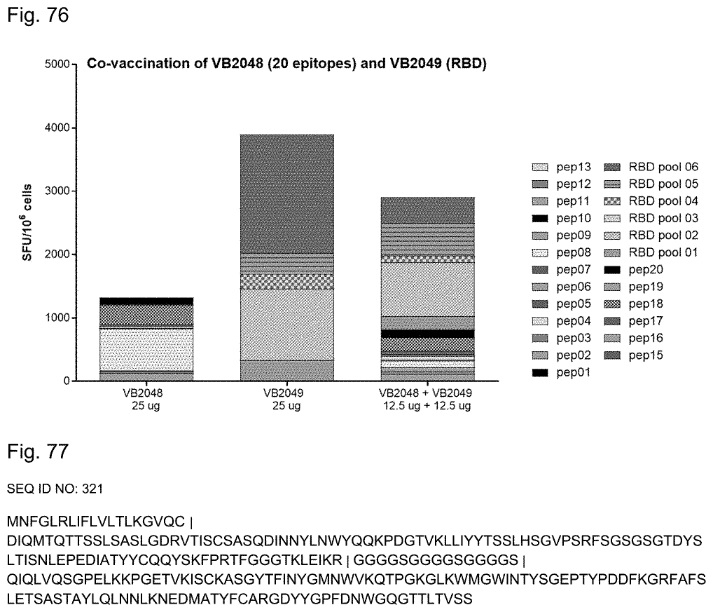

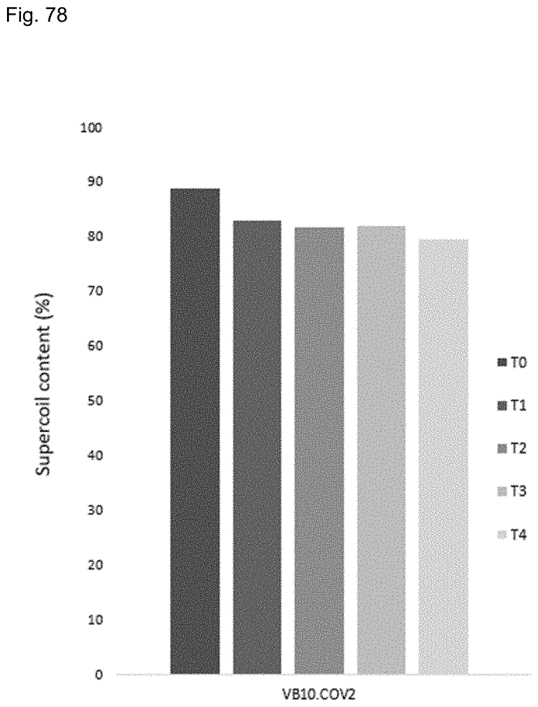

One aspect of the invention relates to a vaccine, wherein the at least one betacoronavirus epitope is the full-length spike protein of a betacoronavirus. The spike protein is one of the virus' structural proteins and forms, together with the envelope and membrane proteins, the viral envelope. The interaction between viral spike protein and angiotensin-converting enzyme 2 (ACE2) on host cell surface allows the virus to attach to and fuse with the membrane of the host cell, enter the cell and thus initiates the infection process. The spike protein is a major antigen inducing neutralizing antibodies, and thus it is considered as an antigen for vaccine design. The spike protein is prone to mutation and as such several variants exist of the spike protein and the RBD domain comprised in the spike protein (FIG. 2.)

In another embodiment the at least one betacoronavirus epitope comprises an amino acid sequence having at least 70% sequence identity to the amino acid sequence of SEQ ID NO: 230, such as at least 75%, such as at least 77%, such as at least 80%, such as at least 85%, such as at least 90%, such as at least 91%, such as at least 92%, such as at least 93%, such as at least 94%, such as at least 95%, such as at least 96%, such as at least 97%, such as at least 98% or such as at least 99% sequence identity.

In a preferred embodiment, the at least one betacoronavirus epitope is a part of the spike protein, i.e. the receptor binding domain (RBD) of the spike protein or a part of the RBD. The RBD has been found to contain multiple conformation-dependent epitopes relevant for inducing highly potent neutralizing antibodies. In another embodiment, the at least one betacoronavirus epitope comprises an amino acid sequence having at least 70% sequence identity to the amino acid sequence of SEQ ID NO: 231, such as at least 75%, such as at least 77%, such as at least 80%, such as at least 85%, such as at least 90%, such as at least 91%, such as at least 92%, such as at least 93%, such as at least 94%, such as at least 95%, such as at least 96%, such as at least 97%, such as at least 98% or such as at least 99% sequence identity.

In another embodiment, the at least one betacoronavirus epitope comprises an amino acid sequence having at least 70% sequence identity to the amino acid sequence of SEQ ID NO: 802, such as at least 75%, such as at least 77%, such as at least 80%, such as at least 85%, such as at least 90%, such as at least 91%, such as at least 92%, such as at least 93%, such as at least 94%, such as at least 95%, such as at least 96%, such as at least 97%, such as at least 98% or such as at least 99% sequence identity.

In one embodiment, the at least one betacoronavirus epitope has the amino acid sequence of SEQ ID NO: 802.

In yet another embodiment, the at least one betacoronavirus epitope comprises an amino acid sequence having at least 70% sequence identity to the amino acid sequence of SEQ ID NO: 803, such as at least 75%, such as at least 77%, such as at least 80%, such as at least 85%, such as at least 90%, such as at least 91%, such as at least 92%, such as at least 93%, such as at least 94%, such as at least 95%, such as at least 96%, such as at least 97%, such as at least 98% or such as at least 99% sequence identity.

In one embodiment, the at least one betacoronavirus epitope has the amino acid sequence of SEQ ID NO: 803.

In yet another embodiment, the at least one betacoronavirus epitope comprises an amino acid sequence having at least 70% sequence identity to the amino acid sequence of SEQ ID NO: 804, such as at least 75%, such as at least 77%, such as at least 80%, such as at least 85%, such as at least 90%, such as at least 91%, such as at least 92%, such as at least 93%, such as at least 94%, such as at least 95%, such as at least 96%, such as at least 97%, such as at least 98% or such as at least 99% sequence identity.

In one embodiment, the at least one betacoronavirus epitope has the amino acid sequence of SEQ ID NO: 804.

In yet another embodiment, the at least one betacoronavirus epitope comprises an amino acid sequence having at least 70% sequence identity to the amino acid sequence of SEQ ID NO: 805, such as at least 75%, such as at least 77%, such as at least 80%, such as at least 85%, such as at least 90%, such as at least 91%, such as at least 92%, such as at least 93%, such as at least 94%, such as at least 95%, such as at least 96%, such as at least 97%, such as at least 98% or such as at least 99% sequence identity.

In one embodiment, the at least one betacoronavirus epitope has the amino acid sequence of SEQ ID NO: 805.

In a preferred embodiment, the at least one betacoronavirus epitope comprises an amino acid sequence having at least 70% sequence identity to the amino acid sequence of SEQ ID NO: 246, such as at least 75%, such as at least 77%, such as at least 80%, such as at least 85%, such as at least 90%, such as at least 91%, such as at least 92%, such as at least 93%, such as at least 94%, such as at least 95%, such as at least 96%, such as at least 97%, such as at least 98% or such as at least 99% sequence identity.

In one embodiment, the at least one betacoronavirus epitope has the amino acid sequence of SEQ ID NO: 246.

In another preferred embodiment, the at least one betacoronavirus epitope comprises an amino acid sequence having at least 70% sequence identity to the amino acid sequence of amino acids 243 to 465 of SEQ ID NO: 255, such as at least 75%, such as at least 77%, such as at least 80%, such as at least 85%, such as at least 90%, such as at least 91%, such as at least 92%, such as at least 93%, such as at least 94%, such as at least 95%, such as at least 96%, such as at least 97%, such as at least 98% or such as at least 99% sequence identity.

In one embodiment, the at least one betacoronavirus epitope has the amino acid sequence of amino acids 243 to 465 of SEQ ID NO: 255.

In a further embodiment, the antigenic unit comprises multiple copies of the RBD and/or parts thereof, e.g. 2, 3, 4 or 5 copies, wherein the copies are not identical and comprise mutations, e.g. 1, 2, 3, 4, 5 or more mutations.

As an example, the antigenic unit may comprise two RBDs or parts thereof, e.g. the RBD of the spike protein of the Wuhan strain of SARS-CoV-2 or a part thereof and the RBD of the spike protein of the South African variant B.1.351 of SARS-CoV-2 or a part thereof. As a further example, the antigenic unit may the RBD of the spike protein of the South African variant B.1.351 of SARS-CoV-2 or a part thereof and the RBD of the spike protein of the UK variant B.1.1.7 of SARS-CoV-2 or a part thereof and the RBD of the spike protein of the Californian variant B.1.427 of SARS-CoV-2 or a part thereof. In a preferred embodiment, the copies are separated by linkers.

In another embodiment, the at least one betacoronavirus epitope is a variant causing significantly reduced neutralizing titer of prototype sera from patients or vaccines compared to the new variant strain. In one embodiment, the variant is causing a 2-4 fold or more reduced neutralizing titer, i.e. calculated by serum titer of prototype strain divided by titer against new variant strain. In another embodiment this may be all variants with RBD mutation in E484 (e.g. B.1.351, P.1, B.1.429 etc. in Greaney et al.), L452 (Cherian et al. 2021) and Q498 (Zahradnik et al 2021, and PHE, 22 Apr. 2021 VOC Tech briefing).

In another embodiment, the at least one betacoronavirus epitope is a part of the spike protein, i.e. the heptad repeat 1 (HR1) or heptad repeat 2 (HR2) domain of the spike protein. After binding of the spike protein on the virion to the ACE2 receptor on the host cell, the HR1 and HR2 domain interact with each other to form a six-helix bundle (6-HB) fusion core, bringing viral and cellular membranes into close proximity for fusion and infection. In one embodiment, the at least one betacoronavirus epitope is the HR1 domain of the spike protein, in another embodiment, the at least one betacoronavirus epitope is the HR2 domain of the spike protein.

In another embodiment the at least one betacoronavirus epitope comprises an amino acid sequence having at least 70% sequence identity to the amino acid sequence of SEQ ID NO: 232 such as at least 75%, such as at least 77%, such as at least 80%, such as at least 85%, such as at least 90%, such as at least 91%, such as at least 92%, such as at least 93%, such as at least 94%, such as at least 95%, such as at least 96%, such as at least 97%, such as at least 98% or such as at least 99% sequence identity.

In yet another embodiment the at least one betacoronavirus epitope comprises an amino acid sequence having at least 70% sequence identity to the amino acid sequence of SEQ ID NO: 249 such as at least 75%, such as at least 77%, such as at least 80%, such as at least 85%, such as at least 90%, such as at least 91%, such as at least 92%, such as at least 93%, such as at least 94%, such as at least 95%, such as at least 96%, such as at least 97%, such as at least 98% or such as at least 99% sequence identity.

In another embodiment, the at least one betacoronavirus epitope comprises at least a part of the spike protein, preferably at least a B cell epitope comprised in the spike protein or more preferably several of such B cell epitopes.

Thus, in one aspect, the invention relates to a vaccine comprising an immunologically effective amount of:

(i) a polynucleotide comprising a nucleotide sequence encoding a targeting unit, a dimerization unit and an antigenic unit, wherein the antigenic unit comprises the full-length spike protein or at least a part of the full-length spike protein of a betacoronavirus or at least a B cell epitope comprised in the spike protein; or

(ii) a polypeptide encoded by the polynucleotide as defined in (i), or

(iii) a dimeric protein consisting of two polypeptides encoded by the polynucleotide as defined in (i); and

a pharmaceutically acceptable carrier.

In a preferred embodiment, the at least one part of the spike protein is the receptor binding domain (RBD). In another preferred embodiment, the at least one part of the spike protein is the HR1 domain or the HR2 domain. In yet another preferred embodiment, the at least one part of the spike protein is the HR2 domain.

In another preferred embodiment, the antigenic unit comprises at least a B cell epitope comprised in the spike protein of a betacoronavirus, preferably comprises several B cell epitopes comprised in the spike protein of a betacoronavirus or a part thereof, e.g. the receptor binding domain, the HR1 domain or the HR2 domain.

Antibody response is more important in a prophylactic than therapeutic setting since it can block the virus and prevent that the virus infects host cells. For SARS-CoV and CoV-2, infection of human cells can occur through binding of the spike protein of the virus to the ACE2 receptor on human lung epithelia.

Another approach is a vaccine, wherein the at least one betacoronavirus epitope is a betacoronavirus T cell epitope. The present disclosure reveals that conserved parts of the genome among betacoronaviruses comprise T cell epitopes capable of initiating an immune response. Thus, one aspect of the invention relates to a vaccine comprising at least one T cell epitope, preferably at least one T cell epitope that is conserved between several species or strains of betacoronaviruses, e.g. conserved between SARS-Cov2 and SARS-CoV.

The T cell epitopes may be comprised in any of the virus' proteins, i.e. in viral surface proteins but also in the nucleocapsid protein or replicase polyproteins or in other structural and non-structural proteins.

Several of the T cell epitopes that have been found to be reactive in humans are also in the non-structural proteins and open reading frames, where functions may not have been fully elucidated but could still have a critical function for the virus (see e.g. Tarke et al 2021 Table in Suppl where genes and epitopes are listed).

Thus, in another aspect, the invention relates to a vaccine comprising an immunologically effective amount of:

(i) a polynucleotide comprising a nucleotide sequence encoding a targeting unit, a dimerization unit and an antigenic unit, wherein the antigenic unit comprises at least one T cell epitope of a betacoronavirus; or

(ii) a polypeptide encoded by the polynucleotide as defined in (i), or

(iii) a dimeric protein consisting of two polypeptides encoded by the polynucleotide as defined in (i); and

a pharmaceutically acceptable carrier.

In a preferred embodiment, the antigenic unit comprises several T cell epitopes of a betacoronavirus, preferably several T cell epitopes that are conserved between several species or strains of betacoronaviruses. In one embodiment, the antigenic unit comprises 2 to 50 T cell epitopes, e.g. 3 to 45 T cell epitopes, e.g. 4 to 40 T cell epitopes, e.g. 5 to 35 T cell epitopes, e.g. 6 to 30 T cell epitopes, e.g. 7 to 25 T cell epitopes, such as 7, 8, 9, 10, 11, 12, 13, 14, 15, 16, 17, 18, 19, 20, 21, 22, 23, 24 or 25 T cell epitopes.

A vaccine comprising T cell epitopes from conserved regions of betacoronaviruses will provide protection against several species/strains of betacoronaviruses, e.g. against several strains of SARS-CoV, e.g. against SARS-CoV and SARS-CoV-2. Such a vaccine will also provide protection against multiple variants of a betacoronavirus, e.g. variants of the SARS-CoV virus or variants of the SARS-CoV-2 virus, which is important for the efficacy of such a vaccine against future mutated viruses. Viruses are known to mutate, e.g. undergo viral antigen drift or antigen shift. The finding of conserved regions across the betacoronavirus genus makes it likely that these conserved regions are needed to maintain essential structures or functions, thus it is anticipated that future mutations will take place in the less-conserved regions. By raising an immune response against the conserved regions, the vaccinated individual will be protected also against mutated (and thus novel) strains of the future.

In one embodiment of the present invention, the vaccine is therefore designed to evoke a cell-mediated immune response through activation of T cells against betacoronavirus epitopes. T cells recognize epitopes when they have been processed and presented complexed to an MHC molecule.

There are two primary classes of major histocompatibility complex (MHC) molecules, MHC I and MHC II. The terms MHC (class) I and MHC (class) II are interchangeably used herein with HLA (class) I and HLA (class) II. Human leukocyte antigen (HLA) is a major histocompatibility complex in humans.

The T cell epitope comprised in the antigenic unit of a vaccine of the invention which only comprises T cell epitopes or in the antigenic unit of a vaccine of the invention which comprises T cell epitopes but further comprises at least one betacoronavirus epitope which is a full-length viral surface protein or a part thereof, the T cell epitope has a length of from 7 to about 200 amino acids, with the longer T cell epitopes possibly including hotspots of minimal epitopes. A hotspots of minimal epitopes is a region that contains several minimal epitopes (e.g. having a length of from 8-15 amino acids) that are predicted to be presented by different HLA alleles to cover a broad range of world population

In one embodiment, the antigenic unit of such a vaccine comprises T cell epitopes with a length of from 7 to 150 amino acids, preferably of from 7 to 100 amino acids, e.g. from about 10 to about 100 amino acids or from about 15 to about 100 amino acids or from about 20 to about 75 amino acids or from about 25 to about 50 amino acids.

In a preferred embodiment, the antigenic unit of such a vaccine comprises T cell epitopes having a length suitable for specific presentation on MHC I or MHC II. In one embodiment, the T cell epitope has a length of from 7 to 11 amino acids for MHCI presentation. In another embodiment, the T cell epitope sequence has a length of from 9-60 amino acids, such as from 9 to 30 amino acids, such as 15-60 amino acids, such as 15-30 for MHCII presentation. In a preferred embodiment the T cell epitope has a length of 15 amino acids for MHC II presentation.

In another preferred embodiment, the T cell epitope is selected based on the predicted ability to bind to HLA class I/II alleles. In yet another embodiment, the T cell epitope is known to be immunogenic, e.g. its immunogenicity has been confirmed by appropriate methods and the results have been published, e.g. in a scientific publication.

In another embodiment of the invention the antigenic unit includes multiple T cell epitopes that are known to be immunogenic or predicted to bind to HLA class I/II alleles. The latter T cell epitopes are selected in silico on the basis of predictive HLA-binding algorithms. After having identified all relevant epitopes, the epitopes are ranked according to their ability to bind to HLA class I/II alleles and the epitopes that are predicted to bind best are selected to be included in the antigenic unit.

Any suitable HLA-binding algorithm may be used, such as one of the following: Available software analysis of peptide-MHC binding (IEDB, NetMHCpan and NetMHCIIpan) may be downloaded or used online from the following websites:

http://www.iedb.org/

https://services.healthtech.dtu.dk/service.php?NetMHCpan-4.0

https://services.healthtech.dtu.dk/service.php?NetMHCIIpan-3.2

Commercially available advanced software to predict optimal sequences for vaccine design are found here:

http://www.oncoimmunity.com/

https://omictools.com/t-cell-epitopes-category

https://github.com/griffithlab/pVAC-Seq

http://crdd.osdd.net/raghava/cancertope/help.php

http://www.epivax.com/tag/neoantigen/

In another embodiment, each T cell epitope is ranked with respect to its predicted binding affinity and/or antigenicity, and the predicted most antigenic epitopes are selected and preferably optimally arranged in the antigenic unit.

In an embodiment of the present invention, the T cell epitope sequence is a part of the sequence of the spike protein or the membrane protein or the envelope protein or the nucleocapsid protein or the ORF1a/b or ORF3a protein. In another embodiment, the T cell epitope sequence is part of the following genes/proteins: NCAP, AP3A, spike, ORF1a/b, ORF3a, VME1 and VEMP.

One embodiment of the invention relates to a method of identifying T cell epitopes that are conserved between betacoronaviruses, e.g. between betacoronaviruses of the same subgenus, e.g. between SARS-CoV-2 and SARS-CoV comprising:
Identification of sets of HLA class I and II alleles that are specific for a specific population or a specific ethnic group or a specific geographic region
Identification of genomic regions in the conserved viral sequence of SARS-CoV-2 that contain hotspots of minimal epitopes, i.e. minimal epitopes predicted to be presented by different HLA class I and II alleles to cover a broad range of the world's population
Selection of SARS-CoV-2 T cell epitopes in the hotspots that cover the highest number of different HLA class I and II alleles
From the selected T cell epitopes, identifying those that are conserved between SARS-CoV and SARS-CoV-2
Checking the selected T cell epitopes for similarity to sequences found in the normal human proteome and removing those T cell epitopes with a high number of matches to such sequences
From the remaining selected T cell epitopes, identifying those that match or have a high similarity to minimal epitopes already described to be immunogenic

In another embodiment the invention relates to a method of identifying T cell epitopes that are conserved between betacoronaviruses, e.g. between betacoronaviruses of the same subgenus, e.g. between SARS-CoV-2 and SARS-CoV comprising:
Identification of sets of HLA class I and II alleles that are specific for a specific population or a specific ethnic group or a specific geographic region
Identification of genomic regions in the viral sequence of SARS-CoV-2 that contain hotspots of minimal epitopes
Selection of the optimal set of hotspots that covers the highest number of SARS-CoV and SARS-CoV-2 variants, as well as the highest number of different HLA class I and II alleles
Checking the selected T cell epitopes for similarity to sequences found in the normal human proteome and removing those T cell epitopes with a high number of matches to such sequences
From the remaining selected T cell epitopes, identifying those that match or have a high similarity to minimal epitopes already described to be immunogenic

In this method, the selection of the optimal set of hotspots is implemented as an optimization algorithm (maximum set coverage) so both HLA coverage and pathogen conservation are optimized at the same time.

Specific T cell epitopes have been identified by the disclosed procedure. In an embodiment of the present invention the T cell epitope is selected from the epitopes listed in Example 1. In a preferred embodiment, the T cell epitope is selected from the list consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229 and SEQ ID NOs: 322-444.

In preferred embodiment, the T cell epitope is selected from the list consisting of: SEQ ID NO: 67, SEQ ID NO: 19, SEQ ID NO: 78, SEQ ID NO: 57, SEQ ID NO: 50, SEQ ID NO: 55, SEQ ID NO: 64, SEQ ID NO: 22, SEQ ID NO: 87, SEQ ID NO: 62, SEQ ID NO: 39, SEQ ID NO: 59, SEQ ID NO: 26, SEQ ID NO: 53, SEQ ID NO: 32, SEQ ID NO: 38, SEQ ID NO: 30, SEQ ID NO: 40, SEQ ID NO: 42, SEQ ID NO: 35, SEQ ID NO: 71, SEQ ID NO: 9, SEQ ID NO: 21, SEQ ID NO: 85, SEQ ID NO: 75, SEQ ID NO: 23, SEQ ID NO: 34, SEQ ID NO: 36, SEQ ID NO: 77 and SEQ ID NO: 20.

In another embodiment, the T cell epitope is selected from the list consisting of: SEQ ID NO: 67, SEQ ID NO: 19, SEQ ID NO: 78, SEQ ID NO: 57, SEQ ID NO: 50, SEQ ID NO: 55, SEQ ID NO: 64, SEQ ID NO: 22, SEQ ID NO: 87 and SEQ ID NO: 62.

In yet another embodiment, the T cell epitopes is selected from the list consisting of pep1-pep20 disclosed in Table 1 and SEQ ID NO: 75. In yet another embodiment, the T cell epitope is one that has been confirmed to be immunogenic in a clinical trial or is validated in human patients having had an infection with a betacoronavirus.

In yet another aspect the invention relates to a vaccine comprising an immunologically effective amount of:

(i) a polynucleotide comprising a nucleotide sequence encoding a targeting unit, a dimerization unit and an antigenic unit, wherein the antigenic unit comprises a) a full-length viral surface protein of a betacoronavirus or a part thereof and b) at least one betacoronavirus T cell epitope; or

(ii) a polypeptide encoded by the polynucleotide as defined in (i), or

(iii) a dimeric protein consisting of two polypeptides encoded by the polynucleotide as defined in (i); and

a pharmaceutically acceptable carrier.

In one embodiment, the full-length protein is selected from the group consisting of envelope protein, spike protein, membrane protein and hemagglutinin esterase.

In another embodiment, the antigenic unit comprises at least a B cell epitope comprised in such full-length viral surface protein of a betacoronavirus, e.g. comprised in any of the aforementioned proteins and preferably comprises several B cell epitopes comprised in such full-length viral surface protein of a betacoronavirus, e.g. comprised in any of the aforementioned proteins.

In yet another aspect the invention relates to a vaccine comprising an immunologically effective amount of:

(i) a polynucleotide comprising a nucleotide sequence encoding a targeting unit, a dimerization unit and an antigenic unit, wherein the antigenic unit comprises a) the full-length spike protein of a betacoronavirus or a part thereof or at least one betacoronavirus B cell epitope comprised in the spike protein and b) at least one betacoronavirus T cell epitope; or

(ii) a polypeptide encoded by the polynucleotide as defined in (i), or

(iii) a dimeric protein consisting of two polypeptides encoded by the polynucleotide as defined in (i); and

a pharmaceutically acceptable carrier.

In one embodiment, the antigenic unit comprises the full-length spike protein. As such, the antigenic unit comprises an amino acid sequence having at least 70% sequence identity to the amino acid sequence of SEQ ID NO: 230, such as at least 75%, such as at least 77%, such as at least 80%, such as at least 85%, such as at least 90%, such as at least 91%, such as at least 92%, such as at least 93%, such as at least 94%, such as at least 95%, such as at least 96%, such as at least 97%, such as at least 98% or such as at least 99% sequence identity.

In one embodiment, the antigenic unit of the aforementioned vaccine comprises a) the receptor binding domain of the spike protein of a betacoronavirus and b) at least one betacoronavirus T cell epitope.

In one embodiment, the antigenic unit comprises an amino acid sequence having at least 70% sequence identity to the amino acid sequence of SEQ ID NO: 231, such as at least 75%, such as at least 77%, such as at least 80%, such as at least 85%, such as at least 90%, such as at least 91%, such as at least 92%, such as at least 93%, such as at least 94%, such as at least 95%, such as at least 96%, such as at least 97%, such as at least 98% or such as at least 99% sequence identity.

In one embodiment, the antigenic unit comprises the amino acid sequence of SEQ ID NO: 231.

In another embodiment, the antigenic unit comprises an amino acid sequence comprises an amino acid sequence having at least 70% sequence identity to the amino acid sequence of SEQ ID NO: 802, such as at least 75%, such as at least 77%, such as at least 80%, such as at least 85%, such as at least 90%, such as at least 91%, such as at least 92%, such as at least 93%, such as at least 94%, such as at least 95%, such as at least 96%, such as at least 97%, such as at least 98% or such as at least 99% sequence identity.

In one embodiment, the antigenic unit comprises the amino acid sequence of SEQ ID NO: 802.

In yet another embodiment, the antigenic unit comprises an amino acid sequence having at least 70% sequence identity to the amino acid sequence of SEQ ID NO: 803, such as at least 75%, such as at least 77%, such as at least 80%, such as at least 85%, such as at least 90%, such as at least 91%, such as at least 92%, such as at least 93%, such as at least 94%, such as at least 95%, such as at least 96%, such as at least 97%, such as at least 98% or such as at least 99% sequence identity.

In one embodiment, the antigenic unit comprises the amino acid sequence of SEQ ID NO: 803.

In yet another embodiment, the antigenic unit comprises an amino acid sequence having at least 70% sequence identity to the amino acid sequence of SEQ ID NO: 804, such as at least 75%, such as at least 77%, such as at least 80%, such as at least 85%, such as at least 90%, such as at least 91%, such as at least 92%, such as at least 93%, such as at least 94%, such as at least 95%, such as at least 96%, such as at least 97%, such as at least 98% or such as at least 99% sequence identity.

In one embodiment, the antigenic unit comprises the amino acid sequence of SEQ ID NO: 804.

In yet another embodiment, the antigenic unit comprises an amino acid sequence having at least 70% sequence identity to the amino acid sequence of SEQ ID NO: 805, such as at least 75%, such as at least 77%, such as at least 80%, such as at least 85%, such as at least 90%, such as at least 91%, such as at least 92%, such as at least 93%, such as at least 94%, such as at least 95%, such as at least 96%, such as at least 97%, such as at least 98% or such as at least 99% sequence identity.

In one embodiment, the antigenic unit comprises the amino acid sequence of SEQ ID NO: 805.

In a preferred embodiment, the antigenic unit comprises an amino acid sequence having at least 70% sequence identity to the amino acid sequence of SEQ ID NO: 246, such as at least 75%, such as at least 77%, such as at least 80%, such as at least 85%, such as at least 90%, such as at least 91%, such as at least 92%, such as at least 93%, such as at least 94%, such as at least 95%, such as at least 96%, such as at least 97%, such as at least 98% or such as at least 99% sequence identity.

In one embodiment, the antigenic unit comprises the amino acid sequence of SEQ ID NO: 246.

In another preferred embodiment, the antigenic unit comprises an amino acid sequence having at least 70% sequence identity to the amino acid sequence of amino acids 243 to 455 of SEQ ID NO: 255, such as at least 75%, such as at least 77%, such as at least 80%, such as at least 85%, such as at least 90%, such as at least 91%, such as at least 92%, such as at least 93%, such as at least 94%, such as at least 95%, such as at least 96%, such as at least 97%, such as at least 98% or such as at least 99% sequence identity.

In one embodiment, the antigenic unit comprises the amino acid sequence of amino acids 243 to 455 of SEQ ID NO: 255.

In a further embodiment, the antigenic unit comprises multiple copies of the RBD and/or parts thereof, e.g. 2, 3, 4 or 5 copies, wherein the copies are not identical and comprise mutations, e.g. 1, 2, 3, 4, 5 or more mutations.

As an example, the antigenic unit may comprise two RBDs or parts thereof, e.g. the RBD of the spike protein of the Wuhan strain of SARS-CoV-2 or a part thereof and the RBD of the spike protein of the South African variant B.1.351 of SARS-CoV-2 or a part thereof domains. As a further example, the antigenic unit may the RBD of the spike protein of the South African variant B.1.351 of SARS-CoV-2 or a part thereof and the RBD of the spike protein of the UK variant B.1.1.7 of SARS-CoV-2 or a part thereof and the RBD of the spike protein of the Californian variant B.1.427 of SARS-CoV-2 or a part thereof.

In another embodiment, the antigenic unit of the aforementioned vaccine comprises a) the HR1 domain or HR2 domain of the spike protein of a betacoronavirus and b) at least one betacoronavirus T cell epitope. In yet another embodiment, the antigenic unit of the aforementioned vaccine comprises a) the HR2 domain of the spike protein of a betacoronavirus and b) at least one betacoronavirus T cell epitope.

In a preferred embodiment, the at least one T cell epitope is selected from the list consisting of SEQ ID NO: 1-SEQ ID NO: 444, preferably at least one T cell epitope selected from the list consisting of SEQ ID NO: 67, SEQ ID NO: 19, SEQ ID NO: 78, SEQ ID NO: 57, SEQ ID NO: 50, SEQ ID NO: 55, SEQ ID NO: 64, SEQ ID NO: 22, SEQ ID NO: 87, SEQ ID NO: 62, SEQ ID NO: 39, SEQ ID NO: 59, SEQ ID NO: 26, SEQ ID NO: 53, SEQ ID NO: 32, SEQ ID NO: 38, SEQ ID NO: 30, SEQ ID NO: 40, SEQ ID NO: 42, SEQ ID NO: 35, SEQ ID NO: 71, SEQ ID NO: 9, SEQ ID NO: 21, SEQ ID NO: 85, SEQ ID NO: 75, SEQ ID NO: 23, SEQ ID NO: 34, SEQ ID NO: 36, SEQ ID NO: 77 and SEQ ID NO: 20, more preferably at least one T cell epitope selected from the list consisting of SEQ ID NO: 67, SEQ ID NO: 19, SEQ ID NO: 78, SEQ ID NO: 57, SEQ ID NO: 50, SEQ ID NO: 55, SEQ ID NO: 64, SEQ ID NO: 22, SEQ ID NO: 87 and SEQ ID NO: 62.

The length of the antigenic unit is primarily determined by the length of the epitope sequences comprised therein as well as their numbers. In one embodiment, the epitopes are separated from each other by linkers, which also contribute to the length of the antigenic unit.

In one embodiment, the antigenic unit comprises up to 3500 amino acids, such as from 21 to 3500 amino acids, preferably from about 30 amino acids to about 2000 amino acids such as from about 50 to about 1500 amino acids, more preferably from about 100 to about 1500 amino acids, such as from about 100 to about 1000 amino acids or from about 100 to about 500 amino acids or from about 100 to about 300 amino acids.

Although it is possible to obtain a relevant immune response if the betacoronavirus epitopes are randomly arranged in the antigenic unit, it is preferred to follow at least one of the following methods for arranging T cell epitopes and/or B cell epitopes, preferably linear B cell epitopes (in the following denoted “epitopes”) in the antigenic unit in order to enhance the immune response.

The antigenic unit can be described as a polypeptide having an N-terminal start and a C-terminal end. The antigenic unit is connected to the dimerization unit, preferably via a unit linker. The antigenic unit is either located at the COOH-terminal end or the NH2-terminal end of the polypeptide/dimeric protein. It is preferred that the antigenic unit is in the COOH-terminal end of the polypeptide/dimeric protein.

In one embodiment, the epitopes are arranged in the order of from most antigenic to least antigenic in the direction from the dimerization unit towards the end of the antigenic unit, i.e. the terminal epitope.

In another embodiment, in particular if the hydrophilicity/hydrophobicity varies greatly among the epitopes, it is preferred that the most hydrophobic epitope(s) is/are positioned substantially in the middle of the antigenic unit and the most hydrophilic epitope(s) is/are positioned at the beginning and/or end of the antigenic unit.

Since a true positioning in the middle of the antigenic unit is only possible if the antigenic unit comprises an odd number of epitopes, the term “substantially” in this context refers to antigenic units comprising an even number of epitopes, wherein the most hydrophobic epitopes are positioned as closed to the middle as possible.

By way of example, an antigenic unit comprises 5 epitopes which are arranged as follows: 1-2-3*-4-5; with 1, 2, 3*, 4 and 5 each being an epitope and * indicates the most hydrophobic epitope, which is positioned in the middle of the antigenic unit.

In another example, an antigenic unit comprises 6 epitopes which are arranged as follows: 1-2-3*-4-5-6 or, alternatively, as follows: 1-2-4-3*-5-6; with 1, 2, 3*, 4, 5 and 6 each being an epitope and * indicates the most hydrophobic epitope, which is positioned substantially in the middle of the antigenic unit.

Alternatively, the epitopes may be arranged alternating between a hydrophilic and a hydrophobic antigen sequence.

Furthermore, GC rich epitopes should not be arranged adjacent to each other to avoid GC clusters. In a preferred embodiment, one GC rich epitope is followed by at least one non-GC rich epitope before a second GC rich epitope follows.

In one embodiment, the vaccine according to the invention comprises an antigenic unit which comprises 1 to 50 epitopes. In a preferred embodiment, said epitopes are T cell epitopes.

In one embodiment from 3 to 50 epitopes are included in the antigenic unit, such as from 3 to 30 epitopes, such as from 3 to 20 epitopes, such as from 3 to 15 epitopes, or such as from 3 to 10 epitopes. In a preferred embodiment, said epitopes are T cell epitopes.

In another embodiment 5 to 50 epitopes are included in the antigenic unit, such as from 5 to 30 epitopes, such as for example from 5 to 25 epitopes, such as from 5 to 20 epitopes, such as from 5 to 15 epitopes or such as from 5 to 10 epitopes. In a preferred embodiment, said epitopes are T cell epitopes.

In a further embodiment 10 to 50 epitopes are included in the antigenic unit, such as from 10 to 40 epitopes, such as from 10 to 30 epitopes, such as from 10 to 25 epitopes, such as from 10 to 20 epitopes or such as from 10 to 15 epitopes. In a preferred embodiment, said epitopes are T cell epitopes.

In a preferred embodiment the antigenic unit consists of 10, 20, 30 or 50 epitopes. In a preferred embodiment, said epitopes are T cell epitopes.

The antigenic unit may further comprise one or more linkers, which separate one epitope or several epitopes from one other epitopes or several other epitopes and a linker, which connects the antigenic unit to the dimerization unit (hereinafter called the unit linker). The one or more linkers ensure that the epitopes are presented in an optimal way to the immune system, which increases the vaccine's efficacy. For vaccines wherein the antigenic unit comprises a full-length protein of the betacoronavirus or a part of such protein, the presence of a linker may also ensure that the protein is folding correctly.

The one or more linkers are preferably designed to be non-immunogenic and are preferably also flexible. In a vaccine comprising a full-length viral surface protein or a part thereof, e.g. the spike protein or parts thereof, the linkers allow for the protein to fold correctly and thus optimize presentation of the included B cell epitopes to B cells. In addition, the linkers allow effective secretion of a functional vaccine protein that is effectively delivered to antigen presenting cells and thus increases presentation of T cell epitopes to T cells, even if the antigenic unit comprises a high number of epitopes. Preferably, the length of the one or more linkers is from 4 to 20 amino acids to secure flexibility. In another preferred embodiment, the length of the one or more linkers is from 8 to 20 amino acids, such as from 8 to 15 amino acids, for example 8 to 12 amino acids or such as for example from 10 to 15 amino acids. In a particular embodiment, the length of the one or more linkers is 10 amino acids.

The one or more linkers have preferably all the same nucleotide or amino acid sequence. If, however, one or more of the epitopes comprise an amino acid motif similar to the linker, it may be an advantage to substitute the neighboring linkers of that epitope with linker of a different sequence. Further, if an epitope/linker junction is predicted to constitute an epitope in itself, then a linker of a different sequence might be used.

The one or more linkers are preferably serine (S)-glycine (G) linkers which comprise several serine and/or several glycine residues. Preferred examples are GGGGS (SEQ ID NO: 806), GGGSS (SEQ ID NO: 807), GGGSG (SEQ ID NO: 808), GGGGS or multiple variants thereof such as GGGGSGGGGS (SEQ ID NO: 809) or (GGGGS)ₘ, (GGGSS)ₘ, (GGGSG)ₘ, where m is an integer from 1 to 5, from 1 to 4 or from 1 to 3. In a preferred embodiment, m is 2.

In a preferred embodiment, the serine-glycine linker further comprises at least one leucine (L) residue, such as at least 2 or at least 3 leucines. The serine-glycine linker may for example comprise 1, 2, 3 or 4 leucine. Preferably, the serine-glycine linker comprises 1 leucine or 2 leucines.

In one embodiment, the one or more linkers comprise or consist of the sequence LGGGS (SEQ ID NO: 810), GLGGS (SEQ ID NO: 811), GGLGS (SEQ ID NO: 812), GGGLS (SEQ ID NO: 813) or GGGGL (SEQ ID NO: 814). In another embodiment, the one or more linkers comprise or consist of the sequence LGGSG (SEQ ID NO: 815), GLGSG (SEQ ID NO: 816), GGLSG (SEQ ID NO: 817), GGGLG (SEQ ID NO: 818) or GGGSL. In yet another embodiment, the one or more linkers comprise or consist of the sequence LGGSS (SEQ ID NO: 819), GLGSS (SEQ ID NO: 820), GGLSS (SEQ ID NO: 821), GGGLS or GGGSL (SEQ ID NO: 822).

In yet another embodiment, the one or more linkers comprise or consist of the sequence LGLGS (SEQ ID NO: 823), GLGLS (SEQ ID NO: 824), GLLGS (SEQ ID NO: 825), LGGLS (SEQ ID NO: 826) or GLGGL (SEQ ID NO: 827). In yet another embodiment, one or more linkers comprise or consist of the sequence LGLSG (SEQ ID NO: 828), GLLSG (SEQ ID NO: 829), GGLSL (SEQ ID NO: 830), GGLLG (SEQ ID NO: 831) or GLGSL (SEQ ID NO: 832). In yet another embodiment, the one or more linkers comprise or consist of the sequence LGLSS (SEQ ID NO: 833), GLGLS, GGLLS (SEQ ID NO: 834), GLGSL or GLGSL.

In another embodiment, the one or more linkers are serine-glycine linkers that have a length of 10 amino acids and comprise 1 leucine or 2 leucines.

In one embodiment, the one or more linkers comprise or consist of the sequence LGGGSGGGGS (SEQ ID NO: 835), GLGGSGGGGS (SEQ ID NO: 836), GGLGSGGGGS (SEQ ID NO: 837), GGGLSGGGGS (SEQ ID NO: 838) or GGGGLGGGGS (SEQ ID NO: 839). In another embodiment, the one or more linkers comprise or consist of the sequence LGGSGGGGSG (SEQ ID NO: 840), GLGSGGGGSG (SEQ ID NO: 841), GGLSGGGGSG (SEQ ID NO: 842), GGGLGGGGSG (SEQ ID NO: 843) or GGGSLGGGSG (SEQ ID NO: 844). In yet another embodiment, the one or more linkers comprise or consist of the sequence LGGSSGGGSS (SEQ ID NO: 845), GLGSSGGGSS (SEQ ID NO: 846), GGLSSGGGSS (SEQ ID NO: 847), GGGLSGGGSS (SEQ ID NO: 848) or GGGSLGGGSS (SEQ ID NO: 849).

In a further embodiment, the one or more linkers comprise or consist of the sequence LGGGSLGGGS (SEQ ID NO: 850), GLGGSGLGGS (SEQ ID NO: 851), GGLGSGGLGS (SEQ ID NO: 852), GGGLSGGGLS (SEQ ID NO: 853) or GGGGLGGGGL (SEQ ID NO: 854). In another embodiment, the one or more linkers comprise or consist of the sequence LGGSGLGGSG (SEQ ID NO: 855), GLGSGGLGSG (SEQ ID NO: 856), GGLSGGGLSG (SEQ ID NO: 857), GGGLGGGGLG (SEQ ID NO: 858) or GGGSLGGGSL (SEQ ID NO: 859). In yet another embodiment, the one or more linkers comprise or consist of the sequence LGGSSLGGSS (SEQ ID NO: 860), GLGSSGLGSS (SEQ ID NO: 861), GGLSSGGLSS (SEQ ID NO: 862), GGGLSGGGLS or GGGSLGGGSL.

In further embodiments, the one or more linkers comprise or consist of the sequence TQKSLSLSPGKGLGGL (SEQ ID NO: 863). In another embodiment, the one or more linkers comprise or consist of the sequence SLSLSPGKGLGGL (SEQ ID NO: 864).

For a vaccine comprising an antigenic unit comprising a full-length protein of the betacoronavirus or a part of such protein and one or more T cell epitopes, in one embodiment, the linker separating the T cell epitopes and the protein has a length of from 10 to 60 amino acids, e.g. from 11 to 50 amino acid or from 12 to 45 amino acids or from 13 to 40 amino acids.

Also such linkers are preferably non-immunogenic. Examples of such linkers are glycine-serine rich linkers or glycine-serine-leucine rich linkers as described above, GSAT (SEQ ID NO: 865) linkers comprising or consisting of the sequence GGSAGGSGSGSSGGSSGASGTGTAGGTGSGSGTGSG (SEQ ID NO: 866). In another embodiment, such linkers are SEG linkers comprising or consisting of the sequence GGSGGGSEGGGSEGGGSEGGGSEGGGSEGGGSGGGS (SEQ ID NO: 867). Further, protein modelling may be used to model 3D structures/conformations of the protein connected to the linker to determine, which length and amino acid sequence promotes correct folding.

In one embodiment, the antigenic unit comprises from 10 to 20 or from 10 to 25 epitopes and a plurality of linkers, which separate each of the epitopes or separate several epitopes from several other epitopes. Preferably, said linkers have a length of 10 amino acids. The linkers may also have any length as defined herein above, such as for example from 5 to 12 amino acids.

Alternatively, the one or more linkers may be selected from the group consisting of GSAT linkers, i.e. a linker comprising one or more glycine, serine, alanine and threonine residues, and SEG linkers, i.e. a linker comprising one or more serine, glutamic acid and glycine residues, or multiple variants thereof.

The antigenic unit and the dimerization unit are preferably connected by a unit linker. The unit linker may comprise a restriction site in order to facilitate the construction of the polynucleotide. It is preferred that the unit linker is a GLGGL linker or a GLSGL (SEQ ID NO: 868) linker.

Examples of further sequences of linkers are disclosed in paragraphs [0098]-[0099] and in the recited sequences of WO 2020/176797A1, which is incorporated herein by reference and in paragraphs [0135] to [0139] of US 2019/0022202A1, which is incorporated herein by reference.

The term “targeting unit” as used herein refers to a unit that delivers the polypeptide/dimeric protein (encoded by the polynucleotide as) comprised in the vaccine with its antigenic unit to an antigen presenting cell.

Due to the targeting unit, the polypeptide/dimeric protein comprised in the vaccine of the invention attracts dendritic cells (DCs), neutrophils and other immune cells. Thus, the polypeptide/dimeric protein/vaccine comprising the targeting unit will not only target the antigenic unit comprised therein to specific cells, but in addition facilitates a response-amplifying effect (adjuvant effect) by recruiting specific immune cells to the administration site of the vaccine. This unique mechanism is of great importance in a clinical setting where patients can receive the vaccine of the invention without any additional adjuvants since the vaccine itself provides the adjuvant effect.

The targeting unit is connected through the dimerization unit to the antigenic unit, wherein the latter is in either the COOH-terminal or the NH₂-terminal end of the polypeptide/dimeric protein. It is preferred that the antigenic unit is in the COOH-terminal end of the polypeptide/dimeric protein.

The targeting unit is designed to target the polypeptide/dimeric protein/vaccine of the invention to surface molecules expressed on the APCs, such as molecules expressed exclusively on subsets of DCs.

Examples of such surface molecules on APCs are HLA, cluster of differentiation 14 (CD14), cluster of differentiation 40 (CD40), chemokine receptors and Toll-like receptors (TLRs). Chemokine receptors include C—C motif chemokine receptor 1 (CCR1), C—C motif chemokine receptor 3 (CCR3) and C—C motif chemokine receptor 5 (CCR5) and XCR1. Toll-like receptors include TLR-2, TLR-4 and TLR-5.

The targeting unit is or comprises a moiety that interacts with surface molecules. Thus, the targeting unit comprises or consists of an antibody-binding region with specificity for HLA, CD14, CD40, or Toll-like receptors. In another embodiment, the targeting unit comprises or consists of a synthetic or natural ligand. Examples include soluble CD40 ligand, natural ligands like chemokines, e.g. chemokine ligand 5, also called C—C motif ligand 5 (CCL5 or RANTES), macrophage inflammatory protein alpha (CCL3 or MIP-1α), chemokine motif ligand 1 or 2 (XCL1 or XCL2) and bacterial antigens like for example flagellin.

In one aspect of the invention the targeting unit comprises antibody binding regions with specificity for surface receptors on antigen presenting cells, such as CD14, CD40, Toll-like receptors such as TLR-2, TLR-4 and/or TLR-5, chemokine receptors such as CCR1, CCR3, CCR5 or MHC class I and II proteins.

In another embodiment, the targeting unit has affinity for a surface molecule selected from the group consisting of CD40, TLR-2, TLR-4 and TLR-5. Thus, in one embodiment the targeting unit comprises or consist of the antibody variable domains (VL and VH) with specificity for anti-CD40, anti-TLR-2, anti-TLR-4 or anti-TLR-5. In yet another embodiment, targeting unit comprises or consists of flagellin, which has affinity for TLR-5.

In one embodiment, the targeting unit has affinity for an MHC class II protein. Thus, in one embodiment, the targeting unit comprises or consists of the antibody variable domains (VL and VH) with specificity for MHC class II proteins selected from the group consisting of anti-HLA-DP, anti-HLA-DR and anti-pan HLA class II.

In a preferred embodiment of the invention, the targeting unit has affinity for a chemokine receptor selected from CCR1, CCR3 and CCR5, preferably for a chemokine receptor selected from CCR1 and CCR5. In another preferred embodiment of the invention, the targeting unit has an affinity for MHC class II proteins, preferably MHC class II proteins, selected from the group consisting of anti-HLA-DP, anti-HLA-DR and anti-pan HLA class II. More specifically in one embodiment the targeting unit comprises anti-pan HLA class II and MIP-1α.

In one embodiment the binding of the targeting unit to its cognate receptors leads to internalization of the polypeptide/dimeric protein/vaccine into the APC and degradation thereof into small peptides that are loaded onto MHC molecules and presented to CD4+ and CD8+ T cells to induce specific immune responses. Peptides loaded onto MHC II molecules can be recognized by antigen-specific CD4+ T helper cells, whereas peptides loaded on MHC I molecules can be recognized by antigen-specific CD8+ T cells, leading to proliferation and activation of cytotoxic function. Presentation of internalized antigens on MHC I molecules is a process termed cross-presentation. Once stimulated, and with help from activated CD4+ T cells, CD8+ T cells will target and kill cells expressing the same antigens.

In one aspect of the invention, the targeting unit comprises or is MIP-1α, preferably human MIP-1α (h MIP-1α, also called LD78β). Not only does MIP-1α attract APC to the vaccine through its chemotactic ability, it also causes internalisation of the polypeptide/dimeric protein vaccine construct through both the classical and cross-presentation pathway, whereby the epitopes are processed by enzymes and presented on the cell surface to raise the T cell response, particularly Th1 CD4+ responses and CD8+ T cell responses. MIP-1α is also capable of supporting the induction of antibody responses, in particular IgG2a, which is important for protection against betacoronavirus infection.

In one embodiment of the present invention, the targeting unit comprises an amino acid sequence having at least 80% sequence identity to the amino acid sequence 24-93 of SEQ ID NO: 234. In a preferred embodiment, the targeting unit comprises an amino acid sequence having at least 85% sequence identity to the amino acid sequence 24-93 of SEQ ID NO: 234, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% sequence identity. In yet another preferred embodiment, the targeting unit comprises the amino acid sequence 24-93 of SEQ ID NO: 234.

In a more preferred embodiment the targeting unit consists of an amino acid sequence having at least 80% sequence identity to the amino acid sequence 24-93 of SEQ ID NO:1, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as at least 100% sequence identity to the amino acid sequence 24-93 of SEQ ID NO: 234.

In one embodiment the targeting unit comprises or is anti-pan HLA class II. This targeting unit induces rapid and strong antibody responses with mixed IgG1 and IgG2a antibodies. Moreover, this targeting unit induces a significant cellular response (CD4+ and CD8+ type T cells).

One aspect of the invention relates to a vaccine that comprises an immunologically effective amount of:

(i) a polynucleotide comprising a nucleotide sequence encoding a targeting unit comprising anti-pan HLA class II, a dimerization unit and an antigenic unit, wherein the antigenic unit comprises a full-length viral surface protein of a betacoronavirus or a part thereof, preferably a protein selected from the group consisting of envelope protein, spike protein, membrane protein and hemagglutinin esterase; or

(ii) a polypeptide encoded by the polynucleotide as defined in (i), or

(iii) a dimeric protein consisting of two polypeptides encoded by the polynucleotide as defined in (i); and

a pharmaceutically acceptable carrier.

In one embodiment, the antigenic unit of the above-mentioned vaccine comprises at least a B cell epitope comprised in a full-length viral surface protein of a betacoronavirus, e.g. comprised in any of the aforementioned proteins and preferably comprises several B cell epitopes comprised in a full-length viral surface protein of a betacoronavirus, e.g. comprised in any of the aforementioned proteins.

One other aspect of the invention relates to a vaccine that comprises an immunologically effective amount of:

(i) a polynucleotide comprising a nucleotide sequence encoding a targeting unit comprising anti-pan HLA class II, a dimerization unit and an antigenic unit, wherein the antigenic unit comprises the full-length spike protein of a betacoronavirus or a part thereof or at least a B cell epitope comprised in the spike protein or part thereof; or

(ii) a polypeptide encoded by the polynucleotide as defined in (i), or

(iii) a dimeric protein consisting of two polypeptides encoded by the polynucleotide as defined in (i); and

a pharmaceutically acceptable carrier.

One further aspect of the invention relates to a vaccine that comprises an immunologically effective amount of:

(i) a polynucleotide comprising a nucleotide sequence encoding a targeting unit comprising hMIP-1α, a dimerization unit and an antigenic unit, wherein the antigenic unit comprises the full-length spike protein of a betacoronavirus or a part thereof or at least a B cell epitope comprised in the spike protein or part thereof; or

(ii) a polypeptide encoded by the polynucleotide as defined in (i), or

(iii) a dimeric protein consisting of two polypeptides encoded by the polynucleotide as defined in (i); and

a pharmaceutically acceptable carrier.

In one embodiment, the antigenic unit of the above-mentioned vaccine comprises the receptor binding domain of the spike protein or a part thereof or at least a B cell epitope comprised therein. In another embodiment, the antigenic unit of the above-mentioned vaccine comprises the HR1 domain or HR2 domain of the spike protein or a part thereof or at least a B cell epitope comprised therein. In yet another embodiment, the antigenic unit of the above-mentioned vaccine comprises the HR2 domain of the spike protein or a part thereof or at least a B cell epitope comprised therein.

Once administered, this vaccine elicits a strong humoral response and potentially also a cellular response.

Another aspect of the invention relates to a vaccine that comprises an immunologically effective amount of:

(i) a polynucleotide comprising a nucleotide sequence encoding a targeting unit comprising hMIP-1α, a dimerization unit and an antigenic unit, wherein the antigenic unit comprises at least one betacoronavirus T cell epitope, preferably several T cell epitopes that are conserved among betacoronaviruses; or

(ii) a polypeptide encoded by the polynucleotide as defined in (i), or

(iii) a dimeric protein consisting of two polypeptides encoded by the polynucleotide as defined in (i); and

a pharmaceutically acceptable carrier.

This vaccine, once administered, elicits a T cell response, i.e. a strong cellular response, which is particularly important in a therapeutic setting, as the CD8+ T cells can kill virus-infected cells and thus eliminate the virus. If the vaccine comprises T cell epitopes that are conserved among betacoronaviruses, it could provide protection against multiple variants betacoronaviruses, e.g. multiple variants of SARS-CoV viruses, which is important for potential efficacy also against future betacoronavirus variants wherein mutations occur in the non-conserved regions.

A particular aspect relates to a vaccine that comprises an immunologically effective amount of:

(i) a polynucleotide comprising a nucleotide sequence encoding a targeting unit, a dimerization unit and an antigenic unit, wherein the antigenic unit comprises a) the full-length spike protein or of a betacoronavirus or a part thereof or at least one B cell epitope comprised in the spike protein or part thereof and b) at least one betacoronavirus T cell epitope; or

(ii) a polypeptide encoded by the polynucleotide as defined in (i), or

(iii) a dimeric protein consisting of two polypeptides encoded by the polynucleotide as defined in (i); and

a pharmaceutically acceptable carrier.

In one embodiment, the antigenic unit of the above-mentioned vaccine comprises the receptor binding domain of the spike protein or a part thereof or at least a B cell epitope comprised therein. In another embodiment, the antigenic unit of the above-mentioned vaccine comprises the HR1 domain or HR2 domain of the spike protein or a part thereof or at least a B cell epitope comprised therein. In yet another embodiment, the antigenic unit of the above-mentioned vaccine comprises the HR2 domain of the spike protein or a part thereof or at least a B cell epitope comprised therein.

Such a vaccine will, once administered, elicit a T cell response and a B cell response. In a pandemic or an epidemic situation, it is not time efficient to first diagnose an individual to determine if he or she needs primarily a B or T cell response, neither whether prophylactic or therapeutic treatment is the highest medical need. Less so, as the determination of whether or not an individual is infected can be difficult due to lack of (sufficient) applicable tests. Thus, being able to protect and cure at the same time is important. By combining the full-length or part of the spike protein or several B cell epitopes present in the spike protein and the conserved T cell epitopes, both a strong humoral and cellular response is elicited once the vaccine is administered. The response can be more humoral or more cellular, depending on the selected targeting unit.

The vaccine above preferably comprises a targeting unit comprising MIP-1α or anti-pan HLA class II.

Thus, one aspect of the present invention relates to a vaccine that comprises an immunologically effective amount of:

(i) a polynucleotide comprising a nucleotide sequence encoding a targeting unit comprising MIP-1α, a dimerization unit and an antigenic unit, wherein the antigenic unit comprises a) the full-length spike protein of a betacoronavirus or a part thereof or at least one B cell epitope comprised in the spike protein or part thereof and b) at least one betacoronavirus T cell epitope; or

(ii) a polypeptide encoded by the polynucleotide as defined in (i), or

(iii) a dimeric protein consisting of two polypeptides encoded by the polynucleotide as defined in (i); and

a pharmaceutically acceptable carrier.

In one embodiment, the antigenic unit of the above-mentioned vaccine comprises the receptor binding domain of the spike protein or a part thereof or at least a B cell epitope comprised therein. In another embodiment, the antigenic unit of the above-mentioned vaccine comprises the HR1 domain or HR2 domain of the spike protein or a part thereof or at least a B cell epitope comprised therein. In yet another embodiment, the antigenic unit of the above-mentioned vaccine comprises the HR2 domain of the spike protein or a part thereof or at least a B cell epitope comprised therein.

In another embodiment the targeting unit comprises anti-pan HLA class II. This targeting unit induce rapid and strong antibody responses with mixed IgG1 and IgG2a antibodies. Moreover, this targeting unit induces a significant cellular response (CD4+ and CD8+ type T cells).

Thus, one embodiment of the present invention discloses a vaccine that comprises an immunologically effective amount of:

(i) a polynucleotide comprising a nucleotide sequence encoding a targeting unit comprising anti-pan HLA class II, a dimerization unit and an antigenic unit, wherein the antigenic unit comprises a) the full-length spike protein of a betacoronavirus or a part thereof or at least one B cell epitope comprised in the spike protein or part thereof and b) at least one betacoronavirus T cell epitope; or

(ii) a polypeptide encoded by the polynucleotide as defined in (i), or

(iii) a dimeric protein consisting of two polypeptides encoded by the polynucleotide as defined in (i); and

a pharmaceutically acceptable carrier.

In one embodiment, the antigenic unit of the above-mentioned vaccine comprises the receptor binding domain of the spike protein or a part thereof or at least a B cell epitope comprised therein. In another embodiment, the antigenic unit of the above-mentioned vaccine comprises the HR1 domain or HR2 domain of the spike protein or a part thereof or at least a B cell epitope comprised therein. In yet another embodiment, the antigenic unit of the above-mentioned vaccine comprises the HR2 domain of the spike protein or a part thereof or at least a B cell epitope comprised therein.

In further embodiments of the present invention, the antigenic unit comprises sets of 10, 14, 20, 24 and 30 T cell epitopes and the RBD and linkers between the epitopes. In one embodiment the antigenic unit comprises 10 T cell epitopes and the RBD and 10 T cell epitopes. In another embodiment the antigenic unit comprises the RBD and 20 epitopes. In a further embodiment the antigenic unit comprises 20 T cell epitopes and RBD with no linkers. In another embodiment the antigenic unit comprises 20 T cell epitopes.

In further embodiments of the present invention, the antigenic unit comprises sets of 10, 14, 20, 24 and 30 T cell epitopes and the HR1 domain or HR2 domain, preferably the HR2 domain, and linkers between the epitopes. In one embodiment the antigenic unit comprises 10 T cell epitopes and the HR1 domain or HR2 domain, preferably the HR2 domain and 10 T cell epitopes. In another embodiment the antigenic unit comprises the HR1 domain or HR2 domain, preferably the HR2 domain and 20 epitopes. In a further embodiment the antigenic unit comprises 20 T cell epitopes and HR1 domain or HR2 domain, preferably the HR2 domain with no linkers.

In yet another embodiment, the antigenic unit comprises 1, 2, 3, 4, 5, 6, 7, 8, 9 or 10 T cell epitopes and the full-length of the spike protein or a part thereof, preferably the RBD or a part thereof. In yet another embodiment, the 2-10 T cell epitopes are separated from each other by linkers and the full-length of the spike protein or a part thereof, preferably the RBD or a part thereof, is separated from the final T cell epitope by a linker. In yet another embodiment, the antigenic unit comprises 1-3 T cell epitopes and the full-length of the spike protein or a part thereof, preferably the RBD or a part thereof. In yet another embodiment, the 2 or 3 T cell epitopes are separated from each other by linkers and the full-length of the spike protein or a part thereof, preferably the RBD or a part thereof, is separated from the one or the final T cell epitope by a linker.

In one embodiment, the antigenic unit comprises an amino acid sequence having at least 70% sequence identity to the amino acid sequence of SEQ ID NO: 265 or SEQ ID NO: 267 or SEQ ID NO: 269 or SEQ ID NO: 271 or SEQ ID NO: 273 or SEQ ID NO: 275 or SEQ ID NO: 277 or SEQ ID NO: 279 or SEQ ID NO: 281 or SEQ ID NO: 283 or SEQ ID NO: 285 or SEQ ID NO: 287 or SEQ ID NO: 289 or SEQ ID NO: 291 or SEQ ID NO: 293, such as at least 75%, such as at least 77%, such as at least 80%, such as at least 85%, such as at least 90%, such as at least 91%, such as at least 92%, such as at least 93%, such as at least 94%, such as at least 95%, such as at least 96%, such as at least 97%, such as at least 98% or such as at least 99% sequence identity.

In one embodiment, the antigenic unit comprises the amino acid sequence of SEQ ID NO: 265 or SEQ ID NO: 267 or SEQ ID NO: 269 or SEQ ID NO: 271 or SEQ ID NO: 273 or SEQ ID NO: 275 or SEQ ID NO: 277 or SEQ ID NO: 279 or SEQ ID NO: 281 or SEQ ID NO: 283 or SEQ ID NO: 285 or SEQ ID NO: 287 or SEQ ID NO: 289 or SEQ ID NO: 291 or SEQ ID NO: 293.

In a preferred embodiment the 10, 14, 20, 24 and 30 T cell epitopes are selected from the group consisting of: SEQ ID NO: 67, SEQ ID NO: 19, SEQ ID NO: 78, SEQ ID NO: 57, SEQ ID NO: 50, SEQ ID NO: 55, SEQ ID NO: 64, SEQ ID NO: 22, SEQ ID NO: 87, SEQ ID NO: 62, SEQ ID NO: 39, SEQ ID NO: 59, SEQ ID NO: 26, SEQ ID NO: 53, SEQ ID NO: 32, SEQ ID NO: 38, SEQ ID NO: 30, SEQ ID NO: 40, SEQ ID NO: 42, SEQ ID NO: 35, SEQ ID NO: 71, SEQ ID NO: 9, SEQ ID NO: 21, SEQ ID NO: 85, SEQ ID NO: 75, SEQ ID NO: 23, SEQ ID NO: 34, SEQ ID NO: 36, SEQ ID NO: 77 and SEQ ID NO: 20.

In a another preferred embodiment of the present invention, the antigenic unit is selected from the group consisting of: SEQ ID NO: 237, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248. SEQ ID NO: 249, SEQ ID NO: 250 and SEQ ID NO: 251.

The vaccine of the invention comprises a dimerization unit. The term “dimerization unit” as used herein, refers to a sequence of nucleotides or amino acids between the antigenic unit and the targeting unit. Thus, the dimerization unit serves to connect the antigenic unit and the targeting unit and facilitates dimerization of two monomeric polypeptides into a dimeric protein. Furthermore, the dimerization unit also provides the flexibility in the polypeptide/dimeric protein to allow optimal binding of the targeting unit to the surface molecules on the APCs, even if they are located at variable distances. The dimerization unit may be any unit that fulfils these requirements.

Accordingly, in one embodiment dimerization unit comprises a hinge region. In another embodiment, the dimerization unit comprises a hinge region and another domain that facilitates dimerization. In one embodiment, the hinge region and the other domain are connected through a linker, i.e. a dimerization unit linker. In yet another embodiment, the dimerization unit comprises a hinge region, a dimerization unit linker and another domain that facilitates dimerization, wherein the dimerization unit linker is located between the hinge region and the other domain that facilitates dimerization.

The term “hinge region” refers to an amino acid sequence comprised in the dimeric protein that contributes to joining two polypeptides, i.e. contributes to the formation of a dimeric protein. Moreover, the hinge region functions as a flexible spacer between the polypeptides, allowing the two targeting units of the dimeric protein to bind simultaneously to two surface molecules on APCs, even if they are expressed with variable distances. The hinge region may be Ig derived, such as derived from IgG3. The hinge region may contribute to the dimerization through the formation of covalent bond(s), e.g. disulfide bridge(s) between cysteines. Thus, in one embodiment the hinge region has the ability to form one or more covalent bonds. Preferably, the covalent bond is a disulfide bridge.

In one embodiment, the dimerization unit comprises a hinge exon h1 and hinge exon h4 (human hinge region 1 and human hinge region 4) having an amino acid sequence having at least 80% sequence identity to the amino acid sequence 94-120 of SEQ ID NO: 233.

In a preferred embodiment, the dimerization unit comprises a hinge exon h1 and hinge exon h4 with an amino acid sequence having at least 85% sequence identity to the amino acid sequence 94-120 of SEQ ID NO: 233, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99% sequence identity.

In a preferred embodiment, the dimerization unit comprises a hinge exon h1 and hinge exon h4 with the amino acid sequence 94-120 of SEQ ID NO: 233.

In one embodiment, the dimerization unit comprises another domain that facilitates dimerization, said other domain is an immunoglobulin domain, such as an immunoglobulin constant domain (C domain), such as a carboxyterminal C domain (i.e. a CH3 domain), a CH1 domain or a CH2 domain, or a sequence that is substantially identical to the C domain or a variant thereof. Preferably, the other domain that facilitates dimerization is a carboxyterminal C domain derived from IgG. More preferably, the other domain that facilitates dimerization is a carboxyterminal C domain derived from IgG3.

The immunoglobulin domain contributes to dimerization through non-covalent interactions, e.g. hydrophobic interactions. For example, the immunoglobulin domain has the ability to form dimers via noncovalent interactions. Thus, in one embodiment, the immunoglobulin domain has the ability to form dimers via noncovalent interactions. Preferably, the noncovalent interactions are hydrophobic interactions.

It is preferred that if the dimerization unit comprises a CH3 domain, it does not comprise a CH2 domain. Further, it is preferred that if the dimerization unit comprises a CH2 domain, it does not comprise a CH3 domain.

In one embodiment, the dimerization unit comprises a carboxyterminal C domain derived from IgG3 with an amino acid sequence having at least 80% sequence identity to the amino acid sequence 131-237 of SEQ ID NO: 233.

In a preferred embodiment, the dimerization unit comprises a carboxyterminal C domain derived from IgG3 with an amino acid sequence having at least 85% sequence identity to the amino acid sequence 131-237 of SEQ ID NO: 233, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99% sequence identity.

In a preferred embodiment, the dimerization unit comprises a carboxyterminal C domain derived from IgG3 with the amino acid sequence 131-237 of SEQ ID NO: 233.

In a preferred embodiment, the dimerization unit comprises a hinge exon h1, a hinge exon h4, a dimerization unit linker and a CH3 domain of human IgG3. In a further preferred embodiment, the dimerization unit comprises a polypeptide consisting of hinge exon h1, hinge exon h4, a dimerization unit linker and a CH3 domain of human IgG3.

In another preferred embodiment, the dimerization unit consists of hinge exon h1 and hinge exon h4 connected through a dimerization unit linker to a CH3 domain of human IgG3.

In one embodiment of the present invention, the dimerization unit comprises an amino acid sequence having at least 80% sequence identity to the amino acid sequence 94-237 of SEQ ID NO: 233. In a preferred embodiment, the dimerization unit comprises an amino acid sequence having at least 85% sequence identity to the amino acid sequence 94-237 of SEQ ID NO: 233, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% sequence identity.

In one embodiment of the present invention, the dimerization unit comprises the amino acid sequence 94-237 of SEQ ID NO: 233

In a more preferred embodiment the dimerization unit consists of an amino acid sequence having at least 80% sequence identity to the amino acid sequence 94-237 of SEQ ID NO: 233, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity to the amino acid sequence 94-237 of SEQ ID NO: 233. In an even more preferred embodiment, the dimerization unit consists of the amino acid sequence 94-237 of SEQ ID NO: 233.

In one embodiment the dimerization unit linker, i.e. linker connecting the hinge region to the other domain, is present in the dimerization unit. In another embodiment, the linker is present and is a glycine-serine rich linker, preferably G3S2G3SG linker (GGGSSGGGSG).

The dimerization unit has any orientation with respect to antigenic unit and targeting unit. In one embodiment, the antigenic unit is in the COOH-terminal end of the dimerization unit with the targeting unit in the N-terminal end of the dimerization unit. As such, the antigenic unit is connected to the C-terminal end of the dimerization unit (e.g. via a unit linker) with the targeting unit being connected to the N-terminal end of the dimerization unit. In another embodiment, the antigenic unit is in the N-terminal end of the dimerization unit with the targeting unit in the COOH-terminal end of the dimerization unit. As such, the antigenic unit is connected to the N-terminal end of the dimerization unit (e.g. via a unit linker) with the targeting unit being connected to the C-terminal end of the dimerization unit. It is preferred that the antigenic unit is in the COOH end of the dimerization unit, i.e. the antigenic unit is connected to the C-terminal end of the dimerization unit, preferably via the unit linker, and the targeting unit is connected to the N-terminal end of the dimerization unit.

In a preferred embodiment, the antigenic unit is connected to the dimerization unit by a unit linker. Thus, in one embodiment, the polynucleotide/polypeptide/dimeric protein comprises a nucleotide sequence encoding a unit linker or an amino acid sequence being the unit liker that connects the antigenic unit to the dimerization unit.

The unit linker may comprise a restriction site in order to facilitate the construction of the polynucleotide. In a preferred embodiment, unit linker is GLGGL or GLSGL.

In a preferred embodiment, the vaccine of the invention comprises a polynucleotide of which further comprises a nucleotide sequence encoding a signal peptide. The signal peptide is either located at the N-terminal end of the targeting unit or the C-terminal end of the targeting unit, depending on the orientation of the targeting unit in the polypeptide. The signal peptide is designed and constructed to allow secretion of the polypeptide encoded by the polynucleotide in the cells transfected with said polynucleotide.

Any suitable signal peptide may be used. Examples of suitable peptides are a human Ig VH signal peptide, such as a signal peptide comprising an amino acid sequence having at least 80% sequence identity to the amino acid sequence of SEQ ID NO: 235, a human TPA signal peptide, such as SEQ ID NO: 236 and a signal peptide comprising an amino acid sequence having at least 80% sequence identity to the amino acid sequence 1-23 of SEQ ID NO: 234, i.e. a human MIP1-α signal peptide.

In a preferred embodiment, the polynucleotide comprises a targeting unit which is hMIP1-α and a nucleic acid sequence which encodes for a human MIP1-α signal peptide.

In another preferred embodiment, the polynucleotide comprises a targeting unit which is human anti-pan HLA class II and a nucleic acid sequence which encodes for an Ig VH signal peptide.

In a preferred embodiment, the signal peptide comprises an amino acid sequence having at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity to the amino acid sequence of SEQ ID NO: 235.

In a more preferred embodiment, the signal peptide consists of an amino acid sequence having at least 80%, preferably at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity to the amino acid sequence of SEQ ID NO: 235.

In a preferred embodiment, the signal peptide comprises an amino acid sequence having at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity to the amino acid sequence 1-23 of SEQ ID NO: 234.

In a more preferred embodiment, the signal peptide consists of an amino acid sequence having at least 80%, preferably at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity to the amino acid sequence 1-23 of SEQ ID NO: 234.

Sequence identity may be determined as follows: A high level of sequence identity indicates likelihood that a second sequence is derived from a first sequence. Amino acid sequence identity requires identical amino acid sequences between two aligned sequences. Thus, a candidate sequence sharing 70% amino acid identity with a reference sequence requires that, following alignment, 70% of the amino acids in the candidate sequence are identical to the corresponding amino acids in the reference sequence. Identity may be determined by aid of computer analysis, such as, without limitations, the ClustalW computer alignment program (Higgins D., Thompson J., Gibson T., Thompson J. D., Higgins D. G., Gibson T. J., 1994. CLUSTAL W: improving the sensitivity of progressive multiple sequence alignment through sequence weighting, position-specific gap penalties and weight matrix choice. Nucleic Acids Res. 22:4673-4680), and the default parameters suggested therein. Using this program with its default settings, the mature (bioactive) part of a query and a reference polypeptide are aligned. The number of fully conserved residues is counted and divided by the length of the reference polypeptide. In doing so, any tags or fusion protein sequences, which form part of the query sequence, are disregarded in the alignment and subsequent determination of sequence identity.

The ClustalW algorithm may similarly be used to align nucleotide sequences. Sequence identities may be calculated in a similar way as indicated for amino acid sequences.

Another preferred mathematical algorithm utilized for the comparison of sequences is the algorithm of Myers and Miller, CABIOS (1989). Such an algorithm is incorporated into the ALIGN program (version 2.0) which is part of the FASTA sequence alignment software package (Pearson W R, Methods Mol Biol, 2000, 132:185-219). Align calculates sequence identities based on a global alignment. Align0 does not penalize to gaps in the end of the sequences. When utilizing the ALIGN and Align0 program for comparing amino acid sequences, a BLOSUM50 substitution matrix with gap opening/extension penalties of −12/−2 is preferably used.

Another preferred mathematical algorithm utilized for the comparison of sequences is the implementation of local pairwise alignment algorithm of BioPython called “Smith-Waterman algorithm”.

One aspect of the invention relates to a polypeptide with the amino acid sequence of SEQ ID NO: 253, construct VB2049, or a polynucleotide encoding same, which comprises a humanMIP-1α targeting unit and an antigenic unit comprising a short form of the SARS-CoV-2 RBD (“RBD short”, amino acids 331-524, i.e. 193 amino acids). This construct is capable of raising anti-RBD IgG antibodies with neutralizing effects. It is also capable of inducing strong T cell responses against epitopes comprised in the RBD.

One aspect of the invention relates to a polypeptide with the amino acid sequence of SEQ ID NO: 255, construct VB2060, or a polynucleotide encoding same, which comprises a human MIP-1α as targeting unit and an antigenic unit comprising a longer version of the SARS-CoV-2 RBD (“RBD long”, amino acids 319-542 i.e. 223 amino acids). It is capable of raising neutralizing anti-RBD IgG antibodies, which are even found in the lungs. This construct is capable of inducing strong T cell responses against RBD within 7 days after vaccination that are long lasting.

One aspect of the invention relates to a polypeptide with the amino acid sequence of SEQ ID NO: 257, construct VB2065, or a polynucleotide encoding same, which comprises a human MIP-1α targeting unit and an antigenic unit comprising the full-length spike protein from SARS-CoV2 strain Wuhan Hu-1. It is capable of raising neutralizing anti-RBD IgG antibodies. The construct is capable of inducing broad and strong T cells responses.

One aspect of the invention relates to a polypeptide with the amino acid sequence of SEQ ID NO: 259, construct VB2048, or a polynucleotide encoding same, comprising a human MIP-1α targeting unit and an antigenic unit comprising 20 immunogenic T cell epitopes (see Table 1) from multiple SARS-CoV2 strains. It is capable of inducing strong T cell responses even when co-administered with other constructs, e.g. VB2049.

One aspect of the invention relates to a polypeptide or a polynucleotide encoding same, comprising a human anti-pan HLA class II targeting unit and an antigenic unit comprising the longer version of the SARS-CoV-2 RBD (“RBD long” amino acids 319-542 i.e. 223 amino acids). The corresponding mouse construct, construct VB2059, comprising an anti-mouse MHCII scFv as targeting unit is capable of raising antibodies against RBD and of inducing a T cell response against RBD.

One aspect of the invention relates to a polypeptide or a polynucleotide encoding same, comprising a human anti-pan HLA class II targeting unit and an antigenic unit comprising the full-length spike protein from SARS-CoV2 strain Wuhan Hu-1. The corresponding mouse construct, construct VB2071, comprising an anti-mouse MHCII scFv as targeting unit is able to induce anti-RBD IgG antibodies and induces broad and strong T cell responses.

One aspect of the invention relates to a polypeptide with the amino acid sequence of SEQ ID NO: 265, construct VB2081, or a polynucleotide encoding same, comprising a humanMIP-1α targeting unit and an antigenic unit comprising one predicted T cell epitope (pep08) and a longer version of the SARS-CoV-2 RBD linked to the T cell epitope with a (GGGGS)₂ linker. This construct raises IgG antibodies against RBD and induces T cell responses against RBD, to the included one T cell epitope.

One aspect of the invention relates to a polypeptide with the amino acid sequence of SEQ ID NO: 267, construct VB2082, or a polynucleotide encoding same, which comprises a human MIP-1α targeting unit and an antigenic unit comprising one predicted T cell epitope (pep18) and a longer version of the SARS-CoV-2 RBD linked to the T cell epitope with a (GGGGS)₂ linker. This construct is able to raise IgG responses against RBD, and to induce T cell response against the included one T cell epitope.

One aspect of the invention relates to a polypeptide with the amino acid sequence of SEQ ID NO: 271, construct VB2084, or a polynucleotide encoding same, which comprises a human MIP-1α targeting unit. It has an antigenic unit comprising three predicted T cell epitopes (pep08, pep18, pep25) and the longer version of the SARS-CoV-2 RBD all linked with a (GGGGS)₂ linker. This construct is capable of inducing T cell response against epitopes in the RBD, as well as against the included three T cell epitopes.

One aspect of the invention relates to a polypeptide with the amino acid sequence of SEQ ID NO: 293, construct VB2097, or a polynucleotide encoding same, which comprises a human MIP-1α targeting unit. The antigenic unit comprises three predicted T cell epitopes (pep08, pep18 and pep25) separated from each other with a (GGGGS)₂ linker and the “RBD long”, which is separated from the T cell epitope by a GSAT linker. Not only did the construct raise IgG antibodies against RBD; it also showed a remarkable strong T cell responses against the RBD and the included T cell epitopes.

One aspect of the invention relates to a polypeptide with the amino acid sequence of SEQ ID NO: 297, construct VB2099, or a polynucleotide encoding same, which comprises a human MIP-1α targeting unit. The antigenic unit comprises 3 predicted T cell epitopes (pep08, pep18 and pep25), separated from each other with a (GGGGS)₂ linker and a longer version of the SARS-CoV-2 RBD (“RBD long”, 223 amino acids) which is connected to the T cell epitope by a SEG linker. It is capable of raising IgG antibodies against RBD. In addition, it is capable of inducing T cell responses against RBD and against the included T cell epitopes.

One aspect of the invention relates to a polypeptide with the amino acid sequence of SEQ ID NO: 295, construct VB2129, or a polypeptide encoding same, which comprises a human MIP-1α targeting unit and an antigenic unit comprising the South African RBD (with 3 mutations characterised in the South African variant B.1.351) in. It is capable of raising IgG responses against RBD and of inducing T cell responses.

In one embodiment of the present invention the targeting unit, dimerization unit and antigenic unit in said polypeptide or dimeric protein are in the N-terminal to C-terminal order of targeting unit, dimerization unit and antigenic unit.

The vaccine of the invention comprises a pharmaceutically acceptable carrier, including but not limited to saline, buffered saline, such as PBS, dextrose, water, glycerol, ethanol, sterile isotonic aqueous buffers, and combinations thereof.

The vaccine may further comprise an adjuvant. Particularly for vaccines comprising polypeptides/proteins, pharmaceutically acceptable adjuvants include, but are not limited to poly-ICLC, 1018 ISS, aluminum salts, Amplivax, AS 15, BCG, CP-870,893, CpG7909, CyaA, dSLIM, GM-CSF, IC30, IC31, Imiquimod, ImuFact EV1 P321, IS Patch, ISS, ISCOMATRIX, JuvImmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PLGA microparticles, resiquimod, SRL172, virosomes and other virus-like particles, YF-17D, VEGF trap, R848, beta-glucan, Pam3Cys, Aquila's QS21 stimulon, vadimezan, and/or AsA404 (DMXAA).

For vaccines comprising polynucleotides, the vaccines may comprise molecules that ease transfection of cells and/or adjuvants in the form of plasmids comprising nucleotide sequences encoding chemokines or cytokines in order to enhance the immune response.

The vaccine may be formulated into any way suitable administration to a subject, e.g. human individual, e.g. such as a liquid formulation for injection, e.g. for intradermal or intramuscular injection.

The vaccine of the invention may be administered in any way suitable for administration to a subject, e.g. human individual, of either a polypeptide/protein vaccine or a polynucleotide vaccine, such as administered by intradermal, intramuscular, intranodal or subcutaneous injection, or by mucosal or epithelial application, such as intranasal, oral, enteral or intravesicular (to the bladder) administration.

In a preferred embodiment, the vaccine comprises a polynucleotide, and is administered by intramuscular or intradermal injection.

The vaccine may comprise one polynucleotide, e.g. in the form of a DNA plasmid or may comprise more than one polynucleotide, e.g. in the form of more than one DNA plasmids. In one embodiment, the vaccine comprises 2 DNA plasmids, one comprising a polynucleotide comprising a nucleotide encoding for an antigenic unit that comprises a full-length surface protein of betacoronavirus or a part thereof, e.g. the RBD and the other comprising a polynucleotide comprising a nucleotide encoding for an antigenic unit that comprises T cell epitopes, preferably conservative T cell epitopes. Due to the “T cell epitope plasmid”, the vaccine will provide protection against several species/strains of betacoronaviruses, e.g. against several strains of SARS-CoV, e.g. against SARS-CoV and SARS-CoV-2. Such a vaccine will also provide protection against multiple variants of a betacoronavirus, e.g. variants of the SARS-CoV virus or variants of the SARS-CoV-2 virus, which is important for the efficacy of such a vaccine against future mutated viruses.

In one embodiment, when the virus mutates, the plasmid comprising a polynucleotide comprising a nucleotide encoding for an antigenic unit that comprises a full-length surface protein of betacoronavirus or a part thereof may be engineered such that it comprise the mutations, while the plasmid comprising a polynucleotide comprising a nucleotide encoding for an antigenic unit that comprises T cell epitopes may be kept as is.

The vaccine of the invention comprises an immunologically effective amount of the polynucleotide/polypeptide or dimeric protein. The term “immunologically effective amount” means an amount inducing an immunoprotective response (for a prophylactic vaccine) or an immunotherapeutic response (for a therapeutic vaccine) in the individual vaccinated with such vaccine, wherein such response is induced by either a single vaccination or several vaccinations, e.g. an initial vaccination and one or several booster vaccinations, adequately spaced in time. Such amount may vary depending upon which specific polynucleotide/polypeptide/dimeric protein is employed. It may also vary depending on whether the vaccine is administered for prophylaxis or treatment, the severity of the disease in individuals infected with betacoronavirus, the age, weight, medical history and pre-existing conditions.

The immunologically effective amount may be an amount effective to reduce or to prevent the incidence of signs/symptoms, to reduce the severity of the incidence of signs/symptoms, to eliminate the incidence of signs/symptoms, to slow the development of the incidence of signs/symptoms, to prevent the development of the incidence of signs/symptoms, and/or effect prophylaxis of the incidence of signs/symptoms.

An immunologically effective amount for a prophylaxis may be an amount effective for prophylaxis of a disease caused by betacoronavirus or prevention of the reoccurrence of such a disease, is sufficient to effect such prophylaxis for the disease or reoccurrence. It may be an amount effective to prevent the incidence of signs and/or symptoms of a betacoronavirus infection.

An immunologically effective amount for a treatment may be an amount effective for arresting, or reducing the development of a disease caused by a betacoronavirus or its clinical symptoms, and/or alleviating or relieving the disease, causing regression of the disease or its clinical symptoms.

The vaccine of the invention typically comprises the polynucleotide in a range of from 0.1 to 10 mg, e.g. about 0.1, 0.2, 0.3, 0.4, 0.5, 0.6, 0.7, 0.8, 0.9 or 1 mg or e.g. 2, 3, 4, 5, 6, 7, 8, 9 or 10 mg. The vaccine of the invention typically comprises the polypeptide/dimeric protein in the range of from 5 μg to 5 mg.

The invention also relates to a polynucleotide as described above. The polynucleotide may comprise a DNA nucleotide sequence or an RNA nucleotide sequence, such as genomic DNA, cDNA, and RNA sequences, either double stranded or single stranded.

It is preferred that the polynucleotide is optimized to the species of the subject to which it is administered. For administration to a human, it is preferred that the polynucleotide sequence is human codon optimized.

In a preferred embodiment, the vaccine is a DNA vaccine, i.e. the polynucleotide is a DNA.

The invention further relates to a polypeptide encoded by the polynucleotide sequence as defined above. The polypeptide may be expressed in vitro for production of the vaccine according to the invention, or the polypeptide may be expressed in vivo as a result of administration of the polynucleotide to a subject, such as a human individual.

Due to the presence of the dimerization unit, dimeric proteins are formed when the polypeptide is expressed. The dimeric protein may be a homodimer, i.e. wherein the two polypeptide chains are identical and consequently comprise identical betacoronavirus epitopes, or the dimeric protein may be a heterodimer comprising two different monomeric polypeptides encoded in the antigenic units. The latter may be relevant if e.g. the number of betacoronavirus epitopes and thus the number of amino acids exceeds the upper limit for inclusion into the antigenic unit. It is however preferred that the dimeric protein is a homodimeric protein.

Furthermore, the invention relates to a vector comprising a polynucleotide sequence (e.g. in the form of a DNA) comprising a nucleotide sequence encoding a targeting unit, a dimerization unit and an antigenic unit, wherein the antigenic unit comprises at least one betacoronavirus epitope.

The vector is for transfecting a host cell and expression of a polypeptide/dimeric protein encoded by the polynucleotide described above, i.e. an expression vector, preferably a DNA plasmid.

It is preferred that the vector allows for easy exchange of the various units described above, particularly the antigenic unit. In one embodiment, the expression vector may be pUMVC4a vector or a vector comprising NTC9385R vector backbones. The antigenic unit may be exchanged with an antigenic unit cassette restricted by the SfiI restriction enzyme cassette where the 5′ site is incorporated in the GLGGL/GLSGL linker and the 3′ site is included after the stop codon in the vector.

The invention also relates to a host cell comprising a polynucleotide comprising a nucleotide sequence encoding a targeting unit, a dimerization unit and an antigenic unit, wherein the antigenic unit comprises at least one betacoronavirus epitope or comprising a vector comprising a polynucleotide sequence comprising a nucleotide sequence encoding a targeting unit, a dimerization unit and an antigenic unit, wherein the antigenic unit comprises at least one betacoronavirus epitope.

Suitable host cells include prokaryotes, yeast, insect or higher eukaryotic cells. In a preferred embodiment, the host cell is a human cell, preferably the cell of a human individual in need of the vaccine of the invention.

In one aspect, the invention relates to the use of the polynucleotide, the polypeptide or the dimeric protein described above as a medicament.

In a specific embodiment of the present invention the polynucleotide or the polypeptide or the dimeric protein are for use in the treatment of an infection with a betacoronavirus. In a preferred embodiment the betacoronavirus is SARS-CoV-2.

Suitable methods for preparing the vaccine according to the invention are disclosed in WO 2004/076489A1, WO 2011/161244A1, WO 2013/092875A1 and WO 2017/118695A1, which are incorporated herein by reference.

In one aspect, the invention relates to a method for preparing the vaccine comprising an immunologically effective amount of the dimeric protein, or the polypeptide as defined above by producing the polypeptides in vitro. The in vitro synthesis of the polypeptides and proteins may be carried out by any suitable method known to the person skilled in the art, such a through peptide synthesis or expression of the polypeptide in any of a variety of expressions systems followed by purification. Accordingly, in one embodiment the invention provides a method for preparing a vaccine comprising
(i) a dimeric protein consisting of two polypeptides encoded by a polynucleotide comprising a nucleotide sequence encoding a targeting unit, a dimerization unit and an antigenic unit, wherein the antigenic unit comprises at least one betacoronavirus epitope; or
(ii) a polypeptide encoded by a polynucleotide comprising a nucleotide sequence encoding a targeting unit, a dimerization unit and an antigenic unit, wherein the antigenic unit comprises at least one betacoronavirus epitope by producing the dimeric protein or polypeptide in vitro, the method comprises
   a) transfecting cells with the polynucleotide;
   b) culturing the cells;
   c) collecting and purifying the dimeric protein or the polypeptide expressed from the cells; and
   d) mixing the dimeric protein or polypeptide obtained from step c) with the pharmaceutically acceptable carrier.

In a preferred embodiment, the dimeric protein or polypeptide obtained from step c) is dissolved in said pharmaceutically acceptable carrier.

The pharmaceutically acceptable carrier is one of the aforementioned pharmaceutically acceptable carriers, e.g. an aqueous pharmaceutically acceptable carrier, such as water or a buffer. In one embodiment, the vaccine comprises further an adjuvant.

Purification may be carried out according to any suitable method, such as chromatography, centrifugation, or differential solubility.

In another aspect the invention relates to a method for preparing the vaccine according to the invention comprising an immunologically effective amount of the polynucleotide as defined above.

Thus, in one embodiment the invention provides a method for preparing a vaccine comprising an immunologically effective amount of a polynucleotide comprising a nucleotide sequence encoding a targeting unit, a dimerization unit and an antigenic unit, wherein the antigenic unit comprises at least one betacoronavirus epitope, the method comprises
a) preparing the polynucleotide;
b) optionally cloning the polynucleotide into an expression vector; and
c) mixing the polynucleotide obtained from step a) or the vector obtained from step b) with the pharmaceutically acceptable carrier.

The polynucleotide may be prepared by any suitable method known to the skilled person. For example, the polynucleotide may be prepared by chemical synthesis using an oligonucleotide synthesizer.

In particular, smaller nucleotide sequences, such as for example nucleotide sequences encoding the targeting unit, the dimerization unit and/or the subunits of the antigenic unit may be synthesized individually and then ligated to produce the final polynucleotide into the vector backbone.

### EXAMPLES

### Example 1: Selection of T Cell Epitopes

The predicted immunogenic epitopes from conserved regions of SARS CoV viruses were identified as follows:

In a first step, the worldwide population of HLA class I and II alleles were identified. For HLA class I, the allele frequency database available at http://www.allelefrequencies.net was used and the identification of the most frequent HLA alleles was conducted in the following manner: a separate search for each locus: A, B and C and a separate search for following regions: Europe, South-East Asia (focus on China) and North America (focus on US) was carried out. The population standard was set to “Gold” to obtain only the high-quality studies. The level of resolution was set to at least 4 digits, for instance: HLA-A*01:01. The sampling year was set to 2005 and later. The top 4 frequent alleles for each study was collected. Among all top 4 for all studies, the top 4-5 frequent alleles for each region (Europe/South-East Asia/North America) was selected. Due to overlap between the regions, the number of the final selected alleles was 10, 10 and 11 for A, B and C, respectively. These 31 HLA class I alleles cover 99.4% of the world population, as estimated by IEDB population coverage estimation tool (http://tools.iedb.org/population/). The coverage in detail was as follows: Europe: 99.9%; North America: 99.2%; South America: 92.7%; East Asia: 98.5%; Southeast Asia: 98.1%; Northeast Asia: 97.4%; South Asia: 93.1%; Southwest Asia: 93.3%; Central Africa: 94.3%; East Africa: 92.3%; North Africa: 96.2%; South Africa: 91.2% and West Africa: 94.3%.

For HLA class II, although not done in this Example 1, the allele frequency may be collected in a similar manner as for HLA class I.

The next step was to identify T cell epitopes for SARS-CoV-2. This was done by obtaining the high-quality SARS-CoV-2 reference amino-acid sequence. The annotated (annotation score 5/5) Uniprot Wuhan strain was downloaded from Uniprot, query SARS-CoV-2 (https://www.uniprot.org/uniprot/?querg=sars-cov-2&fil=organism%3A%22Severe%20acute%20respiratorg%20syndrome%20coronavirus%202%20(2019-nCoV)%20(SARS-CoV-2)%20%5B2697049%5D%22&columns=id%2Centry%20name%2Creviewed%2Cprotein%20names%2Cgenes%2Corganism%2Clength&sort=score). Six proteins were selected: four structural proteins: the spike protein, the envelope protein, the membrane protein, and the nucleocapsid protein and two non-structural proteins: ORF1a/b and ORF3a. A search was carried out for hotspot genomic areas of epitopes predicted to bind to HLA class I alleles in the six protein sequences using NetMHCpan 4.0 (https://services.healthtech.dtu.dk/service.php?NetMHCpan-4.0) and the HLA class I alleles as defined in the initial step. In total 13236 epitopes predicted to bind to at least one HLA class I allele were found. To identify the hotspot areas, filtering was applied to keep only those epitopes that bind to more than 10 different HLA class I alleles and to at least 1 allele from each locus (A/B/C). The remaining high quality 604 epitopes were further processed by merging the overlapping or adjacent epitopes (within 3 amino acids apart) to obtain hotspot epitope regions. The epitopes shorter than 15 amino acids were extended to 15 amino acids. The binding to HLA I and HLA II alleles was predicted using NetMHCpan 4.0 and NetMHCIIpan 3.2 (https://services.healthtech.dtu.dk/service.php?NetMHCIIpan-3.2), respectively, on the final list of merged epitopes.

Up-to-date high-quality annotated sequences from NCBI virus database for SARS-CoV2 and SARS-CoV were then obtained. The homology to these sequences by global alignment (% identity between the strain and epitope sequence) was determined. Up-to-date high-quality annotated human reference protein sequences were obtained from https://www.uniProt.org/proteomes/UP000005640. A summary of all identical matches between the epitopes and the human proteome using 6, 7, 8 and 9 amino acid short sequences was created and a search for substring matches between the epitopes and all epitopes deposited in the Immune Epitope Database (IEDB) shown to elicit T/B cell response or binding to MHC class I was carried out. The final prioritization of epitopes was made based on the collected information:
Maximizing the global population coverage by prioritizing epitopes covering a large set of distinct MHC class I and alleles
Conservation within >200 different SARS CoV-2 strains worldwide from up-to-date NCBI virus database
Conservation within different SARS CoV strains and betacoronaviruses in general
No/minimal numbers of exact match of 6 amino acids to any protein in the human proteome
Identity with an immunodominant SARS-CoV epitope deposited in the Immune Epitope Database (IEDB)

The epitopes with the SEQ ID NO: 1-229 were identified:

| | | Sequence |
|---|---|---|
| Peptide ID | Amino Acid Sequence | Number |
| orf1ab_6411-6454 | HANEYRLYLDAYNMMISAGFSLWVYKQFDTYNL | SEQ ID NO: 1 |
| | WNTFTRLQSL | |
| orf1ab_2844-2905 | YTNDKACPLIAAVITREVGFVVPGLPGTILRTTNGD | SEQ ID NO: 2 |
| | FLHFLPRVFSAVGNICYTPSKLIEY | |
| orf1ab_2936-2969 | NVLEGSVAYESLRPDTRYVLMDGSIIQFPNTYL | SEQ ID NO: 3 |
| orf1ab_5266-5307 | QEYADVFHLYLQYIRKLHDELTGHMLDMYSVMLT | SEQ ID NO: 4 |
| | NDNTSRY | |
| SPIKE_864-906 | LTDEMIAQYTSALLAGTITSGWTFGAGAALQIPFA | SEQ ID NO: 5 |
| | MQMAYRF | |
| orf1ab_1413-1448 | GVVDYGARFYFYTSKTTVASLINTLNDLNETLVTM | SEQ ID NO: 6 |
| orf1ab_3097-3123 | YSFLPGVYSVIYLYLTFYLTNDVSFL | SEQ ID NO: 7 |
| orf1ab_3182-3217 | FLLNKEMYLKLRSDVLLPLTQYNRYLALYNKYKYF | SEQ ID NO: 8 |
| orf1ab_2371-2400 | VQMAPISAMVRMYIFFASFYYVWKSYVHV | SEQ ID NO: 9 |
| orf1ab_1535-1560 | YYTSNPTTFHLDGEVITFDNLKTLL | SEQ ID NO: |
| | | 10 |
| orf1ab_1565-1599 | RTIKVFTTVDNINLHTQVVDMSMTYGQQFGPTYL | SEQ ID NO: |
| | | 11 |
| orf1ab_4751-4781 | NLHSSRLSFKELLVYAADPAMHAASGNLLL | SEQ ID NO: |
| | | 12 |
| orf1ab_3068-3092 | RAFGEYSHWAFNTLLFLMSFTVL | SEQ ID NO: |
| | | 13 |
| orf1ab_2331-2365 | ILFTRFFYVLGLAAIMQLFFSYFAVHFISNSWLM | SEQ ID NO: |
| | | 14 |
| orf1ab_1169-1193 | RTNVYLAVFDKNLYDKLVSSFLEM | SEQ ID NO: |
| | | 15 |
| SPIKE_257-277 | WTAGAAAYYVGYLQPRTFLL | SEQ ID NO: |
| | | 16 |
| orf1ab_3932-3957 | EMLDNRATLQAIASEFSSLPSYAAF | SEQ ID NO: |
| | | 17 |
| orf1ab_1261-1286 | NLHPDSATLVSDIDITFLKKDAPYI | SEQ ID NO: |
| | | 18 |
| orf1ab_5973-5998 | MTYRRLISMMGFKMNYQVNGYPNMF | SEQ ID NO: |
| | | 19 |
| SPIKE_703-726 | SVAYSNNSIAIPTNFTISVTTEI | SEQ ID NO: |
| | | 20 |
| orf1ab_6481-6507 | TVYTKVDGVDVELFENKTTLPVNVAF | SEQ ID NO: |
| | | 21 |
| orf1ab_5613-5638 | HFAIGLALYYPSARIVYTACSHAAV | SEQ ID NO: |
| | | 22 |
| orf1ab_1797-1816 | VQQESPFVMMSAPPAQYEL | SEQ ID NO: |
| | | 23 |
| orf1ab_5462-5489 | KATEETFKLSYGIATVREVLSDRELHL | SEQ ID NO: |
| | | 24 |
| orf1ab_3619-3647 | AMSAFAMMFVKHKHAFLCLFLLPSLATV | SEQ ID NO: |
| | | 25 |
| orf1ab_5072-5100 | SSGDATTAYANSVFNICQAVTANVNALL | SEQ ID NO: |
| | | 26 |
| orf1ab_2771-2796 | QLIKVTLVFLFVAAIFYLITPVHVM | SEQ ID NO: |
| | | 27 |
| orf1ab_3682-3701 | VMYASAVVLLILMTARTVY | SEQ ID NO: |
| | | 28 |
| orf1ab_999-1023 | TTIQTIVEVQPQLEMELTPVVQTI | SEQ ID NO: |
| | | 29 |
| orf1ab_5168-5186 | ASIKNFKSVLYYQNNVFM | SEQ ID NO: |
| | | 30 |
| VEMP_3-21 | FVSEETGTLIVNSVLLFL | SEQ ID NO: |
| | | 31 |
| orf1ab_6093-6117 | MLSDTLKNLSDRVVFVLWAHGFEL | SEQ ID NO: |
| | | 32 |
| orf1ab_676-709 | TFFKLVNKFLALCADSIIIGGAKLKALNLGETF | SEQ ID NO: |
| | | 33 |
| orf1ab_2588-2604 | KMFDAYVNTFSSTFNV | SEQ ID NO: |
| | | 34 |
| VME1_36-51 | FAYANRNRFLYIIKL | SEQ ID NO: |
| | | 35 |
| orf1ab_824-849 | FGDDTVIEVQGYKSVNITFELDERI | SEQ ID NO: |
| | | 36 |
| orf1ab_3604-3619 | FLYENAFLPFAMGII | SEQ ID NO: |
| | | 37 |
| orf1ab_3355-3375 | TANPKTPKYKFVRIQPGQTF | SEQ ID NO: |
| | | 38 |
| NCAP_304-320 | AQFAPSASAFFGMSRI | SEQ ID NO: |
| | | 39 |
| orf1ab_5132-5148 | FVNEFYAYLRKHFSMM | SEQ ID NO: |
| | | 40 |
| orf1ab_2166-2181 | NYMPYFFTLLLQLCT | SEQ ID NO: |
| | | 41 |
| orf1ab_3706-3721 | RVWTLMNVLTLVYKV | SEQ ID NO: |
| | | 42 |
| orf1ab_2241-2262 | SLIYSTAALGVLMSNLGMPSY | SEQ ID NO: |
| | | 43 |
| orf1ab_2611-2633 | LVATAEAELAKNVSLDNVLSTF | SEQ ID NO: |
| | | 44 |
| orf1ab_1760-1776 | TLKGVEAVMYMGTLSY | SEQ ID NO: |
| | | 45 |
| orf1ab_3905-3921 | EAFEKMVSLLSVLLSM | SEQ ID NO: |
| | | 46 |
| orf1ab_1466-1486 | RSLKVPATVSVSSPDAVTAY | SEQ ID NO: |
| | | 47 |
| AP3A_88-107 | TVYSHLLLVAAGLEAPFLY | SEQ ID NO: |
| | | 48 |
| SPIKE_49-70 | STQDLFLPFFSNVTWFHAIHV | SEQ ID NO: |
| | | 49 |
| orf1ab_3651-3666 | MVYMPASWMRIMTW | SEQ ID NO: |
| | | 50 |
| orf1ab_76-92 | RTAPHGHVMVELVAEL | SEQ ID NO: |
| | | 51 |
| orf1ab_3038-3055 | GALDISASIVAGGIVAI | SEQ ID NO: |
| | | 52 |
| orf1ab_5356-5373 | HVISTSHKLVLSVNPYV | SEQ ID NO: |
| | | 53 |
| AP3A_53-68 | AVFQSASKIITLKKR | SEQ ID NO: |
| | | 54 |
| orf1ab_3481-3498 | FLNRFTTTLNDFNLVAM | SEQ ID NO: |
| | | 55 |
| AP3A_203-218 | HSYFTSDYYQLYSTQ | SEQ ID NO: |
| | | 56 |
| orf1ab_5015-5030 | RAMPNMLRIMASLVL | SEQ ID NO: |
| | | 57 |
| SPIKE_686-701 | VASQSIIAYTMSLGA | SEQ ID NO: |
| | | 58 |
| orf1ab_5770-5785 | EIVDTVSALVYDNKL | SEQ ID NO: |
| | | 59 |
| SPIKE_191-210 | FVFKNIDGYFKIYSKHTPI | SEQ ID NO: |
| | | 60 |
| orf1ab_4023-4038 | KVTSAMQTMLFTMLR | SEQ ID NO: |
| | | 61 |
| orf1ab_6850-6865 | YLNTLTLAVPYNMRV | SEQ ID NO: |
| | | 62 |
| SPIKE_108-127 | TLDSKTQSLLIVNNATNVV | SEQ ID NO: |
| | | 63 |
| orf1ab_4824-4842 | SSVELKHFFFAQDGNAAI | SEQ ID NO: |
| | | 64 |
| orf1ab_478-493 | SFSASTSAFVETVKG | SEQ ID NO: |
| | | 65 |
| orf1ab_1674-1689 | YLATALLTLQQIELK | SEQ ID NO: |
| | | 66 |
| SPIKE_814-829 | RSFIEDLLFNKVTLA | SEQ ID NO: |
| | | 67 |
| orf1ab_527-544 | SILSPLYAFASEAARW | SEQ ID NO: |
| | | 68 |
| orf1ab_3129-3144 | VMFTPLVPFWITIAY | SEQ ID NO: |
| | | 69 |
| orf1ab_6748-6763 | LLLDDFVEIIKSQDL | SEQ ID NO: |
| | | 70 |
| orf1ab_4630-4645 | SLLMPILTLTRALTA | SEQ ID NO: |
| | | 71 |
| VME1_94-109 | YFIASFRLFARTRSM | SEQ ID NO: |
| | | 72 |
| orf1ab_7039-7054 | YSLFDMSKFPLKLRG | SEQ ID NO: |
| | | 73 |
| orf1ab_6283-6298 | KAYKIEELFYSYATH | SEQ ID NO: |
| | | 74 |
| VEMP_50-65 | LVKPSFYVYSRVKNL | SEQ ID NO: |
| | | 75 |
| orf1ab_4086-4101 | TTFTYASALWEIQQV | SEQ ID NO: |
| | | 76 |
| SPIKE_633-648 | RVYSTGSNVFQTRAG | SEQ ID NO: |
| | | 77 |
| orf1ab_5245-5260 | LMIERFVSLAIDAYP | SEQ ID NO: |
| | | 78 |
| orf1ab_6153-6168 | HSIGFDYVYNPFMID | SEQ ID NO: |
| | | 79 |
| orf1ab_3541-3557 | RTILGSALLEDEFTPF | SEQ ID NO: |
| | | 80 |
| orf1ab_5532-5547 | VYRGTTTYKLNVGD | SEQ ID NO: |
| | | 81 |
| orf1ab_7063-7078 | QINDMILSLLSKGRL | SEQ ID NO: |
| | | 82 |
| AP3A_222-237 | TGVEHVTFFIYNKIV | SEQ ID NO: |
| | | 83 |
| VEMP_28-43 | VTLAILTALRLCAYC | SEQ ID NO: |
| | | 84 |
| orf1ab_859-874 | YTVELGTEVNEFACV | SEQ ID NO: |
| | | 85 |
| orf1ab_3573-3591 | RTIKGTHHWLLLTILTSL | SEQ ID NO: |
| | | 86 |
| orf1ab_4228-4243 | YFIKGLNNLNRGMVL | SEQ ID NO: |
| | | 87 |
| orf1ab_5380-5405 | VTDVTQLYLGGMSYYCKSHKPPISF | SEQ ID NO: |
| | | 88 |
| orf1ab_6799-6815 | SQAWQPGVAMPNLYKM | SEQ ID NO: |
| | | 89 |
| SPIKE_1053-1068 | QSAPHGWFLHVTYV | SEQ ID NO: |
| | | 90 |
| orf1ab_5719-5736 | YVYIGDPAQLPAPRTLL | SEQ ID NO: |
| | | 91 |
| orf1ab_4513-4528 | YTMADLVYALRHFDE | SEQ ID NO: |
| | | 92 |
| SPIKE_494-513 | YGFQPTNGVGYQPYRVWL | SEQ ID NO: |
| | | 93 |
| orf1ab_4725-4740 | FVDGVPFVVSTGYHF | SEQ ID NO: |
| | | 94 |
| orf1ab_6873-6892 | KGVAPGTAVLRQWLPTGTL | SEQ ID NO: |
| | | 95 |
| orf1ab_904-919 | YYLFDESGEFKLASH | SEQ ID NO: |
| | | 96 |
| orf1ab_4113-4130 | ISMDNSPNLAWPLIVTA | SEQ ID NO: |
| | | 97 |
| orf1ab_1633-1650 | YYHTTDPSFLGRYMSAL | SEQ ID NO: |
| | | 98 |
| orf1ab_873-889 | VVADAVIKTLQPVSEL | SEQ ID NO: |
| | | 99 |
| SPIKE_77-92 | RFDNPVLPFNDGVYF | SEQ ID NO: |
| | | 100 |
| orf1ab_4064-4080 | TTAAKLMVVIPDYNTY | SEQ ID NO: |
| | | 101 |
| orf1ab_561-576 | LQKAAITILDGISQY | SEQ ID NO: |
| | | 102 |
| AP3A_32-47 | ATIPIQASLPFGWLI | SEQ ID NO: |
| | | 103 |
| orf1ab_5212-5229 | KQGDDYVYLPYPDPSRI | SEQ ID NO: |
| | | 104 |
| orf1ab_4709-4724 | STVFPPTSFGPLVRK | SEQ ID NO: |
| | | 105 |
| orf1ab_2425-2444 | IVNGVRRSFYVYANGGKGF | SEQ ID NO: |
| | | 106 |
| orf1ab_2298-2314 | DTYPSLETIQITISSF | SEQ ID NO: |
| | | 107 |
| orf1ab_105-120 | VLVPHVGEIPVAYRK | SEQ ID NO: |
| | | 108 |
| orf1ab_250-266 | LQTPFEIKLAKKFDTF | SEQ ID NO: |
| | | 109 |
| orf1ab_4925-4940 | NVIPTITQMNLKYAI | SEQ ID NO: |
| | | 110 |
| SPIKE_365-380 | SVLYNSASFSTFKCY | SEQ ID NO: |
| | | 111 |
| orf1ab_6831-6846 | ATLPKGIMMNVAKYT | SEQ ID NO: |
| | | 112 |
| orf1ab_4002-4017 | KMADQAMTQMYKQAR | SEQ ID NO: |
| | | 113 |
| orf1ab_6605-6620 | SVGPKQASLNGVTLI | SEQ ID NO: |
| | | 114 |
| SPIKE_432-452 | VIAWNSNNLDSKVGGNYNYL | SEQ ID NO: |
| | | 115 |
| SPIKE_1219-1234 | FIAGLIAIVMVTIML | SEQ ID NO: |
| | | 116 |
| NCAP_330-346 | LTYTGAIKLDDKDPNF | SEQ ID NO: |
| | | 117 |
| orf1ab_1618-1633 | YVLPNDDTLRVEAFE | SEQ ID NO: |
| | | 118 |
| orf1ab_6668-6683 | AMDEFIERYKLEGYA | SEQ ID NO: |
| | | 119 |
| orf1ab_804-819 | NMMVTNNTFTLKGGA | SEQ ID NO: |
| | | 120 |
| orf1ab_5555-5570 | VMPLSAPTLVPQEHY | SEQ ID NO: |
| | | 121 |
| orf1ab_4672-4687 | KLFDRYFKYWDQTYH | SEQ ID NO: |
| | | 122 |
| orf1ab_2269-2284 | YLNSTNVTIATYCTG | SEQ ID NO: |
| | | 123 |
| orf1ab_4997-5012 | YSDVENPHLMGWDYP | SEQ ID NO: |
| | | 124 |
| orf1ab_4904-4919 | RLYYDSMSYEDQDAL | SEQ ID NO: |
| | | 125 |
| SPIKE_220-235 | SALEPLVDLPIGINI | SEQ ID NO: |
| | | 126 |
| orf1ab_2098-2113 | YVDNSSLTIKKPNEL | SEQ ID NO: |
| | | 127 |
| SPIKE_325-340 | IVRFPNITNLCPFGE | SEQ ID NO: |
| | | 128 |
| orf1ab_3751-3766 | MFLARGIVFMCVEYC | SEQ ID NO: |
| | | 129 |
| SPIKE_1135-1150 | TVYDPLQPELDSFKE | SEQ ID NO: |
| | | 130 |
| SPIKE_27-42 | YTNSFTRGVYYPDKV | SEQ ID NO: |
| | | 131 |
| orf1ab_1336-1351 | YTVEEAKTVLKKCKS | SEQ ID NO: |
| | | 132 |
| orf1ab_5314-5329 | AMYTPHTVLQAVGAC | SEQ ID NO: |
| | | 133 |
| orf1ab_6632-6647 | KVDGVVQQLPETYFT | SEQ ID NO: |
| | | 134 |
| orf1ab_4977-4993 | TVVIGTSKFYGGWHNM | SEQ ID NO: |
| | | 135 |
| orf1ab_2140-2156 | YAKPFLNKVVSTTTNI | SEQ ID NO: |
| | | 136 |
| AP3A_5-20 | RIFTIGTVTLKQGEI | SEQ ID NO: |
| | | 137 |
| AP3A_246-263 | HTIDGSSGVVNPVMEPI | SEQ ID NO: |
| | | 138 |
| orf1ab_618-633 | TVYEKLKPVLDWLEE | SEQ ID NO: |
| | | 139 |
| orf1ab_1247-1262 | FLTENLLLYIDINGN | SEQ ID NO: |
| | | 140 |
| orf1ab_6342-6357 | SLYVNKHAFHTPAFD | SEQ ID NO: |
| | | 141 |
| orf1ab_733-748 | KAPKEIIFLEGETLP | SEQ ID NO: |
| | | 142 |
| orf1ab_37-53 | VLSEARQHLKDGTCGL | SEQ ID NO: |
| | | 143 |
| orf1ab_6708-6723 | RFKESPFELEDFIPM | SEQ ID NO: |
| | | 144 |
| SPIKE_936-951 | SLSSTASALGKLQDV | SEQ ID NO: |
| | | 145 |
| orf1ab_6694-6709 | SQLGGLHLLIGLAKR | SEQ ID NO: |
| | | 146 |
| orf1ab_5843-5858 | AVASKILGLPTQTVD | SEQ ID NO: |
| | | 147 |
| orf1ab_1153-1168 | FGADPIHSLRVCVDT | SEQ ID NO: |
| | | 148 |
| orf1ab_272-287 | FVFPLNSIIKTIQPR | SEQ ID NO: |
| | | 149 |
| SPIKE_1094-1109 | FVSNGTHWFVTQRNF | SEQ ID NO: |
| | | 150 |
| VME1_190-205 | SGFAAYSRYRIGNYK | SEQ ID NO: |
| | | 151 |
| orf1ab_6900-6915 | FVSDADSTLIGDCAT | SEQ ID NO: |
| | | 152 |
| orf1ab_5923-5938 | LQAENVTGLFKDCSK | SEQ ID NO: |
| | | 153 |
| VME1_169-184 | VATSRTLSYYKLGAS | SEQ ID NO: |
| | | 154 |
| orf1ab_2216-2233 | YLKSPNFSKLINIIIWF | SEQ ID NO: |
| | | 155 |
| orf1ab_4862-4877 | FVVEVVDKYFDCYDG | SEQ ID NO: |
| | | 156 |
| orf1ab_2075-2090 | ILKPANNSLKITEEV | SEQ ID NO: |
| | | 157 |
| NCAP_158-173 | LQLPQGTTLPKGFYA | SEQ ID NO: |
| | | 158 |
| AP3A_138-153 | LLYDANYFLCWHTNC | SEQ ID NO: |
| | | 159 |
| orf1ab_6763-6778 | SVVSKVVKVTIDYTE | SEQ ID NO: |
| | | 160 |
| SPIKE_914-929 | VLYENQKLIANQFNS | SEQ ID NO: |
| | | 161 |
| SPIKE_950-966 | VVNQNAQALNTLVKQL | SEQ ID NO: |
| | | 162 |
| orf1ab_6245-6260 | LLADKFPVLHDIGNP | SEQ ID NO: |
| | | 163 |
| orf1ab_5515-5530 | KVQIGEYTFEKGDYG | SEQ ID NO: |
| | | 164 |
| AP3A_70-85 | LALSKGVHFVCNLLL | SEQ ID NO: |
| | | 165 |
| orf1ab_3311-3326 | MLNPNYEDLLIRKSN | SEQ ID NO: |
| | | 166 |
| orf1ab_3966-3981 | AVANGDSEWLKKLK | SEQ ID NO: |
| | | 167 |
| SPIKE_1184-1199 | RLNEVAKNLNESLID | SEQ ID NO: |
| | | 168 |
| orf1ab_2552-2567 | SAKSASVYYSQLMCQ | SEQ ID NO: |
| | | 169 |
| orf1ab_4418-4433 | STDWVYRAFDIYNDK | SEQ ID NO: |
| | | 170 |
| SPIKE_780-795 | VFAQVKQIYKTPPIK | SEQ ID NO: |
| | | 171 |
| SPIKE_975-990 | VLNDILSRLDKVEAE | SEQ ID NO: |
| | | 172 |
| orf1ab_640-655 | FLRDGWEIVKFISTC | SEQ ID NO: |
| | | 173 |
| orf1ab_444-459 | GLNDNLLEILQKEKV | SEQ ID NO: |
| | | 174 |
| SPIKE_416-431 | KIADYNYKLPDDFTG | SEQ ID NO: |
| | | 175 |
| NCAP_221-236 | LLLDRLNQLESKMSG | SEQ ID NO: |
| | | 176 |
| orf1ab_2749-2764 | VNVVTTKIALKGGK | SEQ ID NO: |
| | | 177 |
| orf1ab_5110-5125 | YVRNLQHRLYECLYR | SEQ ID NO: |
| | | 178 |
| orf1ab_4162-4177 | CTDDNALAYYNTTKG | SEQ ID NO: |
| | | 179 |
| orf1ab_3163-3178 | RVVFNGVSFSTFEEA | SEQ ID NO: |
| | | 180 |
| orf1ab_5945-5960 | TQAPTHLSVDTKFKT | SEQ ID NO: |
| | | 181 |
| orf1ab_1056-1071 | VNAANVYLKHGGGV | SEQ ID NO: |
| | | 182 |
| orf1ab_6681-6696 | YAFEHIVYGDFSHSQ | SEQ ID NO: |
| | | 183 |
| orf1ab_4846-4861 | YYRYNLPTMCDIRQL | SEQ ID NO: |
| | | 184 |
| orf1ab_2488-2503 | YIVDSVTVKNGSIHL | SEQ ID NO: |
| | | 185 |
| orf1ab_12-27 | HVQLSLPVLQVRDVL | SEQ ID NO: |
| | | 186 |
| SPIKE_583-598 | ILDITPCSFGGVSVI | SEQ ID NO: |
| | | 187 |
| orf1ab_5057-5072 | MVMCGGSLYVKPGGT | SEQ ID NO: |
| | | 188 |
| orf1ab_5571-5586 | RITGLYPTLNISDEF | SEQ ID NO: |
| | | 189 |
| orf1ab_358-373 | YLPQNAWKIYCPAC | SEQ ID NO: |
| | | 190 |
| orf1ab_5655-5670 | RIIPARARVECFDKF | SEQ ID NO: |
| | | 191 |
| orf1ab_1922-1937 | ASFDNFKFVCDNIKF | SEQ ID NO: |
| | | 192 |
| orf1ab_1875-1890 | TIKPVTYKLDGVVCT | SEQ ID NO: |
| | | 193 |
| orf1ab_2468-2483 | VARDLSLQFKRPINP | SEQ ID NO: |
| | | 194 |
| orf1ab_4282-4297 | YLASGGQPITNCVKM | SEQ ID NO: |
| | | 195 |
| orf1ab_3240-3255 | FSNSGSDVLYQPPQT | SEQ ID NO: |
| | | 196 |
| NCAP_265-280 | KAYNVTQAFGRRGPE | SEQ ID NO: |
| | | 197 |
| NCAP_47-62 | NTASWFTALTQHGKE | SEQ ID NO: |
| | | 198 |
| orf1ab_6577-6592 | RVDGQVDLFRNARNG | SEQ ID NO: |
| | | 199 |
| orf1ab_1350-1365 | SAFYILPSIISNEKQ | SEQ ID NO: |
| | | 200 |
| orf1ab_2534-2549 | SLPINVIVFDGKSKC | SEQ ID NO: |
| | | 201 |
| orf1ab_599-614 | YITGGVVQLTSQWLT | SEQ ID NO: |
| | | 202 |
| orf1ab_7019-7034 | YVMHANYIFWRNTNP | SEQ ID NO: |
| | | 203 |
| orf1ab_4356-4371 | ANDPVGFTLKNTVCT | SEQ ID NO: |
| | | 204 |
| orf1ab_4251-4266 | LQAGNATEVPANSTV | SEQ ID NO: |
| | | 205 |
| VME1_147-162 | HLRIAGHHLGRCDIK | SEQ ID NO: |
| | | 206 |
| orf1ab_2515-2530 | HSLSHFVNLDNLRAN | SEQ ID NO: |
| | | 207 |
| orf1ab_5803-5818 | ITHDVSSAINRPQIG | SEQ ID NO: |
| | | 208 |
| orf1ab_140-155 | KSFDLGDELGTDPYE | SEQ ID NO: |
| | | 209 |
| orf1ab_2121-2136 | LATHGLAAVNSVPWD | SEQ ID NO: |
| | | 210 |
| orf1ab_5753-5768 | KTIGPDMFLGTCRRC | SEQ ID NO: |
| | | 211 |
| orf1ab_6958-6973 | LALGGSVAIKITEHS | SEQ ID NO: |
| | | 212 |
| orf1ab_6212-6227 | KRVDWTIEYPIIGDE | SEQ ID NO: |
| | | 213 |
| orf1ab_4197-4212 | KSDGTGTIYTELEPP | SEQ ID NO: |
| | | 214 |
| orf1ab_5678-5693 | YVFCTVNALPETTAD | SEQ ID NO: |
| | | 215 |
| orf1ab_2674-2689 | LTYNKVENMTPRDLG | SEQ ID NO: |
| | | 216 |
| orf1ab_3329-3344 | LVQAGNVQLRVIGHS | SEQ ID NO: |
| | | 217 |
| SPIKE_393-408 | NVYADSFVIRGDEVR | SEQ ID NO: |
| | | 218 |
| orf1ab_4460-4475 | YFVVKRHTFSNYQHE | SEQ ID NO: |
| | | 219 |
| VME1_64-79 | FVLAAVYRINWITGG | SEQ ID NO: |
| | | 220 |
| orf1ab_6048-6063 | YVDTPNNTDFSRVSA | SEQ ID NO: |
| | | 221 |
| orf1ab_3767-3782 | IFFITGNTLQCIMLV | SEQ ID NO: |
| | | 222 |
| orf1ab_5880-5895 | NVNRFNVAITRAKVG | SEQ ID NO: |
| | | 223 |
| orf1ab_4532-4547 | TLKEILVTYNCCDDD | SEQ ID NO: |
| | | 224 |
| orf1ab_1213-1228 | FITESKPSVEQRKQD | SEQ ID NO: |
| | | 225 |
| SPIKE_603-618 | TSNQVAVLYQDVNCT | SEQ ID NO: |
| | | 226 |
| orf1ab_1733-1748 | YLFQHANLDSCKRVL | SEQ ID NO: |
| | | 227 |
| orf1ab_5437-5452 | WTNAGDYILANTCTE | SEQ ID NO: |
| | | 228 |
| orf1ab_5038-5053 | SLSHRFYRLANECAQ | SEQ ID NO: |
| | | 229 |

Other T cell epitopes were predicted by methods based on the above-described method (similar or identical). The following T cell epitopes were identified:

| | | SED |
|---|---|---|
| | | ID |
| Name | Sequence | NO: |
| AP3A_1_21 | DLFMRIFTIGTVTLKQGEIK | 322 |
| AP3A_33_73 | TIPIQASLPFGWLIVGVALLAVFQ | 323 |
| | SASKIITLKKRWQLAL | |
| AP3A_96_120 | VAAGLEAPFLYLYALVYFLQSINF | 324 |
| AP3A_157_192 | IPYNSVTSSIVITSGDGTTSPISE | 325 |
| | HDYQIGGYTEK | |
| AP3A_201_219 | VLHSYFTSDYYQLYSTQL | 326 |
| AP3A_219_235 | STDTGVEHVTFFIYNK | 327 |
| NCAP_44_74 | LPNNTASWFTALTQHGKEDLKFPR | 328 |
| | GQGVPI | |
| NCAP_99_113 | KMKDLSPRWYFYYL | 329 |
| NCAP_153_172 | NAAIVLQLPQGTTLPKGFY | 330 |
| NCAP_215_230 | DAALALLLLDRLNQL | 331 |
| NCAP_248_274 | KSAAEASKKPRQKRTATKAYNVTQ | 332 |
| | AF | |
| NCAP_296_333 | DYKHWPQIAQFAPSASAFFGMSRI | 333 |
| | GMEVTPSGTWLTY | |
| NCAP_337_353 | KLDDKDPNFKDQVILL | 334 |
| NCAP_344_370 | NFKDQVILLNKHIDAYKTFPPTEP | 335 |
| | KK | |
| NCAP_378_411 | TQALPQRQKKQQTVTLLPAADLDD | 336 |
| | FSKQLQQSM | |
| orf1ab_728_742 | LLMPLKAPKEIIFL | 337 |
| orf1ab_824_843 | FGDDTVIEVQGYKSVNITF | 338 |
| orf1ab_1172_11 | VYLAVFDKNLYDKLVSSFLEMKSE | 339 |
| 97 | K | |
| orf1ab_1605_16 | KIKPHNSHEGKTFYVLPNDDTLRV | 340 |
| 34 | EAFEY | |
| orf1ab_2067_20 | TTEVVGDIILKPANNSLKI | 341 |
| 86 | | |
| orf1ab_2165_21 | TNYMPYFFTLLL | 342 |
| 77 | | |
| orf1ab_2370_23 | LVQMAPISAMVRMYIFFASFYYVW | 343 |
| 94 | | |
| orf1ab_2478_25 | RPINPTDQSSYIVDSVT | 344 |
| 11 | | |
| orf1ab_2581_26 | DSAEVAVKMFDAYVNTFSSTFNVP | 345 |
| 13 | MEKLKTLV | |
| orf1ab_2880_29 | FLHFLPRVFSAVGNICYTPSKLIE | 346 |
| 09 | YTDFA | |
| orf1ab_2944_29 | YESLRPDTRYVLMDGSIIQFPNTY | 347 |
| 75 | LEGSVRV | |
| orf1ab_3387_34 | VYQCAMRPNFTIKGSFL | 348 |
| 04 | | |
| orf1ab_4195_42 | FPKSDGTGTIYTELEPPCRF | 349 |
| 15 | | |
| orf1ab_4498_45 | DGDMVPHISRQRLTKYTM | 350 |
| 16 | | |
| orf1ab_4647_46 | HVDTDLTKPYIKWDLLKYDF | 351 |
| 67 | | |
| orf1ab_5014_50 | DRAMPNMLRIMASLVLARKHTTC | 352 |
| 37 | | |
| orf1ab_5244_52 | TLMIERFVSLAIDAYPLTKHPNQE | 353 |
| 92 | YADVFHLYLQYIRKLHDELTGHML | |
| orf1ab_5452_54 | RLKLFAAETLKATEETFKLSYGIA | 354 |
| 81 | TVREV | |
| orf1ab_5556_55 | MPLSAPTLVPQEHYVRITGLY | 355 |
| 77 | | |
| orf1ab_5726_57 | AQLPAPRTLLTKGTL | 356 |
| 41 | | |
| orf1ab_5813_58 | RPQIGVVREFLTRNPAW | 357 |
| 30 | | |
| orf1ab_6498_65 | TTLPVNVAFEL | 358 |
| 09 | | |
| orf1ab_6632_66 | KVDGVVQQLPETYFTQSRNLQEF | 359 |
| 55 | | |
| orf1ab_6792_68 | FYPKLQSSQAWQPGVAMPNLYKMQ | 360 |
| 28 | RMLLEKCDLQNY | |
| orf1ab_6856_68 | LAVPYNMRVIHFGAGSDK | 361 |
| 74 | | |
| SPIKE_108_119 | TLDSKTQSLLI | 362 |
| SPIKE_191_212 | FVFKNIDGYFKIYSKHTPINL | 363 |
| SPIKE_264_279 | YYVGYLQPRTFLLKY | 364 |
| SPIKE_291_320 | ALDPLSETKCTLKSFTVEKGIYQT | 365 |
| | SNFRV | |
| SPIKE_319_338 | VQPTESIVRFPNITNLCPF | 366 |
| SPIKE_341_357 | FNATRFASVYAWNRKR | 367 |
| SPIKE_358_380 | SNCVADYSVLYNSASFSTFKCY | 368 |
| SPIKE_407_425 | RQIAPGQTGKIADYNYKL | 369 |
| SPIKE_525_565 | GPKKSTNLVKNKCVNFNFNGLTGT | 370 |
| | GVLTESNKKFLPFQQF | |
| SPIKE_624_636 | HADQLTPTWRVY | 371 |
| SPIKE_711_741 | IAIPTNFTISVTTEILPVSMTKTS | 372 |
| | VDCTMY | |
| SPIKE_778_797 | QEVFAQVKQIYKTPPIKDF | 373 |
| SPIKE_814_841 | RSFIEDLLFNKVTLADAGFIKQYG | 374 |
| | DCL | |
| SPIKE_891_933 | AALQIPFAMQMAYRFNGIGVTQNV | 375 |
| | LYENQKLIANQFNSAIGK | |
| SPIKE_955_970 | AQALNTLVKQLSSNF | 376 |
| SPIKE_974_991 | SVLNDILSRLDKVEAEV | 377 |
| SPIKE_995_100 | LITGRLQSLQTYV | 378 |
| 8 | | |
| SPIKE_1046_10 | YHLMSFPQSAPHGVVFLHVTYVPA | 379 |
| 75 | QEKNF | |
| SPIKE_1136_11 | VYDPLQPELDSF | 380 |
| 48 | | |
| VEMP_48_69 | VSLVKPSFYVYSRVKNLNSSR | 381 |
| VME1_2_23 | DSNGTITVEELKKLLEQWNLV | 382 |
| VME1_36_54 | FAYANRNRFLYIIKLIFL | 383 |
| VME1_88_116 | GLMWLSYFIASFRLFARTRSMWSF | 384 |
| | NPET | |
| VME1_147_180 | HLRIAGHHLGRCDIKDLPKEITVA | 385 |
| | TSRTLSYYK | |

SPIKE 8_110:
(SEQ ID NO: 386)
PLVSSQCVNLTTRTQLPPAYTNSFTRGVYYPDKVFRSSVLHSTQDLFLPF
FSNVTWFHAIHVSGTNGTKRFDNPVLPFNDGVYFASTEKSNIIRGWIFGT
TL
SPIKE_286_395
(SEQ ID NO: 387)
DAVDCALDPLSETKCTLKSFTVEKGIYQTSNFRVQPTESIVRFPNITNLC
PFGEVFNATRFASVYAWNRKRISNCVADYSVLYNSASFSTFKCYGVSPTK
LNDLCFTNV
SPIKE_483_552
(SEQ ID NO: 388)
EGFNCYFPLQSYGFQPTNGVGYQPYRVVVLSFELLHAPATVCGPKKSTNL
VKNKCVNFNFNGLTGTGVL
NCAP_288_387
(SEQ ID NO: 389)
QELIRQGTDYKHWPQIAQFAPSASAFFGMSRIGMEVTPSGTWLTYTGAIK
LDDKDPNFKDQVILLNKHIDAYKTFPPTEPKKDKKKKADETQALPQRQK
SPIKE_671_750
(SEQ ID NO: 390)
ASYQTQTNSPRRARSVASQSIIAYTMSLGAENSVAYSNNSIAIPTNFTIS
VTTEILPVSMTKTSVDCTMYICGDSTECS
SPIKE_1036_1090
(SEQ ID NO: 391)
SKRVDFCGKGYHLMSFPQSAPHGVVFLHVTYVPAQEKNFTTAPAICHDGK
AHFP
SPIKE_252_295
(SEQ ID NO: 392)
DSSSGWTAGAAAYYVGYLQPRTFLLKYNENGTITDAVDCALDP
SPIKE_805_853
(SEQ ID NO: 393)
LPDPSKPSKRSFIEDLLFNKVTLADAGFIKQYGDCLGDIAARDLICAQ
SPIKE_1180_1232
(SEQ ID NO: 394)
KEIDRLNEVAKNLNESLIDLQELGKYEQYIKWPWYIWLGFIAGLIAIVMV
TI
NCAP_49_129
(SEQ ID NO: 395)
ASWFTALTQHGKEDLKFPRGQGVPINTNSSPDDQIGYYRRATRRIRGGDG
KMKDLSPRWYFYYLGTGPEAGLPYGANKDG
SPIKE_438_479
(SEQ ID NO: 396)
NNLDSKVGGNYNYLYRLFRKSNLKPFERDISTEIYQAGSTP
VME1_153_212
(SEQ ID NO: 397)
HHLGRCDIKDLPKEITVATSRTLSYYKLGASQRVAGDSGFAAYSRYRIGN
YKLNTDHSS
AP3A_164_233
(SEQ ID NO: 398)
SSIVITSGDGTTSPISEHDYQIGGYTEKWESGVKDCVVLHSYFTSDYYQL
YSTQLSTDTGVEHVTFFIY
AP3A_84_132
(SEQ ID NO: 399)
LLFVTVYSHLLLVAAGLEAPFLYLYALVYFLQSINFVRIIMRLWLCWK
SPIKE_126_185
(SEQ ID NO: 400)
VIKVCEFQFCNDPFLGVYYHKNNKSWMESEFRVYSSANNCTFEYVSQPFL
MDLEGKQGN
SPIKE_880_941
(SEQ ID NO: 401)
TITSGWTFGAGAALQIPFAMQMAYRFNGIGVTQNVLYENQKLIANQFNSA
IGKIQDSLSST
VEMP_18_67
(SEQ ID NO: 402)
LFLAFVVFLLVTLAILTALRLCAYCCNIVNVSLVKPSFYVYSRVKNLNS
AP3A_0_33
(SEQ ID NO: 403)
MDLFMRIFTIGTVTLKQGEIKDATPSDFVRATA

| Name/ | |
|---|---|
| SEQ ID NO: | Sequence |
| SPIKE_187_ | NLREFVFKNIDGYFKIYSKHTPINLVRDLPQ |
| 218/404 | |
| SPIKE_1085_ | KAHFPREGVFVSNGTHWFVTQRNFYEPQII |
| 1115/405 | |
| NCAP_243_ | QTVTKKSAAEASKKPRQKRTATKAYNVTQAF |
| 285/406 | GRRGPEQTQGN |
| SPIKE_619_ | VPVAIHADQLTPTWRVYSTGSNVFQTRAGCL |
| 651/407 | I |
| SPIKE_849_ | ICAQKFNGLTVLPPLLTDEMIAQYTSALLAG |
| 890/408 | TITSGWTFGA |
| SPIKE_398_ | SFVIRGDEVRQIAPGQTGKIADYNYKLPDDF |
| 435/409 | TGCVIA |
| AP3A_41_ | PFGWLIVGVALLAVFQSASKIITLKKRWQLA |
| 85/410 | LSKGVHFVCNLLL |
| SPIKE_771_ | VEQDKNTQEVFAQVKQIYKTPPIKDFGGFNF |
| 805/411 | SQI |
| VME1_85_ | CLVGLMWLSYFIASFRLFARTRSMWSFNPET |
| 120/412 | NILL |
| NCAP_206_ | PARMAGNGGDAALALLLLDRLNQLESKMSGK |
| 242/413 | GQQQQ |
| VME1_0_ | MADSNGTITVEELKKLLEQWNLVIGFLFLTW |
| 32/414 | I |
| VME1_30_ | WICLLQFAYANRNRFLYIIKLIFLWLLWPVT |
| 63/415 | LA |
| NCAP_ | AQFAPSASAFFGMSRI |
| 304-320/416 | |
| orf1ab | FGDDTVIEVQGYKSVNITFELDERI |
| 824-849/417 | |
| orf1ab | YTVELGTEVNEFACV |
| 859-874/418 | |
| orf1ab_1797- | VQQESPFVMMSAPPAQYEL |
| 1816/419 | |
| orf1ab_2371- | VQMAPISAMVRMYIFFASFYYVWKSYVHV |
| 2400/420 | |
| orf1ab_2588- | KMFDAYVNTFSSTFNV |
| 2604/421 | |
| orf1ab_3355- | TANPKTPKYKFVRIQPGQTF |
| 3375/422 | |
| orf1ab_3481- | FLNRFTTTLNDFNLVAM |
| 3498/423 | |
| orf1ab_3651- | MVYMPASWVMRIMTW |
| 3666/424 | |
| orf1ab_3706- | RVWTLMNVLTLVYKV |
| 3721/425 | |
| orf1ab_4228- | YFIKGLNNLNRGMVL |
| 4243/426 | |
| orf1ab_4630- | SLLMPILTLTRALTA |
| 4645/427 | |
| orf1ab_4824- | SSVELKHFFFAQDGNAAI |
| 4842/428 | |
| orf1ab_5015- | RAMPNMLRIMASLVL |
| 5030/429 | |
| orf1ab_5072- | SSGDATTAYANSVFNICQAVTANVNALL |
| 5100/430 | |
| orf1ab_5132- | FVNEFYAYLRKHFSMM |
| 5148/431 | |
| orf1ab_5168- | ASIKNFKSVLYYQNNVFM |
| 5186/432 | |
| orf1ab_5245- | LMIERFVSLAIDAYP |
| 5260/433 | |
| orf1ab_5356- | HVISTSHKLVLSVNPYV |
| 5373/434 | |
| orf1ab_5613- | HFAIGLALYYPSARIVYTACSHAAV |
| 5638/435 | |
| orf1ab_5770- | EIVDTVSALVYDNKL |
| 5785/436 | |
| orf1ab_5973- | MTYRRLISMMGFKMNYQVNGYPNMF |
| 5998/437 | |
| orf1ab_6093- | MLSDTLKNLSDRVVFVLWAHGFEL |
| 6117/438 | |
| orf1ab_6481- | TVYTKVDGVDVELFENKTTLPVNVAF |
| 6507/439 | |
| orf1ab_6850- | YLNTLTLAVPYNMRV |
| 6865/440 | |
| SPIKE_633- | RVYSTGSNVFQTRAG |
| 648/441 | |
| SPIKE_703- | SVAYSNNSIAIPTNFTISVTTEI |
| 726/442 | |
| VEMP | RSFIEDLLFNKVTLA |
| 50-65/443 | |
| VME1 | LVKPSFYVYSRVKNL |
| 36-51/444 | |

### Example 2: Construction and Expression of the Vaccines

All gene sequences of tested constructs (VB10.COV2) were ordered from Genscript (860 Centennial Ave., Piscataway, N.J. 08854, USA) and cloned into the expression vector pUMVC4a.

All constructs were transfected into HEK293 cells and verified expression of intact vaccibody proteins were performed by sandwich ELISA of the supernatant. In addition, western blot analysis was performed with some constructs to verify conformation and size of vaccibody proteins.

### Example 3a: Design, Production and Characterisation of Various DNA and Protein Vaccibody Vaccines (Called VB10.COV2)

Multiple VB10.COV2 DNA vaccines were designed (FIG. 53):

VB2049 (SEQ ID NO: 252, FIG. 23A), encoding a MIP-1α targeting unit, a dimerization unit and an antigenic unit comprising a short form of the SARS-CoV-2 RBD (“RBD short”, amino acids 331-524, i.e. 193 amino acids).

VB2060 (SEQ ID NO: 254, FIG. 24A), encoding a MIP-1α targeting unit, a dimerization unit and an antigenic unit comprising a longer version of the SARS-CoV-2 RBD (“RBD long”, amino acids 319-542 i.e. 223 amino acids)

VB2065 (SEQ ID NO: 256, FIG. 25A), encoding a MIP-1α targeting unit, a dimerization unit and an antigenic unit comprising the spike protein (codon optimized to enable the expression of a full-length prefusion stabilized spike protein, removing the polybasic cleavage site recognized by furin, and adding stabilizing mutations, see Wrapp et al., Science 367, (2020), 1260-1263) from SARS-CoV2 strain Wuhan Hu-1.

VB2048 (SEQ ID NO: 258, FIG. 26A), encoding a MIP-1α targeting unit, a dimerization unit and an antigenic unit comprising 20 immunogenic T cell epitopes (see Table 1 below) from multiple SARS-CoV2 strains designed to induce protective immunity and predicted as described in Example 1.

TABLE 1
T cell epitopes VB2048

| | Sequence |
|---|---|
| pep1 | RSFIEDLLFNKVTLA |
| pep2 | MTYRRLISMMGFKMNYQVNG |
| | YPNMF |
| pep3 | LMIERFVSLAIDAYP |
| pep4 | RAMPNMLRIMASLVL |
| pep5 | MVYMPASWVMRIMTW |
| pep6 | FLNRFTTTLNDFNLVAM |
| pep7 | SSVELKHFFFAQDGNAAI |
| pep8 | HFAIGLALYYPSARIVYTACSHA |
| | AV |
| pep9 | YFIKGLNNLNRGMVL |
| pep10 | YLNTLTLAVPYNMRV |
| pep11 | AQFAPSASAFFGMSRI |
| pep12 | EIVDTVSALVYDNKL |
| pep13 | SSGDATTAYANSVFNICQAVT |
| | ANVNALL |
| pep14 | HVISTSHKLVLSVNPYV |
| pep15 | MLSDTLKNLSDRVVFVLWAHG |
| | FEL |
| pep16 | TANPKTPKYKFVRIQPGQTF |
| pep17 | ASIKNFKSVLYYQNNVFM |
| pep18 | FVNEFYAYLRKHFSMM |
| pep19 | RVWTLMNVLTLVYKV |
| pep20 | FAYANRNRFLYIIKL |

VB2059 (SEQ ID NO: 260, FIG. 27), encoding an anti-mouse MHCII scFv targeting unit, a dimerization unit and an antigenic unit comprising a longer version of the SARS-CoV-2 RBD (“RBD long”, amino acids 319-542 i.e. 223 amino acids).

VB2071 (SEQ ID NO: 262, FIG. 28), encoding an anti-mouse MHCII scFv targeting unit, a dimerization unit and an antigenic unit comprising the spike protein (codon optimized to enable the expression of a full-length prefusion stabilized spike protein, removing the polybasic cleavage site recognized by furin, and adding stabilizing mutations, see Wrapp et al., Science 367, (2020), 1260-1263) from SARS-CoV2 strain Wuhan Hu-1.

The predicted T cell epitopes pep08 and pep18 included in the below-described constructs VB2081-VB2099 are identical to the corresponding epitopes in table 1. pep25 has the amino acid sequence of SEQ ID NO: 75 identified in Example 1.

VB2081 (SEQ ID NO: 264, FIG. 29), encoding a MIP-1α targeting unit, a dimerization unit and an antigenic unit consisting of 1 predicted T cell epitope (pep08) and a longer version of the SARS-CoV-2 RBD (“RBD long”, amino acids 319-542 i.e. 223 amino acids), linked with a (GGGGS)₂ linker.

VB2082 (SEQ ID NO: 266, FIG. 30), encoding a MIP-1α targeting unit, a dimerization unit and an antigenic unit consisting of 1 predicted T cell epitope (pep18) and a longer version of the SARS-CoV-2 RBD (“RBD long”, amino acids 319-542 i.e. 223 amino acids), linked with a (GGGGS)₂ linker.

VB2083 (SEQ ID NO: 268, FIG. 31), encoding a MIP-1α targeting unit, a dimerization unit and an antigenic unit consisting of 2 predicted T cell epitopes (pep08+pep18 with a (GGGGS)₂ linker in between epitopes) and a longer version of the SARS-CoV-2 RBD (“RBD long”, amino acids 319-542 i.e. 223 amino acids), linked with a (GGGGS)₂ linker.

VB2084 (SEQ ID NO: 270, FIG. 32), encoding a MIP-1α targeting unit, a dimerization unit and an antigenic unit consisting of 3 predicted T cell epitopes (pep08, pep18+pep25 with a (GGGGS)₂ linker in between epitopes) and a longer version of the SARS-CoV-2 RBD (“RBD long”, amino acids 319-542 i.e. 223 amino acids), linked with a (GGGGS)₂ linker.

VB2085 (SEQ ID NO: 272, FIG. 33), encoding a MIP-1α targeting unit, a dimerization unit and an antigenic unit consisting of 1 predicted T cell epitope (pep08) and a longer version of the SARS-CoV-2 RBD (“RBD long”, amino acids 319-542 i.e. 223 amino acids), linked with a GLGGL linker.

VB2086 (SEQ ID NO: 274, FIG. 34), encoding a MIP-1α targeting unit, a dimerization unit and an antigenic unit consisting of 1 predicted T cell epitope (pep08) and a longer version of the SARS-CoV-2 RBD (“RBD long”, amino acids 319-542 i.e. 223 amino acids), linked with a (GLGGL)₂ linker.

VB2087 (SEQ ID NO: 276, FIG. 35), encoding a MIP-1α targeting unit, a dimerization unit and an antigenic unit consisting of 1 predicted T cell epitope (pep18) and a longer version of the SARS-CoV-2 RBD (“RBD long”, amino acids 319-542 i.e. 223 amino acids), linked with a GLGGL linker.

VB2088 (SEQ ID NO: 278, FIG. 36), encoding a MIP-1α targeting unit, a dimerization unit and an antigenic unit consisting of 2 predicted T cell epitopes (pep08+pep18 with a (GGGGS)₂ linker in between epitopes) and a longer version of the SARS-CoV-2 RBD (“RBD long”, amino acids 319-542 i.e. 223 amino acids), linked with a GLGGL linker.

VB2089 (SEQ ID NO: 280, FIG. 37), encoding a MIP-1α targeting unit, a dimerization unit and an antigenic unit consisting of 3 predicted T cell epitopes (pep08, pep18+pep25 with a (GGGGS)₂ linker in between epitopes) and a longer version of the SARS-CoV-2 RBD (“RBD long”, amino acids 319-542 i.e. 223 amino acids), linked with a GLGGL linker.

VB2091 (SEQ ID NO: 282, FIG. 38), encoding a MIP-1α targeting unit, a dimerization unit and an antigenic unit consisting of 1 predicted T cell epitope (pep08) and a longer version of the SARS-CoV-2 RBD (“RBD long”, amino acids 319-542 i.e. 223 amino acids), linked with a TQKSLSLSPGKGLGGL linker.

VB2092 (SEQ ID NO: 284, FIG. 39), encoding a MIP-1α targeting unit, a dimerization unit and an antigenic unit consisting of 3 predicted T cell epitopes (pep08, pep18 and pep25 with a (GGGGS)₂ linker in between epitopes) and a longer version of the SARS-CoV-2 RBD (“RBD long”, amino acids 319-542 i.e. 223 amino acids), linked with a TQKSLSLSPGKGLGGL linker.

VB2094 (SEQ ID NO: 286, FIG. 40), encoding a MIP-1α targeting unit, a dimerization unit and an antigenic unit consisting of 1 predicted T cell epitope (pep08) and a longer version of the SARS-CoV-2 RBD (“RBD long”, amino acids 319-542 i.e. 223 amino acids), linked with a SLSLSPGKGLGGL linker.

VB2095 (SEQ ID NO: 288, FIG. 41), encoding a MIP-1α targeting unit, a dimerization unit and an antigenic unit consisting of 3 predicted T cell epitopes (pep08, pep18 and pep25 with a (GGGGS)₂ linker in between epitopes) and a longer version of the SARS-CoV-2 RBD (“RBD long”, amino acids 319-542 i.e. 223 amino acids), linked with a SLSLSPGKGLGGL linker.

VB2097 (SEQ ID NO: 290, FIG. 42), encoding a MIP-1α targeting unit, a dimerization unit and an antigenic unit consisting of 3 predicted T cell epitopes (pep08, pep18 and pep25 with a (GGGGS)₂ linker in between epitopes) and a longer version of the SARS-CoV-2 RBD (“RBD long”, amino acids 319-542 i.e. 223 amino acids), linked with a GSAT linker.

VB2099 (SEQ ID NO: 292, FIG. 43), encoding a MIP-1α targeting unit, a dimerization unit and an antigenic unit consisting of 3 predicted T cell epitopes (pep08, pep18 and pep25 with a (GGGGS)₂ linker in between epitopes) and a longer version of the SARS-CoV-2 RBD (“RBD long”, amino acids 319-542 i.e. 223 amino acids), linked with a SEG linker.

VB2129 (SEQ ID NO: 294, FIG. 44), encoding a MIP-1α targeting unit, a dimerization unit and an antigenic unit comprising a longer version of the SARS-CoV-2 RBD (“RBD long”, amino acids 319-542 i.e. 223 amino acids) with 3 mutations characterised in the South African variant B.1.351.

VB2131, encoding a MIP-1α targeting unit, a dimerization unit and an antigenic unit comprising of 2 longer version of the SARS-CoV-2 RBD (“RBD long”, amino acids 319-542 i.e. 223 amino acids) from the Wuhan strain and the South African variant B.1.351, linked with a SEG linker (amino acid sequence: SEQ ID NO: 296, FIG. 45).

VB2132, encoding a MIP-1α targeting unit, a dimerization unit and an antigenic unit comprising of 2 longer version of the SARS-CoV-2 RBD (“RBD long”, amino acids 319-542 i.e. 223 amino acids) from the Wuhan strain and the South African variant B.1.351, linked with a GSAT linker (amino acid sequence: SEQ ID NO: 297, FIG. 46).

VB2133, encoding a MIP-1α targeting unit, a dimerization unit and an antigenic unit comprising of 2 longer version of the SARS-CoV-2 RBD (“RBD long”, amino acids 319-542 i.e. 223 amino acids) from the Wuhan strain and the South African variant B.1.351, linked with a TQKSLSLSPGKGLGGL linker (amino acid sequence: SEQ ID NO: 298, FIG. 47).

VB2134, encoding a MIP-1α targeting unit, a dimerization unit and an antigenic unit comprising of 2 longer version of the SARS-CoV-2 RBD (“RBD long”, amino acids 319-542 i.e. 223 amino acids) from the Wuhan strain and the South African variant B.1.351, linked with a SLSLSPGKGLGGL linker (amino acid sequence: SEQ ID NO: 299, FIG. 48).

VB2135, encoding a MIP-1α targeting unit, a dimerization unit and an antigenic unit comprising of 2 longer version of the SARS-CoV-2 RBD (“RBD long”, amino acids 319-542 i.e. 223 amino acids) from the South African variant B.1.351 and the UK variant B.1.1.7, linked with a SEG linker (amino acid sequence: SEQ ID NO: 300, FIG. 49).

VB2136, encoding a MIP-1α targeting unit, a dimerization unit and an antigenic unit comprising of 2 longer version of the SARS-CoV-2 RBD (“RBD long”, amino acids 319-542 i.e. 223 amino acids) from the South African variant B.1.351 and the UK variant B.1.1.7, linked with a GSAT linker (amino acid sequence: SEQ ID NO: 301, FIG. 50).

VB2137, encoding a MIP-1α targeting unit, a dimerization unit and an antigenic unit comprising of 2 longer version of the SARS-CoV-2 RBD (“RBD long”, amino acids 319-542 i.e. 223 amino acids) from the South African variant B.1.351 and the Californian variant B.1.427, linked with a SEG linker (amino acid sequence: SEQ ID NO: 302, FIG. 51).

VB2138, encoding a MIP-1α targeting unit, a dimerization unit and an antigenic unit comprising of 2 longer version of the SARS-CoV-2 RBD (“RBD long”, amino acids 319-542 i.e. 223 amino acids) from the South African variant B.1.351 and the Californian variant B.1.427, linked with a GSAT linker (amino acid sequence: SEQ ID NO: 303, FIG. 52).

### Example 3b: In Vitro Characterization of the VB10.COV2 Protein Expression Level Post Transient Transfection of Mammalian Cells with VB10.COV2 DNA Plasmids

The purpose of this study was to do an in vitro characterization of the VB10.COV2 protein expression level post transient transfection of mammalian cells with the VB10.COV2 DNA plasmids, by measuring the presence of functional VB10.COV2 proteins in the cell supernatant by an ELISA assay using binding of specific antibodies to the targeting, dimerization and antigenic units of the protein. In addition, a western blot analysis was performed to verify conformation and size of the protein encoded by VB2060.

The VB10.COV2 DNA constructs were synthesized, cloned and produced by Genscript. The resulting constructs encoded for homodimeric proteins with MIP-1α and other targeting units and RBD/spike and/or T cell epitopes as antigenic unit, connected via a dimerization unit consisting of human hinge exons h1 and h4 and CH3 domain of IgG3. Genscript also performed DNA plasmid preparation (0.5-1.0 mg).

HEK293 cells were obtained from ATCC. HEK293 cells were transiently transfected with VB10.COV2 DNA plasmids. Briefly, 2×10⁵ cells/well were plated in 24-well tissue culture plates with 10% FBS growth medium and transfected with 1 μg VB10.COV2 DNA plasmid using Lipofectamine® 2000 reagent under the conditions suggested by the manufacturer (Invitrogen, Thermo Fischer Scientific). The transfected cells were then maintained for up to 6 days at 37° C. with 5% CO₂ and the cell supernatant was harvested for characterization of the VB10.COV2 protein.

ELISA was performed to verify the amount of VB10.COV2 protein produced by the HEK293 cells and secreted into the cell supernatant. Briefly, MaxiSorp Nunc-immuno plates were coated with 1 μg/ml of anti-CH3 (MCA878G, BioRad) in 1×PBS with 100 μl/well and plates were incubated overnight at 4° C. The microtiter wells were blocked by the addition of 200 μl/well 4% BSA in 1×PBS. 100 μl of cell supernatant from transfected HEK293 cells containing VB10.COV2 proteins were added to the plates. For detection antibody, either anti-human MIP-1α biotinylated (R&D Systems), anti-human IgG biotinylated (Thermo Fischer Scientific) or SARS-CoV-2/2019-nCoV spike/RBD Antibody (1:1000) (Sino Biologic) was added and incubated. Thereafter, strep-HRP (1:3000) or anti-Rabbit IgG-HRP (1:5000) was added and incubated. Unless specified, all incubations were carried out at 37° C. for 1 h, followed by 3× washing with PBS-Tween. Afterwards, 100 μl/well of TMB solution was added and color development was stopped after 5-15 min adding 100 μl/well of 1 M HCl. The optical density at 450 nm was determined on an automated plate reader (Thermo Scientific Multiscan GO).

In addition, western blot analysis was performed to verify the amount of VB10.COV2 protein produced by the HEK293 cells and secreted into the cell supernatant. Briefly, samples were prepared by mixing 24 μl supernatant from transfected HEK293 cells with 8 μl of Novex Bolt LDS sample buffer 4× (Invitrogen) with or without 3 μl of reducing agent added (Invitrogen). Samples (reduced or non-reduced) were boiled at 95° C. for 4-5 minutes before added to 4%-12% Novex Tris-glycine precast gels (Invitrogen). SDS-PAGE was performed in Novex Bolt SDS running buffer with a SeeBlue Plus2 pre-stained standard (Invitrogen). Proteins were transferred to EtOH-activated PVDF membranes by using the Tran-Blot Turbo system (Bio-Rad). PVDF membranes were blocked with 3% BSA PBST, and proteins were detected with spike-RBD Rabbit pAb (Sino Biological)—Goat anti-rabbit-AP (Sigma). Bands were developed with BCIP/NBT-Purple Liquid substrate system for membranes until color development.

FIGS. 54, 55 and 56 show the successful expression and secretion of the following functional VB10.COV2 proteins:

VB2049 (SEQ ID NO: 253, FIG. 23B), VB2060 (SEQ ID NO: 255, FIG. 24B), VB2065 (SEQ ID NO: 257, FIG. 25B), VB2048 (SEQ ID NO: 259, FIG. 26B), VB2059 (SEQ ID NO: 261, FIG. 27B), VB2071 (SEQ ID NO: 263, FIG. 28B), VB2081 (SEQ ID NO: 265, FIG. 29B), VB2082 (SEQ ID NO: 267, FIG. 30B), VB2083 (SEQ ID NO: 269, FIG. 31B), VB2084 (SEQ ID NO: 271, FIG. 32B), VB2085 (SEQ ID NO: 273, FIG. 33B), VB2086 (SEQ ID NO: 275, FIG. 34B), VB2087 (SEQ ID NO: 277, FIG. 35B), VB2088 (SEQ ID NO: 279, FIG. 36B), VB2089 (SEQ ID NO: 281, FIG. 37B), VB2091 (SEQ ID NO: 283, FIG. 38B), VB2092 (SEQ ID NO: 285, FIG. 39B), VB2094 (SEQ ID NO: 287, FIG. 40B), VB2095 (SEQ ID NO: 289, FIG. 41B), VB2097 (SEQ ID NO: 291, FIG. 42B), VB2099 (SEQ ID NO: 293, FIG. 43B), VB2129 (SEQ ID NO: 295, FIG. 44B), VB2131 (SEQ ID NO: 296, FIG. 45), VB2132 (SEQ ID NO: 297, FIG. 46), VB2133 (SEQ ID NO: 298, FIG. 47), VB2134 (SEQ ID NO: 299, FIG. 48), VB2135 (SEQ ID NO: 300, FIG. 49), VB2136 (SEQ ID NO: 301, FIG. 50), VB2137 (SEQ ID NO: 302, FIG. 51) and VB2138 (SEQ ID NO: 303, FIG. 52).

Conformational integrity of the VB10.COV2 proteins was confirmed by binding to antibodies specific for anti hIgG (CH3 domain) (as capture antibody), hMIP-1α, the RBD domain or spike protein in ELISA and western blot analysis.

In ELISA, the expression level was found to vary between high, medium and low expression between the various VB10.COV2 constructs depending on the molecule structure.

The constructs containing the longer RBD domain (VB2059 and VB2060) were expressed at the highest levels compared to the construct with the short RBD domain (VB2049) (FIG. 54 and FIG. 55B). Introducing mutations into the longer RBD domain did slightly change the expression level, as observed when comparing VB2060 with VB2129 (FIG. 55D). The constructs containing the spike protein (VB2065 and VB2071) were expressed at lower levels than the RBD constructs (FIG. 54 and FIG. 55E). The constructs containing the same antigenic unit (either the long RBD or the spike protein) but with different targeting units (human MIP1α or anti-mouse MHCII scFv) had no significant differences in expression levels (FIG. 54 and FIG. 55E).

For the constructs containing the combination of predicted T cell epitopes and the long RBD domain in the antigenic unit, differences in expression level are observed dependent on the T cell epitopes and linkers included. The expression level was highest for constructs containing pep18 (VB2082 and VB2087) compared to constructs containing pep08 (VB2081). When constructs comprised 3 T cell epitopes (pep08, pep18 and pep25), constructs with a SEG or GSAT linker were significantly better expressed compared to the constructs with other linkers between the last of the 3 T cell epitopes and the long RBD domain (FIG. 55C).

When co-transfecting HEK293 cells with 2 plasmids, VB2048 and VB2049, the expression levels were similar as when they were transfected with one of the plasmids alone (FIG. 55E and FIG. 54/55B).

In western blot analysis (FIG. 56), a strong band was detected for VB2060 at approximately 95 kDa under non-reducing conditions, indicating the presence of VB2060 homodimeric proteins. No bands were detected at approximately half the size under non-reducing conditions, suggesting that the encoded VB2060 polypeptides expressed from HEK293E cells, form homodimers in the supernatant. Under reducing conditions, a band at approximately 50 kDa was observed indicating the reduction of covalent disulfide bridges in the hinge region of VB2060, and formation of monomeric molecules. Lack of observed bands in the lipofectamine control lane indicates high specificity and low cross-reactivity of the detection antibody (FIG. 56A).

In conclusion, Example 3 shows that constructs that are expressed in HEK293 cells which may indicate that they could also be secreted at higher levels in vivo, i.e. from myocytes after intramuscular vaccination.

### Example 4: Anti-RBD Immune Responses in Mice Immunized with VB10.COV2 Vaccibody DNA Vaccines

For all experiments with mice (Examples 4-8), the following study design was applied: Female, 6-8-week-old BALE/c mice were obtained from Janvier Labs (France). All animals were housed in the animal facility at the Radium Hospital (Oslo, Norway) or the University of Oslo (Norway). All animal protocols were approved by the Norwegian Food Safety Authority (Oslo, Norway). For the studies, the mice were vaccinated with the DNA vaccines as described in table 2 below. The vaccine was administrated to each tibialis anterior (TA) muscle by needle injection (25 μl solution of vaccibody DNA plasmids in sterile PBS in each leg) followed by AgilePulse in vivo electroporation (EP) (BTX, U. S.). The AgilePulse EP delivery consists of 3 sets of pulses with 110-450 voltage. The first set, there are 1 50 μs pulse with a 0.2 ms delay; the second set is 1 50 μs pulse with a 50 ms delay and the third set is 8 pulses with 10 ms pulse and 20 ms delay. Sera samples, samples collected from the lungs by bronchoalveolar lavage (BAL) and spleens were collected as described in table 2 below.

TABLE 2
Mouse studies: vaccination, frequency and dose, samples
collection, reference to Examples and Figures

| | | | | | | |
|---|---|---|---|---|---|---|
| | | | | | | Example |
| | | Vaccination | Termination | Sera on | Spleen | and |
| Group | Conc. [μg] | on day | on day | day | on day | FIGS. |

Experiment 1

| | | | | | | |
|---|---|---|---|---|---|---|
| Low dose | 2.5 | 0 | 14 | NA | 14 | Ex. 9 FIG. |
| | | | | | | 73 |
| Low dose | 2.5 | 0, 21 | 14, 28 | 0, 7, 14, | 14, 28 | Ex. 4-8 |
| | | | | 20, 28 | | FIGS. 57A, |
| | | | | | | 58, 59, 61, |
| | | | | | | 64, 66 |
| Medium dose 1 | 25 | 0 | 7 | NA | 7 | Ex 6 FIG. |
| | | | | | | 61A |
| Medium dose 2 | 25 | 0 | 14 | NA | 14 | Ex. 9 FIG. |
| | | | | | | 73 |
| Medium dose 3 | 25 | 0, 21 | 14, 28, 90 | 0, 7, 14, | 14, 28, | Ex. 4-6, 8, 9 |
| | | | | 20, 28, | 90 | FIGS. 57A, |
| | | | | 42, 56, | | 58, 59, 62, |
| | | | | 70, 90 | | 67, 68, |
| | | | | | | 73-74 |
| Medium dose 4 | 25 | 0, 21, 89 | 99 | 0, 7, 14, | 99 | Ex. 4-6, 8 |
| | | | | 20, 28, | | FIGS. 57A, |
| | | | | 42, 56, | | 58-60, 62, |
| | | | | 70, 90, | | 57A, 69-70 |
| | | | | 99 | | |
| High dose 1 | 50 | 0 | 90, 99 | 0, 7, 14, | 90/99 | Ex. 4-6, 8 |
| | | | | 20, 28, | | FIGS. 57A, |
| | | | | 42, 56, | | 58, 62, 57, |
| | | | | 70, 90, | | 67-68 |
| | | | | 99 | | |
| High dose 2 | 50 | 0, 21 (or 89) | 28, 90 | 0, 7, 14, | 28, 90 | Ex. 4-6, 8 |
| | | | | 20, 28, | | FIGS. 57A/B, |
| | | | | 42, 56, | | 58, 62, 63, |
| | | | | 70, 90 | | 57, 67-70 |
| High dose 3 | 50 | 0, 21, 89 | 99 | 0, 7, 14, | 99 | Ex. 4-6, 8 |
| | | | | 20, 28, | | FIGS. 57A, |
| | | | | 42, 56, | | 58, 62, 57, |
| | | | | 70, 90, | | 69-70 |
| | | | | 99 | | |

Experiment 2

| | | | | | | |
|---|---|---|---|---|---|---|
| Treatment 1 | 3.0 | 0, (21) | 28 + BAL | 0, 7, 14, | NA | FIGS. 57C/D |
| | | | | 21, 28 | | |
| Treatment 2 | 6.25 | 0, (21) | 28 + BAL | 0, 7, 14, | NA | FIGS. 57C/D |
| | | | | 21, 28 | | |
| Treatment 3 | 12.5 | 0, (21) | 28 + BAL | 0, 7, 14, | NA | FIGS. 57C/D |
| | | | | 21, 28 | | |
| Treatment 4 | 25 | 0, (21) | 28 + BAL | 0, 7, 14, | NA | FIGS. 57C/D |
| | | | | 21, 28 | | |

Experiment 3

| | | | | | | |
|---|---|---|---|---|---|---|
| Treatment 1 | 1.0 | 0 | 7 + 14 | 7 + 14 | 7 + 14 | Ex. 6 |
| | | | | | | FIG. 63 |
| Treatment 2 | 6.25 | 0 | 7 + 14 | 7 + 14 | 7 + 14 | Ex. 6 |
| | | | | | | FIG. 63 |
| Treatment 3 | 12.5 | 0 | 7 + 14 | 7 + 14 | 7 + 14 | Ex. 6 |
| | | | | | | FIG. 63 |
| Treatment 4 | 25 | 0 | 7 + 14 | 7 + 14 | 7 + 14 | Ex. 6 |
| | | | | | | FIG. 63 |

The purpose of the study of Example 4 was to evaluate the humoral immune response induced in mice against RBD when vaccinated with VB10.COV2 vaccibody DNA vaccines as a function of the dose and number of doses of DNA vaccine administered.

The humoral immune response was evaluated in sera collected from vaccinated mice by an ELISA assay detecting total IgG in the sera binding to RBD from SARS-CoV2. Nunc ELISA plates were coated with 1 μg/ml recombinant protein antigen in D-PBS overnight at 4° C. Plates were blocked with 4% BSA in D-PBS for 1 hour at RT. Plates were then incubated with serial dilutions of mouse sera and incubated for 2 h at 37° C. Plates were washed 3× and incubated with 1:50 000 dilution of HRP-anti-mouse IgG secondary antibody (Southern Biotech) and incubated for 1 h at 37° C. After final wash plates were developed using TMB substrate (Merck, cat. CL07-1000). Plates were read at 450 nm wavelength within 30 min using a Multiscan GO (Thermo Fischer Scientific). Binding antibody endpoint titers were calculated. Binding antigens tested included SARS-CoV-2 antigens: RBD (Sino Biological 40592-V08H).

The four DNA vaccines (VB2049, VB2060, VB2065 and VB2071) were compared for the ability to induce anti-RBD IgG, and VB2060 was superior compared to VB2049 (FIG. 57A/B, the two graphs in FIG. 57A present the same data in different ways).

VB2060 demonstrated a consistent dose-response with specific anti-RBD IgG as early as day 7 post a single vaccination; even at the lowest doses (FIGS. 57A and 57C). The antibody levels peaked at day 28 (105 endpoint titer) after a single dose and persisted for at least 90 days (FIGS. 57A and 57E). For VB2060, the peak and durability of the response were further increased (>10⁶ endpoint titer) following a two-dose regimen (days 0 and 21) compared to the single dose group. Limited added benefit was observed at day 99 in mice that received a boost vaccination at day 89 (FIGS. 57A and 57E).

A second experiment confirmed a dose-dependent response in the range of 3.0, 6.25, 12.5 and 25 μg of VB2060 (FIG. 57C), in particular on day 7, however already at day 14 levels reached ˜10⁵ endpoint titer at all doses.

Furthermore, the kinetics of RBD-specific IgG were tested in bronchoalveolar lavage (BAL) from mice having been vaccinated once or twice with different doses of VB2060 (FIG. 57D). RBD-specific IgG in the lung which may contribute to local virus neutralization as a first line of protection against respiratory tract infection. RBD-specific IgG was found in BAL at the earliest time point tested (day 14) even with the lowest dose. The levels increased with dose and over time.

VB2065 and VB2071 (spike protein) also induced strong IgG responses against RBD, however, these appeared to be weaker than the RBD-based construct VB2060 (FIG. 57 B). This finding may probably be explained with the lower secretion of the vaccine protein (see FIG. 54). VB2059 (long RBD) and VB2071 (spike protein) with anti-mouse MHCII scFv targeting also induced strong IgG responses against RBD. However, these appeared weaker to be weaker than the MIP1α targeted RBD-based construct VB2060 (FIGS. 57B and 57E). VB2059 demonstrated a consistent dose-response with specific anti-RBD IgG as early as day 7 post a single vaccination; even at the lowest doses (FIG. 57E). However, the antibody levels peaked at a later time point with a lower response in comparison to VB2060 (day 56, 10⁵ endpoint titer) (FIGS. 57A and 57E). This finding clearly shows that the MIP1α targeting unit is superior compared to the anti-mouse MHCII scFv targeting at eliciting rapid and long-lasting high levels of anti-RBD antibodies with the VB10.COV2 vaccines comprising such targeting unit.

For the constructs containing a combination of predicted T cell epitopes and the long RBD domain in the antigenic unit, constructs VB2097 (3 epitopes+GSAT linker) and VB2099 (3 epitopes+SEG linker) induced stronger IgG responses against RBD than the constructs comprising 3 epitopes with other linkers (FIG. 57F). As for the constructs with 1 epitope, VB2082 and VB2087 (including pep18) induced stronger anti-RBD IgG responses compared to constructs comprising the pep08 epitope (VB2081) (FIG. 57F). These findings may probably be explained with the lower secretion of the vaccine proteins (see FIG. 55C). The best constructs containing a combination of predicted T cell epitopes and the long RBD, VB2097 and VB2087 induce similar immune responses compared to VB2060, comprising only the long RBD (FIGS. 57A and 57E).

The VB10.COV2 DNA vaccine containing the long RBD domain with the 3 South African variant mutations, VB2129, demonstrated a specific anti-RBD IgG as early as day 7 post a single vaccination; even at a low dose (FIG. 57G). The antibody levels further increased until day 14 (10⁴ endpoint titer) at all doses.

When co-vaccinating mice with 2 plasmids, VB2048 and VB2049, in one combined DNA vaccine solution with 12.5 μg of each plasmid, the data shows that a strong anti-RBD IgG response is elicited already at day 14 (FIG. 57H).

### Example 5: VB10.COV2 Vaccibody DNA Vaccines Elicit Strong Neutralizing Antibody Responses in Mice

The purpose of this study was to evaluate the extent of neutralizing antibody response induced in mice against live SARS-CoV-2 virus, when vaccinated with VB10.COV2 vaccibody DNA constructs VB2049, VB2060 and VB2065 as a function of the dose and number of doses of DNA vaccine given to the mice.

Live virus microneutralization assays (MNA) were performed at Public Health England (Porton Down, UK) as described in Folegatti et al., Lancet 396 (10249), 2020, 467-478. Neutralising virus titers were measured in heat-inactivated (56° C. for 30 min) serum samples. Diluted SARS-CoV-2 (Australia/VIC01/20202) was mixed 50:50 in 1% FCS/MEM with doubling serum dilutions in a 96-well V-bottomed plate and incubated at 37° C. in a humidified box for 1 h. The virus/serum mixtures were then transferred to washed Vero E6 (ECACC 85020206) cell monolayers in 96-well flat-bottomed plates, allowed to adsorb at 37° C. for a further hour, before removal of the virus inoculum and replacement with overlay (1% w/v CMC in complete media). The box was resealed and incubated for 24 hours prior to fixing with 8% (w/v) formaldehyde solution in PBS. Microplaques were detected using a SARS-CoV-2 antibody specific for the SARS-CoV-2 RBD spike protein and a rabbit HRP conjugate, infected foci were detected using TrueBlue™ substrate. Stained microplaques were counted using ImmunoSpot® S6 Ultra-V Analyzer and resulting counts analysed in SoftMax Pro v7.0 software. International Standard 20/130 (human anti-SARS-CoV-2 antibody from human convalescent plasma, NIBSC, UK) was used for comparison as a positive control.

Sera from mice vaccinated with the VB10.COV2 vaccibody DNA constructs VB2049, VB2060 and VB2065 were assessed the live virus neutralization assay and neutralizing antibody responses were seen for all the constructs.

A dose-dependent response was observed, with a low dose of VB2060 (2.5 μg) being sufficient to induce notable neutralizing activity at day 28. Also, a single high dose of VB2060 (50 μg) was able to induce neutralizing activity already at day 7, which peaked at day 28 with no signs of decline at day 90, comparable or higher to levels observed in convalescent plasma from recovered COVID-19 patients (NIBSC standard 20/130). Independent of the dose, the strongest response was observed at day 99 (after boost at day 89), showing induction of long-lasting, neutralising antibody responses with VB2060.

Two and three doses of 25 or 50 μg VB2049 did induce modest levels of neutralizing antibody responses at days 90 and 99 as did two doses of 50 μg VB2065 at day 28.

A second experiment confirmed a dose-dependent response in the range of 3.0, 6.25, 12.5 and 25 μg of VB2060 (FIG. 58B), in particular on day 7, however already at day 14 levels reached ˜10³ endpoint titer at all doses. In this experiment, one vaccination with the highest dose of VB2060 (25 μg) was able to induce strong neutralizing activity already at day 7, which peaked at day 28 (no boost), comparable or higher to levels observed in convalescent plasma from recovered COVID-19 patients (NIBSC standard 20/130).

From the above results, VB2060 appears superior to VB2065 and VB2049 in inducing rapid and high levels of neutralizing antibodies even with only one dose. The results show that VB2060 is a potent DNA vaccine which is capable of eliciting virus neutralizing activity already at day 7 after vaccination (FIG. 58).

### Example 6: Evaluation of the Magnitude and Specificity of T Cell Responses after Vaccination with VB10.COV2 Vaccibody DNA Vaccines

The purpose of this study was to evaluate the cellular immune response against RBD/spike in splenocytes from mice vaccinated with VB10.COV2 vaccibody DNA constructs, evaluated as a function of the dose and number of doses administered. Splenocytes from vaccinated mice were analyzed in IFN-γ ELISpot assay detecting RBD/spike specific cellular responses. Briefly, the animals were sacrificed at days shown in Table 2 and the spleens were harvested aseptically. The spleens were mashed, cell suspensions were incubated with 1×ACK buffer, washed and re-suspended to a cell concentration of 6×10⁶ cells. In some experiments, CD4+ or CD8+ T cell populations were depleted from the total splenocyte population using the Dynabead (catalog no. 11447D or 11445D; Thermo Fischer Scientific) magnetic bead system according to the manufacturer's recommended procedures. Cells were then re-suspended in complete medium at 6×10⁶ cells/ml for the ELISpot assay. Depletion was confirmed by flow cytometry analysis. Furthermore, the cells were plated in triplicates (6×10⁵ cells/well) and stimulated with 2 μg/ml of RBD/spike peptide pools (Tables 3 and 4 below) or individual peptides (15-mer peptides overlapping by 12 amino acids spanning the entire RBD, 61 peptides in total, or the entire spike protein, 296 peptides in total) for 24 h. No-peptide-stimulation was used as negative control. The stimulated splenocytes were analysed for IFN-γ responses using the IFN-γ ELISpot Plus kit (Mabtech AB, Sweden). Spot-forming cells were measured in a CTL ELISpot reader, ImmunoSpot 5.0.3 from Cellular Technology. Results are shown as the mean number of IFN-γ+spots/10⁶ splenocytes. Tables 3: RBD pools and peptides

| Pool ID | Composition |
|---|---|
| RBD pool 1 | RBD 1, 2, 3, 4, 5, 6, 7, 8, 9, 10 |
| RBD pool 2 | RBD 11, 12, 13, 14, 15, 16, 17, 18, 19, 24 |
| RBD pool 3 | RBD 20, 21, 22, 23, 25, 26, 27, 28, 29, 30 |
| RBD pool 4 | RBD 31, 32, 33, 34, 35, 36, 37, 38, 39, 40 |
| RBD pool 5 | RBD 41, 42, 43, 44, 45, 46, 47, 48, 49, 50, 51 |
| RBD pool 6 | RBD 52, 53, 54, 55, 56, 57, 58, 59, 60, 61 |

TABLES 4
Spike pools and peptides

| RBD | |
|---|---|
| Peptide | |
| No./ | |
| SEQ ID | |
| NO: | Sequence |
| 1/445 | NITNLCPFGEVFNAT |
| 2/446 | NLCPFGEVFNATRFA |
| 3/447 | PFGEVFNATRFASVY |
| 4/448 | EVFNATRFASVYAWN |
| 5/449 | NATRFASVYAWNRKR |
| 6/450 | RFASVYAWNRKRISN |
| 7/451 | SVYAWNRKRISNCVA |
| 8/452 | AWNRKRISNCVADYS |
| 9/453 | RKRISNCVADYSVLY |
| 10/454 | ISNCVADYSVLYNSA |
| 11/455 | CVADYSVLYNSASFS |
| 12/456 | DYSVLYNSASFSTFK |
| 13/457 | VLYNSASFSTFKCYG |
| 14/458 | NSASFSTFKCYGVSP |
| 15/459 | SFSTFKCYGVSPTKL |
| 16/460 | TFKCYGVSPTKLNDL |
| 17/461 | CYGVSPTKLNDLCFT |
| 18/462 | VSPTKLNDLCFTNVY |
| 19/463 | TKLNDLCFTNVYADS |
| 20/464 | NDLCFTNVYADSFVI |
| 21/465 | CFTNVYADSFVIRGD |
| 22/466 | NVYADSFVIRGDEVR |
| 23/467 | ADSFVIRGDEVRQIA |
| 24/468 | FVIRGDEVRQIAPGQ |
| 25/469 | RGDEVRQIAPGQTGK |
| 26/470 | EVRQIAPGQTGKIAD |
| 27/471 | QIAPGQTGKIADYNY |
| 28/472 | PGQTGKIADYNYKLP |
| 29/473 | TGKIADYNYKLPDDF |
| 30/474 | IADYNYKLPDDFTGC |
| 31/475 | YNYKLPDDFTGCVIA |
| 32/476 | KLPDDFTGCVIAWNS |
| 33/477 | DDFTGCVIAWNSNNL |
| 34/478 | TGCVIAWNSNNLDSK |
| 35/479 | VIAWNSNNLDSKVGG |
| 36/480 | WNSNNLDSKVGGNYN |
| 37/481 | NNLDSKVGGNYNYLY |
| 38/482 | DSKVGGNYNYLYRLF |
| 39/483 | VGGNYNYLYRLFRKS |
| 40/484 | NYNYLYRLFRKSNLK |
| 41/485 | YLYRLFRKSNLKPFE |
| 42/486 | RLFRKSNLKPFERDI |
| 43/487 | RKSNLKPFERDISTE |
| 44/488 | NLKPFERDISTEIYQ |
| 45/489 | PFERDISTEIYQAGS |
| 46/490 | RDISTEIYQAGSTPC |
| 47/491 | STEIYQAGSTPCNGV |
| 48/492 | IYQAGSTPCNGVEGF |
| 49/493 | AGSTPCNGVEGFNCY |
| 50/494 | TPCNGVEGFNCYFPL |
| 51/495 | NGVEGFNCYFPLQSY |
| 52/496 | EGFNCYFPLQSYGFQ |
| 53/497 | NCYFPLQSYGFQPTN |
| 54/498 | FPLQSYGFQPTNGVG |
| 55/499 | QSYGFQPTNGVGYQP |
| 56/500 | GFQPTNGVGYQPYRV |
| 57/501 | PTNGVGYQPYRVVVL |
| 58/502 | GVGYQPYRVVVLSFE |
| 59/503 | YQPYRVVVLSFELLH |
| 60/504 | YRVVVLSFELLHAPA |
| 61/505 | VVLSFELLHAPAT |

| Pool ID | Composition |
|---|---|
| Spike pool 1 | Peptides 1-12 |
| Spike pool 2 | Peptides 13-24 |
| Spike pool 3 | Peptides 25-35, 37 |
| Spike pool 4 | Peptides 38-49 |
| Spike pool 5 | Peptides 50-61 |
| Spike pool 6 | Peptides 62-73 |
| Spike pool 7 | Peptides 74-80, 82-84, 87, 88 |
| Spike pool 8 | Peptides 85, 86, 89-93, 98-101, 108 |
| Spike pool 9 | Peptides 94-97, 102-105, 109-112 |
| Spike pool 10 | Peptides 106, 107, 113-122 |
| Spike pool 11 | Peptides 123-134 |
| Spike pool 12 | Peptides 135-146 |
| Spike pool 13 | Peptides 147-156, 159-161 |
| Spike pool 14 | Peptides 156-158, 162-169, 172 |
| Spike pool 15 | Peptides 170, 171, 173, 174, 177-180, |
| | 186, 188, 189, 191 |
| Spike pool 16 | Peptides 175, 176, 181, 182, 193-195, 197-200 |
| Spike pool 17 | Peptides 183-185, 187, 190, 192, 201-206 |
| Spike pool 18 | Peptides 196, 207-217 |
| Spike pool 19 | Peptides 218-230 |
| Spike pool 20 | Peptides 231-238, 240-242, 244, 245 |
| Spike pool 21 | Peptides 23, 243, 246-256 |
| Spike pool 22 | Peptides 257-269 |
| Spike pool 23 | Peptides 270-276, 278, 280-283 |
| Spike pool 24 | Peptides 284-287, 289-296 |

| Peptide | |
|---|---|
| no./SEQ | |
| ID NO: | Sequence |
| 1/506 | VNLTTRTQLPPAYTN |
| 2/507 | TRTQLPPAYTNSFTR |
| 3/508 | LPPAYTNSFTRGVYY |
| 4/509 | YTNSFTRGVYYPDKV |
| 5/510 | FTRGVYYPDKVFRSS |
| 6/511 | VYYPDKVFRSSVLHS |
| 7/512 | DKVFRSSVLHSTQDL |
| 8/513 | RSSVLHSTQDLFLPF |
| 9/514 | LHSTQDLFLPFFSNV |
| 10/515 | QDLFLPFFSNVTWFH |
| 11/516 | LPFFSNVTWFHAIHV |
| 12/517 | SNVTWFHAIHVSGTN |
| 13/518 | WFHAIHVSGTNGTKR |
| 14/519 | IHVSGTNGTKRFDNP |
| 15/520 | GTNGTKRFDNPVLPF |
| 16/521 | TKRFDNPVLPFNDGV |
| 17/522 | DNPVLPFNDGVYFAS |
| 18/523 | LPFNDGVYFASTEKS |
| 19/524 | DGVYFASTEKSNIIR |
| 20/525 | FASTEKSNIIRGWIF |
| 21/526 | EKSNIIRGWIFGTTL |
| 22/527 | IIRGWIFGTTLDSKT |
| 23/528 | WIFGTTLDSKTQSLL |
| 24/529 | TTLDSKTQSLLIVNN |
| 25/530 | SKTQSLLIVNNATNV |
| 26/531 | SLLIVNNATNVVIKV |
| 27/532 | VNNATNVVIKVCEFQ |
| 28/533 | TNVVIKVCEFQFCND |
| 29/534 | IKVCEFQFCNDPFLG |
| 30/535 | EFQFCNDPFLGVYYH |
| 31/536 | CNDPFLGVYYHKNNK |
| 32/537 | FLGVYYHKNNKSWME |
| 33/538 | YYHKNNKSWMESEFR |
| 34/539 | NNKSWMESEFRVYSS |
| 35/540 | WMESEFRVYSSANNC |
| 36/541 | EFRVYSSANNCTFEY |
| 37/542 | YSSANNCTFEYVSQP |
| 38/543 | NNCTFEYVSQPFLMD |
| 39/544 | FEYVSQPFLMDLEGK |
| 40/545 | SQPFLMDLEGKQGNF |
| 41/546 | LMDLEGKQGNFKNLR |
| 42/547 | EGKQGNFKNLREFVF |
| 43/548 | GNFKNLREFVFKNID |
| 44/549 | NLREFVFKNIDGYFK |
| 45/550 | FVFKNIDGYFKIYSK |
| 46/551 | NIDGYFKIYSKHTPI |
| 47/552 | YFKIYSKHTPINLVR |
| 48/553 | YSKHTPINLVRDLPQ |
| 49/554 | TPINLVRDLPQGFSA |
| 50/555 | LVRDLPQGFSALEPL |
| 51/556 | LPQGFSALEPLVDLP |
| 52/557 | FSALEPLVDLPIGIN |
| 53/558 | EPLVDLPIGINITRF |
| 54/559 | DLPIGINITRFQTLL |
| 55/560 | GINITRFQTLLALHR |
| 56/561 | TRFQTLLALHRSYLT |
| 57/562 | TLLALHRSYLTPGDS |
| 58/563 | LHRSYLTPGDSSSGW |
| 59/564 | YLTPGDSSSGWTAGA |
| 60/565 | GDSSSGWTAGAAAYY |
| 61/566 | SGWTAGAAAYYVGYL |
| 62/567 | AGAAAYYVGYLQPRT |
| 63/568 | AYYVGYLQPRTFLLK |
| 64/569 | GYLQPRTFLLKYNEN |
| 65/570 | PRTFLLKYNENGTIT |
| 66/571 | LLKYNENGTITDAVD |
| 67/572 | NENGTITDAVDCALD |
| 68/573 | TITDAVDCALDPLSE |
| 69/574 | AVDCALDPLSETKCT |
| 70/575 | ALDPLSETKCTLKSF |
| 71/576 | LSETKCTLKSFTVEK |
| 72/577 | KCTLKSFTVEKGIYQ |
| 73/578 | KSFTVEKGIYQTSNF |
| 74/579 | VEKGIYQTSNFRVQP |
| 75/580 | IYQTSNFRVQPTESI |
| 76/581 | SNFRVQPTESIVRFP |
| 77/582 | VQPTESIVRFPNITN |
| 78/583 | ESIVRFPNITNLCPF |
| 79/584 | RFPNITNLCPFGEVF |
| 80/585 | ITNLCPFGEVFNATR |
| 81/586 | CPFGEVFNATRFASV |
| 82/587 | EVFNATRFASVYAWN |
| 83/588 | ATRFASVYAWNRKRI |
| 84/589 | ASVYAWNRKRISNCV |
| 85/590 | AWNRKRISNCVADYS |
| 86/591 | KRISNCVADYSVLYN |
| 87/592 | NCVADYSVLYNSASF |
| 88/593 | DYSVLYNSASFSTFK |
| 89/594 | LYNSASFSTFKCYGV |
| 90/595 | ASFSTFKCYGVSPTK |
| 91/596 | TFKCYGVSPTKLNDL |
| 92/597 | YGVSPTKLNDLCFTN |
| 93/598 | PTKLNDLCFTNVYAD |
| 94/599 | NDLCFTNVYADSFVI |
| 95/600 | FTNVYADSFVIRGDE |
| 96/601 | YADSFVIRGDEVRQI |
| 97/602 | FVIRGDEVRQIAPGQ |
| 98/603 | GDEVRQIAPGQTGKI |
| 99/604 | RQIAPGQTGKIADYN |
| 100/605 | PGQTGKIADYNYKLP |
| 101/606 | GKIADYNYKLPDDFT |
| 102/607 | DYNYKLPDDFTGCVI |
| 103/608 | KLPDDFTGCVIAWNS |
| 104/609 | DFTGCVIAWNSNNLD |
| 105/610 | CVIAWNSNNLDSKVG |
| 106/611 | WNSNNLDSKVGGNYN |
| 107/612 | NLDSKVGGNYNYLYR |
| 108/613 | KVGGNYNYLYRLFRK |
| 109/614 | NYNYLYRLFRKSNLK |
| 110/615 | LYRLFRKSNLKPFER |
| 111/616 | FRKSNLKPFERDIST |
| 112/617 | NLKPFERDISTEIYQ |
| 113/618 | FERDISTEIYQAGST |
| 114/619 | ISTEIYQAGSTPCNG |
| 115/620 | IYQAGSTPCNGVEGF |
| 116/621 | GSTPCNGVEGFNCYF |
| 117/622 | CNGVEGFNCYFPLQS |
| 118/623 | EGFNCYFPLQSYGFQ |
| 119/624 | CYFPLQSYGFQPTNG |
| 120/625 | LQSYGFQPTNGVGYQ |
| 121/626 | GFQPTNGVGYQPYRV |
| 122/627 | TNGVGYQPYRVVVLS |
| 123/628 | GYQPYRVVVLSFELL |
| 124/629 | YRVVVLSFELLHAPA |
| 125/630 | VLSFELLHAPATVCG |
| 126/631 | ELLHAPATVCGPKKS |
| 127/632 | APATVCGPKKSTNLV |
| 128/633 | VCGPKKSTNLVKNKC |
| 129/634 | KKSTNLVKNKCVNFN |
| 130/635 | NLVKNKCVNFNFNGL |
| 131/636 | NKCVNFNFNGLTGTG |
| 132/637 | NFNFNGLTGTGVLTE |
| 133/638 | NGLTGTGVLTESNKK |
| 134/639 | GTGVLTESNKKFLPF |
| 135/640 | LTESNKKFLPFQQFG |
| 136/641 | NKKFLPFQQFGRDIA |
| 137/642 | LPFQQFGRDIADTTD |
| 138/643 | QFGRDIADTTDAVRD |
| 139/644 | DIADTTDAVRDPQTL |
| 140/645 | TTDAVRDPQTLEILD |
| 141/646 | VRDPQTLEILDITPC |
| 142/647 | QTLEILDITPCSFGG |
| 143/648 | ILDITPCSFGGVSVI |
| 144/649 | TPCSFGGVSVITPGT |
| 145/650 | FGGVSVITPGTNTSN |
| 146/651 | SVITPGTNTSNQVAV |
| 147/652 | PGTNTSNQVAVLYQD |
| 148/653 | TSNQVAVLYQDVNCT |
| 149/654 | VAVLYQDVNCTEVPV |
| 150/655 | YQDVNCTEVPVAIHA |
| 151/656 | NCTEVPVAIHADQLT |
| 152/657 | VPVAIHADQLTPTWR |
| 153/658 | IHADQLTPTWRVYST |
| 154/659 | QLTPTWRVYSTGSNV |
| 155/660 | TWRVYSTGSNVFQTR |
| 156/661 | YSTGSNVFQTRAGCL |
| 157/662 | SNVFQTRAGCLIGAE |
| 158/663 | QTRAGCLIGAEHVNN |
| 159/664 | GCLIGAEHVNNSYEC |
| 160/665 | GAEHVNNSYECDIPI |
| 161/666 | VNNSYECDIPIGAGI |
| 162/667 | YECDIPIGAGICASY |
| 163/668 | IPIGAGICASYQTQT |
| 164/669 | AGICASYQTQTNSPR |
| 165/670 | ASYQTQTNSPRRARS |
| 166/671 | TQTNSPRRARSVASQ |
| 167/672 | SPRRARSVASQSIIA |
| 168/673 | ARSVASQSIIAYTMS |
| 169/674 | ASQSIIAYTMSLGAE |
| 170/675 | IIAYTMSLGAENSVA |
| 171/676 | TMSLGAENSVAYSNN |
| 172/677 | GAENSVAYSNNSIAI |
| 173/678 | SVAYSNNSIAIPTNF |
| 174/679 | SNNSIAIPTNFTISV |
| 175/680 | IAIPTNFTISVTTEI |
| 176/681 | TNFTISVTTEILPVS |
| 177/682 | ISVTTEILPVSMTKT |
| 178/683 | TEILPVSMTKTSVDC |
| 179/684 | PVSMTKTSVDCTMYI |
| 180/685 | TKTSVDCTMYICGDS |
| 181/686 | VDCTMYICGDSTECS |
| 182/687 | MYICGDSTECSNLLL |
| 183/688 | GDSTECSNLLLQYGS |
| 184/689 | ECSNLLLQYGSFCTQ |
| 185/690 | LLLQYGSFCTQLNRA |
| 186/691 | YGSFCTQLNRALTGI |
| 187/692 | CTQLNRALTGIAVEQ |
| 188/693 | NRALTGIAVEQDKNT |
| 189/694 | TGIAVEQDKNTQEVF |
| 190/695 | VEQDKNTQEVFAQVK |
| 191/696 | KNTQEVFAQVKQIYK |
| 192/697 | EVFAQVKQIYKTPPI |
| 193/698 | QVKQIYKTPPIKDFG |
| 194/699 | IYKTPPIKDFGGFNF |
| 195/700 | PPIKDFGGFNFSQIL |
| 196/701 | DFGGFNFSQILPDPS |
| 197/702 | FNFSQILPDPSKPSK |
| 198/703 | QILPDPSKPSKRSFI |
| 199/704 | DPSKPSKRSFIEDLL |
| 200/705 | PSKRSFIEDLLFNKV |
| 201/706 | SFIEDLLFNKVTLAD |
| 202/707 | DLLFNKVTLADAGFI |
| 203/708 | NKVTLADAGFIKQYG |
| 204/709 | LADAGFIKQYGDCLG |
| 205/710 | GFIKQYGDCLGDIAA |
| 206/711 | QYGDCLGDIAARDLI |
| 207/712 | CLGDIAARDLICAQK |
| 208/713 | IAARDLICAQKFNGL |
| 209/714 | DLICAQKFNGLTVLP |
| 210/715 | AQKFNGLTVLPPLLT |
| 211/716 | NGLTVLPPLLTDEMI |
| 212/717 | VLPPLLTDEMIAQYT |
| 213/718 | LLTDEMIAQYTSALL |
| 214/719 | EMIAQYTSALLAGTI |
| 215/720 | QYTSALLAGTITSGW |
| 216/721 | ALLAGTITSGWTFGA |
| 217/722 | GTITSGWTFGAGAAL |
| 218/723 | SGWTFGAGAALQIPF |
| 219/724 | FGAGAALQIPFAMQM |
| 220/725 | AALQIPFAMQMAYRF |
| 221/726 | IPFAMQMAYRFNGIG |
| 222/727 | MQMAYRFNGIGVTQN |
| 223/728 | YRFNGIGVTQNVLYE |
| 224/729 | GIGVTQNVLYENQKL |
| 225/730 | TQNVLYENQKLIANQ |
| 226/731 | LYENQKLIANQFNSA |
| 227/732 | QKLIANQFNSAIGKI |
| 228/733 | ANQFNSAIGKIQDSL |
| 229/734 | NSAIGKIQDSLSSTA |
| 230/735 | GKIQDSLSSTASALG |
| 231/736 | DSLSSTASALGKLQD |
| 232/737 | STASALGKLQDVVNQ |
| 233/738 | ALGKLQDVVNQNAQA |
| 234/739 | LQDVVNQNAQALNTL |
| 235/740 | VNQNAQALNTLVKQL |
| 236/741 | AQALNTLVKQLSSNF |
| 237/742 | NTLVKQLSSNFGAIS |
| 238/743 | KQLSSNFGAISSVLN |
| 239/744 | SNFGAISSVLNDILS |
| 240/745 | AISSVLNDILSRLDK |
| 241/746 | VLNDILSRLDKVEAE |
| 242/747 | ILSRLDKVEAEVQID |
| 243/748 | LDKVEAEVQIDRLIT |
| 244/749 | EAEVQIDRLITGRLQ |
| 245/750 | QIDRLITGRLQSLQT |
| 246/751 | LITGRLQSLQTYVTQ |
| 247/752 | RLQSLQTYVTQQLIR |
| 248/753 | LQTYVTQQLIRAAEI |
| 249/754 | VTQQLIRAAEIRASA |
| 250/755 | LIRAAEIRASANLAA |
| 251/756 | AEIRASANLAATKMS |
| 252/757 | ASANLAATKMSECVL |
| 253/758 | LAATKMSECVLGQSK |
| 254/759 | KMSECVLGQSKRVDF |
| 255/760 | CVLGQSKRVDFCGKG |
| 256/761 | QSKRVDFCGKGYHLM |
| 257/762 | VDFCGKGYHLMSFPQ |
| 258/763 | GKGYHLMSFPQSAPH |
| 259/764 | HLMSFPQSAPHGVVF |
| 260/765 | FPQSAPHGVVFLHVT |
| 261/766 | APHGVVFLHVTYVPA |
| 262/767 | VVFLHVTYVPAQEKN |
| 263/768 | HVTYVPAQEKNFTTA |
| 264/769 | VPAQEKNFTTAPAIC |
| 265/770 | EKNFTTAPAICHDGK |
| 266/771 | TTAPAICHDGKAHFP |
| 267/772 | AICHDGKAHFPREGV |
| 268/773 | DGKAHFPREGVFVSN |
| 269/774 | HFPREGVFVSNGTHW |
| 270/775 | EGVFVSNGTHWFVTQ |
| 271/776 | VSNGTHWFVTQRNFY |
| 272/777 | THWFVTQRNFYEPQI |
| 273/778 | VTQRNFYEPQIITTD |
| 274/779 | NFYEPQIITTDNTFV |
| 275/780 | PQIITTDNTFVSGNC |
| 276/781 | TTDNTFVSGNCDVVI |
| 277/782 | TFVSGNCDVVIGIVN |
| 278/783 | GNCDVVIGIVNNTVY |
| 279/784 | VVIGIVNNTVYDPLQ |
| 280/785 | IVNNTVYDPLQPELD |
| 281/786 | TVYDPLQPELDSFKE |
| 282/787 | PLQPELDSFKEELDK |
| 283/788 | ELDSFKEELDKYFKN |
| 284/789 | FKEELDKYFKNHTSP |
| 285/790 | LDKYFKNHTSPDVDL |
| 286/791 | FKNHTSPDVDLGDIS |
| 287/792 | TSPDVDLGDISGINA |
| 288/793 | VDLGDISGINASVVN |
| 289/794 | DISGINASVVNIQKE |
| 290/795 | INASVVNIQKEIDRL |
| 291/796 | VVNIQKEIDRLNEVA |
| 292/797 | QKEIDRLNEVAKNLN |
| 293/798 | DRLNEVAKNLNESLI |
| 294/799 | EVAKNLNESLIDLQE |
| 295/800 | NLNESLIDLQELGKY |
| 296/801 | SLIDLQELGKYEQYI |

Overall, the VB10.COV2 constructs all induced strong, dose-dependent T cell responses after vaccination, which increased over time. The responses were dominated by CD8⁺ T cells and accompanied by significant, but weaker CD4+ T cell responses.

Strong T cell responses against the RBD domain of SARS-CoV-2 were detected in spleens from mice vaccinated with one or two doses of both 2.5 μg or 25 μg VB2049 (FIG. 59). Depending on dose level and the number of doses, the response ranged from ˜1800 to 6000 SFU per 10⁶ cells in splenocytes sampled 2 weeks after 1^{st} dose or 1st week post-boost-vaccination at day 21 and stimulated separately with 6 peptide pools spanning RBD. The response was strong already 14 days post 1^{st} vaccination even with a low dose (2.5 μg DNA) and was boosted by day 28 in groups receiving a 2^{nd} vaccination at day 21 (in a dose-dependent manner, FIG. 59).

The epitopes recognized by the T cells by stimulating with individual 15-mers overlapping with 12 amino acids in splenocytes depleted for either CD4 or CD8 T cell populations were characterised. Strong (up to ˜4000 SFU/10⁶ cells) CD8⁺ T cell responses against 9 peptides were observed. RBD-specific CD4⁺ responses were also detected against 7 peptides, but of a lower magnitude and fewer epitopes (up to ˜1000 SFU/10⁶ cells) (FIGS. 60A and 60E). The amino acid sequence of the overlapping peptides indicated a reactivity against 4 distinct MHC class I-restricted epitopes and 3 MHC class II-restricted epitopes (FIG. 60C) in RBD.

The kinetics of the early T cell responses induced by either 1 dose (day 0) or 2 rapid doses (day 0+7) of VB2060 was examined. Vaccination with 1×25 μg of VB2060 induced T cell responses as early as at day 7 (˜550 spots per 10⁶ splenocytes) with a peak response at day 14 (˜2750 spots per 10⁶ splenocytes. An additional boost vaccination at day 7 did not increase the T cell responses compared to the single dose vaccine regime (FIG. 61A). In a separate experiment, the T cell responses were still found to persist for at least 90 days after vaccination with 50 μg of VB2060 (˜5000 SFU/10⁶ splenocytes), with a strong boost effect at day 99; 10 days after a booster dose at day 89 (˜20 000 SFU/10⁶ splenocytes) (FIG. 62). When comparing T cell responses induced by two doses of 2.5 μg 7 days post boost vaccination at day 21 of either VB2060, VB2049 or VB2059, VB2049 induced stronger responses than VB2060 and significantly stronger than VB2059 (˜3800 versus ˜2600 SFU/10⁶ cells versus ˜1000 SFU/10⁶ cells, respectively) (FIG. 61B). This finding clearly shows that the MIP1α targeting unit (VB2049 and VB2060) is superior compared to the anti-mouse MHCII scFv targeting unit (VB2059) at eliciting high levels of RBD-specific T cell responses with the VB10.COV2 vaccines comprising such targeting unit.

An IFN-γ ELISpot assay was performed on fresh splenocytes from mice vaccinated with VB2065 and VB2071 DNA vaccine containing the spike protein to evaluate the vaccine dose T cell response effect. As predicted, both VB2065 and VB2071 induced a broader, stronger total T cell response than VB2049, VB2060 and VB2059 due to the larger antigen (FIG. 63). The animals were sacrificed on day 28, i.e. 7 days post boost-vaccination at day 21. The spleens were harvested and the splenocytes isolated before stimulation with spike peptide pools. CD4 and CD8 cell populations were depleted in the splenocyte total cell population by using beads to evaluate the specific CD8+ and CD4+ RBD specific immune responses. Both VB2065 and VB2071 induced strong, CD8+ dominating T cell responses, accompanied by broad, weaker CD4+ responses.

A single vaccination and dose-dependent early T cell response kinetics induced by VB2129, containing the long RBD domain with the 3 mutations from the South African virus variant, was examined. Vaccination with 1×1.0, 6.25, 12.5 or 25 μg of VB2129 induced T cell responses as early as at day 7 at a low dose (˜500 spots per 10⁶ splenocytes for 6.25 μg dose) with a significant increase in the response by day 14 (˜2750 spots per 10⁶ splenocytes for 25 μg dose) (FIG. 63B). The data in this experiment is comparable to the data for VB2060 in similar experiments (FIG. 61).

### Example 7: VB10.COV2 DNA Vaccines Induce Predominantly Th1 Response Against RBD/Spike Protein in Mice

The purpose of this study was to analyze the Th1/2 profile of the cellular response elicited in mice after two doses of VB10.COV2 DNA vaccine.

The animals were vaccinated with two doses of 2.5 μg of VB10.COV2 vaccibody DNA constructs VB2049, VB2059 and VB2060 or two doses of 50 μg VB2065 and VB2071 on days 0 and 21 and sacrificed 28 days post primary vaccination. The spleens were removed aseptically, mashed to obtain cell suspensions with splenocytes, and 1×ACK buffer was used to remove erythrocytes. The splenocytes were than washed, plated (1.5×10⁶ cells/well in 24 well plate) and stimulated for 24 h with 2 μg/ml of RBD peptide pools or selected spike peptide pools (Tables 3 and 4). Cell culture supernatant was harvested and analysed for cytokine presence. In short, 50 μl of the cell culture supernatant was used in using ProcartaPlex Immunoassay as described in the supplier's protocol (Thermo Fisher). Presence of IFN-γ, TNF-α and IL-12p70 in the supernatant defined Th1 response. The Th2 response was defined through production of IL-4 and IL-5 and partially through presence of IL-6.

Characterization of the Th1 (IFNγ, TNFα, IL-12) and Th2 (IL-4, IL-5) cytokines in cell culture supernatant of splenocytes from vaccinated mice re-stimulated with RBD or spike peptide pools showed that for VB2060, the response was IFNγ and TNFα dominated and minor quantities of IL-6, IL-12 p70, IL-4 or IL-5 were detected. This indicates that T cell responses showed strong Th1 bias when characterized one month after vaccination, while the Th2 responses were minimal. For VB2049 and VB2059, minor IL-6 responses were observed and no significant responses for IL-12 p70, IL-4 or IL-5 (FIG. 64A).

The same pattern was observed for VB2065 and VB2071 (spike), where IL-6 was to some extent detected for one of the pooled peptides (peptides 5 and 6) (FIG. 64B).

Overall, this is consistent with a Th1-biased response induced by the vaccines. A Th1-biased response is preferable to avoid potential vaccine-related enhancement of disease which has been observed for some SARS-CoV vaccines; likely involving a Th2-biased response. Example 8: RBD specific cell mediated immune response to VB10.COV2 DNA vaccines

### Example 8: RBD Specific Cell Mediated Immune Response to VB10.COV2 DNA Vaccines

The purpose of this study was to evaluate T cell responses, on a single cell level, in mice vaccinated with two doses of VB10.COV2 vaccibody DNA constructs. The multi flow cytometry was developed to assess T cell subsets in mice vaccinated with VB2049 or VB2060 DNA vaccines. The T cells were defined with CD3, CD4, CD8 and γδ TCR lineage markers. The in-depth analysis of IFN-γ, TNF-α, IL-2, IL-4, IL-17 and FoxP3 expression allowed evaluation of T helper (Th) 1 and 2 type responses, Th17, and regulatory T cells (Treg).

BALB/c mice were vaccinated with either low (2.5 μg), medium (25 μg) or high (50 μg) dose VB2049 or VB2060 DNA vaccine one-, two- or three times as described in Table 2. Splenocytes from vaccinated mice were isolated as previously described. The splenocytes were than washed, plated (2×10⁶ cells/well in 24 well plate) and stimulated for 16 h with 2 μg/ml of RBD peptide. For detection of cytokines with flow cytometry 1× monensin and 1× brefeldin were added to the wells during the incubation. Following stimulation with RBD peptide pools, cells were harvested, washed, and stained with viability die, followed by staining with extracellular antibodies (anti-CD3, anti-CD4, anti-CD8 and gdTCR), fixed and permeabilized, and then stained for detection of TNFα, IFNγ, IL-2 (if assessed), IL-4, IL-17 and FoxP3. The stained cells were run in BD FACSymphony A5 and analysed using FlowJo software.

The RBD stimulated mice splenocyte T cells were defined through exclusion of dead cells, doublets and CD3-non-T cells (FIG. 65A-D). CD3+ T cells were then analysed for presence of γδ TCR T cells, and these cells were further removed from the analysis (FIG. 65E). The remainder of the T cells was then examined for CD4 and CD8 makers, thus defining CD4+ and CD8+ T cells (FIG. 65F). Both populations were examined for individual expression of IFN-γ, TNF-α, IL-2, IL-4, IL-17 or FoxP3 and gates were set to define positive cells. These positive cells were further analysed using Boolean gating algorithm in FlowJo software, calculating all possible combinations of cytokines produced by each cell, thus allowing analysis of multifunctional T cells on a single cell level.

Flow cytometry analysis of T cells in VB2060-vaccinated mice (low dose) showed responses by CD4+ T cells and CD8+ T cells to RBD stimulation (FIGS. 66A and 66B). The CD4+ RBD specific T cells produced IFN-γ, TNFα, or a combination of these cytokines, a cytokine profile typical for Th1 responses. Presence of other markers like IL-4 (Th2 polarization), IL-17 (Th17) and FoxP3 (Treg) was also seen in a population of CD4+ T cells. Analysis of CD8+ T cells showed responses dominated by IFN-γ, TNFα or a combination of the 2. A minor population of CD8+ T cells also expressed IL-17 and FoxP3. IL-2 expression was not examined.

The same analysis of RBD specific T cells in VB2049 vaccinated mice (low dose) showed responses of CD4+ T cells and CD8+ T cells (FIGS. 66C and 66D). The CD4+ T cells expressed IFN-γ, TNF-α, or a combination of the two cytokines. A portion of CD4+ T cells also expressed IL-4, a Th2 cell cytokine and IL-17, a Th17 cytokine, thus, revealing a mixture of Th1, Th2, Th17 and a Treg responses. CD8+ T cell responses, however, were dominated by combined production of IFN-γ and TNF-α while the remainder of RBD specific cells produced one of these cytokines.

Thus, analysis of RBD specific CD4+ T cells in VB10.COV2-vaccinated mice (low dose) showed Th1 responses (defined by combined IFN-γ/TNFα production), and a mixture of Th2, Th17 and Treg responses. The CD8+ T cell were dominated by IFN-γ and TNFα presence, indicating that VB10.COV2 induces a cytotoxic T cell response specific for SARS-CoV-2.

To examine durability of T cell responses in mice vaccinated with VB2060 (medium and high dose), splenocytes were analysed on day 90. A dose dependent response was observed which was dominated by polyfunctional CD4+ T cells that produced IFN-γ, TNFα, IL-2 or combination of these cytokines (FIG. 67). Only a minor population of CD4+ T cells produced IL-17. Similarly, CD8+ T cell responses were also dose dependent and dominated by IFN-γ, TNFα (FIG. 68). These results confirmed the initial findings in mice vaccinated with a low dose of VB2060, i.e. that vaccination with VB2060 DNA vaccine elicits a combination of Th1 and Th17 T cell responses, and a cytotoxic T cell response specific for SARS-CoV-2. The results show a dose depended effect on T cells which persists for 90 days after the initial vaccination.

The animals were boost vaccinated on day 89 and subsequent T cell responses were analyzed on day 99. The CD4+ T cells produced IFN-γ and TNFα as previously observed. These cells also produced increased amounts of IL-2 indicating T cell survival and proliferation. A portion of CD4+ T cells also produces IL-17 (FIG. 69). The CD8+ T cells responses, similarly to the previous findings, were dominated by IFN-γ and to some extent TNFα (FIG. 70). In conclusion these data show that VB2060 DNA vaccine induces durable, Th1, Th17 and cytotoxic T cell responses which last for at least 100 days.

In addition, the early T cell responses in draining lymph nodes were evaluated on day 7 and day 28 after the first vaccination, i.e. seven days following the vaccination and seven days following the boost vaccination. Cells from the draining lymph nodes were stimulated with RBD peptides and then analysed using multi-color flow cytometry. We evaluated CD4⁺ and CD8⁺ T cells and a subset of CD8⁺ T cells called resident memory T cells (Trm). To evaluate activation status and type of response we analysed expression of TNF-α, IFN-γ, IL-2 and granzyme B (FIG. 71 and FIG. 72).

Seven days after the vaccination, we analysed mice vaccinated with 25 μg VB2060 and compared to the control group (PBS). We observed strong CD8 T cell responses defined by presence of granzyme B. The Trm subset of CD8 T cell mainly expressed IFN-γ alone or in combination with granzyme B, indicating cytotoxic responses to RBD peptide (FIG. 71 A-C). At the same time point, the CD4⁺ T cell produced IL-2, TNF-α, or a combination of the two cytokines.

Seven days post the boost vaccination, we evaluated T cell responses in mice vaccinated with 3.0 μg, 6.25 μg, 12.5 μg and 25 μg VB2060. This analysis revealed dose dependent strong CD8⁺ T cell responses accompanied by production of granzyme B, TNF-α IFN-γ or a combination of these. Similar results were observed for resident memory T cells (FIGS. 71E and F); additionally, this subset was increased in lymph nodes after the boost vaccination (FIG. 72B). After the boost vaccination, CD4+ T cells produced TNF-α, IFN-γ, IL-2 or a combination of these cytokines in a dose dependent manner.

Taken together these data show strong dose dependent T cell response dominated by cytotoxic T cells and accompanied by the Th1 polarized CD4⁺ T cells.

### Example 9: Induction of Specific Cellular Responses to Predicted T Cell Epitopes by VB2048 DNA Vaccination

The purpose of this study was to evaluate the cellular immune response against predicted T cell epitopes in splenocytes from mice vaccinated with VB2048 DNA vaccine, evaluated as a function of the dose and number of doses administered.

Splenocytes from vaccinated mice were analyzed in IFN-γ ELISpot assay detecting predicted epitopes specific cellular responses. Briefly, the animals were sacrificed at day 14 or day 28 and the spleens were harvested aseptically. The spleens were mashed, cell suspensions were incubated with 1×ACK buffer, washed and re-suspended to a cell concentration of 6×10⁵ cells. Furthermore, the cells were plated in triplicates (6×10⁵ cells/well) and stimulated with 2 μg/ml of individual peptides (T cell epitopes included in VB2048) for 24 h. No-peptide-stimulation was used as negative control. The stimulated splenocytes were analysed for IFN-γ responses using the IFN-γ ELISpot Plus kit (Mabtech AB, Sweden). Spot-forming cells were measured in a CTL ELISpot reader, ImmunoSpot 5.0.3 from Cellular Technology. Results are shown as the mean number of IFN-γ+spots/10⁶ splenocytes.

Strong T cell responses against the predicted epitopes from multiple SARS-CoV-2 strains were detected in spleens from mice vaccinated with one or two doses of either 2.5 μg or 25 μg VB2048 DNA vaccine. Depending on dose level and number of doses, the response ranged from ˜1500 to 2200 SFC per 10⁶ cells in splenocytes sampled 2 weeks after the first dose or one week post boost-vaccination at day 21. The response was strong already 14 days post first dose, even with a low dose (2.5 μg DNA) and was boosted at day 28 after a second vaccination at day 21 with the high dose (25 μg) (FIG. 73).

In splenocytes depleted for either CD4 or CD8 cell populations, strong (up to ˜2200 SFU/10⁶ cells), CD8+ dominated T cell responses against one dominating peptide (pep08) is observed (FIG. 74). T cell epitope specific CD4+ responses were also detected against 2 predicted peptides (pep02 and pep18), but of a lower magnitude (up to ˜460 SFU/10⁶ cells).

### Example 10: Induction of Specific Cellular Responses to Both Predicted T Cell Epitopes and RBD by DNA Vaccination with Constructs Containing Both T Cell Epitopes and the Long RBD Domain

The purpose of this study was to evaluate the cellular immune response against both predicted T cell epitopes and the RBD domain in splenocytes from mice vaccinated with VB10.COV2 DNA vaccine containing both T cell epitopes and the long RBD domain.

Splenocytes from vaccinated mice were analysed in IFN-γ ELISpot assay detecting predicted epitopes and RBD-specific cellular responses. Briefly, the animals were sacrificed at day 14 and the spleens were harvested aseptically. The spleens were mashed, cell suspensions were incubated with 1×ACK buffer, washed and re-suspended to a cell concentration of 6×10⁵ cells. Furthermore, the cells were plated in triplicates (6×10⁵ cells/well) and stimulated with 2 μg/ml of individual peptides (T cell epitopes included in the respective constructs) and 2 μg/ml of RBD peptide pools (Table 3) for 24 h. No-peptide-stimulation was used as negative control. The stimulated splenocytes were analysed for IFN-γ responses using the IFN-γ ELISpot Plus kit (Mabtech AB, Sweden). Spot-forming cells were measured in a CTL ELISpot reader, ImmunoSpot 5.0.3 from Cellular Technology. Results are shown as the mean number of IFN-γ+spots/10⁶ splenocytes.

Strong T cell responses against the predicted epitopes from multiple SARS-CoV-2 strains were detected in spleens at day 14 from mice vaccinated once with 25 μg of constructs containing either one or three predicted T cell epitopes. VB2097 (3 epitopes+GSAT linker) induced a stronger T cell specific response (˜1250 SFC per 10⁶ cells) than the other constructs comprising 3 epitopes with other linkers. In addition, all constructs were also able to elicit a strong RBD-specific cellular response. VB2097 and VB2087 elicited the strongest responses against RBD at a similar level to VB2060 (FIG. 75).

### Example 11: Induction of Specific Cellular Responses to Both Predicted T Cell Epitopes and RBD by Vaccination with a Vaccine Containing Two VB10.COV2 Constructs

The purpose of this study was to evaluate the cellular immune response against both predicted T cell epitopes and the RBD domain in splenocytes from mice vaccinated with a VB10.COV2 DNA vaccine comprising 2 plasmids, VB2048 (20 T cell epitopes) and VB2049 (short RBD domain), with 12.5 μg of each plasmid.

Splenocytes from vaccinated mice were analysed in IFN-γ ELISpot assay detecting predicted epitopes and RBD-specific cellular responses. Briefly, the animals were sacrificed at day 14 and the spleens were harvested aseptically. The spleens were mashed, cell suspensions were incubated with 1×ACK buffer, washed and re-suspended to a cell concentration of 6×10⁵ cells. Furthermore, the cells were plated in triplicates (6×10⁵ cells/well) and stimulated with 2 μg/ml of 20 individual peptides and 2 μg/ml of RBD peptide pools (Table 3) for 24 h. No-peptide-stimulation was used as negative control. The stimulated splenocytes were analysed for IFN-γ responses using the IFN-γ ELISpot Plus kit (Mabtech AB, Sweden). Spot-forming cells were measured in a CTL ELISpot reader, ImmunoSpot 5.0.3 from Cellular Technology. Results are shown as the mean number of IFN-γ+spots/10⁶ splenocytes.

Strong T cell responses against the predicted epitopes from multiple SARS-CoV-2 strains were detected in spleens at day 14 from mice vaccinated once with a vaccine comprising a pharmaceutically acceptable carrier and 12.5 μg of each plasmid (VB2048 and VB2049). In addition, the vaccine was also able to elicit a strong RBD-specific cellular response. When vaccinating mice with the aforementioned vaccine, the total immune responses against both the predicted T cell epitopes and the RBD domain were similar as when mice were vaccinated with a vaccine containing either of the constructs (i.e. either VB2048 or VCB2049), taking the dose into consideration (FIG. 76).

### Example 12: VB10.COV2 DNA Vaccine VB2060 Stability Data

The purpose of the study was to determine the % supercoil DNA content as a stability indicating parameter after storage of VB10.COV2 DNA vaccine VB2060 at elevated temperature (37° C.) for up to 4 weeks.

0.5 ml of a sterile solution of VB2060 plasmid (3 mg/ml formulated in D-PBS), was filled with into 2 ml, clear type I glass vials (Adelphi/Schott, VCDIN2R), sealed with 13 mm FluroTec® injection stopper (Adelphi/West, 7001-8021/INJ13TB3WRS) and capped with 13 mm white Flip-off overseals (Adelphi/West, 5921-9826/FOT13W5117). The vials were stored upright in an incubator at 37° C. for 4 weeks. Vials were tested for plasmid topology forms by HPLC at the beginning of the study and every week throughout the study. The HPLC method was performed with column TSKgel DNA-NPR (Tosoh Bioscience/Y0064), mobile phase A; 2.4 TRIS-Bas in 1000 ml water and pH adjustment to pH 9 by HCl and mobile phase B; 29.22 g NaCl in 500 ml mobile phase A at flow of 0.75 ml/min. Column temperature was 5° C. and sample injection volume was 1.5 μl. Topology is known as the most sensitive stability indicating parameters for plasmid DNA.

The supercoiling degree of plasmid VB2060 at the start of the study was determined to be approximately 90%. After one week the supercoiling degree had decreased to approximately 80%. In the following weeks, the plasmid topology did not materially change and only showed a minor further degradation. This shows that the VB10.COV2 DNA vaccine VB2060 is highly stable, even when stored at elevated temperatures.

### Summary of the Examples

Using VB2060 as an example, we show, that dimeric molecules are formed (Example 3b). We also show, using VB2060, VB2129 and VB2132 as examples, that monomeric proteins have a molecular weight expected from the size of their constructs, as we start to add several RBD units to the protein (Example 3b).

We show that with vaccibodies comprising an antigenic unit comprising a short form of the SARS-CoV-2 RBD (VB2049), a longer version of the SARS-CoV-2 RBD (VB2060), a longer version of the SARS-CoV-2 RBD with 3 mutations found in the South African variant B.1.351 (VB2129) and the spike protein (VB2065 and VB2071) we induce anti-RBD IgG formation (Example 4). We show, that this response is unchanged, when adding predicted T cell epitopes to the construct (VB2081, VB2082, VB2087, VB2097 and VB2099). Thus, the post-translational modifications to the RBD protein (such as glycosylation and its correct folding which is needed to induce humoral response are unaffected by the addition of further amino acids (Example 4). We show, that the antibodies raised by the vaccibodies are effective in the live virus microneutralization assays (Example 5).

In addition to the raised antibodies, we show, that a cytotoxic T cell response is elicited against the RBD protein by constructs containing the RBD unit, and the RBD unit with the 3 mutations from the South African SARS-CoV-2 virus variant. This response is early (after only seven days) and long lasting (Example 6). We also raise a cytotoxic T cell response against the spike protein (Example 6). The majority of the T cell response is Th1 mediated (Example 7).

Thus, with a single vaccine, we can not only induce the required B cell response to obtain immunity against an infection with a betacoronavirus, but we also obtain T cells to attack an existing infection and help the patient recover.

We have developed a method to predict T cell epitopes from the betacoronavirus, and present those in Example 1. We show, that they elicit a strong T cell response (Example 9). When plasmids of constructs comprising predicted T cell epitopes are co-administered with plasmids of constructs comprising an RBD unit, we see that the T cell responses are similar to those elicited by each plasmid alone (Example 11). When these predicted T cell epitopes are combined with the RBD unit in the same construct, they still raise T cell specific responses, while the T cell specific response of the RBD unit is maintained (Example 10).

Conclusion from the Conducted Experiments

As a conclusion from the Examples 3-12, as detected by ELISA, the expression level was found to vary between high, medium and low expression between the various VB10.COV2 constructs depending on the molecule structure.

The VB10.COV2 vaccines induced rapid and dose-dependent RBD IgG antibody responses that persisted up to at least 3 months after a single dose of the vaccine in mice. For VB2060, neutralizing antibody titers against live virus were detected from day 7 after one dose. All tested dose regimens reached higher or comparable titers to sera from human convalescent COVID-19 patients from day 28. Strong T cell responses were established detected already at day 7 with VB2060 and VB2129, and were subsequently characterized for both VB2049 and VB2060 to be multifunctional CD8+ and Th1 dominated CD4+ T cells. Responses remained at sustained high levels until at least 3 months after a single vaccination, being further strongly boosted by a second vaccination at day 89.

The MIP1α targeting was superior compared to the anti-mouse MHCII scFv targeting at eliciting both stronger anti-RBD IgG responses and higher levels of RBD-specific T cell responses with the VB10.COV2 vaccines.

It has also been shown that eliciting both strong RBD-specific antibody and T cell responses in addition to specific T cell responses against predicted T cell epitopes from the SARS-COV2 genome is feasible with two different strategies. One successful strategy is to combine the predicted T cell epitopes with the RBD domain in the antigenic unit in one VB10.COV2 construct while another successful strategy is to vaccinate with a combination of two separate plasmids (one plasmid containing predicted T cell epitopes and one plasmid containing the RBD domain in the antigenic unit) in one vaccine solution.

These findings, together with simple administration and storage stability even at elevated temperatures, suggest that the VB10.COV2 DNA vaccines are promising future candidates to prevent and treat Covid-19.

### Embodiments A

1. A vaccine comprising an immunologically effective amount of:
   (i) a polynucleotide comprising a nucleotide sequence encoding a targeting unit, a dimerization unit and an antigenic unit, wherein the antigenic unit comprises at least one betacoronavirus epitope; or
   (ii) a polypeptide encoded by the polynucleotide as defined in (i), or
   (iii) a dimeric protein consisting of two polypeptides encoded by the polynucleotide as defined in (i); and
   a pharmaceutically acceptable carrier.
2. The vaccine according to embodiment A1, wherein the vaccine once administered to a human individual elicits a humoral response through generation of antibodies by B cells.
3. The vaccine according to embodiment A1, wherein the vaccine once administered to a human individual elicits a cellular immune response through generation of T cells.
4. The vaccine according to embodiment A1, wherein the vaccine once administered to a human individual elicits a humoral and a cellular immune response.
5. The vaccine according to any of the preceding embodiment A2 to A4, wherein the human individual suffers from a betacoronavirus infection and the vaccine is a therapeutic vaccine.
6. The vaccine according to any of the preceding embodiment A2 to A4, wherein the human individual is a healthy individual and the vaccine is a prophylactic vaccine.
7. The vaccine according to any of the preceding embodiments, wherein the at least one betacoronavirus epitope is a full-length viral surface protein of a betacoronavirus or a part thereof.
8. The vaccine according to embodiment A7, wherein the viral surface protein is selected from the group consisting of envelope protein, spike protein, membrane protein and hemagglutinin esterase.
9. The vaccine according to any of embodiments A7 to A8, wherein the viral surface protein is the spike protein.
10. The vaccine according to any of embodiments A7 to A9, wherein the viral surface protein is the full-length spike protein.
11. The vaccine according to any of embodiments A7 to A10, wherein the viral surface protein is a part of the spike protein.
12. The vaccine according to any of embodiments A7 to A11, wherein the at least one betacoronavirus epitope is a part of the spike protein selected from the group consisting of receptor binding domain (RBD), heptad repeat 1 (HR1) domain and heptad repeat 2 (HR2) domain.
13. The vaccine according to any of embodiments A7 to A12, wherein the at least one betacoronavirus epitope is the RBD.
14. The vaccine according to any of embodiments A7 to A12, wherein the at least one betacoronavirus epitope is the HR1 domain or the HR2 domain, preferably the HR2 domain.
15. The vaccine according to any of embodiments A7 to A14, wherein the at least one betacoronavirus epitope is a B cell epitope comprised in the viral surface protein or part thereof.
16. The vaccine according to any of embodiments A7 to A15, wherein the antigenic unit comprises multiple B cell epitopes comprised in the viral surface protein or part thereof.
17. The vaccine according to any of embodiments A1 to A6, wherein the at least one betacoronavirus epitope is a T cell epitope.
18. The vaccine according to embodiment A17, wherein the T cell epitope is conserved between different species and/or different strains of betacoronaviruses.
19. The vaccine according to any of embodiments A17 or A18, wherein the T cell epitope is conserved between SARS-Cov2 and SARS-CoV.
20. The vaccine according to any of embodiments A17 to A19, wherein the T cell epitope has a length suitable for presentation by HLA class I/II alleles, preferably a length of from 7 to 30 amino acids.
21. The vaccine according to any of embodiments A17 to A20, wherein the T cell epitope is selected based on the predicted ability to bind to HLA class I/II alleles.
22. The vaccine according to any of embodiments A17 to A21, wherein the antigenic unit includes multiple T cell epitopes, preferably multiple T cell epitopes that are predicted to bind to HLA class I/II alleles.
23. The vaccine according to any of embodiments A17 to A22, wherein the T cell epitope is selected from the list consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223.
24. The vaccine according to any of embodiments A17 to A23, wherein the T cell epitope is selected from the list consisting of SEQ ID NO: 67, SEQ ID NO: 19, SEQ ID NO: 78, SEQ ID NO: 57, SEQ ID NO: 50, SEQ ID NO: 55, SEQ ID NO: 64, SEQ ID NO: 22, SEQ ID NO: 87, SEQ ID NO: 62, SEQ ID NO: 39, SEQ ID NO: 59, SEQ ID NO: 26, SEQ ID NO: 53, SEQ ID NO: 32, SEQ ID NO: 38, SEQ ID NO: 30, SEQ ID NO: 40, SEQ ID NO: 42, SEQ ID NO: 35, SEQ ID NO: 71, SEQ ID NO: 9, SEQ ID NO: 21, SEQ ID NO: 85, SEQ ID NO: 75, SEQ ID NO: 23, SEQ ID NO: 34, SEQ ID NO: 36, SEQ ID NO: 77 and SEQ ID NO: 20.
25. The vaccine according to any of embodiments A17 to A24, wherein the T cell epitope is selected from the list consisting of SEQ ID NO: 67, SEQ ID NO: 19, SEQ ID NO: 78, SEQ ID NO: 57, SEQ ID NO: 50, SEQ ID NO: 55, SEQ ID NO: 64, SEQ ID NO: 22, SEQ ID NO: 87 and SEQ ID NO: 62.
26. The vaccine according to any of preceding embodiments A17 to A25, wherein the antigenic unit comprises multiple T cell epitopes.
27. The vaccine according to any of embodiments A17 to A26, wherein said antigenic unit further comprises a full-length viral surface protein of a betacoronavirus or a part thereof.
28. The vaccine according to embodiment A27, wherein the viral surface protein is selected from the group consisting of envelope protein, spike protein, membrane protein and hemagglutinin esterase.
29. The vaccine according to any of embodiments A27 or A28, wherein the viral surface protein is the spike protein.
30. The vaccine according to any of embodiments A27 to A29, wherein the viral surface protein is the full-length spike protein.
31. The vaccine according to any of embodiments A27 to A30, wherein the viral surface protein is a part of the spike protein.
32. The vaccine according to any of embodiments A27 to A31, wherein said antigenic unit further comprises a part of the spike protein selected from the group consisting of receptor binding domain (RBD), heptad repeat 1 (HR1) domain and heptad repeat 2 (HR2) domain.
33. The vaccine according to any of embodiments A27 to A32, wherein said antigenic unit further comprises the RBD.
34. The vaccine according to any of embodiments A27 to A33, wherein said antigenic unit further comprises the HR1 domain or the HR2 domain, preferably the HR2 domain.
35. The vaccine according to any of embodiments A27 to A34, wherein said antigenic unit further comprises a B cell epitope comprised in the viral surface protein or part thereof.
36. The vaccine according to any of embodiments A27 to A35, wherein said antigenic unit further comprises multiple B cell epitopes comprised in the viral surface protein or part thereof.
37. The vaccine according to any of the preceding embodiments, wherein the antigenic unit comprises from 21 to 2000 amino acids, preferably from about 30 amino acids to about a 1500 amino acids, more preferably from about 50 to about 1000 amino acids, such as from about 100 to about 500 amino acids or from about 100 to about 400 amino acids or from about 100 to about 300 amino acids.
38. The vaccine according to any of the preceding embodiments, wherein the antigenic unit comprises one or more linkers, preferably one or more non-immunogenic and/or flexible linkers.
39. The vaccine according to any of the preceding embodiments, wherein the antigenic unit comprises 10, 20, 30 or 50 epitopes, preferably T cell epitopes.
40. The vaccine according to any of the preceding embodiments, wherein said targeting unit comprises antibody binding regions with specificity for surface receptors on antigen presenting cells (APCs), preferably CD14, CD40, Toll-like receptor, CCR1, CCR3, CCR5, MHC class I proteins or MHC class II proteins.
41. The vaccine according to any of the preceding embodiments, wherein the targeting unit has affinity for a chemokine receptor selected from CCR1, CCR3 and CCR5.
42. The vaccine according to any of the preceding embodiments, wherein said targeting unit has an affinity for MHC class II proteins, preferably MHC class II proteins, selected from the group consisting of anti-HLA-DP, anti-HLA-DR and anti-pan HLA class II.
43. The vaccine according to any of the preceding embodiments, wherein the targeting unit is selected from anti-pan HLA class II and MIP-1α.
44. The vaccine according to any of the preceding embodiments, wherein the targeting unit is MIP-1α.
45. The vaccine according to any of the preceding embodiments, wherein the targeting unit is anti-pan HLA class II.
46. The vaccine according to any of the preceding embodiments, wherein the dimerization unit comprises a hinge region and optionally another domain that facilitates dimerization, optionally connected through a linker.
47. The vaccine according to any of the preceding embodiments, wherein said polynucleotide further encodes a signal peptide.
48. The vaccine according to any of the preceding embodiments, wherein said targeting unit, dimerization unit and antigenic unit in said peptide are in the N-terminal to C-terminal order of targeting unit, dimerization unit and antigenic unit.
49. The vaccine according to any of the preceding embodiments, wherein said betacoronavirus is one selected from the group consisting of SARS-CoV, MERS-CoV, SARS-CoV-2, HCoV-OC43 and HCoV-HKU1, preferably selected from the group consisting of SARS-CoV and SARS-CoV2.
50. A polynucleotide as defined in any of the embodiments A1-A49.
51. A vector comprising the polynucleotide according to embodiment A50.
52. A host cell comprising the polynucleotide according to embodiment A50 or comprising the vector according to embodiment A51.
53. The polynucleotide according to embodiment A50 formulated for administration to a human individual.
54. A polypeptide encoded by the polynucleotide sequence according to embodiment A50.
55. A dimeric protein consisting of two polypeptides according to embodiment A54.
56. The dimeric protein according to embodiment A55, being a homodimeric protein.
57. The polynucleotide according to embodiment A50 or the polypeptide according to embodiment 53 or the dimeric protein according to any of embodiments A55 or A56 for use as a medicament.
58. The polynucleotide according to embodiment A50 or the polypeptide according to embodiment A54 or the dimeric protein according to any of embodiments A55 or A56 for use in the treatment of an infection with a betacoronavirus or for use in the prevention of a betacoronavirus infection.
59. The polynucleotide according to embodiment A50 or the polypeptide according to embodiment 54 or the dimeric protein according to any of embodiments 55 or 56 for use in the treatment of an infection or for use in the prevention of an infection with SARS-CoV, MERS-CoV, SARS-CoV-2, HCoV-OC43 or HCoV-HKU1, preferably SARS-CoV or SARS-CoV2.
60. A method of preparing a vaccine according to any one of the preceding embodiments A1 to A49, wherein said method comprises:
   a) transfecting cells with the polynucleotide as defined in any of the embodiments A1-A49;
   b) culturing the cells;
   c) collecting and purifying the dimeric protein or the polypeptide expressed from the cells; and
   d) mixing the dimeric protein or polypeptide obtained from step c) with a pharmaceutically acceptable carrier.
61. A polypeptide comprising an amino acid sequence from the list consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223.
62. A polypeptide comprising an amino acid sequence from the list consisting of SEQ ID NO: 67, SEQ ID NO: 19, SEQ ID NO: 78, SEQ ID NO: 57, SEQ ID NO: 50, SEQ ID NO: 55, SEQ ID NO: 64, SEQ ID NO: 22, SEQ ID NO: 87, SEQ ID NO: 62, SEQ ID NO: 39, SEQ ID NO: 59, SEQ ID NO: 26, SEQ ID NO: 53, SEQ ID NO: 32, SEQ ID NO: 38, SEQ ID NO: 30, SEQ ID NO: 40, SEQ ID NO: 42, SEQ ID NO: 35, SEQ ID NO: 71, SEQ ID NO: 9, SEQ ID NO: 21, SEQ ID NO: 85, SEQ ID NO: 75, SEQ ID NO: 23, SEQ ID NO: 34, SEQ ID NO: 36, SEQ ID NO: 77 and SEQ ID NO: 20.
63. A polypeptide comprising an amino acid sequence from the list consisting of SEQ ID NO: 67, SEQ ID NO: 19, SEQ ID NO: 78, SEQ ID NO: 57, SEQ ID NO: 50, SEQ ID NO: 55, SEQ ID NO: 64, SEQ ID NO: 22, SEQ ID NO: 87 and SEQ ID NO: 62.

### Embodiments B

1. A vaccine comprising an immunologically effective amount of:
   (iv) a polynucleotide comprising a nucleotide sequence encoding a targeting unit, a dimerization unit and an antigenic unit, wherein the antigenic unit comprises at least one betacoronavirus epitope; or
   (v) a polypeptide encoded by the polynucleotide as defined in (i), or
   (vi) a dimeric protein consisting of two polypeptides encoded by the polynucleotide as defined in (i); and
   a pharmaceutically acceptable carrier.
   2. The vaccine according to embodiment B1, wherein the at least one betacoronavirus epitope is a full-length viral surface protein of a betacoronavirus or a part thereof.
   3. The vaccine according to embodiment B2, wherein the viral surface protein is selected from the group consisting of envelope protein, spike protein, membrane protein and hemagglutinin esterase.
   4. The vaccine according to any of embodiments B2 to B3 wherein the at least one betacoronavirus epitope comprises or is the spike protein.
   5. The vaccine according to any of embodiments B2 to B4, wherein the at least one betacoronavirus epitope comprises or is the full-length spike protein.
   6. The vaccine according to embodiment B5, wherein the at least one betacoronavirus epitope comprises or consists of an amino acid sequence having at least 70% sequence identity to the amino acid sequence of SEQ ID NO: 230, such as at least 75%, such as at least 77%, such as at least 80%, such as at least 85%, such as at least 90%, such as at least 91%, such as at least 92%, such as at least 93%, such as at least 94%, such as at least 95%, such as at least 96%, such as at least 97%, such as at least 98% or such as at least 99% sequence identity or such as 100% sequence identity.
   7. The vaccine according to embodiment B5, wherein the at least one betacoronavirus epitope comprises or consists of an amino acid sequence having at least 70% sequence identity to the amino acid sequence 243 to 1437 of SEQ ID NO: 275, such as at least 75%, such as at least 77%, such as at least 80%, such as at least 85%, such as at least 90%, such as at least 91%, such as at least 92%, such as at least 93%, such as at least 94%, such as at least 95%, such as at least 96%, such as at least 97%, such as at least 98% or such as at least 99% sequence identity or such as 100% sequence identity.
   8. The vaccine according to any of embodiments B2 to B4 wherein the at least one betacoronavirus epitope comprises or is a part of the spike protein.
   9. The vaccine according to embodiment B8, wherein the part of the spike protein is one selected from the group consisting of receptor binding domain (RBD), heptad repeat 1 (HR1) domain and heptad repeat 2 (HR2) domain.
   10. The vaccine according to embodiment B9, wherein the at least one betacoronavirus epitope comprises or is the RBD or part of the RBD.
   11. The vaccine according to embodiment B10, wherein the at least one betacoronavirus epitope comprises or consists of an amino acid sequence having at least 70% sequence identity to the amino acid sequence of SEQ ID NO: 231 or SEQ ID NO: 802, or SEQ ID NO: 803 or SEQ ID NO: 804 or SEQ ID NO: 805, such as at least 75%, such as at least 77%, such as at least 80%, such as at least 85%, such as at least 90%, such as at least 91%, such as at least 92%, such as at least 93%, such as at least 94%, such as at least 95%, such as at least 96%, such as at least 97%, such as at least 98% or such as at least 99% sequence identity or such as 100% sequence identity.
   12. The vaccine according to embodiment B10, wherein the at least one betacoronavirus epitope comprises or consists of an amino acid sequence having at least 70% sequence identity to the amino acid sequence 243 to 465 of SEQ ID NO: 255, such as at least 75%, such as at least 77%, such as at least 80%, such as at least 85%, such as at least 90%, such as at least 91%, such as at least 92%, such as at least 93%, such as at least 94%, such as at least 95%, such as at least 96%, such as at least 97%, such as at least 98% or such as at least 99% sequence identity or such as 100% sequence identity.
   13. The vaccine according to embodiment B10, wherein the at least one betacoronavirus epitope comprises or consists of an amino acid sequence having at least 70% sequence identity to the amino acid sequence of SEQ ID NO: 246, such as at least 75%, such as at least 77%, such as at least 80%, such as at least 85%, such as at least 90%, such as at least 91%, such as at least 92%, such as at least 93%, such as at least 94%, such as at least 95%, such as at least 96%, such as at least 97%, such as at least 98% or such as at least 99% sequence identity or such as 100% sequence identity.
   14. The vaccine according to any of embodiments B10 to B13, wherein the antigenic unit comprises multiple copies of the RBD or parts thereof which copies are identical or differ in their amino acid sequences.
   15. The vaccine according to embodiment B14, wherein the antigenic unit comprises of from 1 to 5 copies.
   16. The vaccine according to embodiment B8, wherein the at least one betacoronavirus epitope comprises or is the HR1 domain or the HR2 domain, preferably the HR2 domain.
   17. The vaccine according to any of embodiments B2 to B16, wherein the at least one betacoronavirus epitope is a B cell epitope comprised in the viral surface protein or a part thereof.
   18. The vaccine according to any of embodiments B2 to B17, wherein the antigenic unit comprises multiple B cell epitopes comprised in the viral surface protein or a part thereof.
   19. The vaccine according to embodiment 1, wherein the at least one betacoronavirus epitope is a T cell epitope.
   20. The vaccine to embodiment B19, wherein the antigenic unit comprises multiple T cell epitopes.
   21. The vaccine according to any of embodiments B19 to B20, wherein the T cell epitope is comprised in a structural protein or in a non-structural protein.
   22. The according to any of embodiments B19 to B21 wherein the T cell epitope is comprised in a surface protein, in the nucleocapsid protein or in a replicase polyprotein.
   23. The vaccine according to any of embodiments B19 to B22, wherein the T cell epitope is conserved between different genus and/or species and/or strains of betacoronaviruses.
   24. The vaccine according to any of embodiments B19 to B23, wherein the T cell epitope is conserved between SARS-Cov2 and SARS-CoV.
   25. The vaccine according to any of embodiments B19 to B24, wherein the T cell epitope has a length of from 7 to about 200 amino acids, preferably of from 7 to 100 amino acids or the T cell epitope has a length suitable for presentation by HLA class I/II alleles, preferably a length of from 7 to 30 amino acids, more preferably a length of from 8 to 15 amino acids.
   26. The vaccine according to any of embodiments B19 to B25, wherein the T cell epitope is known to be immunogenic or is selected based on the predicted ability to bind to HLA class I/II alleles.
   27. The vaccine according to any of embodiments B19 to B26, wherein the antigenic unit comprises multiple T cell epitopes that are known to be immunogenic or predicted to bind to HLA class I/II alleles.
   28. The vaccine according to any of embodiments B19 to B27, wherein the T cell epitope is selected from epitopes having an amino acid sequence of any of SEQ ID NO: 1 to SEQ ID NO: 444.
   29. The vaccine according to embodiment B28, wherein the T cell epitope is selected from the list consisting of SEQ ID NO: 67, SEQ ID NO: 19, SEQ ID NO: 78, SEQ ID NO: 57, SEQ ID NO: 50, SEQ ID NO: 55, SEQ ID NO: 64, SEQ ID NO: 22, SEQ ID NO: 87, SEQ ID NO: 62, SEQ ID NO: 39, SEQ ID NO: 59, SEQ ID NO: 26, SEQ ID NO: 53, SEQ ID NO: 32, SEQ ID NO: 38, SEQ ID NO: 30, SEQ ID NO: 40, SEQ ID NO: 42, SEQ ID NO: 35, SEQ ID NO: 71, SEQ ID NO: 9, SEQ ID NO: 21, SEQ ID NO: 85, SEQ ID NO: 75, SEQ ID NO: 23, SEQ ID NO: 34, SEQ ID NO: 36, SEQ ID NO: 77 and SEQ ID NO: 20.
   30. The vaccine according to embodiment B28, wherein the T cell epitope is selected from the list consisting of SEQ ID NO: 67, SEQ ID NO: 19, SEQ ID NO: 78, SEQ ID NO: 57, SEQ ID NO: 50, SEQ ID NO: 55, SEQ ID NO: 64, SEQ ID NO: 22, SEQ ID NO: 87 and SEQ ID NO: 62.
   31. The vaccine according to any of embodiments B19 to B27, wherein the T cell epitope is selected from T cell epitopes that are comprised in the antigenic unit having an amino acid sequence of SEQ ID NO: 245, wherein the sequence GGGGSGGGGS is a linker and not a T cell epitope.
   32. The vaccine according to any of embodiments B19 to B27, wherein the T cell epitope is selected from the list consisting of RSFIEDLLFNKVTLA, MTYRRLISMMGFKMNYQVNGYPNMF, LMIERFVSLAIDAYP, RAMPNMLRIMASLVL, MVYMPASWVMRIMTW, FLNRFTTTLNDFNLVAM, SSVELKHFFFAQDGNAAI, HFAIGLALYYPSARIVYTACSHAAV, YFIKGLNNLNRGMVL, YLNTLTLAVPYNMRV, AQFAPSASAFFGMSRI, EIVDTVSALVYDNKL, SSGDATTAYANSVFNICQAVTANVNALL, HVISTSHKLVLSVNPYV, MLSDTLKNLSDRVVFVLWAHGFEL, TANPKTPKYKFVRIQPGQTF, ASIKNFKSVLYYQNNVFM, FVNEFYAYLRKHFSMM, RVWTLMNVLTLVYKV, FAYANRNRFLYIIKL and LVKPSFYVYSRVKNL.
   33. The vaccine according to any of embodiments B19 to B27, wherein the antigenic unit comprises one or more T cell epitopes selected from the list consisting of RAMPNMLRIMASLVL, HVISTSHKLVLSVNPYV and LVKPSFYVYSRVKNL.
   34. The vaccine according to any of embodiment B19 to B33, wherein the antigenic unit further comprises at least one betacoronavirus epitope which is a full-length viral surface protein of a betacoronavirus or a part thereof.
   35. The vaccine according to embodiment B34, wherein the viral surface protein is selected from the group consisting of envelope protein, spike protein, membrane protein and hemagglutinin esterase.
   36. The vaccine according to any of embodiments B34 to B35 wherein the at least one betacoronavirus epitope comprises or is the spike protein.
   37. The vaccine according to any of embodiments B34 to B36, wherein the at least one betacoronavirus epitope comprises or is the full-length spike protein.
   38. The vaccine according to embodiment B37, wherein the at least one betacoronavirus epitope comprises or consists of an amino acid sequence having at least 70% sequence identity to the amino acid sequence of SEQ ID NO: 230, such as at least 75%, such as at least 77%, such as at least 80%, such as at least 85%, such as at least 90%, such as at least 91%, such as at least 92%, such as at least 93%, such as at least 94%, such as at least 95%, such as at least 96%, such as at least 97%, such as at least 98% or such as at least 99% sequence identity or such as 100% sequence identity.
   39. The vaccine according to embodiment B37, wherein the at least one betacoronavirus epitope comprises or consists of an amino acid sequence having at least 70% sequence identity to the amino acid sequence 243 to 1437 of SEQ ID NO: 275, such as at least 75%, such as at least 77%, such as at least 80%, such as at least 85%, such as at least 90%, such as at least 91%, such as at least 92%, such as at least 93%, such as at least 94%, such as at least 95%, such as at least 96%, such as at least 97%, such as at least 98% or such as at least 99% sequence identity or such as 100% sequence identity.
   40. The vaccine according to any of embodiments B34 to B36 wherein the at least one betacoronavirus epitope comprises or is a part of the spike protein.
   41. The vaccine according to embodiment B40, wherein the part of the spike protein is one selected from the group consisting of receptor binding domain (RBD), heptad repeat 1 (HR1) domain and heptad repeat 2 (HR2) domain.
   42. The vaccine according to embodiment 41, wherein the at least one betacoronavirus epitope comprises or is the RBD.
   43. The vaccine according to embodiment B42, wherein the at least one betacoronavirus epitope comprises or consists of an amino acid sequence having at least 70% sequence identity to the amino acid sequence of SEQ ID NO: 231 or SEQ ID NO: 802, or SEQ ID NO: 803 or SEQ ID NO: 804 or SEQ ID NO: 805, such as at least 75%, such as at least 77%, such as at least 80%, such as at least 85%, such as at least 90%, such as at least 91%, such as at least 92%, such as at least 93%, such as at least 94%, such as at least 95%, such as at least 96%, such as at least 97%, such as at least 98% or such as at least 99% sequence identity or such as 100% sequence identity.
   44. The vaccine according to embodiment B42, wherein the at least one betacoronavirus epitope comprises or consists of an amino acid sequence having at least 70% sequence identity to the amino acid sequence 243 to 465 of SEQ ID NO: 255, such as at least 75%, such as at least 77%, such as at least 80%, such as at least 85%, such as at least 90%, such as at least 91%, such as at least 92%, such as at least 93%, such as at least 94%, such as at least 95%, such as at least 96%, such as at least 97%, such as at least 98% or such as at least 99% sequence identity or such as 100% sequence identity.
   45. The vaccine according to embodiment B42, wherein the at least one betacoronavirus epitope comprises or consists of an amino acid sequence having at least 70% sequence identity to the amino acid sequence of SEQ ID NO: 246, such as at least 75%, such as at least 77%, such as at least 80%, such as at least 85%, such as at least 90%, such as at least 91%, such as at least 92%, such as at least 93%, such as at least 94%, such as at least 95%, such as at least 96%, such as at least 97%, such as at least 98% or such as at least 99% sequence identity or such as 100% sequence identity.
   46. The vaccine according to any of embodiments B42 to B45, wherein the antigenic unit comprises multiple copies of the RBD or parts thereof which copies are identical or differ in their amino acid sequences.
   47. The vaccine according to embodiment B46, wherein the antigenic unit comprises of from 1 to 5 copies.
   48. The vaccine according to embodiment B40, wherein the at least one betacoronavirus epitope comprises or is the HR1 domain or the HR2 domain, preferably the HR2 domain.
   49. The vaccine according to any of embodiments B34 to B48, wherein the at least one betacoronavirus epitope is a B cell epitope comprised in the viral surface protein or a part thereof.
   50. The vaccine according to any of embodiments B34 to B49, wherein the antigenic unit comprises multiple B cell epitopes comprised in the viral surface protein or a part thereof.
   51. The vaccine according to embodiment B34, wherein the antigenic unit comprises a T cell epitope selected from the list consisting of RSFIEDLLFNKVTLA, MTYRRLISMMGFKMNYQVNGYPNMF, LMIERFVSLAIDAYP, RAMPNMLRIMASLVL, MVYMPASWVMRIMTW, FLNRFTTTLNDFNLVAM, SSVELKHFFFAQDGNAAI, HFAIGLALYYPSARIVYTACSHAAV, YFIKGLNNLNRGMVL, YLNTLTLAVPYNMRV, AQFAPSASAFFGMSRI, EIVDTVSALVYDNKL, SSGDATTAYANSVFNICQAVTANVNALL, HVISTSHKLVLSVNPYV, MLSDTLKNLSDRVVFVLWAHGFEL, TANPKTPKYKFVRIQPGQTF, ASIKNFKSVLYYQNNVFM, FVNEFYAYLRKHFSMM, RVWTLMNVLTLVYKV, FAYANRNRFLYIIKL and LVKPSFYVYSRVKN and wherein the antigenic unit further comprises an amino acid sequence having at least 70% sequence identity to the amino acid sequence of SEQ ID NO: 231 or SEQ ID NO: 802, or SEQ ID NO: 803 or SEQ ID NO: 804 or SEQ ID NO: 805, such as at least 75%, such as at least 77%, such as at least 80%, such as at least 85%, such as at least 90%, such as at least 91%, such as at least 92%, such as at least 93%, such as at least 94%, such as at least 95%, such as at least 96%, such as at least 97%, such as at least 98% or such as at least 99% sequence identity or such as 100% sequence identity.
   52. The vaccine according to embodiment B34, wherein the antigenic unit comprises a T cell epitope selected from the list consisting of RSFIEDLLFNKVTLA, MTYRRLISMMGFKMNYQVNGYPNMF, LMIERFVSLAIDAYP, RAMPNMLRIMASLVL, MVYMPASWVMRIMTW, FLNRFTTTLNDFNLVAM, SSVELKHFFFAQDGNAAI, HFAIGLALYYPSARIVYTACSHAAV, YFIKGLNNLNRGMVL, YLNTLTLAVPYNMRV, AQFAPSASAFFGMSRI, EIVDTVSALVYDNKL, SSGDATTAYANSVFNICQAVTANVNALL, HVISTSHKLVLSVNPYV, MLSDTLKNLSDRVVFVLWAHGFEL, TANPKTPKYKFVRIQPGQTF, ASIKNFKSVLYYQNNVFM, FVNEFYAYLRKHFSMM, RVWTLMNVLTLVYKV, FAYANRNRFLYIIKL and LVKPSFYVYSRVKN and wherein the antigenic unit further comprises an amino acid sequence having at least 70% sequence identity to the amino acid sequence 243 to 465 of SEQ ID NO: 255, such as at least 75%, such as at least 77%, such as at least 80%, such as at least 85%, such as at least 90%, such as at least 91%, such as at least 92%, such as at least 93%, such as at least 94%, such as at least 95%, such as at least 96%, such as at least 97%, such as at least 98% or such as at least 99% sequence identity or such as 100% sequence identity.
   53. The vaccine according to embodiment B34, wherein the antigenic unit comprises a T cell epitope selected from the list consisting of RSFIEDLLFNKVTLA, MTYRRLISMMGFKMNYQVNGYPNMF, LMIERFVSLAIDAYP, RAMPNMLRIMASLVL, MVYMPASWVMRIMTW, FLNRFTTTLNDFNLVAM, SSVELKHFFFAQDGNAAI, HFAIGLALYYPSARIVYTACSHAAV, YFIKGLNNLNRGMVL, YLNTLTLAVPYNMRV, AQFAPSASAFFGMSRI, EIVDTVSALVYDNKL, SSGDATTAYANSVFNICQAVTANVNALL, HVISTSHKLVLSVNPYV, MLSDTLKNLSDRVVFVLWAHGFEL, TANPKTPKYKFVRIQPGQTF, ASIKNFKSVLYYQNNVFM, FVNEFYAYLRKHFSMM, RVWTLMNVLTLVYKV, FAYANRNRFLYIIKL and LVKPSFYVYSRVKN and wherein the antigenic unit further comprises an amino acid sequence having at least 70% sequence identity to the amino acid sequence of SEQ ID NO: 246, such as at least 75%, such as at least 77%, such as at least 80%, such as at least 85%, such as at least 90%, such as at least 91%, such as at least 92%, such as at least 93%, such as at least 94%, such as at least 95%, such as at least 96%, such as at least 97%, such as at least 98% or such as at least 99% sequence identity or such as 100% sequence identity.
   54. The vaccine according to embodiment B34, wherein the antigenic unit comprises a T cell epitope selected from the list consisting of RSFIEDLLFNKVTLA, MTYRRLISMMGFKMNYQVNGYPNMF, LMIERFVSLAIDAYP, RAMPNMLRIMASLVL, MVYMPASWVMRIMTW, FLNRFTTTLNDFNLVAM, SSVELKHFFFAQDGNAAI, HFAIGLALYYPSARIVYTACSHAAV, YFIKGLNNLNRGMVL, YLNTLTLAVPYNMRV, AQFAPSASAFFGMSRI, EIVDTVSALVYDNKL, SSGDATTAYANSVFNICQAVTANVNALL, HVISTSHKLVLSVNPYV, MLSDTLKNLSDRVVFVLWAHGFEL, TANPKTPKYKFVRIQPGQTF, ASIKNFKSVLYYQNNVFM, FVNEFYAYLRKHFSMM, RVWTLMNVLTLVYKV, FAYANRNRFLYIIKL and LVKPSFYVYSRVKN and wherein the antigenic unit further comprises an amino acid sequence having at least 70% sequence identity to the amino acid sequence of SEQ ID NO: 246, such as at least 75%, such as at least 77%, such as at least 80%, such as at least 85%, such as at least 90%, such as at least 91%, such as at least 92%, such as at least 93%, such as at least 94%, such as at least 95%, such as at least 96%, such as at least 97%, such as at least 98% or such as at least 99% sequence identity or such as 100% sequence identity.
   55. The vaccine according to embodiment B34, wherein the antigenic unit comprises one or more T cell epitopes selected from the list consisting of RAMPNMLRIMASLVL, HVISTSHKLVLSVNPYV and LVKPSFYVYSRVKNL and wherein the antigenic unit further comprises an amino acid sequence having at least 70% sequence identity to the amino acid sequence 243 to 465 of SEQ ID NO: 255, such as at least 75%, such as at least 77%, such as at least 80%, such as at least 85%, such as at least 90%, such as at least 91%, such as at least 92%, such as at least 93%, such as at least 94%, such as at least 95%, such as at least 96%, such as at least 97%, such as at least 98% or such as at least 99% sequence identity or such as 100% sequence identity.
   56. The vaccine according to embodiment B34, wherein the antigenic unit comprises one or more T cell epitopes selected from the list consisting of RAMPNMLRIMASLVL, HVISTSHKLVLSVNPYV and LVKPSFYVYSRVKNL and wherein the antigenic unit further comprises an amino acid sequence having at least 70% sequence identity to the amino acid sequence of SEQ ID NO: 246, such as at least 75%, such as at least 77%, such as at least 80%, such as at least 85%, such as at least 90%, such as at least 91%, such as at least 92%, such as at least 93%, such as at least 94%, such as at least 95%, such as at least 96%, such as at least 97%, such as at least 98% or such as at least 99% sequence identity or such as 100% sequence identity.
   57. The vaccine according to any of the preceding embodiments, wherein the antigenic unit comprises up to 3500 amino acids, such as from 21 to 3500 amino acids, preferably from about 30 amino acids to about 2000 amino acids such as from about 50 to about 1500 amino acids, more preferably from about 100 to about 1500 amino acids, such as from about 100 to about 1000 amino acids or from about 100 to about 500 amino acids or from about 100 to about 300 amino acids.
   58. The vaccine according to any of the preceding embodiments, wherein the antigenic unit comprises one or more linkers, preferably one or more non-immunogenic and/or flexible linkers.
   59. The vaccine according to embodiment B58, wherein the antigenic unit comprises multiple T cell epitopes which are separated by a non-immunogenic and/or flexible linker, preferably a linker consisting of from 4 to 20 amino acids, e.g. from 5 to 20 amino acids or 5 to 15 amino acids or 8 to 20 amino acids or 8 to 15 amino acids 10 to 15 amino acids or 8 to 12 amino acids, more preferably a linker selected from the group consisting of serine and/or glycine rich linker which optionally comprises at least one leucine residue, GSAT liker and SEG linker.
   60. The vaccine according to embodiment B58, wherein the antigenic unit comprises at least one T cell epitope and a full-length protein of the betacoronavirus, or a part thereof, the at least one T cell epitope and the full-length protein of the betacoronavirus or the part thereof are separated by a non-immunogenic and/or flexible linker, preferably a linker consisting of from 10 to 60 amino acids, e.g. from 11 to 50 amino acids or 20 to 50 amino acids or 25 to 45 amino acids or 12 to 45 amino acids or 13 to 40 amino acids or 30 to 40 amino acids, more preferably a linker selected from the group consisting of serine and/or glycine rich linker which optionally comprises at least one leucine residue, TQKSLSLSPGKGLGGL, SLSLSPGKGLGGL, GSAT liker such as GGSAGGSGSGSSGGSSGASGTGTAGGTGSGSGTGSG and SEG linker such as GGSGGGSEGGGSEGGGSEGGGSEGGGSEGGGSGGGS.
   61. The vaccine according to any of the preceding embodiments, wherein the antigenic unit comprises 10, 20, 30, 40 or 50 epitopes, preferably T cell epitopes.
   62. The vaccine according to any of the preceding embodiments, wherein said targeting unit comprises antibody binding regions with specificity for surface molecules or receptors on antigen presenting cells (APCs), preferably specificity for CD14, CD40, Toll-like receptor, CCR1, CCR3, CCR5, MHC class I proteins or MHC class II proteins.
   63. The vaccine according to any of the preceding embodiments, wherein the targeting unit has affinity for a chemokine receptor selected from CCR1, CCR3 and CCR5.
   64. The vaccine according to any of embodiments B62 to B63, wherein said targeting unit has affinity for MHC class II proteins, preferably MHC class II proteins selected from the group consisting of anti-HLA-DP, anti-HLA-DR and anti-pan HLA class II.
   65. The vaccine according to any of the preceding embodiments, wherein the targeting unit is selected from anti-pan HLA class II and MIP-1α and preferably selected from anti-pan HLA class II and human MIP-1α.
   66. The vaccine according to embodiment B65, wherein the targeting unit is MIP-1α, preferably human MIP-1α.
   67. The vaccine according to embodiment B66, wherein the targeting unit comprises or consists of an amino acid sequence having at least 85% sequence identity to the amino acid sequence 24-93 of SEQ ID NO: 233, such as at least 86% or at least 87%, 88%, 89%, 90%, 91%, 92%, 93%, 94%, 95%, 96%, 97%, 98%, at least 99% or 100% sequence identity.
   68. The vaccine according to embodiment B65, wherein the targeting unit is anti-pan HLA class II.
   69. The vaccine according to embodiment B68, wherein the targeting unit comprises an amino acid sequence having at least 85% sequence identity to the amino acid sequence 20-260 of SEQ ID NO: 321, such as at least 86% or at least 87%, 88%, 89%, 90%, 91%, 92%, 93%, 94%, 95%, 96%, 97%, 98%, at least 99% or 100% sequence identity.
   70. The vaccine according to any of the preceding embodiments, wherein the dimerization unit comprises a hinge region.
   71. The vaccine according to embodiment B70, wherein the hinge region has the ability to form one or more covalent bonds.
   72. The vaccine according to any of embodiments B70 to B71, wherein the hinge region is Ig derived.
   73. The vaccine according to any of embodiments B70 to B72, wherein the dimerization unit further comprises another domain that facilitates dimerization.
   74. The vaccine according to embodiment B73, wherein the other domain is an immunoglobulin domain, preferably an immunoglobulin constant domain.
   75. The vaccine according to any of embodiments B73 and B74, wherein the other domain is a carboxyterminal C domain derived from IgG, preferably from IgG3.
   76. The vaccine according to any of embodiments B70 to B75, wherein the dimerization unit further comprises a dimerization unit linker.
   77. The vaccine according to embodiment B76, wherein the dimerization unit linker connects the hinge region and the other domain that facilitates dimerization.
   78. The vaccine according to any of embodiments B70 to B77, wherein the dimerization unit comprises hinge exon h1 and hinge exon h4, a dimerization unit linker and a CH3 domain of human IgG3.
   79. The vaccine according to embodiment B78, wherein the dimerization unit comprises or consists of an amino acid sequence having at least 85% sequence identity to the amino acid sequence 94 to 237 of SEQ ID NO: 233, such as at least 86% or at least 87%, 88%, 89%, 90%, 91%, 92%, 93%, 94%, 95%, 96%, 97%, 98%, at least 99% or 100% sequence identity.
   80. The vaccine according to any of the preceding embodiments, wherein the vaccine comprises the polynucleotide (i).
   81. The vaccine according to embodiment B80, wherein the polynucleotide is an RNA or DNA, preferably a DNA.
   82. The vaccine according to any of embodiments B80 to B81, wherein the polynucleotide further comprises a nucleotide sequence encoding a signal peptide.
   83. The vaccine according to embodiment B82, wherein the signal peptide is an Ig VH signal peptide, a human TPA signal peptide or a human MIP1-α signal peptide.
   84. The vaccine according to embodiment B83, wherein the signal peptide comprises or consists of an amino acid sequence having at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% or 100% sequence identity to the amino acid sequence 1-23 of SEQ ID NO: 233.
   85. The vaccine according to embodiment B84, wherein the targeting unit is human MIP-1α.
   86. The vaccine according to embodiment B83, wherein the signal peptide comprises or consists of an amino acid sequence having at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% or 100% sequence identity to the amino acid sequence 1-19 of SEQ ID NO: 321.
   87. The vaccine according to embodiment B86, wherein the targeting unit is anti-pan HLA class II.
   88. The vaccine according to any of the preceding embodiments, wherein the vaccine comprises the polypeptide or the dimeric protein and said targeting unit, dimerization unit and antigenic unit in said peptide or dimeric protein are in the N-terminal to C-terminal order of targeting unit, dimerization unit and antigenic unit.
   89. The vaccine according to any of the preceding embodiments, wherein said betacoronavirus is one selected from the group consisting of SARS-CoV, MERS-CoV, SARS-CoV-2, HCoV-OC43 and HCoV-HKU1, preferably selected from the group consisting of SARS-CoV and SARS-CoV.
   90. The vaccine according to embodiment B89, wherein said betacoronavirus is SARS-CoV-2.
   91. The vaccine according to any of the preceding embodiments, wherein the pharmaceutically acceptable carrier is selected from the group consisting of saline, buffered saline, PBS, dextrose, water, glycerol, ethanol, sterile isotonic aqueous buffers, and combinations thereof.
   92. A polynucleotide as defined in any of the embodiments B1 to B90.
   93. A vector comprising the polynucleotide according to embodiment B92.
   94. A host cell comprising the polynucleotide as defined in any of the embodiments B1 to B90 or comprising the vector according to embodiment B93.
   95. A polypeptide encoded by the polynucleotide as defined in embodiment B92.
   96. A dimeric protein consisting of two polypeptides as defined in embodiment B95.
   97. The dimeric protein according to embodiment B96, wherein the dimeric protein is a homodimeric protein.
   98. The polynucleotide according to embodiment B92 or the polypeptide according to embodiment B95 or the dimeric protein according to any of embodiments B96 or B97 for use as a medicament.
   99. The polynucleotide according to embodiment B92 or the polypeptide according to embodiment B95 or the dimeric protein according to any of embodiments B96 or B97 for use in the treatment of an infection with a betacoronavirus or for use in the prevention of a betacoronavirus infection.
   100. The polynucleotide according to embodiment B92 or the polypeptide according to embodiment B95 or the dimeric protein according to any of embodiments B96 or B97 for use in the treatment of an infection or for use in the prevention of an infection with SARS-CoV, MERS-CoV, SARS-CoV-2, HCoV-OC43 or HCoV-HKU1, preferably SARS-CoV or SARS-CoV, more preferably SARS-CoV-2.
   101. A method of preparing the vaccine according to any one of the preceding embodiments B1 to B79 and B88 to B91, wherein the vaccine comprises the polypeptide or the dimeric protein, wherein said method comprises:
   a) transfecting cells with the polynucleotide as defined in any of the embodiments B1 to B90;
   b) culturing the cells;
   c) collecting and purifying the dimeric protein or the polypeptide expressed from the cells; and
   d) mixing the dimeric protein or polypeptide obtained from step c) with the pharmaceutically acceptable carrier.
   102. A method for preparing the vaccine according to any one of the preceding embodiments B1 to B87 and B89 to B91, wherein the vaccine comprises the polynucleotide, the method comprises:
   a) preparing the polynucleotide;
   b) optionally cloning the polynucleotide into an expression vector; and
   c) mixing the polynucleotide obtained from step a) or the vector obtained from step b) with the pharmaceutically acceptable carrier.
   103. A method for treating a subject having suffering from a betacoronavirus infection or being in need of prevention thereof, the method comprising administering to the subject the vaccine as defined in any of embodiments B1 to B91.
   104. A vaccine as defined in any of embodiments B1 to B91 for use in the treatment of an infection with a betacoronavirus or for use in the prevention of a betacoronavirus infection.

1. A vaccine comprising an immunologically effective amount of:
(i) a polynucleotide comprising a nucleotide sequence encoding a targeting unit, a dimerization unit and an antigenic unit, wherein the antigenic unit comprises at least one betacoronavirus epitope; or
(ii) a polypeptide encoded by the polynucleotide as defined in (i), or
(iii) a dimeric protein consisting of two polypeptides encoded by the polynucleotide as defined in (i); and
a pharmaceutically acceptable carrier.

2. The vaccine according to claim 1, wherein the at least one betacoronavirus epitope is a full-length viral surface protein of a betacoronavirus or a part thereof.

3. The vaccine according to claim 2, wherein the viral surface protein is selected from the group consisting of envelope protein, spike protein, membrane protein and hemagglutinin esterase.

4. The vaccine according to any of claims 2 to 3 wherein the at least one betacoronavirus epitope comprises or is the spike protein.

5. The vaccine according to any of claims 2 to 4, wherein the at least one betacoronavirus epitope comprises or is the full-length spike protein.

6. The vaccine according to claim 5, wherein the at least one betacoronavirus epitope comprises or consists of an amino acid sequence having at least 70% sequence identity to the amino acid sequence 243 to 1437 of SEQ ID NO: 275, such as at least 75%, such as at least 77%, such as at least 80%, such as at least 85%, such as at least 90%, such as at least 91%, such as at least 92%, such as at least 93%, such as at least 94%, such as at least 95%, such as at least 96%, such as at least 97%, such as at least 98% or such as at least 99% sequence identity or such as 100% sequence identity.

7. The vaccine according to any of claims 2 to 4, wherein the at least one betacoronavirus epitope comprises or is a part of the spike protein.

8. The vaccine according to claim 7, wherein the part of the spike protein is one selected from the group consisting of receptor binding domain (RBD), heptad repeat 1 (HR1) domain and heptad repeat 2 (HR2) domain.

9. The vaccine according to claim 8, wherein the at least one betacoronavirus epitope comprises or is the RBD or a part of the RBD.

10. The vaccine according to claim 9, wherein the at least one betacoronavirus epitope comprises or consists of an amino acid sequence having at least 70% sequence identity to the amino acid sequence of SEQ ID NO: 231 or SEQ ID NO: 802, or SEQ ID NO: 803 or SEQ ID NO: 804 or SEQ ID NO: 805, such as at least 75%, such as at least 77%, such as at least 80%, such as at least 85%, such as at least 90%, such as at least 91%, such as at least 92%, such as at least 93%, such as at least 94%, such as at least 95%, such as at least 96%, such as at least 97%, such as at least 98% or such as at least 99% sequence identity or such as 100% sequence identity.

11. The vaccine according to claim 9, wherein the at least one betacoronavirus epitope comprises or consists of an amino acid sequence having at least 70% sequence identity to the amino acid sequence 243 to 465 of SEQ ID NO: 255, such as at least 75%, such as at least 77%, such as at least 80%, such as at least 85%, such as at least 90%, such as at least 91%, such as at least 92%, such as at least 93%, such as at least 94%, such as at least 95%, such as at least 96%, such as at least 97%, such as at least 98% or such as at least 99% sequence identity or such as 100% sequence identity.

12. The vaccine according to claim 9, wherein the at least one betacoronavirus epitope comprises or consists of an amino acid sequence having at least 70% sequence identity to the amino acid sequence of SEQ ID NO: 246, such as at least 75%, such as at least 77%, such as at least 80%, such as at least 85%, such as at least 90%, such as at least 91%, such as at least 92%, such as at least 93%, such as at least 94%, such as at least 95%, such as at least 96%, such as at least 97%, such as at least 98% or such as at least 99% sequence identity or such as 100% sequence identity.

13. The vaccine according to any of claims 9 to 12, wherein the antigenic unit comprises multiple copies of the RBD or parts thereof which copies are identical or differ in their amino acid sequences.

14. The vaccine according to any of claims 2 to 13, wherein the at least one betacoronavirus epitope is a B cell epitope comprised in the viral surface protein or a part thereof.

15. The vaccine according to any of claims 2 to 14, wherein the antigenic unit comprises multiple B cell epitopes comprised in the viral surface protein or a part thereof.

16. The vaccine according to claim 1, wherein the at least one betacoronavirus epitope is a T cell epitope.

17. The vaccine to claim 16, wherein the antigenic unit comprises multiple T cell epitopes.

18. The vaccine according to any of claims 16 to 17, wherein the T cell epitope is conserved between different genus and/or species and/or strains of betacoronaviruses, preferably between SARS-Cov2 and SARS-CoV.

19. The vaccine according to any of claims 16 to 18, wherein the T cell epitope has a length of from 7 to about 200 amino acids, preferably of from 7 to 100 amino acids or the T cell epitope has a length suitable for presentation by HLA class I/II alleles, preferably a length of from 7 to 30 amino acids, more preferably a length of from 8 to 15 amino acids.

20. The vaccine according to any of claims 16 to 19, wherein the T cell epitope is known to be immunogenic or is selected based on the predicted ability to bind to HLA class I/II alleles.

21. The vaccine according to any of claims 16 to 20, wherein the T cell epitope is selected from epitopes having an amino acid sequence of any of SEQ ID NO: 1 to SEQ ID NO: 444.

22. The vaccine according to any of claims 16 to 20, wherein the T cell epitope is selected from the list consisting of RSFIEDLLFNKVTLA, MTYRRLISMMGFKMNYQVNGYPNMF, LMIERFVSLAIDAYP, RAMPNMLRIMASLVL, MVYMPASWVMRIMTW, FLNRFTTTLNDFNLVAM, SSVELKHFFFAQDGNAAI, HFAIGLALYYPSARIVYTACSHAAV, YFIKGLNNLNRGMVL, YLNTLTLAVPYNMRV, AQFAPSASAFFGMSRI, EIVDTVSALVYDNKL, SSGDATTAYANSVFNICQAVTANVNALL, HVISTSHKLVLSVNPYV, MLSDTLKNLSDRVVFVLWAHGFEL, TANPKTPKYKFVRIQPGQTF, ASIKNFKSVLYYQNNVFM, FVNEFYAYLRKHFSMM, RVWTLMNVLTLVYKV, FAYANRNRFLYIIKL and LVKPSFYVYSRVKNL.

23. The vaccine according to any of claims 16 to 20, wherein the T cell epitope is selected from the list consisting of RAMPNMLRIMASLVL, HVISTSHKLVLSVNPYV and LVKPSFYVYSRVKNL.

24. The vaccine according to any of claim 16 to 23, wherein the antigenic unit further comprises at least one betacoronavirus epitope which is a full-length viral surface protein of a betacoronavirus or a part thereof.

25. The vaccine according to claim 24, wherein the viral surface protein is selected from the group consisting of envelope protein, spike protein, membrane protein and hemagglutinin esterase.

26. The vaccine according to any of claims 24 to 25, wherein the at least one betacoronavirus epitope comprises or is the spike protein.

27. The vaccine according to any of claims 24 to 26, wherein the at least one betacoronavirus epitope comprises or is the full-length spike protein.

28. The vaccine according to claim 27, wherein the at least one betacoronavirus epitope comprises or consists of an amino acid sequence having at least 70% sequence identity to the amino acid sequence 243 to 1437 of SEQ ID NO: 275, such as at least 75%, such as at least 77%, such as at least 80%, such as at least 85%, such as at least 90%, such as at least 91%, such as at least 92%, such as at least 93%, such as at least 94%, such as at least 95%, such as at least 96%, such as at least 97%, such as at least 98% or such as at least 99% sequence identity or such as 100% sequence identity.

29. The vaccine according to any of claims 24 to 26, wherein the at least one betacoronavirus epitope comprises or is a part of the spike protein.

30. The vaccine according to claim 29, wherein the at least one betacoronavirus epitope comprises or is the RBD or a part thereof.

31. The vaccine according to claim 30, wherein the at least one betacoronavirus epitope comprises or consists of an amino acid sequence having at least 70% sequence identity to the amino acid sequence of SEQ ID NO: 231 or SEQ ID NO: 802, or SEQ ID NO: 803 or SEQ ID NO: 804 or SEQ ID NO: 805, such as at least 75%, such as at least 77%, such as at least 80%, such as at least 85%, such as at least 90%, such as at least 91%, such as at least 92%, such as at least 93%, such as at least 94%, such as at least 95%, such as at least 96%, such as at least 97%, such as at least 98% or such as at least 99% sequence identity or such as 100% sequence identity.

32. The vaccine according to claim 30, wherein the at least one betacoronavirus epitope comprises or consists of an amino acid sequence having at least 70% sequence identity to the amino acid sequence 243 to 465 of SEQ ID NO: 255, such as at least 75%, such as at least 77%, such as at least 80%, such as at least 85%, such as at least 90%, such as at least 91%, such as at least 92%, such as at least 93%, such as at least 94%, such as at least 95%, such as at least 96%, such as at least 97%, such as at least 98% or such as at least 99% sequence identity or such as 100% sequence identity.

33. The vaccine according to claim 30, wherein the at least one betacoronavirus epitope comprises or consists of an amino acid sequence having at least 70% sequence identity to the amino acid sequence of SEQ ID NO: 246, such as at least 75%, such as at least 77%, such as at least 80%, such as at least 85%, such as at least 90%, such as at least 91%, such as at least 92%, such as at least 93%, such as at least 94%, such as at least 95%, such as at least 96%, such as at least 97%, such as at least 98% or such as at least 99% sequence identity or such as 100% sequence identity.

34. The vaccine according to any of claims 30 to 33, wherein the antigenic unit comprises multiple copies of the RBD or parts thereof which copies are identical or differ in their amino acid sequences.

35. The vaccine according to any of claims 24 to 34, wherein the at least one betacoronavirus epitope is a B cell epitope comprised in the viral surface protein or a part thereof.

36. The vaccine according to any of claims 24 to 35, wherein the antigenic unit comprises multiple B cell epitopes comprised in the viral surface protein or a part thereof.

37. The vaccine according to claim 24, wherein the antigenic unit comprises a T cell epitope selected from the list consisting of RSFIEDLLFNKVTLA, MTYRRLISMMGFKMNYQVNGYPNMF, LMIERFVSLAIDAYP, RAMPNMLRIMASLVL, MVYMPASWVMRIMTW, FLNRFTTTLNDFNLVAM, SSVELKHFFFAQDGNAAI, HFAIGLALYYPSARIVYTACSHAAV, YFIKGLNNLNRGMVL, YLNTLTLAVPYNMRV, AQFAPSASAFFGMSRI, EIVDTVSALVYDNKL, SSGDATTAYANSVFNICQAVTANVNALL, HVISTSHKLVLSVNPYV, MLSDTLKNLSDRVVFVLWAHGFEL, TANPKTPKYKFVRIQPGQTF, ASIKNFKSVLYYQNNVFM, FVNEFYAYLRKHFSMM, RVWTLMNVLTLVYKV, FAYANRNRFLYIIKL and LVKPSFYVYSRVKN and wherein the antigenic unit further comprises an amino acid sequence having at least 70% sequence identity to the amino acid sequence of SEQ ID NO: 231 or SEQ ID NO: 802, or SEQ ID NO: 803 or SEQ ID NO: 804 or SEQ ID NO: 805, such as at least 75%, such as at least 77%, such as at least 80%, such as at least 85%, such as at least 90%, such as at least 91%, such as at least 92%, such as at least 93%, such as at least 94%, such as at least 95%, such as at least 96%, such as at least 97%, such as at least 98% or such as at least 99% sequence identity or such as 100% sequence identity.

38. The vaccine according to claim 24, wherein the antigenic unit comprises one or more T cell epitopes selected from the list consisting of RAMPNMLRIMASLVL, HVISTSHKLVLSVNPYV and LVKPSFYVYSRVKNL and wherein the antigenic unit further comprises an amino acid sequence having at least 70% sequence identity to the amino acid sequence 243 to 465 of SEQ ID NO: 255, such as at least 75%, such as at least 77%, such as at least 80%, such as at least 85%, such as at least 90%, such as at least 91%, such as at least 92%, such as at least 93%, such as at least 94%, such as at least 95%, such as at least 96%, such as at least 97%, such as at least 98% or such as at least 99% sequence identity or such as 100% sequence identity.

39. The vaccine according to any of the preceding claims, wherein the antigenic unit comprises up to 3500 amino acids, such as from 21 to 3500 amino acids, preferably from about 30 amino acids to about 2000 amino acids such as from about 50 to about 1500 amino acids, more preferably from about 100 to about 1500 amino acids, such as from about 100 to about 1000 amino acids or from about 100 to about 500 amino acids or from about 100 to about 300 amino acids.

40. The vaccine according to any of the preceding claims, wherein the antigenic unit comprises one or more linkers, preferably one or more non-immunogenic and/or flexible linkers.

41. The vaccine according to any of the preceding claims, wherein the antigenic unit comprises 10, 20, 30, 40 or 50 epitopes, preferably T cell epitopes.

42. The vaccine according to any of the preceding claims, wherein said targeting unit comprises antibody binding regions with specificity for surface molecules or receptors on antigen presenting cells (APCs), preferably specificity for CD14, CD40, Toll-like receptor, CCR1, CCR3, CCR5, MHC class I proteins or MHC class II proteins.

43. The vaccine according to any of the preceding claims, wherein the targeting unit has affinity for a chemokine receptor selected from CCR1, CCR3 and CCR5.

44. The vaccine according to any of claims 42 to 43, wherein said targeting unit has affinity for MHC class II proteins, preferably MHC class II proteins selected from the group consisting of anti-HLA-DP, anti-HLA-DR and anti-pan HLA class II.

45. The vaccine according to any of the preceding claims, wherein the targeting unit is selected from anti-pan HLA class II and MIP-1α and preferably selected from anti-pan HLA class II and human MIP-1α.

46. The vaccine according to claim 45, wherein the targeting unit is MIP-1α, preferably human MIP-1α.

47. The vaccine according to claim 45, wherein the targeting unit is anti-pan HLA class II.

48. The vaccine according to any of the preceding claims, wherein the dimerization unit comprises a hinge region.

49. The vaccine according to any of claim 48, wherein the dimerization unit further comprises another domain that facilitates dimerization.

50. The vaccine according to claim 49, wherein the other domain is an immunoglobulin domain, preferably an immunoglobulin constant domain.

51. The vaccine according to any of claims 48 to 49, wherein the dimerization unit further comprises a dimerization unit linker, which connects the hinge region and the other domain that facilitates dimerization.

52. The vaccine according to any of the preceding claims, wherein the vaccine comprises the polynucleotide.

53. The vaccine according to claim 52, wherein the polynucleotide further comprises a nucleotide sequence encoding a signal peptide.

54. The vaccine according to any of the preceding claims, wherein the vaccine comprises the polypeptide or the dimeric protein and said targeting unit, dimerization unit and antigenic unit in said peptide or dimeric protein are in the N-terminal to C-terminal order of targeting unit, dimerization unit and antigenic unit.

55. The vaccine according to any of the preceding claims, wherein said betacoronavirus is one selected from the group consisting of SARS-CoV, MERS-CoV, SARS-CoV-2, HCoV-OC43 and HCoV-HKU1, preferably selected from the group consisting of SARS-CoV and SARS-CoV.

56. A polynucleotide as defined in any of the claims 1 to 53.

57. A vector comprising the polynucleotide according to claim 56.

58. A host cell comprising the polynucleotide as defined in any of the claims 1 to 53 or comprising the vector according to claim 57.

59. A polypeptide encoded by the polynucleotide as defined in any of claims 1 to 53.

60. A dimeric protein consisting of two polypeptides as defined in claim 59.

61. The dimeric protein according to claim 60, wherein the dimeric protein is a homodimeric protein.

62. The polynucleotide according to claim 56 or the polypeptide according to claim 59 or the dimeric protein according to any of claim 60 or 61 for use as a medicament.

63. A method of preparing the vaccine according to any one of the preceding claims 1 to 51 and 54 to 55, wherein the vaccine comprises the polypeptide or the dimeric protein and wherein said method comprises:
a) transfecting cells with the polynucleotide as defined in any of the claims 1 to 53;
b) culturing the cells;
c) collecting and purifying the dimeric protein or the polypeptide expressed from the cells; and
d) mixing the dimeric protein or polypeptide obtained from step c) with the pharmaceutically acceptable carrier.

64. A method for preparing the vaccine according to any one of the preceding claims 1 to 53 and 55, wherein the vaccine comprises the polynucleotide and wherein the method comprises:
a) preparing the polynucleotide;
b) optionally cloning the polynucleotide into an expression vector; and
c) mixing the polynucleotide obtained from step a) or the vector obtained from step b) with the pharmaceutically acceptable carrier.

65. A method for treating a subject having suffering from a betacoronavirus infection or being in need of prevention thereof, the method comprising administering to the subject the vaccine as defined in any of claims 1 to 55.

66. A vaccine as defined in any of claims 1 to 55 for use in the treatment of an infection with a betacoronavirus or for use in the prevention of a betacoronavirus infection.
